(19) United States
(12) Patent Application Publication
(10) Pub. No.: US 2026/0143965 A1
(43) Pub. Date: May 21, 2026

(54) LIGHT EMITTING ELEMENT, AMINE COMPOUND FOR THE LIGHT EMITTING ELEMENT, AND DISPLAY DEVICE INCLUDING THE LIGHT EMITTING ELEMENT

(71) Applicant: Samsung Display Co., Ltd., Yongin-si, Gyeonggi-do (KR)

(72) Inventor: Takao SAKUMA, Yokohama-shi (JP)

(21) Appl. No.: 19/355,817

(22) Filed: Oct. 10, 2025

(30) Foreign Application Priority Data
Nov. 20, 2024 (JP) 2024-202422

(51) Int. Cl. H10K 85/60; C07D 333/76; C07D 409/12; C07D 409/14; H10K 50/15

(52) U.S. Cl. CPC H10K 85/636; C07D 333/76; C07D 409/12; C07D 409/14; H10K 85/633; H10K 50/15; H10K 85/615; H10K 85/6572; H10K 85/6574; H10K 85/6576

(57) ABSTRACT

A light emitting element and an amine compound for the light emitting element are provided. The light emitting element of one or more embodiments includes a first electrode, a second electrode on the first electrode, and at least one functional layer between the first electrode and the second electrode and including an amine compound represented by Formula 1.

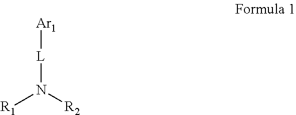

### CROSS-REFERENCE TO RELATED APPLICATION

This application claims priority to an the benefit of Japanese Patent Application No. 2024-202422, filed on Nov. 20, 2024, in the Japanese Intellectual Property Office, the entire content of which is hereby incorporated by reference.

### BACKGROUND

One or more embodiments of the present disclosure relate to a light emitting element, an amine compound used in the light emitting element, and a display device including the light emitting element, and for example, to a light emitting element including a novel amine compound in a functional layer, a display device including the light emitting element, and/or an electronic apparatus including the display device.

In recent years, organic electroluminescence (EL) display devices and similar technologies have been actively developed as image display devices. These organic EL display devices are self-luminous and include light emitting elements that emit light without requiring a backlight. In such devices, holes and electrons are injected from a first electrode and a second electrode, respectively, and recombine in an emission layer to form excitons. These excitons cause a luminescent material in the emission layer to emit light, thereby enabling image display.

For light emitting elements to be effectively applied in display devices, it is desirable for them to exhibit high luminous efficiency and long operational lifespan. Accordingly, there is ongoing development of materials for light emitting elements that may stably achieve these characteristics. In particular, materials utilized in hole transport regions are being developed to provide enhanced hole transport properties and stability, which are important for achieving light emitting elements with extended lifespans.

### SUMMARY

One or more aspects of embodiments of the present disclosure are directed toward a light emitting element exhibiting long lifespan, a display device including the light emitting element, and/or an electronic apparatus including the display device.

One or more aspects of embodiments of the present disclosure also provide an amine compound as a material for a light emitting element exhibiting long lifespan.

Additional aspects will be set forth in part in the description which follows and, in part, will be apparent from the description, or may be learned by practice of the presented embodiments of the disclosure.

One or more embodiments of the disclosure provide a light emitting element including a first electrode, a second electrode arranged on the first electrode, and at least one functional layer arranged between the first electrode and the second electrode and including an amine compound represented by Formula 1.

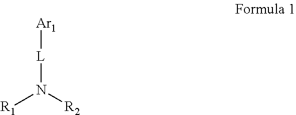

In Formula 1, L may be a direct linkage, a substituted or unsubstituted arylene group having 6 to 30 ring-forming carbon atoms, or a substituted or unsubstituted heteroarylene group having 2 to 30 ring-forming carbon atoms, Ar₁ may be a substituted or unsubstituted aryl group having 6 to 30 ring-forming carbon atoms, or a substituted or unsubstituted heteroaryl group having 2 to 30 ring-forming carbon atoms, where Ar₁ does not include a substituted or unsubstituted benzofuran group and a substituted or unsubstituted benzothiophene group, R₁ may be represented by Formula 2, and R₂ may be represented by Formula 3,

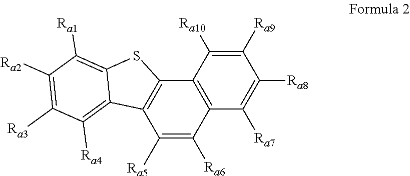

in Formula 2, any one of (e.g., selected from among) Rₐ₁ to Rₐ₄ may be a position connected to Formula 1, and the remainder of (e.g., any remaining) Rₐ₁ to Rₐ₄ may each independently be a hydrogen atom, a deuterium atom, a halogen atom, a cyano group, or a substituted or unsubstituted alkyl group having 1 to 10 carbon atoms, and Rₐ₅ to Rₐ₁₀ may each independently be a hydrogen atom, a deuterium atom, a halogen atom, a cyano group, or a substituted or unsubstituted alkyl group having 1 to 10 carbon atoms, and

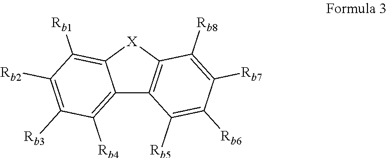

in Formula 3, X may be O, S, or NAr₂, Ar₂ may be a substituted or unsubstituted aryl group having 6 to 30 ring-forming carbon atoms, any one of (e.g., selected from among) R_{b1} to R_{b4} is a position connected to Formula 1, and the remainder of (e.g., any remaining) R_{b1} to R_{b4} is each independently a hydrogen atom, a deuterium atom, a halogen atom, or a substituted or unsubstituted alkyl group having 1 to 10 carbon atoms, any one of (e.g., selected from among) R_{b5} to R_{b8} may be a substituted or unsubstituted aryl group having 6 to 30 ring-forming carbon atoms, where R_{b5} to R_{b8} do not include a substituted or unsubstituted fluorene group, and the remainder of (e.g., any remaining) R_{b5} to R_{b8} may each independently be a hydrogen atom, a deuterium atom, a halogen atom, or a substituted or unsubstituted alkyl group having 1 to 10 carbon atoms.

In one or more embodiments, the at least one functional layer may include an emission layer, a hole transport region arranged between the first electrode and the emission layer, and an electron transport region arranged between the emission layer and the second electrode, and the hole transport region may include the amine compound represented by Formula 1.

In one or more embodiments, the hole transport region may include a hole injection layer arranged on the first electrode, and a hole transport layer arranged on the hole injection layer, and the hole transport layer may include the amine compound represented by Formula 1.

In one or more embodiments, the amine compound represented by Formula 1 may be a monoamine compound.

In one or more embodiments, in Formula 1, L may be a direct linkage, or a substituted or unsubstituted phenylene group.

In one or more embodiments, in Formula 1, Ar₁ may be a substituted or unsubstituted naphthyl group, a substituted or unsubstituted phenanthrene group, a substituted or unsubstituted dibenzofuran group, a substituted or unsubstituted dibenzothiophene group, or a substituted or unsubstituted carbazole group.

In one or more embodiments, the amine compound represented by Formula 1 may be represented by Formula 1-1.

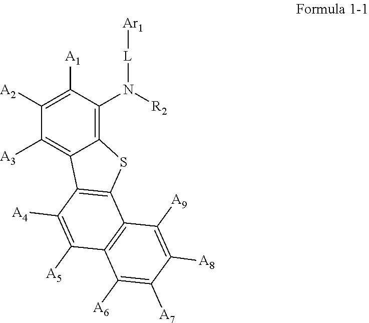

In Formula 1-1, A₁ to A₉ may each independently be a hydrogen atom, a deuterium atom, a halogen atom, or a substituted or unsubstituted alkyl group having 1 to 10 carbon atoms, and L, Ar₁, and R₂ may be the same as defined in Formula 1.

In one or more embodiments, the amine compound represented by Formula 1 may be represented by any one of (e.g., selected from among) Formulas 1-2 to 1-5.

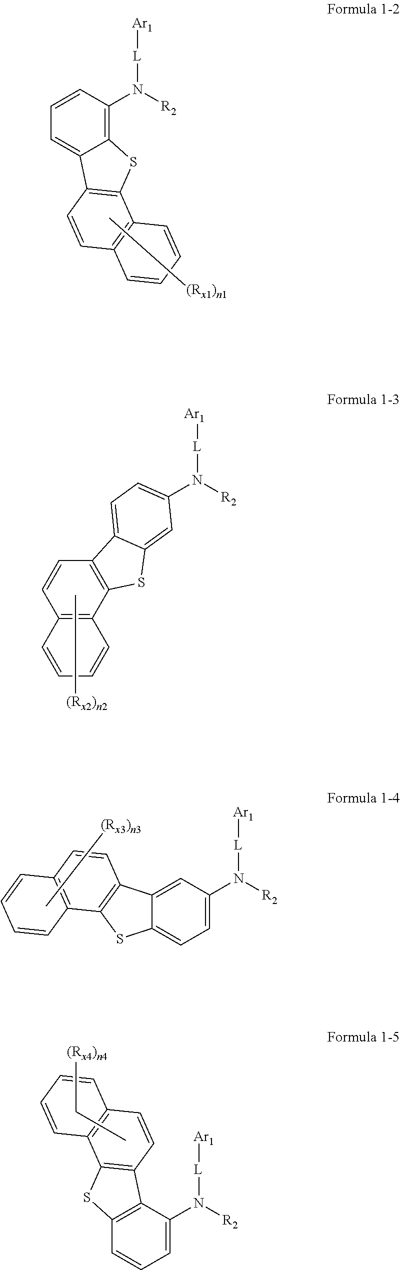

In Formulas 1-2 to 1-5, Rₓ₁ to Rₓ₄ may each independently be a hydrogen atom, a deuterium atom, or a substituted or unsubstituted alkyl group having 1 to 5 carbon atoms, n1 to n4 may each independently be an integer of 0 to 6, and L, Ar₁, and R₂ may be the same as defined in Formula 1.

In one or more embodiments, in Formula 3, any one of (e.g., selected from among) R_{b1} to R_{b4} may be a position connected to Formula 1, and the remainder of (e.g., any remaining) R_{b1} to R_{b4} may each independently be a hydrogen atom or a deuterium atom.

In one or more embodiments, the amine compound represented by Formula 1 may be represented by any one of (e.g., selected from among) Formulas 1-6 to 1-9.

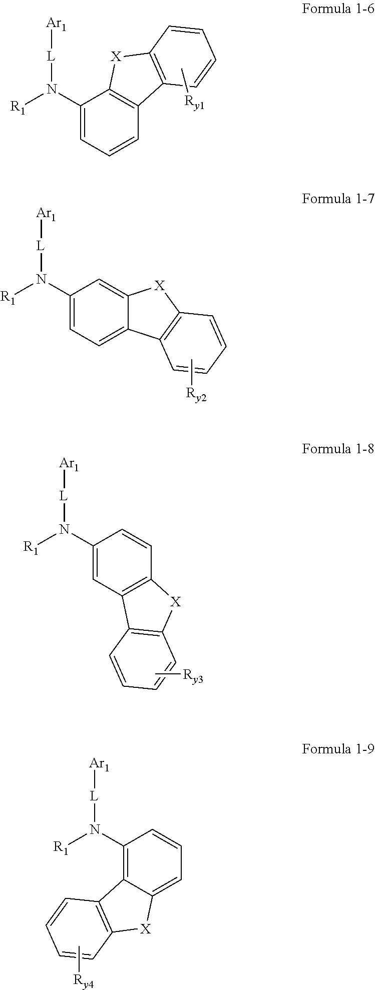

In Formulas 1-6 and 1-7, R_{y1} to R_{y4} may each independently be a substituted or unsubstituted aryl group having 6 to 12 ring-forming carbon atoms, and L, Ar₁, and R₁ may be as defined in Formula 1.

In one or more embodiments, the substituent represented by Formula 3 may be represented by any one of (e.g., selected from among) Formulas 3-1 to 3-3.

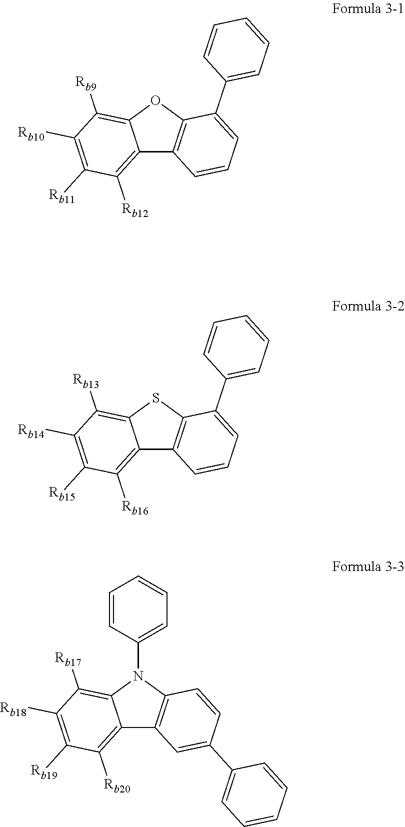

In Formula 3-1, any one of (e.g., selected from among) R_{b9} to R_{b12} may be a position connected to Formula 1, and the remainder of (e.g., any remaining) R_{b9} to R_{b12} may each independently be a hydrogen atom or a deuterium atom, in Formula 3-2, any one of (e.g., selected from among) R_{b13} to R_{b16} may be a position connected to Formula 1, and the remainder of (e.g., any remaining) R_{b13} to R_{b16} may each independently be a hydrogen atom or a deuterium atom, and in Formula 3-3, any one of (e.g., selected from among) R_{b17} to R_{b20} may be a position connected to Formula 1, and the remainder of (e.g., any remaining) R_{b17} to R_{b20} may each independently be a hydrogen atom or a deuterium atom.

In one or more embodiments, the amine compound represented by Formula 1 may be represented by any one of (e.g., selected from among) compounds from Compound Group 1.

In one or more embodiments of the disclosure, an amine compound is represented by Formula 1.

In one or more embodiments of the disclosure, a display device includes a base layer, a circuit layer arranged on the base layer, and a display element layer arranged on the circuit layer and including a light emitting element, wherein the light emitting element includes a first electrode, a second electrode arranged on the first electrode, and an amine compound arranged between the first electrode and the second electrode and represented by Formula 1.

In one or more embodiment, an electronic apparatus includes the display device.

For example, the amine compound represented by Formula 1 should exhibit structural and electronic characteristics that make it suitable or desire for utilization in functional layers of light emitting elements, particularly in regions such as the hole transport layer and/or the emission layer. These compounds may facilitate efficient charge transport and contribute to the thermal and morphological stability of the device, which are for achieving high performance and extended operational lifespans. Representative examples of such compounds are provided in Compound Group 1, illustrating the diversity of molecular configurations that may be employed. When incorporated into display devices or electronic apparatuses, these compounds serve as active materials that enhance the overall efficiency and reliability of the light emitting elements.

### BRIEF DESCRIPTION OF DRAWINGS

The accompanying drawings are included to provide a further understanding of the preceding and other aspects, features, and advantages of certain embodiments of the disclosure, and are incorporated in and constitute a part of this specification. The drawings illustrate embodiments of the disclosure and, together with the description, serve to explain principles of the disclosure. In the drawings:

FIG. 1 is a plan view showing a display device according to one or more embodiments;

FIG. 2 is a cross-sectional view of a display device according to one or more embodiments;

FIG. 3 is a cross-sectional view schematically showing a light emitting element according to one or more embodiments;

FIG. 4 is a cross-sectional view schematically showing a light emitting element according to one or more embodiments;

FIG. 5 is a cross-sectional view schematically showing a light emitting element according to one or more embodiments;

FIG. 6 is a cross-sectional view schematically showing a light emitting element according to one or more embodiments;

FIG. 7 is a cross-sectional view of a display device according to one or more embodiments;

FIG. 8 is a cross-sectional view of a display device according to one or more embodiments;

FIG. 9 is a cross-sectional view showing a display device according to one or more embodiments;

FIG. 10 is a cross-sectional view showing a display device according to one or more embodiments; and

FIG. 11 is a view showing a vehicle in which a display device according to one or more embodiments of the disclosure is arranged.

### DETAILED DESCRIPTION

The disclosure may be modified in one or more suitable manners and have many forms, and thus specific embodiments will be exemplified in the drawings and described in more detail in the detailed description of the invention. It should be understood, however, that it is not intended to limit the disclosure to the particular forms disclosed, but rather, is intended to cover all modifications, equivalents, and alternatives falling within the spirit and scope of the disclosure.

Expressions such as “at least one of,” “one of,” “selected from,” and “selected from among,” when preceding a list of elements, modify the entire list of elements and do not modify the individual elements of the list. For example, throughout the disclosure, the expression “at least one of a, b or c” indicates only a, only b, only c, both (e.g., simultaneously) a and b, both (e.g., simultaneously) a and c, both (e.g., simultaneously) b and c, all of a, b, and c, or variations thereof.

When explaining each of drawings, like reference numbers are used for referring to like elements. In the accompanying drawings, the dimensions of each structure are exaggeratingly illustrated for clarity of the present disclosure. It will be understood that, although the terms “first,” “second,” and/or the like, may be used herein to describe one or more suitable components, these components should not be limited by these terms. These terms are only used to distinguish one component from another. For example, a first component could be termed a second component, and, similarly, a second component could be termed a first component, without departing from the scope of example embodiments of the disclosure. As used herein, the singular forms, “a,” “an,” and “the” are intended to include the plural forms as well, unless the context clearly indicates otherwise.

As used herein, the term “and/or” includes any and all combinations of one or more of the associated listed elements.

In the present application, it will be understood that the terms “including,” “include,” includes,” “has,” “have,” “having”, “comprise,” “comprises,” “comprising, and/or the like specify the presence of features, numbers, steps, operations, component, parts, or combinations thereof disclosed in the specification, but do not exclude the possibility of presence or addition of one or more other features, numbers, steps, operations, component, parts, or combinations thereof. Additionally, the terms “comprise(s)/comprising,” “include(s)/including,” “have/has/having”, or other similar terms include or support the terms “consisting of” and “consisting essentially of,” indicating the presence of stated features, integers, steps, operations, elements, and/or components, without or essentially without the presence of other features, integers, steps, operations, elements, components, and/or groups thereof.

In the present application, if (e.g., when) a layer, a film, a region, or a plate is referred to as being “on” or “in an upper portion of” another layer, film, region, or plate, it may be not only “directly on” the layer, film, region, or plate, but intervening layers, films, regions, or plates may also be present. On the contrary to this, if (e.g., when) a layer, a film, a region, or a plate is referred to as being “below”, “in a lower portion of” another layer, film, region, or plate, it can be not only directly under the layer, film, region, or plate, but intervening layers, films, regions, or plates may also be present. In some embodiments, it will be understood that if (e.g., when) a part is referred to as being “on” another part, it can be arranged above the other part, or arranged under the other part as well.

In some embodiments, the terms “below”, “under”, “on the lower side”, “above”, “over”, “on the upper side”, and/or the like may be used to describe the relationships between the elements illustrated in the drawings. These terms are relative concepts and are described on the basis of the directions indicated in the drawings. In this specification, “arranged on” may refer to being arranged not only on an upper part of one member but also being arranged on a lower part thereof. For example, if the device in the drawings is turned over, elements described as “below” or “beneath” other elements or features would then be oriented “above” or “over” the other elements or features. Thus, the term “below” may encompass both (e.g., simultaneously) an orientation of above and below. The device may be otherwise oriented (rotated 90 degrees or at other orientations), and the spatially relative descriptors used herein should be interpreted accordingly.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

In this context, “consisting essentially of” indicates that any additional components will not materially affect the chemical, physical, optical or electrical properties of the semiconductor film.

As used in this specification, the phrase “on a plane” or “in plan view” refers to a top-down view of a target portion, as if viewed from directly above. The phrase “on a cross-section” or “in cross-sectional view” refers to a side view of a target portion, as seen along a vertical plane that cuts through the structure.

In the specification, the term “substituted or unsubstituted” may refer to substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In some embodiments, each of the substituents exemplified herein may be substituted or unsubstituted. For example, a biphenyl group may be interpreted as an aryl group or a phenyl group substituted with a phenyl group.

In the specification, the phrase “bonded to an adjacent group to form a ring” may refer to that a group is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In some embodiments, the rings formed by being bonded to each other may be connected to another ring to form a spiro structure.

In the specification, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In some embodiments, two methyl groups in 4,5-dimethylphenanthrene may be interpreted as “adjacent groups” to each other.

In the specification, examples of the halogen atom may include a fluorine atom, a chlorine atom, a bromine atom, or an iodine atom.

In the specification, the alkyl group may be linear or branched. The number of carbons in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but the embodiment of the disclosure is not limited thereto.

In the specification, a cycloalkyl group may refer to a cyclic alkyl group. The number of carbons in the cycloalkyl group is 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and/or the like, but the embodiment of the disclosure is not limited thereto.

In the specification, an alkenyl group refers to a hydrocarbon group including at least one carbon double bond in the middle or terminal of an alkyl group having 2 or more carbon atoms. The alkenyl group may be linear or branched. The number of carbon atoms in the alkenyl group is not specifically limited, but is 2 to 30, 2 to 20, or 2 to 10. Examples of the alkenyl group include a vinyl group, a 1-butenyl group, a 1-pentenyl group, a 1,3-butadienyl aryl group, a styrenyl group, a styryl vinyl group, and/or the like, but the embodiment of the disclosure is not limited thereto.

In the specification, an alkynyl group refers to a hydrocarbon group including at least one carbon triple bond in the middle or terminal of an alkyl group having 2 or more carbon atoms. The alkynyl group may be linear or branched. Although the number of carbon atoms is not specifically limited, it is 2 to 30, 2 to 20, or 2 to 10. Specific examples of the alkynyl group may include an ethynyl group, a propynyl group, and/or the like, but are not limited thereto.

In the specification, the hydrocarbon ring group refers to any functional group or substituent derived from an aliphatic hydrocarbon ring. The hydrocarbon ring group may be a saturated hydrocarbon ring group having 5 to 20 ring-forming carbon atoms.

In the specification, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, and/or the like, but the embodiment of the disclosure is not limited thereto.

In the specification, the fluorenyl group may be substituted, and two substituents may be bonded to each other to form a spiro structure. Examples of the substituted fluorenyl group are as follows. However, the embodiment of the disclosure is not limited thereto.

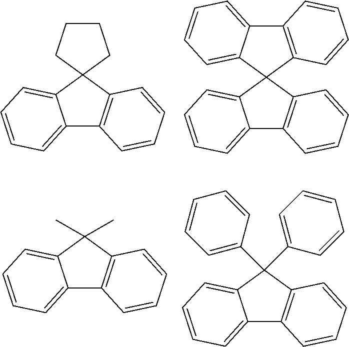

The heterocyclic group herein refers to any functional group or substituent derived from a ring containing at least one of B, O, N, P, Si, or Se as a heteroatom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

In the specification, the heterocyclic group may contain at least one of B, O, N, P, Si or S as a heteroatom. If the heterocyclic group contains two or more heteroatoms, the two or more heteroatoms may be the same as or different from each other. The heterocyclic group may be a monocyclic heterocyclic group or a polycyclic heterocyclic group, and includes a heteroaryl group. The number of ring-forming carbon atoms in the heterocyclic group may be 2 to 30, 2 to 20, or 2 to 10.

In the specification, the aliphatic heterocyclic group may include at least one of B, O, N, P, Si, or S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, and/or the like, but the embodiment of the disclosure is not limited thereto.

In the specification, the heteroaryl group may contain at least one of B, O, N, P, Si, or S as a heteroatom. If the heteroaryl group contains two or more heteroatoms, the two or more heteroatoms may be the same as or different from each other. The heteroaryl group may be a monocyclic heterocyclic group or a polycyclic heterocyclic group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, and/or the like, but the embodiment of the disclosure is not limited thereto.

In the specification, the preceding description of the aryl group may be applied to an arylene group except that the arylene group is a divalent group. The preceding description of the heteroaryl group may be applied to a heteroarylene group except that the heteroarylene group is a divalent group.

In the specification, the silyl group includes an alkylsilyl group and an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and/or the like, but the embodiment of the disclosure is not limited thereto.

In the specification, the number of ring-forming carbon atoms in the carbonyl group is not specifically limited, but may be 1 to 40, 1 to 30, or 1 to 20. For example, the carbonyl group may have the following structures, but the embodiment of the disclosure is not limited thereto.

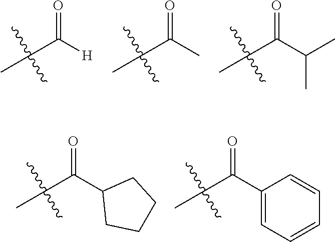

In the specification, the number of carbon atoms in the sulfinyl group and the sulfonyl group is not particularly limited, but may be 1 to 30. The sulfinyl group may include an alkyl sulfinyl group and an aryl sulfinyl group. The sulfonyl group may include an alkyl sulfonyl group and an aryl sulfonyl group.

In the specification, the thio group may include an alkylthio group and an arylthio group. The thio group may refer to that a sulfur atom is bonded to the alkyl group or the aryl group as defined herein. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but the embodiment of the disclosure is not limited thereto.

In the specification, an oxy group may refer to that an oxygen atom is bonded to the alkyl group or the aryl group as defined herein. The oxy group may include an alkoxy group and an aryl oxy group. The alkoxy group may be a linear chain, a branched chain or a ring chain. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and/or the like, but the embodiment of the disclosure is not limited thereto.

The boron group herein may refer to that a boron atom is bonded to the alkyl group or the aryl group as defined herein. The boron group includes an alkyl boron group and an aryl boron group. Examples of the boron group may include a dimethylboron group, a trimethylboron group, a t-butyldimethylboron group, a diphenylboron group, a phenylboron group, and/or the like, but the embodiment of the disclosure is not limited thereto.

In the specification, the alkenyl group may be linear or branched. The number of carbon atoms in the alkenyl group is not specifically limited, but is 2 to 30, 2 to 20, or 2 to 10. Examples of the alkenyl group include a vinyl group, a 1-butenyl group, a 1-pentenyl group, a 1,3-butadienyl aryl group, a styrenyl group, a styryl vinyl group, and/or the like, but the embodiment of the disclosure is not limited thereto.

In the specification, the number of carbon atoms in an amine group is not specifically limited, but may be 1 to 30. The amine group may include an alkyl amine group and an aryl amine group. Examples of the amine group may include a methylamine group, a dimethylamine group, a phenylamine group, a diphenylamine group, a naphthylamine group, a 9-methyl-anthracenylamine group, a triphenylamine group, and/or the like, but the embodiment of the disclosure is not limited thereto.

In the specification, the alkyl group among an alkylthio group, an alkylsulfoxy group, an alkylaryl group, an alkylamino group, an alkyl boron group, an alkyl silyl group, and an alkyl amine group is the same as the examples of the alkyl group described herein.

In the specification, the aryl group among an aryloxy group, an arylthio group, an arylsulfoxy group, an arylamino group, an arylboron group, an arylsilyl group, an arylamine group is the same as the examples of the aryl group described herein.

In the specification, a direct linkage may refer to a single bond.

In the specification,

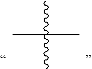

and “-*” refer to a position to be connected.

Hereinafter, embodiments of the disclosure will be described with reference to the accompanying drawings.

### Display Apparatus (Display Device)

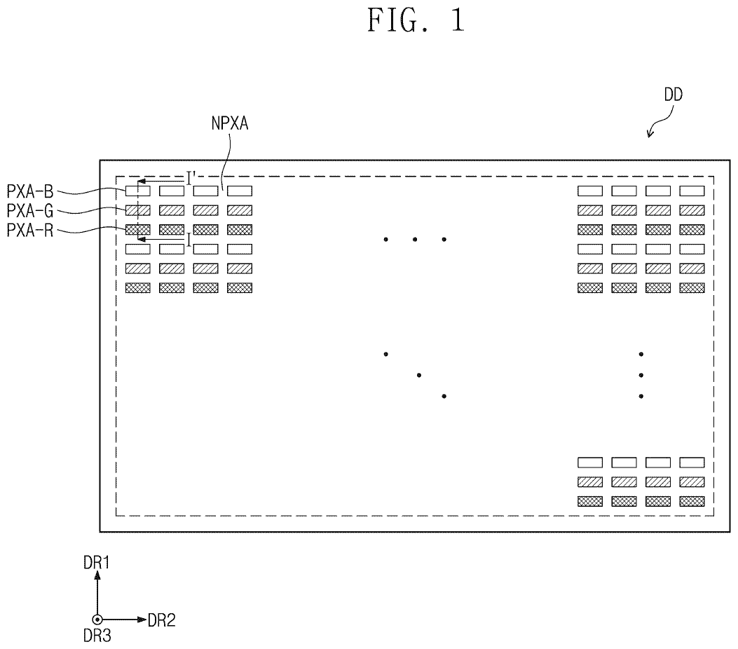

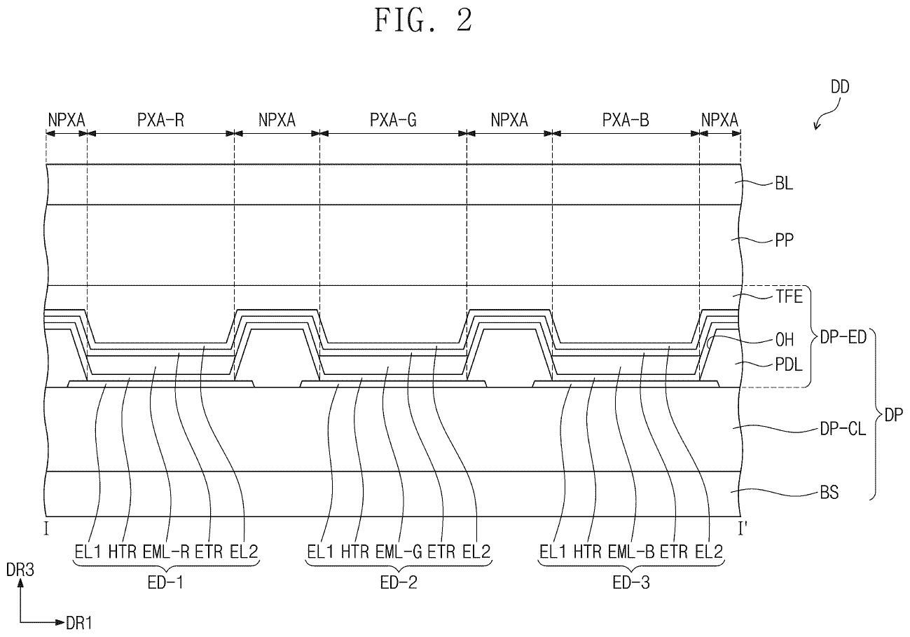

FIG. 1 is a plan view illustrating one or more embodiments of a display apparatus (display device) DD. FIG. 2 is a cross-sectional view of the display apparatus DD of the embodiment. FIG. 2 is a cross-sectional view illustrating a part taken along the line I-I′ of FIG. 1.

The display apparatus DD may include a display panel DP and an optical layer PP arranged on the display panel DP. The display panel DP includes light emitting devices ED-1, ED-2, and ED-3. The display apparatus DD may include a plurality of light emitting devices ED-1, ED-2, and ED-3. The optical layer PP may be arranged on the display panel DP to control reflected light in the display panel DP due to external light. The optical layer PP may include, for example, a polarization layer or a color filter layer. Unlike the configuration illustrated in the drawing, the optical layer PP may not be provided from the display apparatus DD of one or more embodiments.

A base substrate BL may be arranged on the optical layer PP. The base substrate BL may be a member which provides a base surface on which the optical layer PP arranged. The base substrate BL may be a glass substrate, a metal substrate, a plastic substrate, and/or the like. However, the embodiment of the disclosure is not limited thereto, and the base substrate BL may be an inorganic layer, an organic layer, or a composite material layer. In some embodiments, unlike the configuration illustrated, in one or more embodiments, the base substrate BL may not be provided.

The display apparatus DD according to one or more embodiments may further include a filling layer. The filling layer may be arranged between a display device layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and the display device layer DP-ED. The display device layer DP-ED may include a pixel defining film PDL, the light emitting devices ED-1, ED-2, and ED-3 arranged between portions of the pixel defining film PDL, and an encapsulation layer TFE arranged on the light emitting devices ED-1, ED-2, and ED-3.

The base layer BS may be a member which provides a base surface on which the display device layer DP-ED is arranged. The base layer BS may be a glass substrate, a metal substrate, a plastic substrate, and/or the like. However, the embodiment is not limited thereto, and the base layer BS may be an inorganic layer, an organic layer, or a composite material layer.

In one or more embodiments, the circuit layer DP-CL is arranged on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light emitting devices ED-1, ED-2, and ED-3 of the display device layer DP-ED.

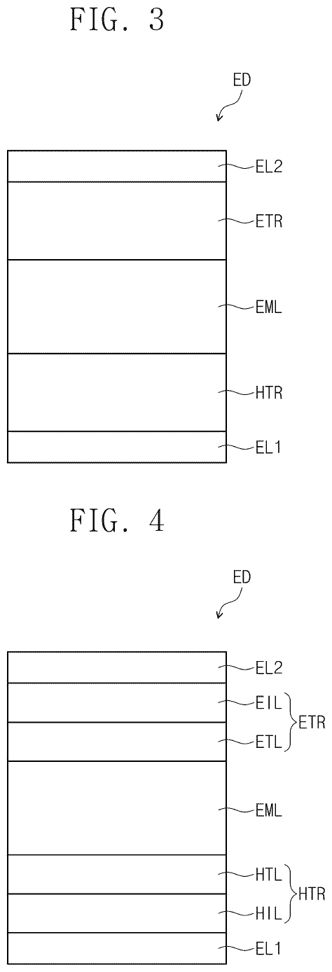

Each of the light emitting devices ED-1, ED-2, and ED-3 may have a structure of each light emitting device ED of embodiments according to FIGS. 3 to 6, which will be described later. Each of the light emitting devices ED-1, ED-2, and ED-3 may include a first electrode EL1, a hole transport region HTR, emission layers EML-R, EML-G, and EML-B, an electron transport region ETR, and a second electrode EL2.

FIG. 2 illustrates one or more embodiments in which the emission layers EML-R, EML-G, and EML-B of the light emitting devices ED-1, ED-2, and ED-3 are arranged in openings OH defined in the pixel defining film PDL, and the hole transport region HTR, the electron transport region ETR, and the second electrode EL2 are provided as a common layer in the entire light emitting devices ED-1, ED-2, and ED-3. However, the embodiment of the disclosure is not limited thereto, and unlike the configuration illustrated in FIG. 2, the hole transport region HTR and the electron transport region ETR in one or more embodiments may be provided by being patterned inside the openings OH defined in the pixel defining film PDL. For example, the hole transport region HTR, the emission layers EML-R, EML-G, and EML-B, and the electron transport region ETR of the light emitting devices ED-1, ED-2, and ED-3 in one or more embodiments may be provided by being patterned in an inkjet printing method.

The encapsulation layer TFE may cover the light emitting devices ED-1, ED-2 and ED-3. The encapsulation layer TFE may seal the display device layer DP-ED. The encapsulation layer TFE may be a thin film encapsulation layer. The encapsulation layer TFE may be formed by laminating one layer or a plurality of layers. The encapsulation layer TFE includes at least one insulation layer. The encapsulation layer TFE according to one or more embodiments may include at least one inorganic film (hereinafter, an encapsulation-inorganic film). The encapsulation layer TFE according to one or more embodiments may also include at least one organic film (hereinafter, an encapsulation-organic film) and at least one encapsulation-inorganic film.

The encapsulation-inorganic film protects the display device layer DP-ED from moisture/oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, and/or the like, but the embodiment of the disclosure is not particularly limited thereto. The encapsulation-organic film may include an acrylic-based compound, an epoxy-based compound, and/or the like. The encapsulation-organic film may include a photopolymerizable organic material, but the embodiment of the disclosure is not particularly limited thereto.

The encapsulation layer TFE may be arranged on the second electrode EL2 and may be arranged filling the opening OH.

Referring to FIGS. 1 and 2, the display apparatus DD may include a non-light emitting region NPXA and light emitting regions PXA-R, PXA-G, and PXA-B. The light emitting regions PXA-R, PXA-G, and PXA-B may be regions in which light generated by the respective light emitting devices ED-1, ED-2, and ED-3 is emitted. The light emitting regions PXA-R, PXA-G, and PXA-B may be spaced and/or apart from each other on a plane.

Each of the light emitting regions PXA-R, PXA-G, and PXA-B may be a region divided (e.g., defined) by the pixel defining film PDL. The non-light emitting areas NPXA may be areas between the adjacent light emitting areas PXA-R, PXA-G, and PXA-B, which correspond to the pixel defining film PDL. In the specification, the light emitting regions PXA-R, PXA-G, and PXA-B may respectively correspond to pixels. The pixel defining film PDL may divide the light emitting devices ED-1, ED-2, and ED-3. The emission layers EML-R, EML-G, and EML-B of the light emitting devices ED-1, ED-2, and ED-3 may be arranged in openings OH defined in the pixel defining film PDL and separated from each other.

The light emitting regions PXA-R, PXA-G, and PXA-B may be divided (e.g., defined) into a plurality of groups according to the color of light generated from the light emitting devices ED-1, ED-2, and ED-3. In the display apparatus DD of one or more embodiments illustrated in FIGS. 1 and 2, three light emitting regions PXA-R, PXA-G, and PXA-B, which emit red light, green light, and blue light, respectively, are exemplarily illustrated. For example, the display device DD of one or more embodiments may include the red light emitting region PXA-R, the green light emitting region PXA-G, and the blue light emitting region PXA-B that are separated from each other.

In the display apparatus DD according to one or more embodiments, the plurality of light emitting devices ED-1, ED-2 and ED-3 may be to emit light beams having wavelengths different from each other. For example, in one or more embodiments, the display apparatus DD may include a first light emitting device ED-1 that emits red light, a second light emitting device ED-2 that emits green light, and a third light emitting device ED-3 that emits blue light. For example, the red light emitting region PXA-R, the green light emitting region PXA-G, and the blue light emitting region PXA-B of the display apparatus DD may correspond to the first light emitting device ED-1, the second light emitting device ED-2, and the third light emitting device ED-3, respectively.

However, the embodiment of the disclosure is not limited thereto, and the first to third light emitting devices ED-1, ED-2, and ED-3 may be to emit light beams in substantially the same wavelength range or at least one light emitting device may be to emit a light beam in a wavelength range different from the others. For example, the first to third light emitting devices ED-1, ED-2, and ED-3 may all emit blue light.

The light emitting regions PXA-R, PXA-G, and PXA-B in the display apparatus DD according to one or more embodiments may be arranged in a stripe form. Referring to FIG. 1, the plurality of red light emitting regions PXA-R, the plurality of green light emitting regions PXA-G, and the plurality of blue light emitting regions PXA-B each may be arranged along a second directional axis DR2. In some embodiments, the red light emitting region PXA-R, the green light emitting region PXA-G, and the blue light emitting region PXA-B may be alternately arranged in this order along a first directional axis DR1.

FIGS. 1 and 2 illustrate that all the light emitting regions PXA-R, PXA-G, and PXA-B have similar area, but the embodiment of the disclosure is not limited thereto. Thus, the light emitting regions PXA-R, PXA-G, and PXA-B may have different areas from each other according to the wavelength range of the emitted light. In this case, the areas of the light emitting regions PXA-R, PXA-G, and PXA-B may refer to areas if (e.g., when) viewed on a plane defined by the first directional axis DR1 and the second directional axis DR2.

An arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality desired or required in the display apparatus DD. For example, the arrangement form of the light emitting regions PXA-R, PXA-G, and PXA-B may be a pentile (PENTILE®^{M}) arrangement form or a diamond (DIAMOND PIXEL™) arrangement form. For example, the arrangement of the first light emitting area PXA-R, the second light emitting area PXA-G, and the third light emitting area PXA-B may be arranged in a PENTILE© form or structure, (e.g., an RGBG matrix, an RGBG structure, or an RGBG matrix structure), for example, a DIAMOND PIXEL™ form or structure, e.g., a display (e.g., an OLED display) containing red, blue, and green (RGB) light emitting regions arranged in the shape of diamonds. PENTILE© and DIAMOND PIXEL™ are trademarks owned by Samsung Display Co., Ltd. However, the disclosure is not limited thereto.

In some embodiments, the areas of the light emitting regions PXA-R, PXA-G, and PXA-B may be different from each other. For example, in one or more embodiments, the area of the green light emitting region PXA-G may be smaller than that of the blue light emitting region PXA-B, but the embodiment of the disclosure is not limited thereto.

Hereinafter, FIG. 3 to FIG. 6 are cross-sectional views schematically showing light emitting devices according to one or more embodiments. The light emitting diode ED according to one or more embodiments of the disclosure includes the amine compound of one or more embodiments described herein in at least one functional layer arranged between the first electrode EL1 and the second electrode EL2, and may thus exhibit improved lifespan characteristics.

The light emitting device ED of one or more embodiments may include a first electrode EL1, a hole transport region HTR, an emission layer EML, an electron transport region ETR, and a second electrode EL2 stacked in order.

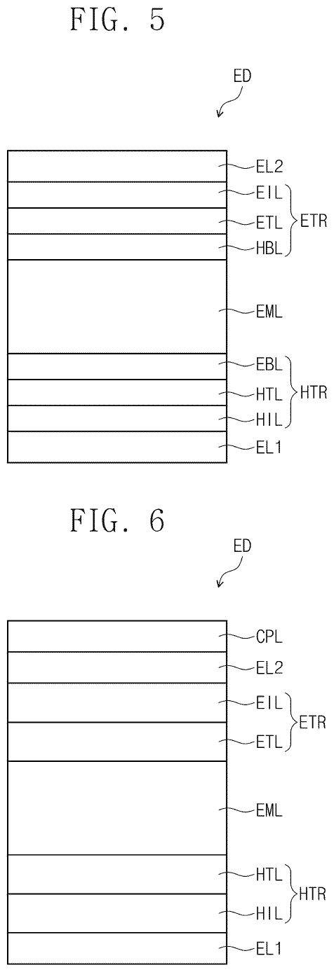

Compared with FIG. 3, FIG. 4 illustrates a cross-sectional view of a light emitting device ED of one or more embodiments, in which a hole transport region HTR includes a hole injection layer HIL and a hole transport layer HTL, and an electron transport region ETR includes an electron injection layer EIL and an electron transport layer ETL. In some embodiments, compared with FIG. 3, FIG. 5 illustrates a cross-sectional view of a light emitting device ED of one or more embodiments, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light emitting device ED of one or more embodiments including a capping layer CPL arranged on a second electrode EL2.

The light emitting element ED of one or more embodiments may include an amine compound of one or more embodiments, which will be described later, in the hole transport region HTR. The light emitting element ED of one or more embodiments may include the amine compound of one or more embodiments in at least one of the hole injection layer HIL, the hole transport layer HTL, or the electron blocking layer EBL of the hole transport region HTR. For example, in the light emitting element ED of one or more embodiments, the hole transport layer HTL may include the amine compound of one or more embodiments.

The first electrode EL1 has conductivity (e.g., is a conductor). The first electrode EL1 may be formed of a metal material, a metal alloy, or a conductive compound. The first electrode EL1 may be an anode or a cathode. However, the embodiment of the disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, and Zn, a compound of two or more selected from among these, a mixture of two or more selected from among these, or an oxide thereof.

If the first electrode EL1 is the transmissive electrode, the first electrode EL1 may include a transparent metal oxide such as indium tin oxide (ITO), indium zinc oxide (IZO), zinc oxide (ZnO), or indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound or mixture thereof (e.g., a mixture of Ag and Mg). In one or more embodiments, the first electrode EL1 may have a multilayer structure including a reflective film or a transflective film formed of the herein-described materials, and a transparent conductive film formed of ITO, IZO, ZnO, ITZO, and/or the like. For example, the first electrode EL1 may have a three-layer structure of ITO/Ag/ITO, but the embodiment of the disclosure is not limited thereto. In some embodiments, the embodiment of the disclosure is not limited thereto, and the first electrode EL1 may include the herein-described metal materials, combinations of at least two metal materials of the herein-described metal materials, oxides of the herein-described metal materials, and/or the like. The thickness of the first electrode EL1 may be from about 700 angstroms (Å) to about 10,000 Å. For example, the thickness of the first electrode EL1 may be from about 1,000 Å to about 3,000 Å.

The hole transport region HTR is provided on the first electrode EL1. The hole transport region HTR may have a single layer formed of a single material, a single layer formed of a plurality of different materials, or a multilayer structure having a plurality of layers formed of a plurality of different materials.

The hole transport region HTR may include at least one among a hole injection layer HIL, a hole transport layer HTL, and an electron blocking layer EBL. In some embodiments, although not shown, the hole transport region HTR may include a plurality of hole transport layers that are stacked.

In some embodiments, the hole transport region HTR may have a single-layer structure formed of the hole injection layer HIL or the hole transport layer HTL, or a single-layer structure formed of a hole injection material or a hole transport material. In one or more embodiments, the hole transport region HTR may have a single-layer structure formed of a plurality of different materials, or a structure in which a hole injection layer HIL/hole transport layer HTL, a hole injection layer HIL/hole transport layer HTL/buffer layer, a hole injection layer HIL/buffer layer, or a hole transport layer HTL/buffer layer are stacked in order from the first electrode EL1, but the embodiment of the disclosure is not limited thereto.

The hole transport region HTR may have, for example, a thickness of about 50 Å to about 15,000 Å. The hole transport region HTR may be formed using one or more suitable methods such as a vacuum deposition method, a spin coating method, a cast method, a Langmuir-Blodgett (LB) method, an inkjet printing method, a laser printing method, and a laser induced thermal imaging (LITI) method.

### Amine Compound

The light emitting element ED of one or more embodiments may include an amine compound of one or more embodiments in the hole transport region HTR. In the light emitting element ED of one or more embodiments, the hole transport region HTR may include an electron injection layer EIL and a hole transport layer HTL, and the hole transport layer HTL may include the amine compound of one or more embodiments. The amine compound of one or more embodiments may be included in a layer adjacent to the emission layer EML among the layers included in the hole transport region HTR.

The amine compound of one or more embodiments includes a structure in which a first substituent, a second substituent, and a third substituent are linked to a core nitrogen atom. The amine compound of one or more embodiments may be a monoamine compound including a single amine group. The amine compound of one or more embodiments may be a compound including a single amine group that does not form a ring within a molecular structure.

The first substituent may include a benzonaphthothiophene moiety. The first substituent may be a substituted or unsubstituted benzonaphthothiophene group. The first substituent may include a benzo[b]naphtho[2,1-d]thiophene moiety. The first substituent may include a moiety represented by Formula S1. The benzonaphthothiophene moiety of the first substituent may include a benzene moiety and a naphthalene moiety that are linked to each other via a sulfur atom. The benzene moiety of the benzonaphthothiophene moiety may be linked to a core nitrogen atom of the amine compound of one or more embodiments. Any carbon atom constituting the benzene moiety of the benzonaphthothiophene moiety may be linked to a core nitrogen atom of the amine compound of one or more embodiments.

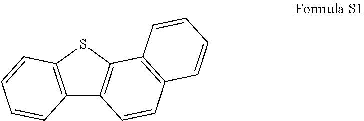 [SMILES: c1ccc2c(c1)ccc1c3ccccc3sc21]

The second substituent may include any one of (e.g., selected from among) a dibenzofuran moiety, a dibenzothiophene moiety, and a carbazole moiety. When the second substituent includes a carbazole moiety, the second substituent may include a 9-phenylcarbazole moiety. The second substituent may include a first benzene moiety and a second benzene moiety that are linked to each other via a first heteroatom. The first heteroatom may be any one of (e.g., selected from among) an oxygen atom (O), a sulfur atom (S), and a nitrogen atom (N). The first benzene moiety of the second substituent may be linked to a core nitrogen atom of the amine compound of one or more embodiments. Any carbon atom constituting the first benzene moiety of the second substituent may be linked to a core nitrogen atom of the amine compound of one or more embodiments.

The second substituent further includes one aryl group having 6 to 30 carbon atoms linked to the second benzene moiety. For example, the second substituent may further include one phenyl group linked to the second benzene moiety. Any one carbon atom constituting the second benzene moiety of the second substituent may be linked to a phenyl group.

The third substituent may be linked to a core nitrogen atom of the amine compound of one or more embodiments via a first linker. The first linker may be a direct linkage, a substituted or unsubstituted arylene group having 6 to 30 ring-forming carbon atoms, or a substituted or unsubstituted heteroarylene group having 2 to 30 ring-forming carbon atoms. The third substituent may be selected from among a substituted or unsubstituted aryl group having 6 to 30 ring-forming carbon atoms, or a substituted or unsubstituted heteroaryl group having 2 to 30 ring-forming carbon atoms. However, a case where the third substituent includes a substituted or unsubstituted benzofuran group and a substituted or unsubstituted benzothiophene group is excluded. For example, the case where the third substituent includes a moiety represented by Formula S3-1 and a moiety represented by Formula S3-2 in the amine compound of one or more embodiments is excluded.

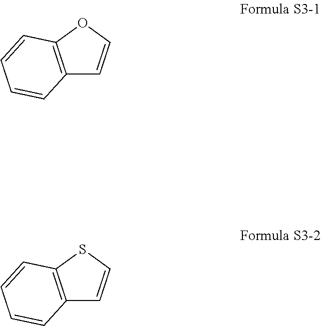 [SMILES: c1ccc2occc2c1 ; c1ccc2sccc2c1]

In one or more embodiments, the amine compound may be represented by Formula 1.

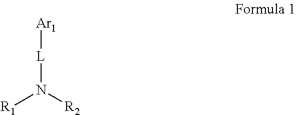

In Formula 1, L may be a direct linkage, a substituted or unsubstituted arylene group having 6 to 30 ring-forming carbon atoms, or a substituted or unsubstituted heteroarylene group having 2 to 30 ring-forming carbon atoms. For example, L may be a direct linkage or a substituted or unsubstituted phenylene group.

In Formula 1, Ar₁ may be a substituted or unsubstituted aryl group having 6 to 30 ring-forming carbon atoms, or a substituted or unsubstituted heteroaryl group having 2 to 30 ring-forming carbon atoms. However, Ar₁ does not include a substituted or unsubstituted benzofuran group and a substituted or unsubstituted benzothiophene group. For example, Ar₁ may be a substituted or unsubstituted naphthyl group, a substituted or unsubstituted phenanthrene group, a substituted or unsubstituted dibenzofuran group, a substituted or unsubstituted dibenzothiophene group, or a substituted or unsubstituted carbazole group.

In Formula 1, R₁ may be represented by Formula 2, and R₂ is represented by Formula 3.

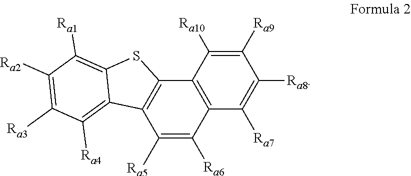

In Formula 2, any one of (e.g., selected from among) Rₐ₁ to Rₐ₄ may be a position connected to Formula 1, and the remainder of (e.g., any remaining) Rₐ₁ to Rₐ₄ may each independently be a hydrogen atom, a deuterium atom, a halogen atom, a cyano group, or a substituted or unsubstituted alkyl group having 1 to 10 carbon atoms. For example, Rₐ₁ may be a position connected to Formula 1, and Rₐ₂ to Rₐ₄ may each independently be a hydrogen atom or a deuterium atom.

In Formula 2, Rₐ₅ to Rₐ₁₀ may each independently be a hydrogen atom, a deuterium atom, a halogen atom, a cyano group, or a substituted or unsubstituted alkyl group having 1 to 10 carbon atoms. For example, Rₐ₅ to Rₐ₁₀ may each independently be a hydrogen atom or a deuterium atom.

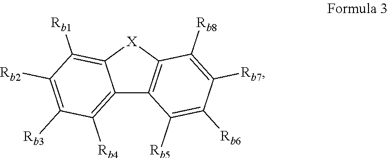

In Formula 3, X may be O, S, or NAr₂.

In Formula 3, Ar₂ may be a substituted or unsubstituted aryl group having 6 to 30 ring-forming carbon atoms. For example, Ar₂ may be a substituted or unsubstituted phenyl group.

In Formula 3, any one of (e.g., selected from among) R_{b1} to R_{b4} may be a position connected to Formula 1, and the remainder of (e.g., any remaining) R_{b1} to R_{b4} may each independently be a hydrogen atom, a deuterium atom, a halogen atom, or a substituted or unsubstituted alkyl group having 1 to 10 carbon atoms, For example, R_{b1} may be a position connected to Formula 1, and R_{b2} to R_{b4} may each independently be a hydrogen atom or a deuterium atom.

In Formula 3, any one of (e.g., selected from among) R_{b5} to R_{b8} may be a substituted or unsubstituted aryl group having 6 to 30 ring-forming carbon atoms. However, R_{b5} to R_{b8} do not include a substituted or unsubstituted fluorene group. The remainder of (e.g., any remaining) R_{b5} to R_{b8} may each independently be a hydrogen atom, a deuterium atom, a halogen atom, or a substituted or unsubstituted alkyl group having 1 to 10 carbon atoms. For example, R_{b8} may be a substituted or unsubstituted phenyl group, and R_{b5} to R_{b7} may each independently be a hydrogen atom or a deuterium atom. In one or more embodiments, R_{b6} may be a substituted or unsubstituted phenyl group, and R_{b4}, R_{b5}, and R_{b7} may each independently be a hydrogen atom or a deuterium atom.

In Formula 1, an N atom may correspond to the core nitrogen atom described, L may correspond to the first linker described herein, and Ar₁ may correspond to the third substituent described herein. A substituent represented by Formula 2 may correspond to the first substituent described herein, and a substituent represented by Formula 3 may correspond to the second substituent described herein.

In one or more embodiments, the amine compound may be represented by Formula 1-1.

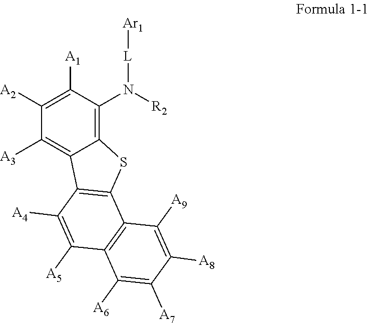

Formula 1-1 shows a case where the connection position of R₁ in Formula 1 is specified if (e.g., when) R₁ is represented by Formula 2. Formula 1-1 shows a case where Rₐ₁ in Formula 2 is connected to Formula 1.

In Formula 1-1, A₁ to A₉ may each independently be a hydrogen atom, a deuterium atom, a halogen atom, or a substituted or unsubstituted alkyl group having 1 to 10 carbon atoms. For example, A₁ to A₉ may each independently be a hydrogen atom or a deuterium atom.

In Formula 1-1, the descriptions as in Formula 1 may also apply to L, Ar₁, and R₂.

In one or more embodiments, the amine compound may be represented by any one of (e.g., selected from among) Formulas 1-2 to 1-5.

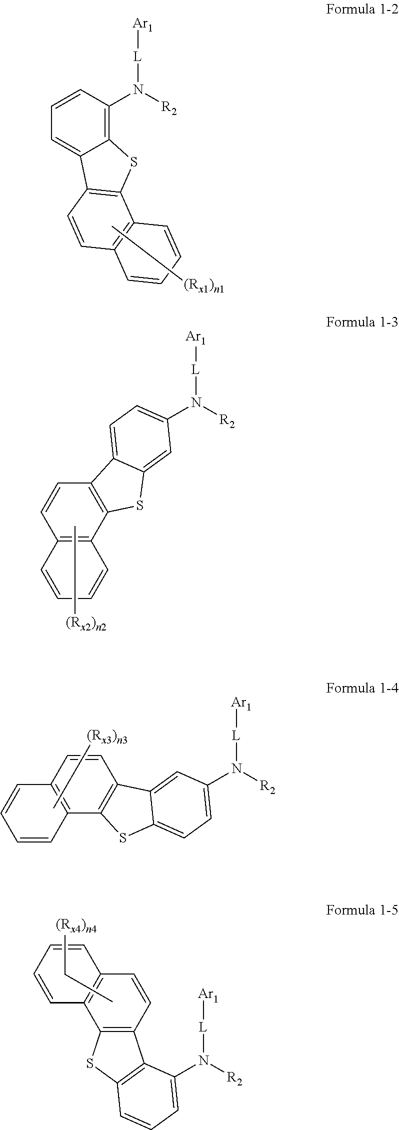

Formulas 1-2 to 1-5 shows cases where the types (kinds) of substituents Rₐ₁ to Rₐ₅ in Formula 2 are specified if (e.g., when) R₁ in Formula 1 is represented by Formula 2. Formula 1-2 shows a case where Rₐ₁ in Formula 2 is connected to Formula 1 and Rₐ₂ to Rₐ₄ correspond to hydrogen atoms, Formula 1-2 shows a case where Rₐ₂ in Formula 2 is connected to Formula 1 and Rₐ₁, Rₐ₃, and Rₐ₄ correspond to hydrogen atoms, Formula 1-4 shows a case where Rₐ₃ in Formula 2 is connected to Formula 1 and Rₐ₁, Rₐ₂, and Rₐ₄ correspond to hydrogen atoms, and Formula 1-5 shows a case where Rₐ₄ is connected to Formula 1 and Rₐ₁ to Rₐ₃ correspond to hydrogen atoms.

In Formulas 1-2 to 1-5, Rₓ₁ to Rₓ₄ may each independently be a hydrogen atom, a deuterium atom, or a substituted or unsubstituted alkyl group having 1 to 5 carbon atoms. For example, Rₓ₁ to Rₓ₄ may each independently be a hydrogen atom or a deuterium atom.

In Formula 1-2, n1 may be an integer of 0 to 6. When n1 is 0, the amine compound of one or more embodiments may not be substituted with Rₓ₁. When n1 is 6, and each Rₓ₁ is a hydrogen atom, the case may be the same as if (e.g., when) n1 is 0. When n1 is an integer of 2 or greater, Rₓ₁ provided in plurality may all be the same, or at least one of the plurality of Rₓ₁'s may be different.

In Formula 1-3, n2 may be an integer of 0 to 6. When n2 is 0, the amine compound of one or more embodiments may not be substituted with Rₓ₂. When n2 is 6, and each Rₓ₂ is a hydrogen atom, the case may be the same as if (e.g., when) n2 is 0. When n2 is an integer of 2 or greater, Rₓ₂ provided in plurality may all be the same, or at least one of the plurality of Rₓ₂'s may be different.

In Formula 1-4, n3 may be an integer of 0 to 6. When n3 is 0, the amine compound of one or more embodiments may not be substituted with Rₓ₃. When n3 is 6, and each Rₓ₄ is a hydrogen atom, the case may be the same as if (e.g., when) n3 is 0. When n3 is an integer of 2 or greater, Rₓ₄ provided in plurality may all be the same, or at least one of the plurality of Rₓ₄'s may be different.

In Formula 1-5, n4 may be an integer of 0 to 6. When n4 is 0, the amine compound of one or more embodiments may not be substituted with Rₓ₄. When n4 is 6, and each Rₓ₄ is a hydrogen atom, the case may be the same as if (e.g., when) n4 is 0. When n4 is an integer of 2 or greater, Rₓ₄ provided in plurality may all be the same, or at least one of the plurality of Rₓ₄'s may be different.

In Formulas 1-2 to 1-5, the descriptions as in Formula 1 may also apply to L, Ar₁, and R₂.

In one or more embodiments, the amine compound may be represented by any one of (e.g., selected from among) Formulas 1-6 to 1-9.

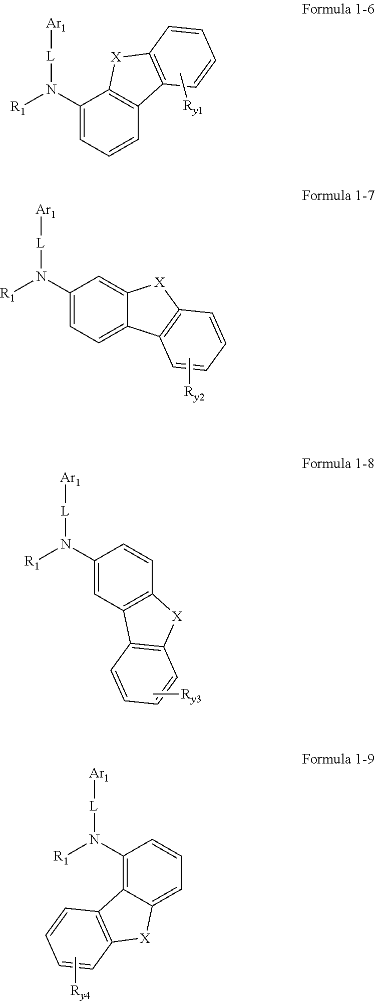

Formulas 1-6 to 1-9 shows cases where the types (kinds) of substituents R_{b1} to R_{b8} in Formula 3 are specified if (e.g., when) R₂ in Formula 1 is represented by Formula 3. Formula 1-6 shows a case where R_{b1} in Formula 3 is connected to Formula 1, R_{b2} to R_{b4} correspond to hydrogen atoms, any one of (e.g., selected from among) R_{b5} to R_{b8} corresponds to R_{y1}, and the reminder of R_{b5} to R_{b8} is a hydrogen atom. Formula 1-7 shows a case where R_{b2} in Formula 3 is connected to Formula 1, R_{b1}, R_{b3}, and R_{b4} correspond to hydrogen atoms, any one of (e.g., selected from among) R_{b5} to R_{b8} corresponds to R_{y2}, and the reminder of R_{b5} to R_{b8} is a hydrogen atom. Formula 1-8 shows a case where R_{b3} in Formula 3 is connected to Formula 1, R_{b1}, R_{b2}, and R_{b4} correspond to hydrogen atoms, any one of (e.g., selected from among) R_{b5} to R_{b8} corresponds to R_{y3}, and the reminder of R_{b5} to R_{b8} is a hydrogen atom. Formula 1-9 shows a case where R_{b4} in Formula 3 is connected to Formula 1, R_{b1} to R_{b8} correspond to hydrogen atoms, any one of (e.g., selected from among) R_{b5} to R_{b8} corresponds to R_{y4}, and the reminder of R_{b5} to R_{b8} is a hydrogen atom.

In Formulas 1-6 to 1-9, R_{y1} to R_{y4} may each independently be a substituted or unsubstituted aryl group having 6 to 12 ring-forming carbon atoms. For example, R_{y1} to R_{y4} may each independently be a substituted or unsubstituted phenyl group.

In Formulas 1-6 to 1-9, the descriptions as in Formula 1 may also apply to L, Ar₁, and R₁.

In one or more embodiments, the substituent represented by Formula 3 in the amine compound may be represented by Formula 3-1.

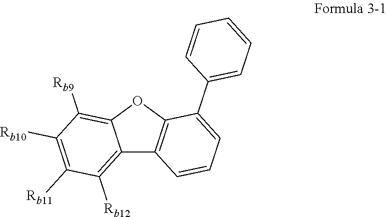

Formula 3-1 shows a case where X in Formula 3 is O, R_{b5} to R_{b7} in Formula 3 are hydrogen atoms, and R_{b8} is an unsubstituted phenyl group.

In Formula 3-1, any one of (e.g., selected from among) R_{b8} to R_{b12} may be a position connected to Formula 1, and the remainder of (e.g., any remaining) R_{b8} to R_{b12} may each independently be a hydrogen atom or a deuterium atom. For example, R_{b10} may be a position connected to Formula 1, and R_{b9}, R_{b11}, and R_{b12} may be hydrogen atoms.

In one or more embodiments, the substituent represented by Formula 3 in the amine compound may be represented by Formula 3-2.

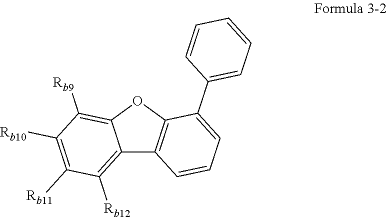

Formula 3-2 shows a case where X in Formula 3 is S, R_{b5} to R_{b7} in Formula 3 are hydrogen atoms, and R_{b8} is an unsubstituted phenyl group.

In Formula 3-2, any one of (e.g., selected from among) R_{b13} to R_{b16} may be a position connected to Formula 1, and the remainder of (e.g., any remaining) R_{b13} to R_{b16} may each independently be a hydrogen atom or a deuterium atom. For example, R_{b13} may be a position connected to Formula 1, and R_{b14} to R_{b16} may be hydrogen atoms.

In one or more embodiments, the substituent represented by Formula 3 in the amine compound may be represented by Formula 3-3.

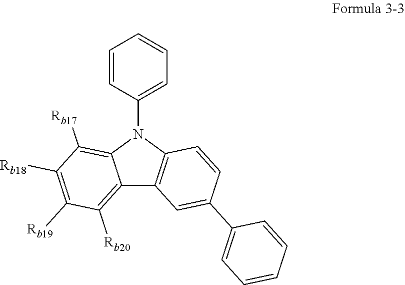

Formula 3-3 shows a case where X in Formula 3 is NAr₂, Ar₂ is an unsubstituted phenyl group, R_{b5}, R_{b7}, and R_{b8} in Formula 3 are hydrogen atoms, and R_{b6} is an unsubstituted phenyl group.

In Formula 3-3, any one of (e.g., selected from among) R_{b17} to R_{b20} may be a position connected to Formula 1, and the remainder of (e.g., any remaining) R_{b17} to R_{b20} may each independently be a hydrogen atom or a deuterium atom. For example, R_{b19} may be a position connected to Formula 1, and R_{b17}, R_{b18}, and R_{b20} may be hydrogen atoms.

In Formulas 3-1 to 3-3, any hydrogen atom may be substituted with a deuterium atom. Formulas 3-1 to 3-3 may include structures in which any hydrogen atom is substituted with a deuterium atom.

The amine compound of one or more embodiments represented by Formula 1 may be represented by any one of (e.g., selected from among) Formulas 1-A-to 1-D.

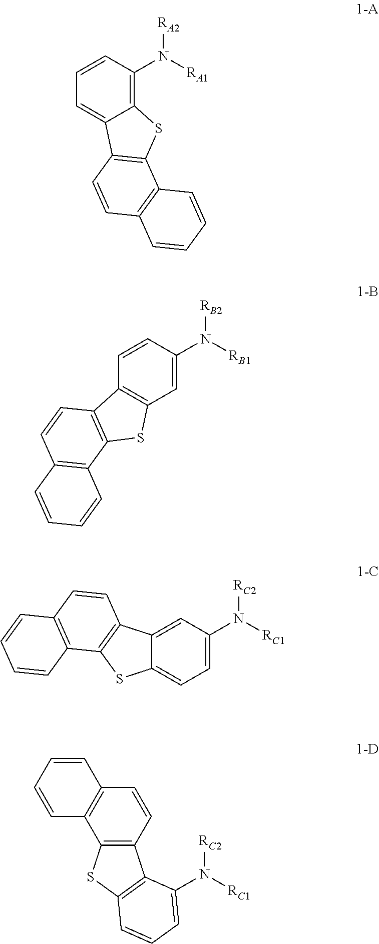

In Formula 1-A, R_{A1} and R_{A2} may each be represented by any one selected from among substituents described in Table 1. The amine compound represented by Formula 1-A may be represented by any one selected from among Compound Aa1 to Compound Av46 by the combination of substituents described in Table 1. In Formula 1-B, R_{B1} and R_{B2} may each be represented by any one selected from among substituents described in Table 2. The amine compound represented by Formula 1-B may be represented by any one selected from among Compound Ba1 to Compound Bv46 by the combination of substituents described in Table 2. In Formula 1-C, R_{C1} and R_{C2} may each be represented by any one selected from among substituents described in Table 3. The amine compound represented by Formula 1-C may be represented by any one selected from among Compound Cal to Compound Cv46 by the combination of substituents described in Table 3. In Formula 1-D, R_{D1} and R_{D2} may each be represented by any one selected from among substituents described in Table 4. The amine compound represented by Formula 1-D may be represented by any one selected from among Compound Dal to Compound Dv46 by the combination of substituents described in Table 4. In Tables 1 to 4, BN1 to BN21 and CN1 to CN46 each indicate a substituent represented as follows. In BN1 to BN21, -* indicates a position connected to an N atom in Formulas 1-A to 1-D, and in CN1 to CN46, [symbol] indicates a position connected to an N atom in Formulas 1-A to 1-D. In other words, Formulas 1-A through 1-D serve as generalized structural templates for a wide range of amine compounds, where the specific identity of each compound is determined by the selection and combination of substituents at defined positions. Tables 1 through 4 provide the substituent options for each formula, and the resulting compounds-labeled Aa1 to Av46, Ba1 to Bv46, Cal to Cv46, and Dal to Dv46-represent concrete examples of how these templates may be populated. The use of BN and CN identifiers in the tables allows for consistent mapping of substituent positions to the nitrogen atoms in the core structures, facilitating systematic variation and comparison of compound properties across the different structural families.

The structural diversity enabled by Formulas 1-A through 1-D, in combination with the substituent options listed in Tables 1 to 4, provides a platform for designing amine compounds with tunable electronic and physical properties. These design variations allow for control over molecular characteristics such as energy levels, charge transport efficiency, and/or thermal or morphological stability-factors that are desired for enhancing the performance and reliability of light emitting elements. The compound groups Aa1 to Av46, Ba1 to Bv46, Cal to Cv46, and Dal to Dv46 provide examples of the breadth of molecular architectures that may be tailored for specific roles within functional layers of display devices or other optoelectronic systems.

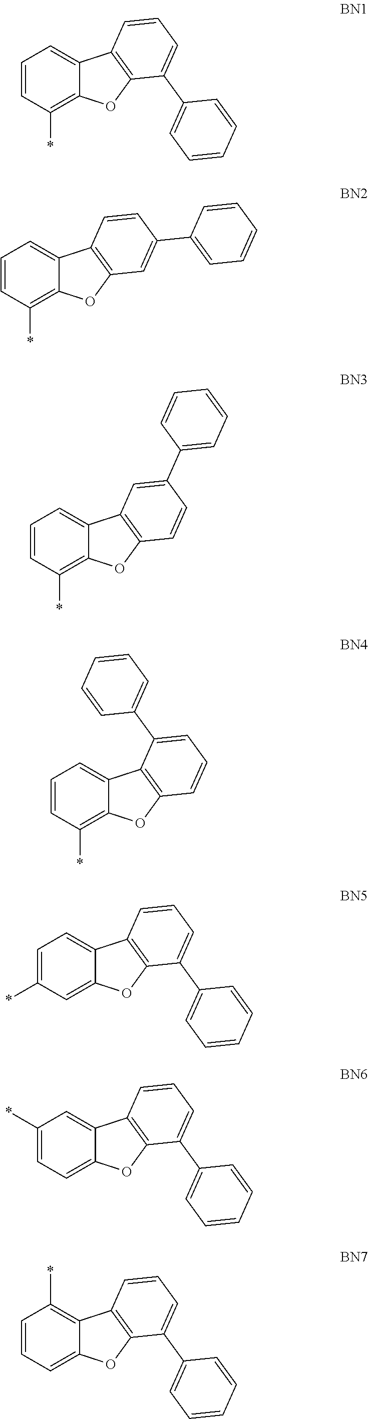

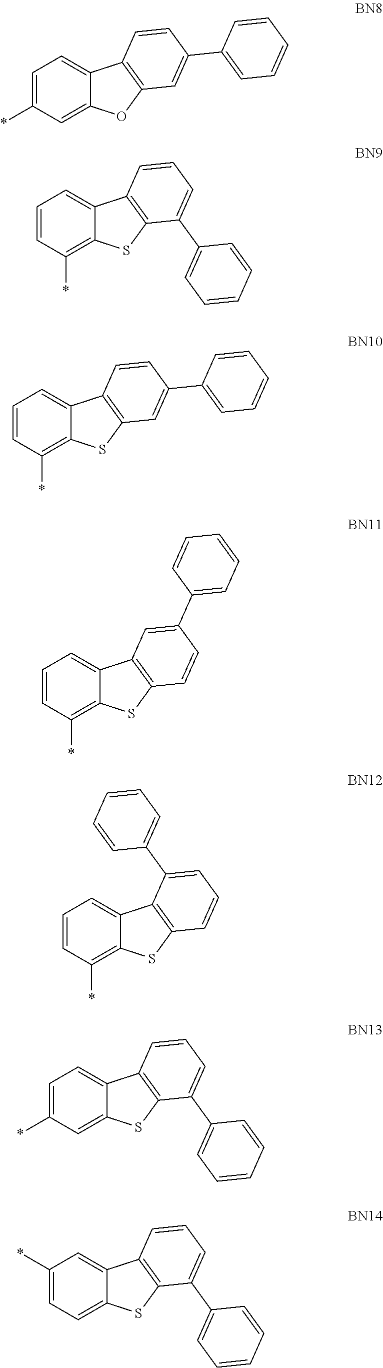

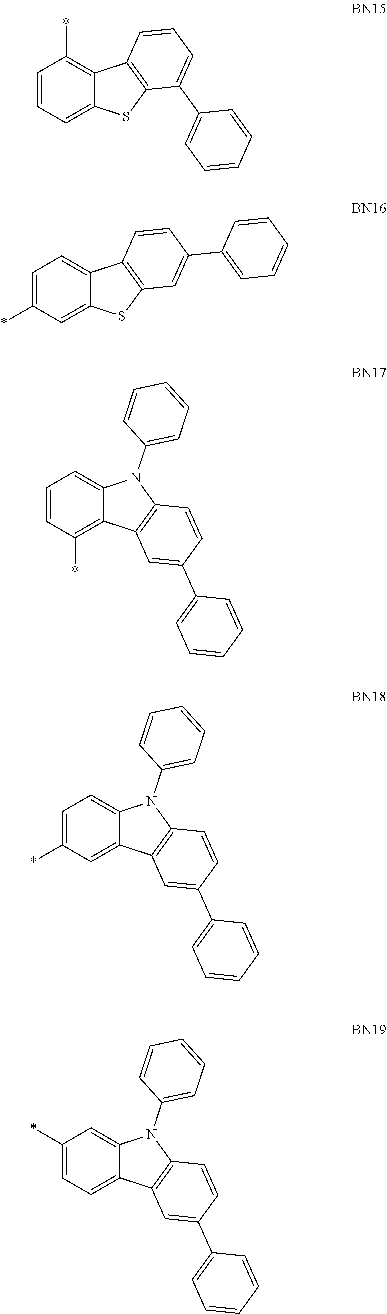 [SMILES: *c1cccc2sc3c(-c4ccccc4)cccc3c12 ; *c1ccc2c(c1)sc1cc(-c3ccccc3)ccc12 ; *c1cccc2c1c1cc(-c3ccccc3)ccc1n2-c1ccccc1 ; *c1ccc2c(c1)c1cc(-c3ccccc3)ccc1n2-c1ccccc1 ; *c1ccc2c3cc(-c4ccccc4)ccc3n(-c3ccccc3)c2c1]

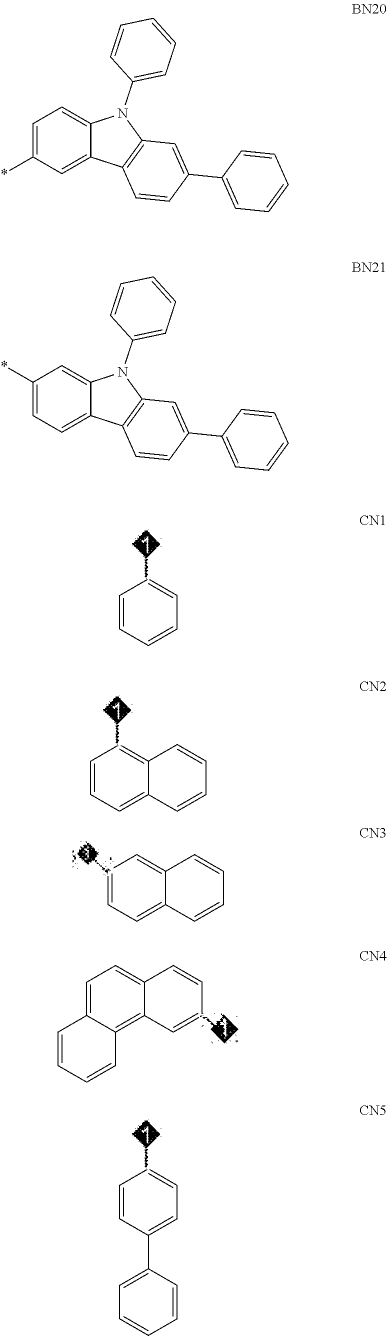

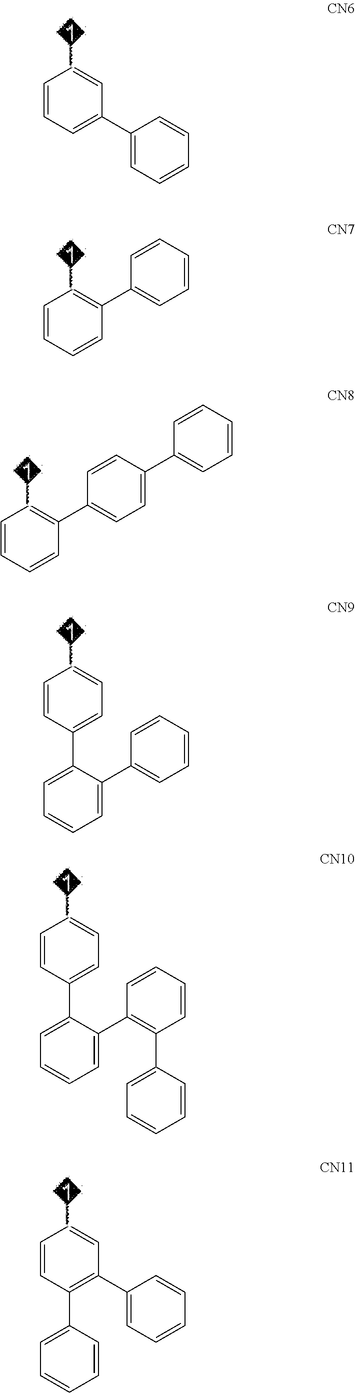

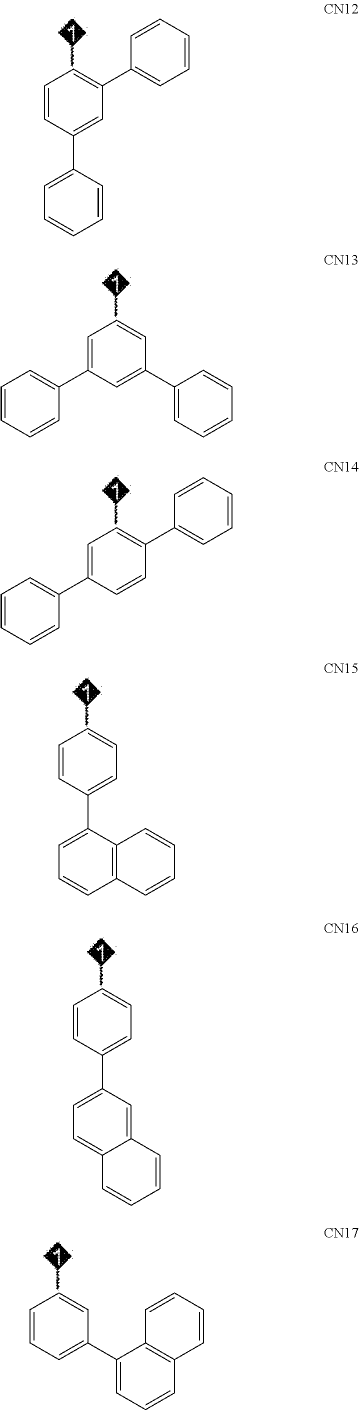

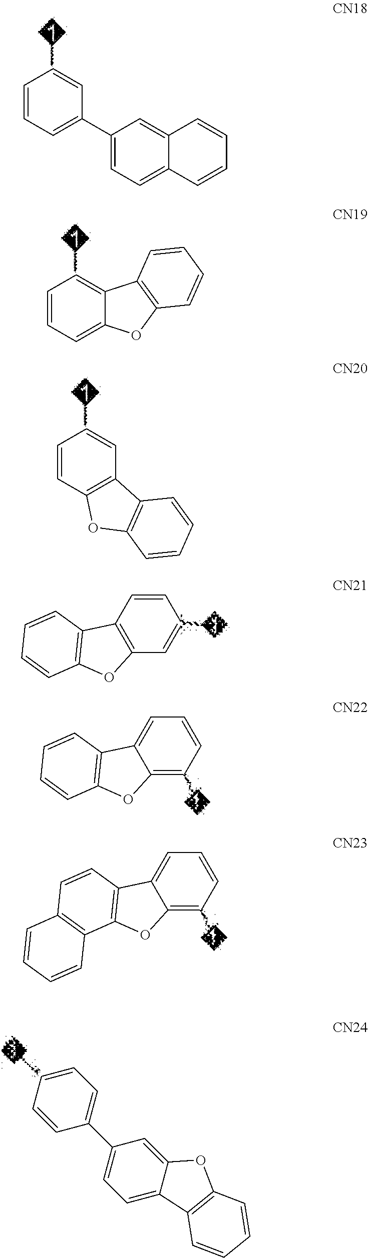

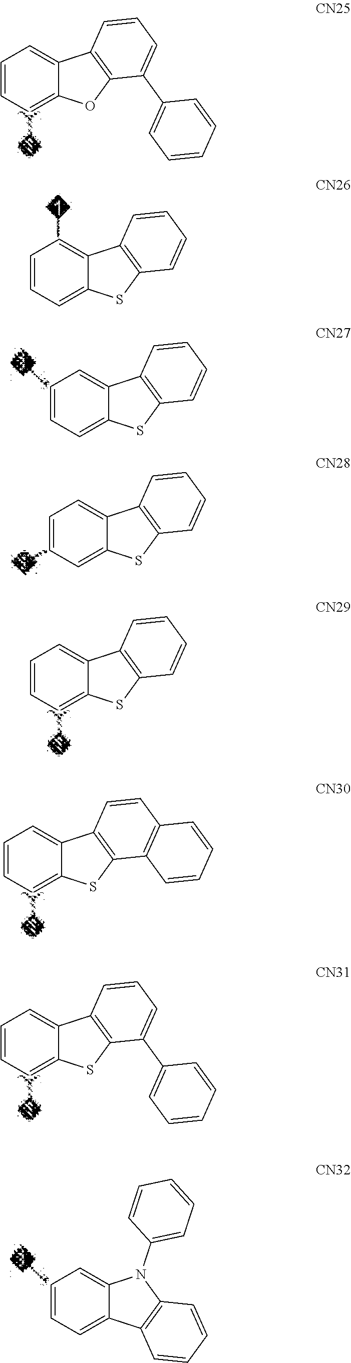

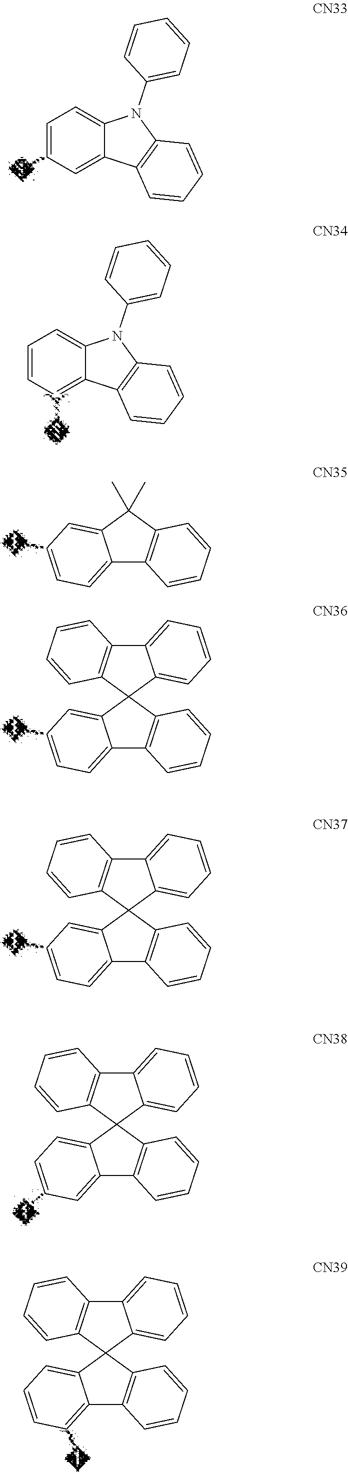

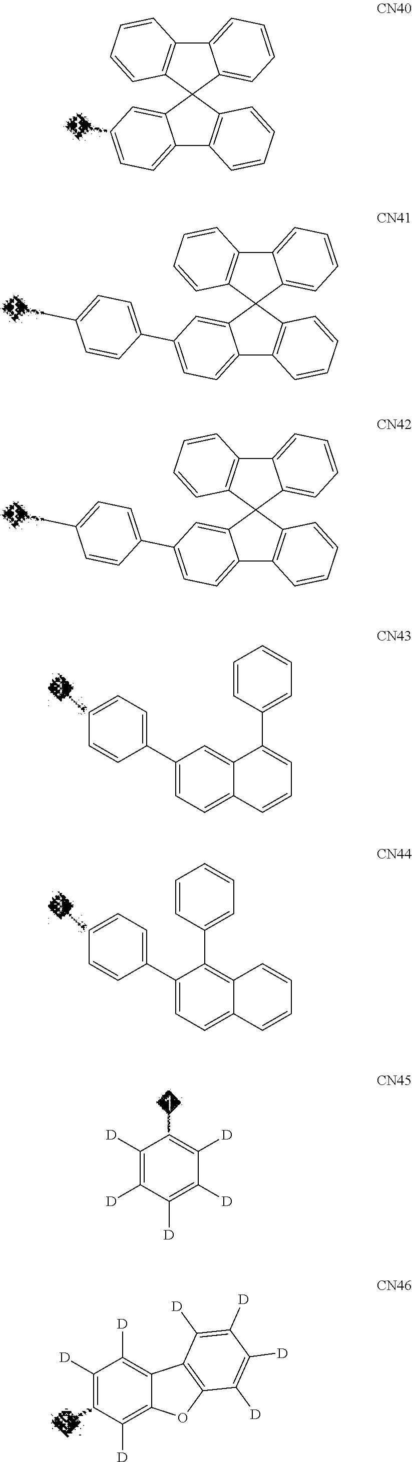

TABLE 1

| Name of | | |
|---|---|---|
| compound | R_{A1} | R_{A2} |
| Aa1 | BN1 | CN1 |
| Aa2 | BN1 | CN2 |
| Aa3 | BN1 | CN3 |
| Aa4 | BN1 | CN4 |
| Aa5 | BN1 | CN5 |
| Aa6 | BN1 | CN6 |
| Aa7 | BN1 | CN7 |
| Aa8 | BN1 | CN8 |
| Aa9 | BN1 | CN9 |
| Aa10 | BN1 | CN10 |
| Aa11 | BN1 | CN11 |
| Aa12 | BN1 | CN12 |
| Aa13 | BN1 | CN13 |
| Aa14 | BN1 | CN14 |
| Aa15 | BN1 | CN15 |
| Aa16 | BN1 | CN16 |
| Aa17 | BN1 | CN17 |
| Aa18 | BN1 | CN18 |
| Aa19 | BN1 | CN19 |
| Aa20 | BN1 | CN20 |
| Aa21 | BN1 | CN21 |
| Aa22 | BN1 | CN22 |
| Aa23 | BN1 | CN23 |
| Aa24 | BN1 | CN24 |
| Aa25 | BN1 | CN25 |
| Aa26 | BN1 | CN26 |
| Aa27 | BN1 | CN27 |
| Aa28 | BN1 | CN28 |
| Aa29 | BN1 | CN29 |
| Aa30 | BN1 | CN30 |
| Aa31 | BN1 | CN31 |
| Aa32 | BN1 | CN32 |
| Aa33 | BN1 | CN33 |
| Aa34 | BN1 | CN34 |
| Aa35 | BN1 | CN35 |
| Aa36 | BN1 | CN36 |
| Aa37 | BN1 | CN37 |
| Aa38 | BN1 | CN38 |
| Aa39 | BN1 | CN39 |
| Aa40 | BN1 | CN40 |
| Aa41 | BN1 | CN41 |
| Aa42 | BN1 | CN42 |
| Aa43 | BN1 | CN43 |
| Aa44 | BN1 | CN44 |
| Aa45 | BN1 | CN45 |
| Aa46 | BN1 | CN46 |
| Ab1 | BN2 | CN1 |
| Ab2 | BN2 | CN2 |
| Ab3 | BN2 | CN3 |
| Ab4 | BN2 | CN4 |
| Ab5 | BN2 | CN5 |
| Ab6 | BN2 | CN6 |
| Ab7 | BN2 | CN7 |
| Ab8 | BN2 | CN8 |
| Ab9 | BN2 | CN9 |
| Ab10 | BN2 | CN10 |
| Ab11 | BN2 | CN11 |
| Ab12 | BN2 | CN12 |
| Ab13 | BN2 | CN13 |
| Ab14 | BN2 | CN14 |
| Ab15 | BN2 | CN15 |
| Ab16 | BN2 | CN16 |
| Ab17 | BN2 | CN17 |
| Ab18 | BN2 | CN18 |
| Ab19 | BN2 | CN19 |
| Ab20 | BN2 | CN20 |
| Ab21 | BN2 | CN21 |
| Ab22 | BN2 | CN22 |
| Ab23 | BN2 | CN23 |
| Ab24 | BN2 | CN24 |
| Ab25 | BN2 | CN25 |
| Ab26 | BN2 | CN26 |
| Ab27 | BN2 | CN27 |
| Ab28 | BN2 | CN28 |
| Ab29 | BN2 | CN29 |
| Ab30 | BN2 | CN30 |
| Ab31 | BN2 | CN31 |
| Ab32 | BN2 | CN32 |
| Ab33 | BN2 | CN33 |
| Ab34 | BN2 | CN34 |
| Ab35 | BN2 | CN35 |
| Ab36 | BN2 | CN36 |
| Ab37 | BN2 | CN37 |
| Ab38 | BN2 | CN38 |
| Ab39 | BN2 | CN39 |
| Ab40 | BN2 | CN40 |
| Ab41 | BN2 | CN41 |
| Ab42 | BN2 | CN42 |
| Ab43 | BN2 | CN43 |
| Ab44 | BN2 | CN44 |
| Ab45 | BN2 | CN45 |
| Ab46 | BN2 | CN46 |
| Ac1 | BN3 | CN1 |
| Ac2 | BN3 | CN2 |
| Ac3 | BN3 | CN3 |
| Ac4 | BN3 | CN4 |
| Ac5 | BN3 | CN5 |
| Ac6 | BN3 | CN6 |
| Ac7 | BN3 | CN7 |
| Ac8 | BN3 | CN8 |
| Ac9 | BN3 | CN9 |
| Ac10 | BN3 | CN10 |
| Ac11 | BN3 | CN11 |
| Ac12 | BN3 | CN12 |
| Ac13 | BN3 | CN13 |
| Ac14 | BN3 | CN14 |
| Ac15 | BN3 | CN15 |
| Ac16 | BN3 | CN16 |
| Ac17 | BN3 | CN17 |
| Ac18 | BN3 | CN18 |
| Ac19 | BN3 | CN19 |
| Ac20 | BN3 | CN20 |
| Ac21 | BN3 | CN21 |
| Ac22 | BN3 | CN22 |
| Ac23 | BN3 | CN23 |
| Ac24 | BN3 | CN24 |
| Ac25 | BN3 | CN25 |
| Ac26 | BN3 | CN26 |
| Ac27 | BN3 | CN27 |
| Ac28 | BN3 | CN28 |
| Ac29 | BN3 | CN29 |
| Ac30 | BN3 | CN30 |
| Ac31 | BN3 | CN31 |
| Ac32 | BN3 | CN32 |
| Ac33 | BN3 | CN33 |
| Ac34 | BN3 | CN34 |
| Ac35 | BN3 | CN35 |
| Ac36 | BN3 | CN36 |
| Ac37 | BN3 | CN37 |
| Ac38 | BN3 | CN38 |
| Ac39 | BN3 | CN39 |
| Ac40 | BN3 | CN40 |
| Ac41 | BN3 | CN41 |
| Ac42 | BN3 | CN42 |
| Ac43 | BN3 | CN43 |
| Ac44 | BN3 | CN44 |
| Ac45 | BN3 | CN45 |
| Ac46 | BN3 | CN46 |
| Ad1 | BN4 | CN1 |
| Ad2 | BN4 | CN2 |
| Ad3 | BN4 | CN3 |
| Ad4 | BN4 | CN4 |
| Ad5 | BN4 | CN5 |
| Ad6 | BN4 | CN6 |
| Ad7 | BN4 | CN7 |
| Ad8 | BN4 | CN8 |
| Ad9 | BN4 | CN9 |
| Ad10 | BN4 | CN10 |
| Ad11 | BN4 | CN11 |
| Ad12 | BN4 | CN12 |
| Ad13 | BN4 | CN13 |
| Ad14 | BN4 | CN14 |
| Ad15 | BN4 | CN15 |
| Ad16 | BN4 | CN16 |
| Ad17 | BN4 | CN17 |
| Ad18 | BN4 | CN18 |
| Ad19 | BN4 | CN19 |
| Ad20 | BN4 | CN20 |
| Ad21 | BN4 | CN21 |
| Ad22 | BN4 | CN22 |
| Ad23 | BN4 | CN23 |
| Ad24 | BN4 | CN24 |
| Ad25 | BN4 | CN25 |
| Ad26 | BN4 | CN26 |
| Ad27 | BN4 | CN27 |
| Ad28 | BN4 | CN28 |
| Ad29 | BN4 | CN29 |
| Ad30 | BN4 | CN30 |
| Ad31 | BN4 | CN31 |
| Ad32 | BN4 | CN32 |
| Ad33 | BN4 | CN33 |
| Ad34 | BN4 | CN34 |
| Ad35 | BN4 | CN35 |
| Ad36 | BN4 | CN36 |
| Ad37 | BN4 | CN37 |
| Ad38 | BN4 | CN38 |
| Ad39 | BN4 | CN39 |
| Ad40 | BN4 | CN40 |
| Ad41 | BN4 | CN41 |
| Ad42 | BN4 | CN42 |
| Ad43 | BN4 | CN43 |
| Ad44 | BN4 | CN44 |
| Ad45 | BN4 | CN45 |
| Ad46 | BN4 | CN46 |
| Ae1 | BN5 | CN1 |
| Ae2 | BN5 | CN2 |
| Ae3 | BN5 | CN3 |
| Ae4 | BN5 | CN4 |
| Ae5 | BN5 | CN5 |
| Ae6 | BN5 | CN6 |
| Ae7 | BN5 | CN7 |
| Ae8 | BN5 | CN8 |
| Ae9 | BN5 | CN9 |
| Ae10 | BN5 | CN10 |
| Ae11 | BN5 | CN11 |
| Ae12 | BN5 | CN12 |
| Ae13 | BN5 | CN13 |
| Ae14 | BN5 | CN14 |
| Ae15 | BN5 | CN15 |
| Ae16 | BN5 | CN16 |
| Ae17 | BN5 | CN17 |
| Ae18 | BN5 | CN18 |
| Ae19 | BN5 | CN19 |
| Ae20 | BN5 | CN20 |
| Ae21 | BN5 | CN21 |
| Ae22 | BN5 | CN22 |
| Ae23 | BN5 | CN23 |
| Ae24 | BN5 | CN24 |
| Ae25 | BN5 | CN25 |
| Ae26 | BN5 | CN26 |
| Ae27 | BN5 | CN27 |
| Ae28 | BN5 | CN28 |
| Ae29 | BN5 | CN29 |
| Ae30 | BN5 | CN30 |
| Ae31 | BN5 | CN31 |
| Ae32 | BN5 | CN32 |
| Ae33 | BN5 | CN33 |
| Ae34 | BN5 | CN34 |
| Ae35 | BN5 | CN35 |
| Ae36 | BN5 | CN36 |
| Ae37 | BN5 | CN37 |
| Ae38 | BN5 | CN38 |
| Ae39 | BN5 | CN39 |
| Ae40 | BN5 | CN40 |
| Ae41 | BN5 | CN41 |
| Ae42 | BN5 | CN42 |
| Ae43 | BN5 | CN43 |
| Ae44 | BN5 | CN44 |
| Ae45 | BN5 | CN45 |
| Ae46 | BN5 | CN46 |
| Af1 | BN6 | CN1 |
| Af2 | BN6 | CN2 |
| Af3 | BN6 | CN3 |
| Af4 | BN6 | CN4 |
| Af5 | BN6 | CN5 |
| Af6 | BN6 | CN6 |
| Af7 | BN6 | CN7 |
| Af8 | BN6 | CN8 |
| Af9 | BN6 | CN9 |
| Af10 | BN6 | CN10 |
| Af11 | BN6 | CN11 |
| Af12 | BN6 | CN12 |
| Af13 | BN6 | CN13 |
| Af14 | BN6 | CN14 |
| Af15 | BN6 | CN15 |
| Af16 | BN6 | CN16 |
| Af17 | BN6 | CN17 |
| Af18 | BN6 | CN18 |
| Af19 | BN6 | CN19 |
| Af20 | BN6 | CN20 |
| Af21 | BN6 | CN21 |
| Af22 | BN6 | CN22 |
| Af23 | BN6 | CN23 |
| Af24 | BN6 | CN24 |
| Af25 | BN6 | CN25 |
| Af26 | BN6 | CN26 |
| Af27 | BN6 | CN27 |
| Af28 | BN6 | CN28 |
| Af29 | BN6 | CN29 |
| Af30 | BN6 | CN30 |
| Af31 | BN6 | CN31 |
| Af32 | BN6 | CN32 |
| Af33 | BN6 | CN33 |
| Af34 | BN6 | CN34 |
| Af35 | BN6 | CN35 |
| Af36 | BN6 | CN36 |
| Af37 | BN6 | CN37 |
| Af38 | BN6 | CN38 |
| Af39 | BN6 | CN39 |
| Af40 | BN6 | CN40 |
| Af41 | BN6 | CN41 |
| Af42 | BN6 | CN42 |
| Af43 | BN6 | CN43 |
| Af44 | BN6 | CN44 |
| Af45 | BN6 | CN45 |
| Af46 | BN6 | CN46 |
| Ag1 | BN7 | CN1 |
| Ag2 | BN7 | CN2 |
| Ag3 | BN7 | CN3 |
| Ag4 | BN7 | CN4 |
| Ag5 | BN7 | CN5 |
| Ag6 | BN7 | CN6 |
| Ag7 | BN7 | CN7 |
| Ag8 | BN7 | CN8 |
| Ag9 | BN7 | CN9 |
| Ag10 | BN7 | CN10 |
| Ag11 | BN7 | CN11 |
| Ag12 | BN7 | CN12 |
| Ag13 | BN7 | CN13 |
| Ag14 | BN7 | CN14 |
| Ag15 | BN7 | CN15 |
| Ag16 | BN7 | CN16 |
| Ag17 | BN7 | CN17 |
| Ag18 | BN7 | CN18 |
| Ag19 | BN7 | CN19 |
| Ag20 | BN7 | CN20 |
| Ag21 | BN7 | CN21 |
| Ag22 | BN7 | CN22 |
| Ag23 | BN7 | CN23 |
| Ag24 | BN7 | CN24 |
| Ag25 | BN7 | CN25 |
| Ag26 | BN7 | CN26 |
| Ag27 | BN7 | CN27 |
| Ag28 | BN7 | CN28 |
| Ag29 | BN7 | CN29 |
| Ag30 | BN7 | CN30 |
| Ag31 | BN7 | CN31 |
| Ag32 | BN7 | CN32 |
| Ag33 | BN7 | CN33 |
| Ag34 | BN7 | CN34 |
| Ag35 | BN7 | CN35 |
| Ag36 | BN7 | CN36 |
| Ag37 | BN7 | CN37 |
| Ag38 | BN7 | CN38 |
| Ag39 | BN7 | CN39 |
| Ag40 | BN7 | CN40 |
| Ag41 | BN7 | CN41 |
| Ag42 | BN7 | CN42 |
| Ag43 | BN7 | CN43 |
| Ag44 | BN7 | CN44 |
| Ag45 | BN7 | CN45 |
| Ag46 | BN7 | CN46 |
| Ah1 | BN8 | CN1 |
| Ah2 | BN8 | CN2 |
| Ah3 | BN8 | CN3 |
| Ah4 | BN8 | CN4 |
| Ah5 | BN8 | CN5 |
| Ah6 | BN8 | CN6 |
| Ah7 | BN8 | CN7 |
| Ah8 | BN8 | CN8 |
| Ah9 | BN8 | CN9 |
| Ah10 | BN8 | CN10 |
| Ah11 | BN8 | CN11 |
| Ah12 | BN8 | CN12 |
| Ah13 | BN8 | CN13 |
| Ah14 | BN8 | CN14 |
| Ah15 | BN8 | CN15 |
| Ah16 | BN8 | CN16 |
| Ah17 | BN8 | CN17 |
| Ah18 | BN8 | CN18 |
| Ah19 | BN8 | CN19 |
| Ah20 | BN8 | CN20 |
| Ah21 | BN8 | CN21 |
| Ah22 | BN8 | CN22 |
| Ah23 | BN8 | CN23 |
| Ah24 | BN8 | CN24 |
| Ah25 | BN8 | CN25 |
| Ah26 | BN8 | CN26 |
| Ah27 | BN8 | CN27 |
| Ah28 | BN8 | CN28 |
| Ah29 | BN8 | CN29 |
| Ah30 | BN8 | CN30 |
| Ah31 | BN8 | CN31 |
| Ah32 | BN8 | CN32 |
| Ah33 | BN8 | CN33 |
| Ah34 | BN8 | CN34 |
| Ah35 | BN8 | CN35 |
| Ah36 | BN8 | CN36 |
| Ah37 | BN8 | CN37 |
| Ah38 | BN8 | CN38 |
| Ah39 | BN8 | CN39 |
| Ah40 | BN8 | CN40 |
| Ah41 | BN8 | CN41 |
| Ah42 | BN8 | CN42 |
| Ah43 | BN8 | CN43 |
| Ah44 | BN8 | CN44 |
| Ah45 | BN8 | CN45 |
| Ah46 | BN8 | CN46 |
| Ai1 | BN9 | CN1 |
| Ai2 | BN9 | CN2 |
| Ai3 | BN9 | CN3 |
| Ai4 | BN9 | CN4 |
| Ai5 | BN9 | CN5 |
| Ai6 | BN9 | CN6 |
| Ai7 | BN9 | CN7 |
| Ai8 | BN9 | CN8 |
| Ai9 | BN9 | CN9 |
| Ai10 | BN9 | CN10 |
| Ai11 | BN9 | CN11 |
| Ai12 | BN9 | CN12 |
| Ai13 | BN9 | CN13 |
| Ai14 | BN9 | CN14 |
| Ai15 | BN9 | CN15 |
| Ai16 | BN9 | CN16 |
| Ai17 | BN9 | CN17 |
| Ai18 | BN9 | CN18 |
| Ai19 | BN9 | CN19 |
| Ai20 | BN9 | CN20 |
| Ai21 | BN9 | CN21 |
| Ai22 | BN9 | CN22 |
| Ai23 | BN9 | CN23 |
| Ai24 | BN9 | CN24 |
| Ai25 | BN9 | CN25 |
| Ai26 | BN9 | CN26 |
| Ai27 | BN9 | CN27 |
| Ai28 | BN9 | CN28 |
| Ai29 | BN9 | CN29 |
| Ai30 | BN9 | CN30 |
| Ai31 | BN9 | CN31 |
| Ai32 | BN9 | CN32 |
| Ai33 | BN9 | CN33 |
| Ai34 | BN9 | CN34 |
| Ai35 | BN9 | CN35 |
| Ai36 | BN9 | CN36 |
| Ai37 | BN9 | CN37 |
| Ai38 | BN9 | CN38 |
| Ai39 | BN9 | CN39 |
| Ai40 | BN9 | CN40 |
| Ai41 | BN9 | CN41 |
| Ai42 | BN9 | CN42 |
| Ai43 | BN9 | CN43 |
| Ai44 | BN9 | CN44 |
| Ai45 | BN9 | CN45 |
| Ai46 | BN9 | CN46 |
| Aj1 | BN10 | CN1 |
| Aj2 | BN10 | CN2 |
| Aj3 | BN10 | CN3 |
| Aj4 | BN10 | CN4 |
| Aj5 | BN10 | CN5 |
| Aj6 | BN10 | CN6 |
| Aj7 | BN10 | CN7 |
| Aj8 | BN10 | CN8 |
| Aj9 | BN10 | CN9 |
| Aj10 | BN10 | CN10 |
| Aj11 | BN10 | CN11 |
| Aj12 | BN10 | CN12 |
| Aj13 | BN10 | CN13 |
| Aj14 | BN10 | CN14 |
| Aj15 | BN10 | CN15 |
| Aj16 | BN10 | CN16 |
| Aj17 | BN10 | CN17 |
| Aj18 | BN10 | CN18 |
| Aj19 | BN10 | CN19 |
| Aj20 | BN10 | CN20 |
| Aj21 | BN10 | CN21 |
| Aj22 | BN10 | CN22 |
| Aj23 | BN10 | CN23 |
| Aj24 | BN10 | CN24 |
| Aj25 | BN10 | CN25 |
| Aj26 | BN10 | CN26 |
| Aj27 | BN10 | CN27 |
| Aj28 | BN10 | CN28 |
| Aj29 | BN10 | CN29 |
| Aj30 | BN10 | CN30 |
| Aj31 | BN10 | CN31 |
| Aj32 | BN10 | CN32 |
| Aj33 | BN10 | CN33 |
| Aj34 | BN10 | CN34 |
| Aj35 | BN10 | CN35 |
| Aj36 | BN10 | CN36 |
| Aj37 | BN10 | CN37 |
| Aj38 | BN10 | CN38 |
| Aj39 | BN10 | CN39 |
| Aj40 | BN10 | CN40 |
| Aj41 | BN10 | CN41 |
| Aj42 | BN10 | CN42 |
| Aj43 | BN10 | CN43 |
| Aj44 | BN10 | CN44 |
| Aj45 | BN10 | CN45 |
| Aj46 | BN10 | CN46 |
| Ak1 | BN11 | CN1 |
| Ak2 | BN11 | CN2 |
| Ak3 | BN11 | CN3 |
| Ak4 | BN11 | CN4 |
| Ak5 | BN11 | CN5 |
| Ak6 | BN11 | CN6 |
| Ak7 | BN11 | CN7 |
| Ak8 | BN11 | CN8 |
| Ak9 | BN11 | CN9 |
| Ak10 | BN11 | CN10 |
| Ak11 | BN11 | CN11 |
| Ak12 | BN11 | CN12 |
| Ak13 | BN11 | CN13 |
| Ak14 | BN11 | CN14 |
| Ak15 | BN11 | CN15 |
| Ak16 | BN11 | CN16 |
| Ak17 | BN11 | CN17 |
| Ak18 | BN11 | CN18 |
| Ak19 | BN11 | CN19 |
| Ak20 | BN11 | CN20 |
| Ak21 | BN11 | CN21 |
| Ak22 | BN11 | CN22 |
| Ak23 | BN11 | CN23 |
| Ak24 | BN11 | CN24 |
| Ak25 | BN11 | CN25 |
| Ak26 | BN11 | CN26 |
| Ak27 | BN11 | CN27 |
| Ak28 | BN11 | CN28 |
| Ak29 | BN11 | CN29 |
| Ak30 | BN11 | CN30 |
| Ak31 | BN11 | CN31 |
| Ak32 | BN11 | CN32 |
| Ak33 | BN11 | CN33 |
| Ak34 | BN11 | CN34 |
| Ak35 | BN11 | CN35 |
| Ak36 | BN11 | CN36 |
| Ak37 | BN11 | CN37 |
| Ak38 | BN11 | CN38 |
| Ak39 | BN11 | CN39 |
| Ak40 | BN11 | CN40 |
| Ak41 | BN11 | CN41 |
| Ak42 | BN11 | CN42 |
| Ak43 | BN11 | CN43 |
| Ak44 | BN11 | CN44 |
| Ak45 | BN11 | CN45 |
| Ak46 | BN11 | CN46 |
| Al1 | BN12 | CN1 |
| Al2 | BN12 | CN2 |
| Al3 | BN12 | CN3 |
| Al4 | BN12 | CN4 |
| Al5 | BN12 | CN5 |
| Al6 | BN12 | CN6 |
| Al7 | BN12 | CN7 |
| AI8 | BN12 | CN8 |
| A19 | BN12 | CN9 |
| Al10 | BN12 | CN10 |
| Al11 | BN12 | CN11 |
| Al12 | BN12 | CN12 |
| AI13 | BN12 | CN13 |
| Al14 | BN12 | CN14 |
| Al15 | BN12 | CN15 |
| Al16 | BN12 | CN16 |
| Al17 | BN12 | CN17 |
| Al18 | BN12 | CN18 |
| Al19 | BN12 | CN19 |
| Al20 | BN12 | CN20 |
| Al21 | BN12 | CN21 |
| Al22 | BN12 | CN22 |
| Al23 | BN12 | CN23 |
| Al24 | BN12 | CN24 |
| Al25 | BN12 | CN25 |
| Al26 | BN12 | CN26 |
| Al27 | BN12 | CN27 |
| Al28 | BN12 | CN28 |
| Al29 | BN12 | CN29 |
| Al30 | BN12 | CN30 |
| Al31 | BN12 | CN31 |
| Al32 | BN12 | CN32 |
| Al33 | BN12 | CN33 |
| Al34 | BN12 | CN34 |
| Al35 | BN12 | CN35 |
| Al36 | BN12 | CN36 |
| Al37 | BN12 | CN37 |
| Al38 | BN12 | CN38 |
| Al39 | BN12 | CN39 |
| Al40 | BN12 | CN40 |
| Al41 | BN12 | CN41 |
| Al42 | BN12 | CN42 |
| Al43 | BN12 | CN43 |
| Al44 | BN12 | CN44 |
| Al45 | BN12 | CN45 |
| Al46 | BN12 | CN46 |
| Am1 | BN13 | CN1 |
| Am2 | BN13 | CN2 |
| Am3 | BN13 | CN3 |
| Am4 | BN13 | CN4 |
| Am5 | BN13 | CN5 |
| Am6 | BN13 | CN6 |
| Am7 | BN13 | CN7 |
| Am8 | BN13 | CN8 |
| Am9 | BN13 | CN9 |
| Am10 | BN13 | CN10 |
| Am11 | BN13 | CN11 |
| Am12 | BN13 | CN12 |
| Am13 | BN13 | CN13 |
| Am14 | BN13 | CN14 |
| Am15 | BN13 | CN15 |
| Am16 | BN13 | CN16 |
| Am17 | BN13 | CN17 |
| Am18 | BN13 | CN18 |
| Am19 | BN13 | CN19 |
| Am20 | BN13 | CN20 |
| Am21 | BN13 | CN21 |
| Am22 | BN13 | CN22 |
| Am23 | BN13 | CN23 |
| Am24 | BN13 | CN24 |
| Am25 | BN13 | CN25 |
| Am26 | BN13 | CN26 |
| Am27 | BN13 | CN27 |
| Am28 | BN13 | CN28 |
| Am29 | BN13 | CN29 |
| Am30 | BN13 | CN30 |
| Am31 | BN13 | CN31 |
| Am32 | BN13 | CN32 |
| Am33 | BN13 | CN33 |
| Am34 | BN13 | CN34 |
| Am35 | BN13 | CN35 |
| Am36 | BN13 | CN36 |
| Am37 | BN13 | CN37 |
| Am38 | BN13 | CN38 |
| Am39 | BN13 | CN39 |
| Am40 | BN13 | CN40 |
| Am41 | BN13 | CN41 |
| Am42 | BN13 | CN42 |
| Am43 | BN13 | CN43 |
| Am44 | BN13 | CN44 |
| Am45 | BN13 | CN45 |
| Am46 | BN13 | CN46 |
| An1 | BN14 | CN1 |
| An2 | BN14 | CN2 |
| An3 | BN14 | CN3 |
| An4 | BN14 | CN4 |
| An5 | BN14 | CN5 |
| An6 | BN14 | CN6 |
| An7 | BN14 | CN7 |
| An8 | BN14 | CN8 |
| An9 | BN14 | CN9 |
| An10 | BN14 | CN10 |
| An11 | BN14 | CN11 |
| An12 | BN14 | CN12 |
| An13 | BN14 | CN13 |
| An14 | BN14 | CN14 |
| An15 | BN14 | CN15 |
| An16 | BN14 | CN16 |
| An17 | BN14 | CN17 |
| An18 | BN14 | CN18 |
| An19 | BN14 | CN19 |
| An20 | BN14 | CN20 |
| An21 | BN14 | CN21 |
| An22 | BN14 | CN22 |
| An23 | BN14 | CN23 |
| An24 | BN14 | CN24 |
| An25 | BN14 | CN25 |
| An26 | BN14 | CN26 |
| An27 | BN14 | CN27 |
| An28 | BN14 | CN28 |
| An29 | BN14 | CN29 |
| An30 | BN14 | CN30 |
| An31 | BN14 | CN31 |
| An32 | BN14 | CN32 |
| An33 | BN14 | CN33 |
| An34 | BN14 | CN34 |
| An35 | BN14 | CN35 |
| An36 | BN14 | CN36 |
| An37 | BN14 | CN37 |
| An38 | BN14 | CN38 |
| An39 | BN14 | CN39 |
| An40 | BN14 | CN40 |
| An41 | BN14 | CN41 |
| An42 | BN14 | CN42 |
| An43 | BN14 | CN43 |
| An44 | BN14 | CN44 |
| An45 | BN14 | CN45 |
| An46 | BN14 | CN46 |
| Ao1 | BN15 | CN1 |
| Ao2 | BN15 | CN2 |
| Ao3 | BN15 | CN3 |
| Ao4 | BN15 | CN4 |
| Ao5 | BN15 | CN5 |
| Ao6 | BN15 | CN6 |
| Ao7 | BN15 | CN7 |
| A08 | BN15 | CN8 |
| Ao9 | BN15 | CN9 |
| Ao10 | BN15 | CN10 |
| Ao11 | BN15 | CN11 |
| Ao12 | BN15 | CN12 |
| Ao13 | BN15 | CN13 |
| Ao14 | BN15 | CN14 |
| Ao15 | BN15 | CN15 |
| Ao16 | BN15 | CN16 |
| Ao17 | BN15 | CN17 |
| Ao18 | BN15 | CN18 |
| Ao19 | BN15 | CN19 |
| Ao20 | BN15 | CN20 |
| Ao21 | BN15 | CN21 |
| Ao22 | BN15 | CN22 |
| Ao23 | BN15 | CN23 |
| Ao24 | BN15 | CN24 |
| Ao25 | BN15 | CN25 |
| Ao26 | BN15 | CN26 |
| Ao27 | BN15 | CN27 |
| Ao28 | BN15 | CN28 |
| Ao29 | BN15 | CN29 |
| Ao30 | BN15 | CN30 |
| Ao31 | BN15 | CN31 |
| Ao32 | BN15 | CN32 |
| Ao33 | BN15 | CN33 |
| Ao34 | BN15 | CN34 |
| Ao35 | BN15 | CN35 |
| Ao36 | BN15 | CN36 |
| Ao37 | BN15 | CN37 |
| Ao38 | BN15 | CN38 |
| Ao39 | BN15 | CN39 |
| Ao40 | BN15 | CN40 |
| Ao41 | BN15 | CN41 |
| Ao42 | BN15 | CN42 |
| Ao43 | BN15 | CN43 |
| Ao44 | BN15 | CN44 |
| Ao45 | BN15 | CN45 |
| Ao46 | BN15 | CN46 |
| Ap1 | BN16 | CN1 |
| Ap2 | BN16 | CN2 |
| Ap3 | BN16 | CN3 |
| Ap4 | BN16 | CN4 |
| Ap5 | BN16 | CN5 |
| Ap6 | BN16 | CN6 |
| Ap7 | BN16 | CN7 |
| Ap8 | BN16 | CN8 |
| Ap9 | BN16 | CN9 |
| Ap10 | BN16 | CN10 |
| Ap11 | BN16 | CN11 |
| Ap12 | BN16 | CN12 |
| Ap13 | BN16 | CN13 |
| Ap14 | BN16 | CN14 |
| Ap15 | BN16 | CN15 |
| Ap16 | BN16 | CN16 |
| Ap17 | BN16 | CN17 |
| Ap18 | BN16 | CN18 |
| Ap19 | BN16 | CN19 |
| Ap20 | BN16 | CN20 |
| Ap21 | BN16 | CN21 |
| Ap22 | BN16 | CN22 |
| Ap23 | BN16 | CN23 |
| Ap24 | BN16 | CN24 |
| Ap25 | BN16 | CN25 |
| Ap26 | BN16 | CN26 |
| Ap27 | BN16 | CN27 |
| Ap28 | BN16 | CN28 |
| Ap29 | BN16 | CN29 |
| Ap30 | BN16 | CN30 |
| Ap31 | BN16 | CN31 |
| Ap32 | BN16 | CN32 |
| Ap33 | BN16 | CN33 |
| Ap34 | BN16 | CN34 |
| Ap35 | BN16 | CN35 |
| Ap36 | BN16 | CN36 |
| Ap37 | BN16 | CN37 |
| Ap38 | BN16 | CN38 |
| Ap39 | BN16 | CN39 |
| Ap40 | BN16 | CN40 |
| Ap41 | BN16 | CN41 |
| Ap42 | BN16 | CN42 |
| Ap43 | BN16 | CN43 |
| Ap44 | BN16 | CN44 |
| Ap45 | BN16 | CN45 |
| Ap46 | BN16 | CN46 |
| Aq1 | BN17 | CN1 |
| Aq2 | BN17 | CN2 |
| Aq3 | BN17 | CN3 |
| Aq4 | BN17 | CN4 |
| Ag5 | BN17 | CN5 |
| Aq6 | BN17 | CN6 |
| Aq7 | BN17 | CN7 |
| Aq8 | BN17 | CN8 |
| Aq9 | BN17 | CN9 |
| Aq10 | BN17 | CN10 |
| Aq11 | BN17 | CN11 |
| Aq12 | BN17 | CN12 |
| Aq13 | BN17 | CN13 |
| Aq14 | BN17 | CN14 |
| Aq15 | BN17 | CN15 |
| Aq16 | BN17 | CN16 |
| Aq17 | BN17 | CN17 |
| Aq18 | BN17 | CN18 |
| Aq19 | BN17 | CN19 |
| Aq20 | BN17 | CN20 |
| Aq21 | BN17 | CN21 |
| Ag22 | BN17 | CN22 |
| Ag23 | BN17 | CN23 |
| Aq24 | BN17 | CN24 |
| Aq25 | BN17 | CN25 |
| Aq26 | BN17 | CN26 |
| Aq27 | BN17 | CN27 |
| Aq28 | BN17 | CN28 |
| Aq29 | BN17 | CN29 |
| Aq30 | BN17 | CN30 |
| Ag31 | BN17 | CN31 |
| Ag32 | BN17 | CN32 |
| Aq33 | BN17 | CN33 |
| Aq34 | BN17 | CN34 |
| Aq35 | BN17 | CN35 |
| Aq36 | BN17 | CN36 |
| Aq37 | BN17 | CN37 |
| Aq38 | BN17 | CN38 |
| Aq39 | BN17 | CN39 |
| Ag40 | BN17 | CN40 |
| Ag41 | BN17 | CN41 |
| Aq42 | BN17 | CN42 |
| Aq43 | BN17 | CN43 |
| Aq44 | BN17 | CN44 |
| Aq45 | BN17 | CN45 |
| Ag46 | BN17 | CN46 |
| As1 | BN18 | CN1 |
| As2 | BN18 | CN2 |
| As3 | BN18 | CN3 |
| As4 | BN18 | CN4 |
| As5 | BN18 | CN5 |
| As6 | BN18 | CN6 |
| As7 | BN18 | CN7 |
| As8 | BN18 | CN8 |
| As9 | BN18 | CN9 |
| As10 | BN18 | CN10 |
| As11 | BN18 | CN11 |
| As12 | BN18 | CN12 |
| As13 | BN18 | CN13 |
| As14 | BN18 | CN14 |
| As15 | BN18 | CN15 |
| As16 | BN18 | CN16 |
| As17 | BN18 | CN17 |
| As18 | BN18 | CN18 |
| As19 | BN18 | CN19 |
| As20 | BN18 | CN20 |
| As21 | BN18 | CN21 |
| As22 | BN18 | CN22 |
| As23 | BN18 | CN23 |
| As24 | BN18 | CN24 |
| As25 | BN18 | CN25 |
| As26 | BN18 | CN26 |
| As27 | BN18 | CN27 |
| As28 | BN18 | CN28 |
| As29 | BN18 | CN29 |
| As30 | BN18 | CN30 |
| As31 | BN18 | CN31 |
| As32 | BN18 | CN32 |
| As33 | BN18 | CN33 |
| As34 | BN18 | CN34 |
| As35 | BN18 | CN35 |
| As36 | BN18 | CN36 |
| As37 | BN18 | CN37 |
| As38 | BN18 | CN38 |
| As39 | BN18 | CN39 |
| As40 | BN18 | CN40 |
| As41 | BN18 | CN41 |
| As42 | BN18 | CN42 |
| As43 | BN18 | CN43 |
| As44 | BN18 | CN44 |
| As45 | BN18 | CN45 |
| As46 | BN18 | CN46 |
| At1 | BN19 | CN1 |
| At2 | BN19 | CN2 |
| At3 | BN19 | CN3 |
| At4 | BN19 | CN4 |
| At5 | BN19 | CN5 |
| At6 | BN19 | CN6 |
| At7 | BN19 | CN7 |
| At8 | BN19 | CN8 |
| At9 | BN19 | CN9 |
| At10 | BN19 | CN10 |
| At11 | BN19 | CN11 |
| At12 | BN19 | CN12 |
| At13 | BN19 | CN13 |
| At14 | BN19 | CN14 |
| At15 | BN19 | CN15 |
| At16 | BN19 | CN16 |
| At17 | BN19 | CN17 |
| At18 | BN19 | CN18 |
| At19 | BN19 | CN19 |
| At20 | BN19 | CN20 |
| At21 | BN19 | CN21 |
| At22 | BN19 | CN22 |
| At23 | BN19 | CN23 |
| At24 | BN19 | CN24 |
| At25 | BN19 | CN25 |
| At26 | BN19 | CN26 |
| At27 | BN19 | CN27 |
| At28 | BN19 | CN28 |
| At29 | BN19 | CN29 |
| At30 | BN19 | CN30 |
| At31 | BN19 | CN31 |
| At32 | BN19 | CN32 |
| At33 | BN19 | CN33 |
| At34 | BN19 | CN34 |
| At35 | BN19 | CN35 |
| At36 | BN19 | CN36 |
| At37 | BN19 | CN37 |
| At38 | BN19 | CN38 |
| At39 | BN19 | CN39 |
| At40 | BN19 | CN40 |
| At41 | BN19 | CN41 |
| At42 | BN19 | CN42 |
| At43 | BN19 | CN43 |
| At44 | BN19 | CN44 |
| At45 | BN19 | CN45 |
| At46 | BN19 | CN46 |
| Au1 | BN20 | CN1 |
| Au2 | BN20 | CN2 |
| Au3 | BN20 | CN3 |
| Au4 | BN20 | CN4 |
| Au5 | BN20 | CN5 |
| Au6 | BN20 | CN6 |
| Au7 | BN20 | CN7 |
| Au8 | BN20 | CN8 |
| Au9 | BN20 | CN9 |
| Au10 | BN20 | CN10 |
| Au11 | BN20 | CN11 |
| Au12 | BN20 | CN12 |
| Au13 | BN20 | CN13 |
| Au14 | BN20 | CN14 |
| Au15 | BN20 | CN15 |
| Au16 | BN20 | CN16 |
| Au17 | BN20 | CN17 |
| Au18 | BN20 | CN18 |
| Au19 | BN20 | CN19 |
| Au20 | BN20 | CN20 |
| Au21 | BN20 | CN21 |
| Au22 | BN20 | CN22 |
| Au23 | BN20 | CN23 |
| Au24 | BN20 | CN24 |
| Au25 | BN20 | CN25 |
| Au26 | BN20 | CN26 |
| Au27 | BN20 | CN27 |
| Au28 | BN20 | CN28 |
| Au29 | BN20 | CN29 |
| Au30 | BN20 | CN30 |
| Au31 | BN20 | CN31 |
| Au32 | BN20 | CN32 |
| Au33 | BN20 | CN33 |
| Au34 | BN20 | CN34 |
| Au35 | BN20 | CN35 |
| Au36 | BN20 | CN36 |
| Au37 | BN20 | CN37 |
| Au38 | BN20 | CN38 |
| Au39 | BN20 | CN39 |
| Au40 | BN20 | CN40 |
| Au41 | BN20 | CN41 |
| Au42 | BN20 | CN42 |
| Au43 | BN20 | CN43 |
| Au44 | BN20 | CN44 |
| Au45 | BN20 | CN45 |
| Au46 | BN20 | CN46 |
| Av1 | BN21 | CN1 |
| Av2 | BN21 | CN2 |
| Av3 | BN21 | CN3 |
| Av4 | BN21 | CN4 |
| Av5 | BN21 | CN5 |
| Av6 | BN21 | CN6 |
| Av7 | BN21 | CN7 |
| Av8 | BN21 | CN8 |
| Av9 | BN21 | CN9 |
| Av10 | BN21 | CN10 |
| Av11 | BN21 | CN11 |
| Av12 | BN21 | CN12 |
| Av13 | BN21 | CN13 |
| Av14 | BN21 | CN14 |
| Av15 | BN21 | CN15 |
| Av16 | BN21 | CN16 |
| Av17 | BN21 | CN17 |
| Av18 | BN21 | CN18 |
| Av19 | BN21 | CN19 |
| Av20 | BN21 | CN20 |
| Av21 | BN21 | CN21 |
| Av22 | BN21 | CN22 |
| Av23 | BN21 | CN23 |
| Av24 | BN21 | CN24 |
| Av25 | BN21 | CN25 |
| Av26 | BN21 | CN26 |
| Av27 | BN21 | CN27 |
| Av28 | BN21 | CN28 |
| Av29 | BN21 | CN29 |
| Av30 | BN21 | CN30 |
| Av31 | BN21 | CN31 |
| Av32 | BN21 | CN32 |
| Av33 | BN21 | CN33 |
| Av34 | BN21 | CN34 |
| Av35 | BN21 | CN35 |
| Av36 | BN21 | CN36 |
| Av37 | BN21 | CN37 |
| Av38 | BN21 | CN38 |
| Av39 | BN21 | CN39 |
| Av40 | BN21 | CN40 |
| Av41 | BN21 | CN41 |
| Av42 | BN21 | CN42 |
| Av43 | BN21 | CN43 |
| Av44 | BN21 | CN44 |
| Av45 | BN21 | CN45 |
| Av46 | BN21 | CN46 |

TABLE 2

| Name of | | |
|---|---|---|
| compound | R_{B1} | R_{B2} |
| Ba1 | BN1 | CN1 |
| Ba2 | BN1 | CN2 |
| Ba3 | BN1 | CN3 |
| Ba4 | BN1 | CN4 |
| Ba5 | BN1 | CN5 |
| Ba6 | BN1 | CN6 |
| Ba7 | BN1 | CN7 |
| Ba8 | BN1 | CN8 |
| Ba9 | BN1 | CN9 |
| Ba10 | BN1 | CN10 |
| Ba11 | BN1 | CN11 |
| Ba12 | BN1 | CN12 |
| Ba13 | BN1 | CN13 |
| Ba14 | BN1 | CN14 |
| Ba15 | BN1 | CN15 |
| Ba16 | BN1 | CN16 |
| Ba17 | BN1 | CN17 |
| Ba18 | BN1 | CN18 |
| Ba19 | BN1 | CN19 |
| Ba20 | BN1 | CN20 |
| Ba21 | BN1 | CN21 |
| Ba22 | BN1 | CN22 |
| Ba23 | BN1 | CN23 |
| Ba24 | BN1 | CN24 |
| Ba25 | BN1 | CN25 |
| Ba26 | BN1 | CN26 |
| Ba27 | BN1 | CN27 |
| Ba28 | BN1 | CN28 |
| Ba29 | BN1 | CN29 |
| Ba30 | BN1 | CN30 |
| Ba31 | BN1 | CN31 |
| Ba32 | BN1 | CN32 |
| Ba33 | BN1 | CN33 |
| Ba34 | BN1 | CN34 |
| Ba35 | BN1 | CN35 |
| Ba36 | BN1 | CN36 |
| Ba37 | BN1 | CN37 |
| Ba38 | BN1 | CN38 |
| Ba39 | BN1 | CN39 |
| Ba40 | BN1 | CN40 |
| Ba41 | BN1 | CN41 |
| Ba42 | BN1 | CN42 |
| Ba43 | BN1 | CN43 |
| Ba44 | BN1 | CN44 |
| Ba45 | BN1 | CN45 |
| Ba46 | BN1 | CN46 |
| Bb1 | BN2 | CN1 |
| Bb2 | BN2 | CN2 |
| Bb3 | BN2 | CN3 |
| Bb4 | BN2 | CN4 |
| Bb5 | BN2 | CN5 |
| Bb6 | BN2 | CN6 |
| Bb7 | BN2 | CN7 |
| Bb8 | BN2 | CN8 |
| Bb9 | BN2 | CN9 |
| Bb10 | BN2 | CN10 |
| Bb11 | BN2 | CN11 |
| Bb12 | BN2 | CN12 |
| Bb13 | BN2 | CN13 |
| Bb14 | BN2 | CN14 |
| Bb15 | BN2 | CN15 |
| Bb16 | BN2 | CN16 |
| Bb17 | BN2 | CN17 |
| Bb18 | BN2 | CN18 |
| Bb19 | BN2 | CN19 |
| Bb20 | BN2 | CN20 |
| Bb21 | BN2 | CN21 |
| Bb22 | BN2 | CN22 |
| Bb23 | BN2 | CN23 |
| Bb24 | BN2 | CN24 |
| Bb25 | BN2 | CN25 |
| Bb26 | BN2 | CN26 |
| Bb27 | BN2 | CN27 |
| Bb28 | BN2 | CN28 |
| Bb29 | BN2 | CN29 |
| Bb30 | BN2 | CN30 |
| Bb31 | BN2 | CN31 |
| Bb32 | BN2 | CN32 |
| Bb33 | BN2 | CN33 |
| Bb34 | BN2 | CN34 |
| Bb35 | BN2 | CN35 |
| Bb36 | BN2 | CN36 |
| Bb37 | BN2 | CN37 |
| Bb38 | BN2 | CN38 |
| Bb39 | BN2 | CN39 |
| Bb40 | BN2 | CN40 |
| Bb41 | BN2 | CN41 |
| Bb42 | BN2 | CN42 |
| Bb43 | BN2 | CN43 |
| Bb44 | BN2 | CN44 |
| Bb45 | BN2 | CN45 |
| Bb46 | BN2 | CN46 |
| Bc1 | BN3 | CN1 |
| Bc2 | BN3 | CN2 |
| Bc3 | BN3 | CN3 |
| Bc4 | BN3 | CN4 |
| Bc5 | BN3 | CN5 |
| Bc6 | BN3 | CN6 |
| Bc7 | BN3 | CN7 |
| Bc8 | BN3 | CN8 |
| Bc9 | BN3 | CN9 |
| Bc10 | BN3 | CN10 |
| Bc11 | BN3 | CN11 |
| Bc12 | BN3 | CN12 |
| Bc13 | BN3 | CN13 |
| Bc14 | BN3 | CN14 |
| Bc15 | BN3 | CN15 |
| Bc16 | BN3 | CN16 |
| Bc17 | BN3 | CN17 |
| Bc18 | BN3 | CN18 |
| Bc19 | BN3 | CN19 |
| Bc20 | BN3 | CN20 |
| Bc21 | BN3 | CN21 |
| Bc22 | BN3 | CN22 |
| Bc23 | BN3 | CN23 |
| Bc24 | BN3 | CN24 |
| Bc25 | BN3 | CN25 |
| Bc26 | BN3 | CN26 |
| Bc27 | BN3 | CN27 |
| Bc28 | BN3 | CN28 |
| Bc29 | BN3 | CN29 |
| Bc30 | BN3 | CN30 |
| Bc31 | BN3 | CN31 |
| Bc32 | BN3 | CN32 |
| Bc33 | BN3 | CN33 |
| Bc34 | BN3 | CN34 |
| Bc35 | BN3 | CN35 |
| Bc36 | BN3 | CN36 |
| Bc37 | BN3 | CN37 |
| Bc38 | BN3 | CN38 |
| Bc39 | BN3 | CN39 |
| Bc40 | BN3 | CN40 |
| Bc41 | BN3 | CN41 |
| Bc42 | BN3 | CN42 |
| Bc43 | BN3 | CN43 |
| Bc44 | BN3 | CN44 |
| Bc45 | BN3 | CN45 |
| Bc46 | BN3 | CN46 |
| Bd1 | BN4 | CN1 |
| Bd2 | BN4 | CN2 |
| Bd3 | BN4 | CN3 |
| Bd4 | BN4 | CN4 |
| Bd5 | BN4 | CN5 |
| Bd6 | BN4 | CN6 |
| Bd7 | BN4 | CN7 |
| Bd8 | BN4 | CN8 |
| Bd9 | BN4 | CN9 |
| Bd10 | BN4 | CN10 |
| Bd11 | BN4 | CN11 |
| Bd12 | BN4 | CN12 |
| Bd13 | BN4 | CN13 |
| Bd14 | BN4 | CN14 |
| Bd15 | BN4 | CN15 |
| Bd16 | BN4 | CN16 |
| Bd17 | BN4 | CN17 |
| Bd18 | BN4 | CN18 |
| Bd19 | BN4 | CN19 |
| Bd20 | BN4 | CN20 |
| Bd21 | BN4 | CN21 |
| Bd22 | BN4 | CN22 |
| Bd23 | BN4 | CN23 |
| Bd24 | BN4 | CN24 |
| Bd25 | BN4 | CN25 |
| Bd26 | BN4 | CN26 |
| Bd27 | BN4 | CN27 |
| Bd28 | BN4 | CN28 |
| Bd29 | BN4 | CN29 |
| Bd30 | BN4 | CN30 |
| Bd31 | BN4 | CN31 |
| Bd32 | BN4 | CN32 |
| Bd33 | BN4 | CN33 |
| Bd34 | BN4 | CN34 |
| Bd35 | BN4 | CN35 |
| Bd36 | BN4 | CN36 |
| Bd37 | BN4 | CN37 |
| Bd38 | BN4 | CN38 |
| Bd39 | BN4 | CN39 |
| Bd40 | BN4 | CN40 |
| Bd41 | BN4 | CN41 |
| Bd42 | BN4 | CN42 |
| Bd43 | BN4 | CN43 |
| Bd44 | BN4 | CN44 |
| Bd45 | BN4 | CN45 |
| Bd46 | BN4 | CN46 |
| Be1 | BN5 | CN1 |
| Be2 | BN5 | CN2 |
| Be3 | BN5 | CN3 |
| Be4 | BN5 | CN4 |
| Be5 | BN5 | CN5 |
| Be6 | BN5 | CN6 |
| Be7 | BN5 | CN7 |
| Be8 | BN5 | CN8 |
| Be9 | BN5 | CN9 |
| Be10 | BN5 | CN10 |
| Be11 | BN5 | CN11 |
| Be12 | BN5 | CN12 |
| Be13 | BN5 | CN13 |
| Be14 | BN5 | CN14 |
| Be15 | BN5 | CN15 |
| Be16 | BN5 | CN16 |
| Be17 | BN5 | CN17 |
| Be18 | BN5 | CN18 |
| Be19 | BN5 | CN19 |
| Be20 | BN5 | CN20 |
| Be21 | BN5 | CN21 |
| Be22 | BN5 | CN22 |
| Be23 | BN5 | CN23 |
| Be24 | BN5 | CN24 |
| Be25 | BN5 | CN25 |
| Be26 | BN5 | CN26 |
| Be27 | BN5 | CN27 |
| Be28 | BN5 | CN28 |
| Be29 | BN5 | CN29 |
| Be30 | BN5 | CN30 |
| Be31 | BN5 | CN31 |
| Be32 | BN5 | CN32 |
| Be33 | BN5 | CN33 |
| Be34 | BN5 | CN34 |
| Be35 | BN5 | CN35 |
| Be36 | BN5 | CN36 |
| Be37 | BN5 | CN37 |
| Be38 | BN5 | CN38 |
| Be39 | BN5 | CN39 |
| Be40 | BN5 | CN40 |
| Be41 | BN5 | CN41 |
| Be42 | BN5 | CN42 |
| Be43 | BN5 | CN43 |
| Be44 | BN5 | CN44 |
| Be45 | BN5 | CN45 |
| Be46 | BN5 | CN46 |
| Bf1 | BN6 | CN1 |
| Bf2 | BN6 | CN2 |
| Bf3 | BN6 | CN3 |
| Bf4 | BN6 | CN4 |
| Bf5 | BN6 | CN5 |
| Bf6 | BN6 | CN6 |
| Bf7 | BN6 | CN7 |
| Bf8 | BN6 | CN8 |
| Bf9 | BN6 | CN9 |
| Bf10 | BN6 | CN10 |
| Bf11 | BN6 | CN11 |
| Bf12 | BN6 | CN12 |
| Bf13 | BN6 | CN13 |
| Bf14 | BN6 | CN14 |
| Bf15 | BN6 | CN15 |
| Bf16 | BN6 | CN16 |
| Bf17 | BN6 | CN17 |
| Bf18 | BN6 | CN18 |
| Bf19 | BN6 | CN19 |
| Bf20 | BN6 | CN20 |
| Bf21 | BN6 | CN21 |
| Bf22 | BN6 | CN22 |
| Bf23 | BN6 | CN23 |
| Bf24 | BN6 | CN24 |
| Bf25 | BN6 | CN25 |
| Bf26 | BN6 | CN26 |
| Bf27 | BN6 | CN27 |
| Bf28 | BN6 | CN28 |
| Bf29 | BN6 | CN29 |
| Bf30 | BN6 | CN30 |
| Bf31 | BN6 | CN31 |
| Bf32 | BN6 | CN32 |
| Bf33 | BN6 | CN33 |
| Bf34 | BN6 | CN34 |
| Bf35 | BN6 | CN35 |
| Bf36 | BN6 | CN36 |
| Bf37 | BN6 | CN37 |
| Bf38 | BN6 | CN38 |
| Bf39 | BN6 | CN39 |
| Bf40 | BN6 | CN40 |
| Bf41 | BN6 | CN41 |
| Bf42 | BN6 | CN42 |
| Bf43 | BN6 | CN43 |
| Bf44 | BN6 | CN44 |
| Bf45 | BN6 | CN45 |
| Bf46 | BN6 | CN46 |
| Bg1 | BN7 | CN1 |
| Bg2 | BN7 | CN2 |
| Bg3 | BN7 | CN3 |
| Bg4 | BN7 | CN4 |
| Bg5 | BN7 | CN5 |
| Bg6 | BN7 | CN6 |
| Bg7 | BN7 | CN7 |
| Bg8 | BN7 | CN8 |
| Bg9 | BN7 | CN9 |
| Bg10 | BN7 | CN10 |
| Bg11 | BN7 | CN11 |
| Bg12 | BN7 | CN12 |
| Bg13 | BN7 | CN13 |
| Bg14 | BN7 | CN14 |
| Bg15 | BN7 | CN15 |
| Bg16 | BN7 | CN16 |
| Bg17 | BN7 | CN17 |
| Bg18 | BN7 | CN18 |
| Bg19 | BN7 | CN19 |
| Bg20 | BN7 | CN20 |
| Bg21 | BN7 | CN21 |
| Bg22 | BN7 | CN22 |
| Bg23 | BN7 | CN23 |
| Bg24 | BN7 | CN24 |
| Bg25 | BN7 | CN25 |
| Bg26 | BN7 | CN26 |
| Bg27 | BN7 | CN27 |
| Bg28 | BN7 | CN28 |
| Bg29 | BN7 | CN29 |
| Bg30 | BN7 | CN30 |
| Bg31 | BN7 | CN31 |
| Bg32 | BN7 | CN32 |
| Bg33 | BN7 | CN33 |
| Bg34 | BN7 | CN34 |
| Bg35 | BN7 | CN35 |
| Bg36 | BN7 | CN36 |
| Bg37 | BN7 | CN37 |
| Bg38 | BN7 | CN38 |
| Bg39 | BN7 | CN39 |
| Bg40 | BN7 | CN40 |
| Bg41 | BN7 | CN41 |
| Bg42 | BN7 | CN42 |
| Bg43 | BN7 | CN43 |
| Bg44 | BN7 | CN44 |
| Bg45 | BN7 | CN45 |
| Bg46 | BN7 | CN46 |
| Bh1 | BN8 | CN1 |
| Bh2 | BN8 | CN2 |
| Bh3 | BN8 | CN3 |
| Bh4 | BN8 | CN4 |
| Bh5 | BN8 | CN5 |
| Bh6 | BN8 | CN6 |
| Bh7 | BN8 | CN7 |
| Bh8 | BN8 | CN8 |
| Bh9 | BN8 | CN9 |
| Bh10 | BN8 | CN10 |
| Bh11 | BN8 | CN11 |
| Bh12 | BN8 | CN12 |
| Bh13 | BN8 | CN13 |
| Bh14 | BN8 | CN14 |
| Bh15 | BN8 | CN15 |
| Bh16 | BN8 | CN16 |
| Bh17 | BN8 | CN17 |
| Bh18 | BN8 | CN18 |
| Bh19 | BN8 | CN19 |
| Bh20 | BN8 | CN20 |
| Bh21 | BN8 | CN21 |
| Bh22 | BN8 | CN22 |
| Bh23 | BN8 | CN23 |
| Bh24 | BN8 | CN24 |
| Bh25 | BN8 | CN25 |
| Bh26 | BN8 | CN26 |
| Bh27 | BN8 | CN27 |
| Bh28 | BN8 | CN28 |
| Bh29 | BN8 | CN29 |
| Bh30 | BN8 | CN30 |
| Bh31 | BN8 | CN31 |
| Bh32 | BN8 | CN32 |
| Bh33 | BN8 | CN33 |
| Bh34 | BN8 | CN34 |
| Bh35 | BN8 | CN35 |
| Bh36 | BN8 | CN36 |
| Bh37 | BN8 | CN37 |
| Bh38 | BN8 | CN38 |
| Bh39 | BN8 | CN39 |
| Bh40 | BN8 | CN40 |
| Bh41 | BN8 | CN41 |
| Bh42 | BN8 | CN42 |
| Bh43 | BN8 | CN43 |
| Bh44 | BN8 | CN44 |
| Bh45 | BN8 | CN45 |
| Bh46 | BN8 | CN46 |
| Bi1 | BN9 | CN1 |
| Bi2 | BN9 | CN2 |
| Bi3 | BN9 | CN3 |
| Bi4 | BN9 | CN4 |
| Bi5 | BN9 | CN5 |
| Bi6 | BN9 | CN6 |
| Bi7 | BN9 | CN7 |
| Bi8 | BN9 | CN8 |
| Bi9 | BN9 | CN9 |
| Bi10 | BN9 | CN10 |
| Bi11 | BN9 | CN11 |
| Bi12 | BN9 | CN12 |
| Bi13 | BN9 | CN13 |
| Bi14 | BN9 | CN14 |
| Bi15 | BN9 | CN15 |
| Bi16 | BN9 | CN16 |
| Bi17 | BN9 | CN17 |
| Bi18 | BN9 | CN18 |
| Bi19 | BN9 | CN19 |
| Bi20 | BN9 | CN20 |
| Bi21 | BN9 | CN21 |
| Bi22 | BN9 | CN22 |
| Bi23 | BN9 | CN23 |
| Bi24 | BN9 | CN24 |
| Bi25 | BN9 | CN25 |
| Bi26 | BN9 | CN26 |
| Bi27 | BN9 | CN27 |
| Bi28 | BN9 | CN28 |
| Bi29 | BN9 | CN29 |
| Bi30 | BN9 | CN30 |
| Bi31 | BN9 | CN31 |
| Bi32 | BN9 | CN32 |
| Bi33 | BN9 | CN33 |
| Bi34 | BN9 | CN34 |
| Bi35 | BN9 | CN35 |
| Bi36 | BN9 | CN36 |
| Bi37 | BN9 | CN37 |
| Bi38 | BN9 | CN38 |
| Bi39 | BN9 | CN39 |
| Bi40 | BN9 | CN40 |
| Bi41 | BN9 | CN41 |
| Bi42 | BN9 | CN42 |
| Bi43 | BN9 | CN43 |
| Bi44 | BN9 | CN44 |
| Bi45 | BN9 | CN45 |
| Bi46 | BN9 | CN46 |
| Bj1 | BN10 | CN1 |
| Bj2 | BN10 | CN2 |
| Bj3 | BN10 | CN3 |
| Bj4 | BN10 | CN4 |
| Bj5 | BN10 | CN5 |
| Bj6 | BN10 | CN6 |
| Bj7 | BN10 | CN7 |
| Bj8 | BN10 | CN8 |
| Bj9 | BN10 | CN9 |
| Bj10 | BN10 | CN10 |
| Bj11 | BN10 | CN11 |
| Bj12 | BN10 | CN12 |
| Bj13 | BN10 | CN13 |
| Bj14 | BN10 | CN14 |
| Bj15 | BN10 | CN15 |
| Bj16 | BN10 | CN16 |
| Bj17 | BN10 | CN17 |
| Bj18 | BN10 | CN18 |
| Bj19 | BN10 | CN19 |
| Bj20 | BN10 | CN20 |
| Bj21 | BN10 | CN21 |
| Bj22 | BN10 | CN22 |
| Bj23 | BN10 | CN23 |
| Bj24 | BN10 | CN24 |
| Bj25 | BN10 | CN25 |
| Bj26 | BN10 | CN26 |
| Bj27 | BN10 | CN27 |
| Bj28 | BN10 | CN28 |
| Bj29 | BN10 | CN29 |
| Bj30 | BN10 | CN30 |
| Bj31 | BN10 | CN31 |
| Bj32 | BN10 | CN32 |
| Bj33 | BN10 | CN33 |
| Bj34 | BN10 | CN34 |
| Bj35 | BN10 | CN35 |
| Bj36 | BN10 | CN36 |
| Bj37 | BN10 | CN37 |
| Bj38 | BN10 | CN38 |
| Bj39 | BN10 | CN39 |
| Bj40 | BN10 | CN40 |
| Bj41 | BN10 | CN41 |
| Bj42 | BN10 | CN42 |
| Bj43 | BN10 | CN43 |
| Bj44 | BN10 | CN44 |
| Bj45 | BN10 | CN45 |
| Bj46 | BN10 | CN46 |
| Bk1 | BN11 | CN1 |
| Bk2 | BN11 | CN2 |
| Bk3 | BN11 | CN3 |
| Bk4 | BN11 | CN4 |
| Bk5 | BN11 | CN5 |
| Bk6 | BN11 | CN6 |
| Bk7 | BN11 | CN7 |
| Bk8 | BN11 | CN8 |
| Bk9 | BN11 | CN9 |
| Bk10 | BN11 | CN10 |
| Bk11 | BN11 | CN11 |
| Bk12 | BN11 | CN12 |
| Bk13 | BN11 | CN13 |
| Bk14 | BN11 | CN14 |
| Bk15 | BN11 | CN15 |
| Bk16 | BN11 | CN16 |
| Bk17 | BN11 | CN17 |
| Bk18 | BN11 | CN18 |
| Bk19 | BN11 | CN19 |
| Bk20 | BN11 | CN20 |
| Bk21 | BN11 | CN21 |
| Bk22 | BN11 | CN22 |
| Bk23 | BN11 | CN23 |
| Bk24 | BN11 | CN24 |
| Bk25 | BN11 | CN25 |
| Bk26 | BN11 | CN26 |
| Bk27 | BN11 | CN27 |
| Bk28 | BN11 | CN28 |
| Bk29 | BN11 | CN29 |
| Bk30 | BN11 | CN30 |
| Bk31 | BN11 | CN31 |
| Bk32 | BN11 | CN32 |
| Bk33 | BN11 | CN33 |
| Bk34 | BN11 | CN34 |
| Bk35 | BN11 | CN35 |
| Bk36 | BN11 | CN36 |
| Bk37 | BN11 | CN37 |
| Bk38 | BN11 | CN38 |
| Bk39 | BN11 | CN39 |
| Bk40 | BN11 | CN40 |
| Bk41 | BN11 | CN41 |
| Bk42 | BN11 | CN42 |
| Bk43 | BN11 | CN43 |
| Bk44 | BN11 | CN44 |
| Bk45 | BN11 | CN45 |
| Bk46 | BN11 | CN46 |
| Bl1 | BN12 | CN1 |
| Bl2 | BN12 | CN2 |
| Bl3 | BN12 | CN3 |
| Bl4 | BN12 | CN4 |
| Bl5 | BN12 | CN5 |
| Bl6 | BN12 | CN6 |
| Bl7 | BN12 | CN7 |
| Bl8 | BN12 | CN8 |
| Bl9 | BN12 | CN9 |
| Bl10 | BN12 | CN10 |
| Bl11 | BN12 | CN11 |
| Bl12 | BN12 | CN12 |
| Bl13 | BN12 | CN13 |
| Bl14 | BN12 | CN14 |
| Bl15 | BN12 | CN15 |
| Bl16 | BN12 | CN16 |
| Bl17 | BN12 | CN17 |
| Bl18 | BN12 | CN18 |
| Bl19 | BN12 | CN19 |
| Bl20 | BN12 | CN20 |
| Bl21 | BN12 | CN21 |
| Bl22 | BN12 | CN22 |
| Bl23 | BN12 | CN23 |
| Bl24 | BN12 | CN24 |
| Bl25 | BN12 | CN25 |
| Bl26 | BN12 | CN26 |
| Bl27 | BN12 | CN27 |
| Bl28 | BN12 | CN28 |
| Bl29 | BN12 | CN29 |
| Bl30 | BN12 | CN30 |
| Bl31 | BN12 | CN31 |
| Bl32 | BN12 | CN32 |
| Bl33 | BN12 | CN33 |
| Bl34 | BN12 | CN34 |
| Bl35 | BN12 | CN35 |
| Bl36 | BN12 | CN36 |
| Bl37 | BN12 | CN37 |
| Bl38 | BN12 | CN38 |
| Bl39 | BN12 | CN39 |
| Bl40 | BN12 | CN40 |
| Bl41 | BN12 | CN41 |
| Bl42 | BN12 | CN42 |
| Bl43 | BN12 | CN43 |
| Bl44 | BN12 | CN44 |
| Bl45 | BN12 | CN45 |
| Bl46 | BN12 | CN46 |
| Bm1 | BN13 | CN1 |
| Bm2 | BN13 | CN2 |
| Bm3 | BN13 | CN3 |
| Bm4 | BN13 | CN4 |
| Bm5 | BN13 | CN5 |
| Bm6 | BN13 | CN6 |
| Bm7 | BN13 | CN7 |
| Bm8 | BN13 | CN8 |
| Bm9 | BN13 | CN9 |
| Bm10 | BN13 | CN10 |
| Bm11 | BN13 | CN11 |
| Bm12 | BN13 | CN12 |
| Bm13 | BN13 | CN13 |
| Bm14 | BN13 | CN14 |
| Bm15 | BN13 | CN15 |
| Bm16 | BN13 | CN16 |
| Bm17 | BN13 | CN17 |
| Bm18 | BN13 | CN18 |
| Bm19 | BN13 | CN19 |
| Bm20 | BN13 | CN20 |
| Bm21 | BN13 | CN21 |
| Bm22 | BN13 | CN22 |
| Bm23 | BN13 | CN23 |
| Bm24 | BN13 | CN24 |
| Bm25 | BN13 | CN25 |
| Bm26 | BN13 | CN26 |
| Bm27 | BN13 | CN27 |
| Bm28 | BN13 | CN28 |
| Bm29 | BN13 | CN29 |
| Bm30 | BN13 | CN30 |
| Bm31 | BN13 | CN31 |
| Bm32 | BN13 | CN32 |
| Bm33 | BN13 | CN33 |
| Bm34 | BN13 | CN34 |
| Bm35 | BN13 | CN35 |
| Bm36 | BN13 | CN36 |
| Bm37 | BN13 | CN37 |
| Bm38 | BN13 | CN38 |
| Bm39 | BN13 | CN39 |
| Bm40 | BN13 | CN40 |
| Bm41 | BN13 | CN41 |
| Bm42 | BN13 | CN42 |
| Bm43 | BN13 | CN43 |
| Bm44 | BN13 | CN44 |
| Bm45 | BN13 | CN45 |
| Bm46 | BN13 | CN46 |
| Bn1 | BN14 | CN1 |
| Bn2 | BN14 | CN2 |
| Bn3 | BN14 | CN3 |
| Bn4 | BN14 | CN4 |
| Bn5 | BN14 | CN5 |
| Bn6 | BN14 | CN6 |
| Bn7 | BN14 | CN7 |
| Bn8 | BN14 | CN8 |
| Bn9 | BN14 | CN9 |
| Bn10 | BN14 | CN10 |
| Bn11 | BN14 | CN11 |
| Bn12 | BN14 | CN12 |
| Bn13 | BN14 | CN13 |
| Bn14 | BN14 | CN14 |
| Bn15 | BN14 | CN15 |
| Bn16 | BN14 | CN16 |
| Bn17 | BN14 | CN17 |
| Bn18 | BN14 | CN18 |
| Bn19 | BN14 | CN19 |
| Bn20 | BN14 | CN20 |
| Bn21 | BN14 | CN21 |
| Bn22 | BN14 | CN22 |
| Bn23 | BN14 | CN23 |
| Bn24 | BN14 | CN24 |
| Bn25 | BN14 | CN25 |
| Bn26 | BN14 | CN26 |
| Bn27 | BN14 | CN27 |
| Bn28 | BN14 | CN28 |
| Bn29 | BN14 | CN29 |
| Bn30 | BN14 | CN30 |
| Bn31 | BN14 | CN31 |
| Bn32 | BN14 | CN32 |
| Bn33 | BN14 | CN33 |
| Bn34 | BN14 | CN34 |
| Bn35 | BN14 | CN35 |
| Bn36 | BN14 | CN36 |
| Bn37 | BN14 | CN37 |
| Bn38 | BN14 | CN38 |
| Bn39 | BN14 | CN39 |
| Bn40 | BN14 | CN40 |
| Bn41 | BN14 | CN41 |
| Bn42 | BN14 | CN42 |
| Bn43 | BN14 | CN43 |
| Bn44 | BN14 | CN44 |
| Bn45 | BN14 | CN45 |
| Bn46 | BN14 | CN46 |
| Bo1 | BN15 | CN1 |
| Bo2 | BN15 | CN2 |
| Bo3 | BN15 | CN3 |
| Bo4 | BN15 | CN4 |
| Bo5 | BN15 | CN5 |
| Bo6 | BN15 | CN6 |
| Bo7 | BN15 | CN7 |
| Bo8 | BN15 | CN8 |
| Bo9 | BN15 | CN9 |
| Bo10 | BN15 | CN10 |
| Bo11 | BN15 | CN11 |
| Bo12 | BN15 | CN12 |
| Bo13 | BN15 | CN13 |
| Bo14 | BN15 | CN14 |
| Bo15 | BN15 | CN15 |
| Bo16 | BN15 | CN16 |
| Bo17 | BN15 | CN17 |
| Bo18 | BN15 | CN18 |
| Bo19 | BN15 | CN19 |
| Bo20 | BN15 | CN20 |
| Bo21 | BN15 | CN21 |
| Bo22 | BN15 | CN22 |
| Bo23 | BN15 | CN23 |
| Bo24 | BN15 | CN24 |
| Bo25 | BN15 | CN25 |
| Bo26 | BN15 | CN26 |
| Bo27 | BN15 | CN27 |
| Bo28 | BN15 | CN28 |
| Bo29 | BN15 | CN29 |
| Bo30 | BN15 | CN30 |
| Bo31 | BN15 | CN31 |
| Bo32 | BN15 | CN32 |
| Bo33 | BN15 | CN33 |
| Bo34 | BN15 | CN34 |
| Bo35 | BN15 | CN35 |
| Bo36 | BN15 | CN36 |
| Bo37 | BN15 | CN37 |
| Bo38 | BN15 | CN38 |
| Bo39 | BN15 | CN39 |
| Bo40 | BN15 | CN40 |
| Bo41 | BN15 | CN41 |
| Bo42 | BN15 | CN42 |
| Bo43 | BN15 | CN43 |
| Bo44 | BN15 | CN44 |
| Bo45 | BN15 | CN45 |
| Bo46 | BN15 | CN46 |
| Bp1 | BN16 | CN1 |
| Bp2 | BN16 | CN2 |
| Bp3 | BN16 | CN3 |
| Bp4 | BN16 | CN4 |
| Bp5 | BN16 | CN5 |
| Bp6 | BN16 | CN6 |
| Bp7 | BN16 | CN7 |
| Bp8 | BN16 | CN8 |
| Bp9 | BN16 | CN9 |
| Bp10 | BN16 | CN10 |
| Bp11 | BN16 | CN11 |
| Bp12 | BN16 | CN12 |
| Bp13 | BN16 | CN13 |
| Bp14 | BN16 | CN14 |
| Bp15 | BN16 | CN15 |
| Bp16 | BN16 | CN16 |
| Bp17 | BN16 | CN17 |
| Bp18 | BN16 | CN18 |
| Bp19 | BN16 | CN19 |
| Bp20 | BN16 | CN20 |
| Bp21 | BN16 | CN21 |
| Bp22 | BN16 | CN22 |
| Bp23 | BN16 | CN23 |
| Bp24 | BN16 | CN24 |
| Bp25 | BN16 | CN25 |
| Bp26 | BN16 | CN26 |
| Bp27 | BN16 | CN27 |
| Bp28 | BN16 | CN28 |
| Bp29 | BN16 | CN29 |
| Bp30 | BN16 | CN30 |
| Bp31 | BN16 | CN31 |
| Bp32 | BN16 | CN32 |
| Bp33 | BN16 | CN33 |
| Bp34 | BN16 | CN34 |
| Bp35 | BN16 | CN35 |
| Bp36 | BN16 | CN36 |
| Bp37 | BN16 | CN37 |
| Bp38 | BN16 | CN38 |
| Bp39 | BN16 | CN39 |
| Bp40 | BN16 | CN40 |
| Bp41 | BN16 | CN41 |
| Bp42 | BN16 | CN42 |
| Bp43 | BN16 | CN43 |
| Bp44 | BN16 | CN44 |
| Bp45 | BN16 | CN45 |
| Bp46 | BN16 | CN46 |
| Bq1 | BN17 | CN1 |
| Bq2 | BN17 | CN2 |
| Bq3 | BN17 | CN3 |
| Bq4 | BN17 | CN4 |
| Bq5 | BN17 | CN5 |
| Bq6 | BN17 | CN6 |
| Bq7 | BN17 | CN7 |
| Bq8 | BN17 | CN8 |
| Bq9 | BN17 | CN9 |
| Bq10 | BN17 | CN10 |
| Bq11 | BN17 | CN11 |
| Bq12 | BN17 | CN12 |
| Bq13 | BN17 | CN13 |
| Bq14 | BN17 | CN14 |
| Bq15 | BN17 | CN15 |
| Bq16 | BN17 | CN16 |
| Bq17 | BN17 | CN17 |
| Bq18 | BN17 | CN18 |
| Bq19 | BN17 | CN19 |
| Bq20 | BN17 | CN20 |
| Bq21 | BN17 | CN21 |
| Bq22 | BN17 | CN22 |
| Bq23 | BN17 | CN23 |
| Bq24 | BN17 | CN24 |
| Bq25 | BN17 | CN25 |
| Bq26 | BN17 | CN26 |
| Bq27 | BN17 | CN27 |
| Bq28 | BN17 | CN28 |
| Bq29 | BN17 | CN29 |
| Bq30 | BN17 | CN30 |
| Bq31 | BN17 | CN31 |
| Bq32 | BN17 | CN32 |
| Bq33 | BN17 | CN33 |
| Bq34 | BN17 | CN34 |
| Bq35 | BN17 | CN35 |
| Bq36 | BN17 | CN36 |
| Bq37 | BN17 | CN37 |
| Bq38 | BN17 | CN38 |
| Bq39 | BN17 | CN39 |
| Bq40 | BN17 | CN40 |
| Bq41 | BN17 | CN41 |
| Bq42 | BN17 | CN42 |
| Bq43 | BN17 | CN43 |
| Bq44 | BN17 | CN44 |
| Bq45 | BN17 | CN45 |
| Bq46 | BN17 | CN46 |
| Bs1 | BN18 | CN1 |
| Bs2 | BN18 | CN2 |
| Bs3 | BN18 | CN3 |
| Bs4 | BN18 | CN4 |
| Bs5 | BN18 | CN5 |
| Bs6 | BN18 | CN6 |
| Bs7 | BN18 | CN7 |
| Bs8 | BN18 | CN8 |
| Bs9 | BN18 | CN9 |
| Bs10 | BN18 | CN10 |
| Bs11 | BN18 | CN11 |
| Bs12 | BN18 | CN12 |
| Bs13 | BN18 | CN13 |
| Bs14 | BN18 | CN14 |
| Bs15 | BN18 | CN15 |
| Bs16 | BN18 | CN16 |
| Bs17 | BN18 | CN17 |
| Bs18 | BN18 | CN18 |
| Bs19 | BN18 | CN19 |
| Bs20 | BN18 | CN20 |
| Bs21 | BN18 | CN21 |
| Bs22 | BN18 | CN22 |
| Bs23 | BN18 | CN23 |
| Bs24 | BN18 | CN24 |
| Bs25 | BN18 | CN25 |
| Bs26 | BN18 | CN26 |
| Bs27 | BN18 | CN27 |
| Bs28 | BN18 | CN28 |
| Bs29 | BN18 | CN29 |
| Bs30 | BN18 | CN30 |
| Bs31 | BN18 | CN31 |
| Bs32 | BN18 | CN32 |
| Bs33 | BN18 | CN33 |
| Bs34 | BN18 | CN34 |
| Bs35 | BN18 | CN35 |
| Bs36 | BN18 | CN36 |
| Bs37 | BN18 | CN37 |
| Bs38 | BN18 | CN38 |
| Bs39 | BN18 | CN39 |
| Bs40 | BN18 | CN40 |
| Bs41 | BN18 | CN41 |
| Bs42 | BN18 | CN42 |
| Bs43 | BN18 | CN43 |
| Bs44 | BN18 | CN44 |
| Bs45 | BN18 | CN45 |
| Bs46 | BN18 | CN46 |
| Bt1 | BN19 | CN1 |
| Bt2 | BN19 | CN2 |
| Bt3 | BN19 | CN3 |
| Bt4 | BN19 | CN4 |
| Bt5 | BN19 | CN5 |
| Bt6 | BN19 | CN6 |
| Bt7 | BN19 | CN7 |
| Bt8 | BN19 | CN8 |
| Bt9 | BN19 | CN9 |
| Bt10 | BN19 | CN10 |
| Bt11 | BN19 | CN11 |
| Bt12 | BN19 | CN12 |
| Bt13 | BN19 | CN13 |
| Bt14 | BN19 | CN14 |
| Bt15 | BN19 | CN15 |
| Bt16 | BN19 | CN16 |
| Bt17 | BN19 | CN17 |
| Bt18 | BN19 | CN18 |
| Bt19 | BN19 | CN19 |
| Bt20 | BN19 | CN20 |
| Bt21 | BN19 | CN21 |
| Bt22 | BN19 | CN22 |
| Bt23 | BN19 | CN23 |
| Bt24 | BN19 | CN24 |
| Bt25 | BN19 | CN25 |
| Bt26 | BN19 | CN26 |
| Bt27 | BN19 | CN27 |
| Bt28 | BN19 | CN28 |
| Bt29 | BN19 | CN29 |
| Bt30 | BN19 | CN30 |
| Bt31 | BN19 | CN31 |
| Bt32 | BN19 | CN32 |
| Bt33 | BN19 | CN33 |
| Bt34 | BN19 | CN34 |
| Bt35 | BN19 | CN35 |
| Bt36 | BN19 | CN36 |
| Bt37 | BN19 | CN37 |
| Bt38 | BN19 | CN38 |
| Bt39 | BN19 | CN39 |
| Bt40 | BN19 | CN40 |
| Bt41 | BN19 | CN41 |
| Bt42 | BN19 | CN42 |
| Bt43 | BN19 | CN43 |
| Bt44 | BN19 | CN44 |
| Bt45 | BN19 | CN45 |
| Bt46 | BN19 | CN46 |
| Bu1 | BN20 | CN1 |
| Bu2 | BN20 | CN2 |
| Bu3 | BN20 | CN3 |
| Bu4 | BN20 | CN4 |
| Bu5 | BN20 | CN5 |
| Bu6 | BN20 | CN6 |
| Bu7 | BN20 | CN7 |
| Bu8 | BN20 | CN8 |
| Bu9 | BN20 | CN9 |
| Bu10 | BN20 | CN10 |
| Bu11 | BN20 | CN11 |
| Bu12 | BN20 | CN12 |
| Bu13 | BN20 | CN13 |
| Bu14 | BN20 | CN14 |
| Bu15 | BN20 | CN15 |
| Bu16 | BN20 | CN16 |
| Bu17 | BN20 | CN17 |
| Bu18 | BN20 | CN18 |
| Bu19 | BN20 | CN19 |
| Bu20 | BN20 | CN20 |
| Bu21 | BN20 | CN21 |
| Bu22 | BN20 | CN22 |
| Bu23 | BN20 | CN23 |
| Bu24 | BN20 | CN24 |
| Bu25 | BN20 | CN25 |
| Bu26 | BN20 | CN26 |
| Bu27 | BN20 | CN27 |
| Bu28 | BN20 | CN28 |
| Bu29 | BN20 | CN29 |
| Bu30 | BN20 | CN30 |
| Bu31 | BN20 | CN31 |
| Bu32 | BN20 | CN32 |
| Bu33 | BN20 | CN33 |
| Bu34 | BN20 | CN34 |
| Bu35 | BN20 | CN35 |
| Bu36 | BN20 | CN36 |
| Bu37 | BN20 | CN37 |
| Bu38 | BN20 | CN38 |
| Bu39 | BN20 | CN39 |
| Bu40 | BN20 | CN40 |
| Bu41 | BN20 | CN41 |
| Bu42 | BN20 | CN42 |
| Bu43 | BN20 | CN43 |
| Bu44 | BN20 | CN44 |
| Bu45 | BN20 | CN45 |
| Bu46 | BN20 | CN46 |
| Bv1 | BN21 | CN1 |
| Bv2 | BN21 | CN2 |
| Bv3 | BN21 | CN3 |
| Bv4 | BN21 | CN4 |
| Bv5 | BN21 | CN5 |
| Bv6 | BN21 | CN6 |
| Bv7 | BN21 | CN7 |
| Bv8 | BN21 | CN8 |
| Bv9 | BN21 | CN9 |
| Bv10 | BN21 | CN10 |
| Bv11 | BN21 | CN11 |
| Bv12 | BN21 | CN12 |
| Bv13 | BN21 | CN13 |
| Bv14 | BN21 | CN14 |
| Bv15 | BN21 | CN15 |
| Bv16 | BN21 | CN16 |
| Bv17 | BN21 | CN17 |
| Bv18 | BN21 | CN18 |
| Bv19 | BN21 | CN19 |
| Bv20 | BN21 | CN20 |
| Bv21 | BN21 | CN21 |
| Bv22 | BN21 | CN22 |
| Bv23 | BN21 | CN23 |
| Bv24 | BN21 | CN24 |
| Bv25 | BN21 | CN25 |
| Bv26 | BN21 | CN26 |
| Bv27 | BN21 | CN27 |
| Bv28 | BN21 | CN28 |
| Bv29 | BN21 | CN29 |
| Bv30 | BN21 | CN30 |
| Bv31 | BN21 | CN31 |
| Bv32 | BN21 | CN32 |
| Bv33 | BN21 | CN33 |
| Bv34 | BN21 | CN34 |
| Bv35 | BN21 | CN35 |
| Bv36 | BN21 | CN36 |
| Bv37 | BN21 | CN37 |
| Bv38 | BN21 | CN38 |
| Bv39 | BN21 | CN39 |
| Bv40 | BN21 | CN40 |
| Bv41 | BN21 | CN41 |
| Bv42 | BN21 | CN42 |
| Bv43 | BN21 | CN43 |
| Bv44 | BN21 | CN44 |
| Bv45 | BN21 | CN45 |
| Bv46 | BN21 | CN46 |

TABLE 3

| Name of | | |
|---|---|---|
| compound | R_{C1} | R_{C2} |
| Ca1 | BN1 | CN1 |
| Ca2 | BN1 | CN2 |
| Ca3 | BN1 | CN3 |
| Ca4 | BN1 | CN4 |
| Ca5 | BN1 | CN5 |
| Ca6 | BN1 | CN6 |
| Ca7 | BN1 | CN7 |
| Ca8 | BN1 | CN8 |
| Ca9 | BN1 | CN9 |
| Ca10 | BN1 | CN10 |
| Ca11 | BN1 | CN11 |
| Ca12 | BN1 | CN12 |
| Ca13 | BN1 | CN13 |
| Ca14 | BN1 | CN14 |
| Ca15 | BN1 | CN15 |
| Ca16 | BN1 | CN16 |
| Ca17 | BN1 | CN17 |
| Ca18 | BN1 | CN18 |
| Ca19 | BN1 | CN19 |
| Ca20 | BN1 | CN20 |
| Ca21 | BN1 | CN21 |
| Ca22 | BN1 | CN22 |
| Ca23 | BN1 | CN23 |
| Ca24 | BN1 | CN24 |
| Ca25 | BN1 | CN25 |
| Ca26 | BN1 | CN26 |
| Ca27 | BN1 | CN27 |
| Ca28 | BN1 | CN28 |
| Ca29 | BN1 | CN29 |
| Ca30 | BN1 | CN30 |
| Ca31 | BN1 | CN31 |
| Ca32 | BN1 | CN32 |
| Ca33 | BN1 | CN33 |
| Ca34 | BN1 | CN34 |
| Ca35 | BN1 | CN35 |
| Ca36 | BN1 | CN36 |
| Ca37 | BN1 | CN37 |
| Ca38 | BN1 | CN38 |
| Ca39 | BN1 | CN39 |
| Ca40 | BN1 | CN40 |
| Ca41 | BN1 | CN41 |
| Ca42 | BN1 | CN42 |
| Ca43 | BN1 | CN43 |
| Ca44 | BN1 | CN44 |
| Ca45 | BN1 | CN45 |
| Ca46 | BN1 | CN46 |
| Cb1 | BN2 | CN1 |
| Cb2 | BN2 | CN2 |
| Cb3 | BN2 | CN3 |
| Cb4 | BN2 | CN4 |
| Cb5 | BN2 | CN5 |
| Cb6 | BN2 | CN6 |
| Cb7 | BN2 | CN7 |
| Cb8 | BN2 | CN8 |
| Cb9 | BN2 | CN9 |
| Cb10 | BN2 | CN10 |
| Cb11 | BN2 | CN11 |
| Cb12 | BN2 | CN12 |
| Cb13 | BN2 | CN13 |
| Cb14 | BN2 | CN14 |
| Cb15 | BN2 | CN15 |
| Cb16 | BN2 | CN16 |
| Cb17 | BN2 | CN17 |
| Cb18 | BN2 | CN18 |
| Cb19 | BN2 | CN19 |
| Cb20 | BN2 | CN20 |
| Cb21 | BN2 | CN21 |
| Cb22 | BN2 | CN22 |
| Cb23 | BN2 | CN23 |
| Cb24 | BN2 | CN24 |
| Cb25 | BN2 | CN25 |
| Cb26 | BN2 | CN26 |
| Cb27 | BN2 | CN27 |
| Cb28 | BN2 | CN28 |
| Cb29 | BN2 | CN29 |
| Cb30 | BN2 | CN30 |
| Cb31 | BN2 | CN31 |
| Cb32 | BN2 | CN32 |
| Cb33 | BN2 | CN33 |
| Cb34 | BN2 | CN34 |
| Cb35 | BN2 | CN35 |
| Cb36 | BN2 | CN36 |
| Cb37 | BN2 | CN37 |
| Cb38 | BN2 | CN38 |
| Cb39 | BN2 | CN39 |
| Cb40 | BN2 | CN40 |
| Cb41 | BN2 | CN41 |
| Cb42 | BN2 | CN42 |
| Cb43 | BN2 | CN43 |
| Cb44 | BN2 | CN44 |
| Cb45 | BN2 | CN45 |
| Cb46 | BN2 | CN46 |
| Cc1 | BN3 | CN1 |
| Cc2 | BN3 | CN2 |
| Cc3 | BN3 | CN3 |
| Cc4 | BN3 | CN4 |
| Cc5 | BN3 | CN5 |
| Cc6 | BN3 | CN6 |
| Cc7 | BN3 | CN7 |
| Cc8 | BN3 | CN8 |
| Cc9 | BN3 | CN9 |
| Cc10 | BN3 | CN10 |
| Cc11 | BN3 | CN11 |
| Cc12 | BN3 | CN12 |
| Cc13 | BN3 | CN13 |
| Cc14 | BN3 | CN14 |
| Cc15 | BN3 | CN15 |
| Cc16 | BN3 | CN16 |
| Cc17 | BN3 | CN17 |
| Cc18 | BN3 | CN18 |
| Cc19 | BN3 | CN19 |
| Cc20 | BN3 | CN20 |
| Cc21 | BN3 | CN21 |
| Cc22 | BN3 | CN22 |
| Cc23 | BN3 | CN23 |
| Cc24 | BN3 | CN24 |
| Cc25 | BN3 | CN25 |
| Cc26 | BN3 | CN26 |
| Cc27 | BN3 | CN27 |
| Cc28 | BN3 | CN28 |
| Cc29 | BN3 | CN29 |
| Cc30 | BN3 | CN30 |
| Cc31 | BN3 | CN31 |
| Cc32 | BN3 | CN32 |
| Cc33 | BN3 | CN33 |
| Cc34 | BN3 | CN34 |
| Cc35 | BN3 | CN35 |
| Cc36 | BN3 | CN36 |
| Cc37 | BN3 | CN37 |
| Cc38 | BN3 | CN38 |
| Cc39 | BN3 | CN39 |
| Cc40 | BN3 | CN40 |
| Cc41 | BN3 | CN41 |
| Cc42 | BN3 | CN42 |
| Cc43 | BN3 | CN43 |
| Cc44 | BN3 | CN44 |
| Cc45 | BN3 | CN45 |
| Cc46 | BN3 | CN46 |
| Cd1 | BN4 | CN1 |
| Cd2 | BN4 | CN2 |
| Cd3 | BN4 | CN3 |
| Cd4 | BN4 | CN4 |
| Cd5 | BN4 | CN5 |
| Cd6 | BN4 | CN6 |
| Cd7 | BN4 | CN7 |
| Cd8 | BN4 | CN8 |
| Cd9 | BN4 | CN9 |
| Cd10 | BN4 | CN10 |
| Cd11 | BN4 | CN11 |
| Cd12 | BN4 | CN12 |
| Cd13 | BN4 | CN13 |
| Cd14 | BN4 | CN14 |
| Cd15 | BN4 | CN15 |
| Cd16 | BN4 | CN16 |
| Cd17 | BN4 | CN17 |
| Cd18 | BN4 | CN18 |
| Cd19 | BN4 | CN19 |
| Cd20 | BN4 | CN20 |
| Cd21 | BN4 | CN21 |
| Cd22 | BN4 | CN22 |
| Cd23 | BN4 | CN23 |
| Cd24 | BN4 | CN24 |
| Cd25 | BN4 | CN25 |
| Cd26 | BN4 | CN26 |
| Cd27 | BN4 | CN27 |
| Cd28 | BN4 | CN28 |
| Cd29 | BN4 | CN29 |
| Cd30 | BN4 | CN30 |
| Cd31 | BN4 | CN31 |
| Cd32 | BN4 | CN32 |
| Cd33 | BN4 | CN33 |
| Cd34 | BN4 | CN34 |
| Cd35 | BN4 | CN35 |
| Cd36 | BN4 | CN36 |
| Cd37 | BN4 | CN37 |
| Cd38 | BN4 | CN38 |
| Cd39 | BN4 | CN39 |
| Cd40 | BN4 | CN40 |
| Cd41 | BN4 | CN41 |
| Cd42 | BN4 | CN42 |
| Cd43 | BN4 | CN43 |
| Cd44 | BN4 | CN44 |
| Cd45 | BN4 | CN45 |
| Cd46 | BN4 | CN46 |
| Ce1 | BN5 | CN1 |
| Ce2 | BN5 | CN2 |
| Ce3 | BN5 | CN3 |
| Ce4 | BN5 | CN4 |
| Ce5 | BN5 | CN5 |
| Ce6 | BN5 | CN6 |
| Ce7 | BN5 | CN7 |
| Ce8 | BN5 | CN8 |
| Ce9 | BN5 | CN9 |
| Ce10 | BN5 | CN10 |
| Ce11 | BN5 | CN11 |
| Ce12 | BN5 | CN12 |
| Ce13 | BN5 | CN13 |
| Ce14 | BN5 | CN14 |
| Ce15 | BN5 | CN15 |
| Ce16 | BN5 | CN16 |
| Ce17 | BN5 | CN17 |
| Ce18 | BN5 | CN18 |
| Ce19 | BN5 | CN19 |
| Ce20 | BN5 | CN20 |
| Ce21 | BN5 | CN21 |
| Ce22 | BN5 | CN22 |
| Ce23 | BN5 | CN23 |
| Ce24 | BN5 | CN24 |
| Ce25 | BN5 | CN25 |
| Ce26 | BN5 | CN26 |
| Ce27 | BN5 | CN27 |
| Ce28 | BN5 | CN28 |
| Ce29 | BN5 | CN29 |
| Ce30 | BN5 | CN30 |
| Ce31 | BN5 | CN31 |
| Ce32 | BN5 | CN32 |
| Ce33 | BN5 | CN33 |
| Ce34 | BN5 | CN34 |
| Ce35 | BN5 | CN35 |
| Ce36 | BN5 | CN36 |
| Ce37 | BN5 | CN37 |
| Ce38 | BN5 | CN38 |
| Ce39 | BN5 | CN39 |
| Ce40 | BN5 | CN40 |
| Ce41 | BN5 | CN41 |
| Ce42 | BN5 | CN42 |
| Ce43 | BN5 | CN43 |
| Ce44 | BN5 | CN44 |
| Ce45 | BN5 | CN45 |
| Ce46 | BN5 | CN46 |
| Cf1 | BN6 | CN1 |
| Cf2 | BN6 | CN2 |
| Cf3 | BN6 | CN3 |
| Cf4 | BN6 | CN4 |
| Cf5 | BN6 | CN5 |
| Cf6 | BN6 | CN6 |
| Cf7 | BN6 | CN7 |
| Cf8 | BN6 | CN8 |
| Cf9 | BN6 | CN9 |
| Cf10 | BN6 | CN10 |
| Cf11 | BN6 | CN11 |
| Cf12 | BN6 | CN12 |
| Cf13 | BN6 | CN13 |
| Cf14 | BN6 | CN14 |
| Cf15 | BN6 | CN15 |
| Cf16 | BN6 | CN16 |
| Cf17 | BN6 | CN17 |
| Cf18 | BN6 | CN18 |
| Cf19 | BN6 | CN19 |
| Cf20 | BN6 | CN20 |
| Cf21 | BN6 | CN21 |
| Cf22 | BN6 | CN22 |
| Cf23 | BN6 | CN23 |
| Cf24 | BN6 | CN24 |
| Cf25 | BN6 | CN25 |
| Cf26 | BN6 | CN26 |
| Cf27 | BN6 | CN27 |
| Cf28 | BN6 | CN28 |
| Cf29 | BN6 | CN29 |
| Cf30 | BN6 | CN30 |
| Cf31 | BN6 | CN31 |
| Cf32 | BN6 | CN32 |
| Cf33 | BN6 | CN33 |
| Cf34 | BN6 | CN34 |
| Cf35 | BN6 | CN35 |
| Cf36 | BN6 | CN36 |
| Cf37 | BN6 | CN37 |
| Cf38 | BN6 | CN38 |
| Cf39 | BN6 | CN39 |
| Cf40 | BN6 | CN40 |
| Cf41 | BN6 | CN41 |
| Cf42 | BN6 | CN42 |
| Cf43 | BN6 | CN43 |
| Cf44 | BN6 | CN44 |
| Cf45 | BN6 | CN45 |
| Cf46 | BN6 | CN46 |
| Cg1 | BN7 | CN1 |
| Cg2 | BN7 | CN2 |
| Cg3 | BN7 | CN3 |
| Cg4 | BN7 | CN4 |
| Cg5 | BN7 | CN5 |
| Cg6 | BN7 | CN6 |
| Cg7 | BN7 | CN7 |
| Cg8 | BN7 | CN8 |
| Cg9 | BN7 | CN9 |
| Cg10 | BN7 | CN10 |
| Cg11 | BN7 | CN11 |
| Cg12 | BN7 | CN12 |
| Cg13 | BN7 | CN13 |
| Cg14 | BN7 | CN14 |
| Cg15 | BN7 | CN15 |
| Cg16 | BN7 | CN16 |
| Cg17 | BN7 | CN17 |
| Cg18 | BN7 | CN18 |
| Cg19 | BN7 | CN19 |
| Cg20 | BN7 | CN20 |
| Cg21 | BN7 | CN21 |
| Cg22 | BN7 | CN22 |
| Cg23 | BN7 | CN23 |
| Cg24 | BN7 | CN24 |
| Cg25 | BN7 | CN25 |
| Cg26 | BN7 | CN26 |
| Cg27 | BN7 | CN27 |
| Cg28 | BN7 | CN28 |
| Cg29 | BN7 | CN29 |
| Cg30 | BN7 | CN30 |
| Cg31 | BN7 | CN31 |
| Cg32 | BN7 | CN32 |
| Cg33 | BN7 | CN33 |
| Cg34 | BN7 | CN34 |
| Cg35 | BN7 | CN35 |
| Cg36 | BN7 | CN36 |
| Cg37 | BN7 | CN37 |
| Cg38 | BN7 | CN38 |
| Cg39 | BN7 | CN39 |
| Cg40 | BN7 | CN40 |
| Cg41 | BN7 | CN41 |
| Cg42 | BN7 | CN42 |
| Cg43 | BN7 | CN43 |
| Cg44 | BN7 | CN44 |
| Cg45 | BN7 | CN45 |
| Cg46 | BN7 | CN46 |
| Ch1 | BN8 | CN1 |
| Ch2 | BN8 | CN2 |
| Ch3 | BN8 | CN3 |
| Ch4 | BN8 | CN4 |
| Ch5 | BN8 | CN5 |
| Ch6 | BN8 | CN6 |
| Ch7 | BN8 | CN7 |
| Ch8 | BN8 | CN8 |
| Ch9 | BN8 | CN9 |
| Ch10 | BN8 | CN10 |
| Ch11 | BN8 | CN11 |
| Ch12 | BN8 | CN12 |
| Ch13 | BN8 | CN13 |
| Ch14 | BN8 | CN14 |
| Ch15 | BN8 | CN15 |
| Ch16 | BN8 | CN16 |
| Ch17 | BN8 | CN17 |
| Ch18 | BN8 | CN18 |
| Ch19 | BN8 | CN19 |
| Ch20 | BN8 | CN20 |
| Ch21 | BN8 | CN21 |
| Ch22 | BN8 | CN22 |
| Ch23 | BN8 | CN23 |
| Ch24 | BN8 | CN24 |
| Ch25 | BN8 | CN25 |
| Ch26 | BN8 | CN26 |
| Ch27 | BN8 | CN27 |
| Ch28 | BN8 | CN28 |
| Ch29 | BN8 | CN29 |
| Ch30 | BN8 | CN30 |
| Ch31 | BN8 | CN31 |
| Ch32 | BN8 | CN32 |
| Ch33 | BN8 | CN33 |
| Ch34 | BN8 | CN34 |
| Ch35 | BN8 | CN35 |
| Ch36 | BN8 | CN36 |
| Ch37 | BN8 | CN37 |
| Ch38 | BN8 | CN38 |
| Ch39 | BN8 | CN39 |
| Ch40 | BN8 | CN40 |
| Ch41 | BN8 | CN41 |
| Ch42 | BN8 | CN42 |
| Ch43 | BN8 | CN43 |
| Ch44 | BN8 | CN44 |
| Ch45 | BN8 | CN45 |
| Ch46 | BN8 | CN46 |
| Ci1 | BN9 | CN1 |
| Ci2 | BN9 | CN2 |
| Ci3 | BN9 | CN3 |
| Ci4 | BN9 | CN4 |
| Ci5 | BN9 | CN5 |
| Ci6 | BN9 | CN6 |
| Ci7 | BN9 | CN7 |
| Ci8 | BN9 | CN8 |
| Ci9 | BN9 | CN9 |
| Ci10 | BN9 | CN10 |
| Ci11 | BN9 | CN11 |
| Ci12 | BN9 | CN12 |
| Ci13 | BN9 | CN13 |
| Ci14 | BN9 | CN14 |
| Ci15 | BN9 | CN15 |
| Ci16 | BN9 | CN16 |
| Ci17 | BN9 | CN17 |
| Ci18 | BN9 | CN18 |
| Ci19 | BN9 | CN19 |
| Ci20 | BN9 | CN20 |
| Ci21 | BN9 | CN21 |
| Ci22 | BN9 | CN22 |
| Ci23 | BN9 | CN23 |
| Ci24 | BN9 | CN24 |
| Ci25 | BN9 | CN25 |
| Ci26 | BN9 | CN26 |
| Ci27 | BN9 | CN27 |
| Ci28 | BN9 | CN28 |
| Ci29 | BN9 | CN29 |
| Ci30 | BN9 | CN30 |
| Ci31 | BN9 | CN31 |
| Ci32 | BN9 | CN32 |
| Ci33 | BN9 | CN33 |
| Ci34 | BN9 | CN34 |
| Ci35 | BN9 | CN35 |
| Ci36 | BN9 | CN36 |
| Ci37 | BN9 | CN37 |
| Ci38 | BN9 | CN38 |
| Ci39 | BN9 | CN39 |
| Ci40 | BN9 | CN40 |
| Ci41 | BN9 | CN41 |
| Ci42 | BN9 | CN42 |
| Ci43 | BN9 | CN43 |
| Ci44 | BN9 | CN44 |
| Ci45 | BN9 | CN45 |
| Ci46 | BN9 | CN46 |
| Cj1 | BN10 | CN1 |
| Cj2 | BN10 | CN2 |
| Cj3 | BN10 | CN3 |
| Cj4 | BN10 | CN4 |
| Cj5 | BN10 | CN5 |
| Cj6 | BN10 | CN6 |
| Cj7 | BN10 | CN7 |
| Cj8 | BN10 | CN8 |
| Cj9 | BN10 | CN9 |
| Cj10 | BN10 | CN10 |
| Cj11 | BN10 | CN11 |
| Cj12 | BN10 | CN12 |
| Cj13 | BN10 | CN13 |
| Cj14 | BN10 | CN14 |
| Cj15 | BN10 | CN15 |
| Cj16 | BN10 | CN16 |
| Cj17 | BN10 | CN17 |
| Cj18 | BN10 | CN18 |
| Cj19 | BN10 | CN19 |
| Cj20 | BN10 | CN20 |
| Cj21 | BN10 | CN21 |
| Cj22 | BN10 | CN22 |
| Cj23 | BN10 | CN23 |
| Cj24 | BN10 | CN24 |
| Cj25 | BN10 | CN25 |
| Cj26 | BN10 | CN26 |
| Cj27 | BN10 | CN27 |
| Cj28 | BN10 | CN28 |
| Cj29 | BN10 | CN29 |
| Cj30 | BN10 | CN30 |
| Cj31 | BN10 | CN31 |
| Cj32 | BN10 | CN32 |
| Cj33 | BN10 | CN33 |
| Cj34 | BN10 | CN34 |
| Cj35 | BN10 | CN35 |
| Cj36 | BN10 | CN36 |
| Cj37 | BN10 | CN37 |
| Cj38 | BN10 | CN38 |
| Cj39 | BN10 | CN39 |
| Cj40 | BN10 | CN40 |
| Cj41 | BN10 | CN41 |
| Cj42 | BN10 | CN42 |
| Cj43 | BN10 | CN43 |
| Cj44 | BN10 | CN44 |
| Cj45 | BN10 | CN45 |
| Cj46 | BN10 | CN46 |
| Ck1 | BN11 | CN1 |
| Ck2 | BN11 | CN2 |
| Ck3 | BN11 | CN3 |
| Ck4 | BN11 | CN4 |
| Ck5 | BN11 | CN5 |
| Ck6 | BN11 | CN6 |
| Ck7 | BN11 | CN7 |
| Ck8 | BN11 | CN8 |
| Ck9 | BN11 | CN9 |
| Ck10 | BN11 | CN10 |
| Ck11 | BN11 | CN11 |
| Ck12 | BN11 | CN12 |
| Ck13 | BN11 | CN13 |
| Ck14 | BN11 | CN14 |
| Ck15 | BN11 | CN15 |
| Ck16 | BN11 | CN16 |
| Ck17 | BN11 | CN17 |
| Ck18 | BN11 | CN18 |
| Ck19 | BN11 | CN19 |
| Ck20 | BN11 | CN20 |
| Ck21 | BN11 | CN21 |
| Ck22 | BN11 | CN22 |
| Ck23 | BN11 | CN23 |
| Ck24 | BN11 | CN24 |
| Ck25 | BN11 | CN25 |
| Ck26 | BN11 | CN26 |
| Ck27 | BN11 | CN27 |
| Ck28 | BN11 | CN28 |
| Ck29 | BN11 | CN29 |
| Ck30 | BN11 | CN30 |
| Ck31 | BN11 | CN31 |
| Ck32 | BN11 | CN32 |
| Ck33 | BN11 | CN33 |
| Ck34 | BN11 | CN34 |
| Ck35 | BN11 | CN35 |
| Ck36 | BN11 | CN36 |
| Ck37 | BN11 | CN37 |
| Ck38 | BN11 | CN38 |
| Ck39 | BN11 | CN39 |
| Ck40 | BN11 | CN40 |
| Ck41 | BN11 | CN41 |
| Ck42 | BN11 | CN42 |
| Ck43 | BN11 | CN43 |
| Ck44 | BN11 | CN44 |
| Ck45 | BN11 | CN45 |
| Ck46 | BN11 | CN46 |
| CI1 | BN12 | CN1 |
| CI2 | BN12 | CN2 |
| CI3 | BN12 | CN3 |
| CI4 | BN12 | CN4 |
| CI5 | BN12 | CN5 |
| CI6 | BN12 | CN6 |
| CI7 | BN12 | CN7 |
| CI8 | BN12 | CN8 |
| CI9 | BN12 | CN9 |
| CI10 | BN12 | CN10 |
| CI11 | BN12 | CN11 |
| CI12 | BN12 | CN12 |
| CI13 | BN12 | CN13 |
| CI14 | BN12 | CN14 |
| CI15 | BN12 | CN15 |
| CI16 | BN12 | CN16 |
| CI17 | BN12 | CN17 |
| CI18 | BN12 | CN18 |
| CI19 | BN12 | CN19 |
| CI20 | BN12 | CN20 |
| CI21 | BN12 | CN21 |
| CI22 | BN12 | CN22 |
| CI23 | BN12 | CN23 |
| CI24 | BN12 | CN24 |
| CI25 | BN12 | CN25 |
| CI26 | BN12 | CN26 |
| CI27 | BN12 | CN27 |
| CI28 | BN12 | CN28 |
| CI29 | BN12 | CN29 |
| CI30 | BN12 | CN30 |
| CI31 | BN12 | CN31 |
| CI32 | BN12 | CN32 |
| CI33 | BN12 | CN33 |
| CI34 | BN12 | CN34 |
| CI35 | BN12 | CN35 |
| CI36 | BN12 | CN36 |
| CI37 | BN12 | CN37 |
| CI38 | BN12 | CN38 |
| CI39 | BN12 | CN39 |
| CI40 | BN12 | CN40 |
| CI41 | BN12 | CN41 |
| CI42 | BN12 | CN42 |
| CI43 | BN12 | CN43 |
| CI44 | BN12 | CN44 |
| CI45 | BN12 | CN45 |
| CI46 | BN12 | CN46 |
| Cm1 | BN13 | CN1 |
| Cm2 | BN13 | CN2 |
| Cm3 | BN13 | CN3 |
| Cm4 | BN13 | CN4 |
| Cm5 | BN13 | CN5 |
| Cm6 | BN13 | CN6 |
| Cm7 | BN13 | CN7 |
| Cm8 | BN13 | CN8 |
| Cm9 | BN13 | CN9 |
| Cm10 | BN13 | CN10 |
| Cm11 | BN13 | CN11 |
| Cm12 | BN13 | CN12 |
| Cm13 | BN13 | CN13 |
| Cm14 | BN13 | CN14 |
| Cm15 | BN13 | CN15 |
| Cm16 | BN13 | CN16 |
| Cm17 | BN13 | CN17 |
| Cm18 | BN13 | CN18 |
| Cm19 | BN13 | CN19 |
| Cm20 | BN13 | CN20 |
| Cm21 | BN13 | CN21 |
| Cm22 | BN13 | CN22 |
| Cm23 | BN13 | CN23 |
| Cm24 | BN13 | CN24 |
| Cm25 | BN13 | CN25 |
| Cm26 | BN13 | CN26 |
| Cm27 | BN13 | CN27 |
| Cm28 | BN13 | CN28 |
| Cm29 | BN13 | CN29 |
| Cm30 | BN13 | CN30 |
| Cm31 | BN13 | CN31 |
| Cm32 | BN13 | CN32 |
| Cm33 | BN13 | CN33 |
| Cm34 | BN13 | CN34 |
| Cm35 | BN13 | CN35 |
| Cm36 | BN13 | CN36 |
| Cm37 | BN13 | CN37 |
| Cm38 | BN13 | CN38 |
| Cm39 | BN13 | CN39 |
| Cm40 | BN13 | CN40 |
| Cm41 | BN13 | CN41 |
| Cm42 | BN13 | CN42 |
| Cm43 | BN13 | CN43 |
| Cm44 | BN13 | CN44 |
| Cm45 | BN13 | CN45 |
| Cm46 | BN13 | CN46 |
| Cn1 | BN14 | CN1 |
| Cn2 | BN14 | CN2 |
| Cn3 | BN14 | CN3 |
| Cn4 | BN14 | CN4 |
| Cn5 | BN14 | CN5 |
| Cn6 | BN14 | CN6 |
| Cn7 | BN14 | CN7 |
| Cn8 | BN14 | CN8 |
| Cn9 | BN14 | CN9 |
| Cn10 | BN14 | CN10 |
| Cn11 | BN14 | CN11 |
| Cn12 | BN14 | CN12 |
| Cn13 | BN14 | CN13 |
| Cn14 | BN14 | CN14 |
| Cn15 | BN14 | CN15 |
| Cn16 | BN14 | CN16 |
| Cn17 | BN14 | CN17 |
| Cn18 | BN14 | CN18 |
| Cn19 | BN14 | CN19 |
| Cn20 | BN14 | CN20 |
| Cn21 | BN14 | CN21 |
| Cn22 | BN14 | CN22 |
| Cn23 | BN14 | CN23 |
| Cn24 | BN14 | CN24 |
| Cn25 | BN14 | CN25 |
| Cn26 | BN14 | CN26 |
| Cn27 | BN14 | CN27 |
| Cn28 | BN14 | CN28 |
| Cn29 | BN14 | CN29 |
| Cn30 | BN14 | CN30 |
| Cn31 | BN14 | CN31 |
| Cn32 | BN14 | CN32 |
| Cn33 | BN14 | CN33 |
| Cn34 | BN14 | CN34 |
| Cn35 | BN14 | CN35 |
| Cn36 | BN14 | CN36 |
| Cn37 | BN14 | CN37 |
| Cn38 | BN14 | CN38 |
| Cn39 | BN14 | CN39 |
| Cn40 | BN14 | CN40 |
| Cn41 | BN14 | CN41 |
| Cn42 | BN14 | CN42 |
| Cn43 | BN14 | CN43 |
| Cn44 | BN14 | CN44 |
| Cn45 | BN14 | CN45 |
| Cn46 | BN14 | CN46 |
| Co1 | BN15 | CN1 |
| Co2 | BN15 | CN2 |
| Co3 | BN15 | CN3 |
| Co4 | BN15 | CN4 |
| Co5 | BN15 | CN5 |
| Co6 | BN15 | CN6 |
| Co7 | BN15 | CN7 |
| Co8 | BN15 | CN8 |
| Co9 | BN15 | CN9 |
| Co10 | BN15 | CN10 |
| Co11 | BN15 | CN11 |
| Co12 | BN15 | CN12 |
| Co13 | BN15 | CN13 |
| Co14 | BN15 | CN14 |
| Co15 | BN15 | CN15 |
| Co16 | BN15 | CN16 |
| Co17 | BN15 | CN17 |
| Co18 | BN15 | CN18 |
| Co19 | BN15 | CN19 |
| Co20 | BN15 | CN20 |
| Co21 | BN15 | CN21 |
| Co22 | BN15 | CN22 |
| Co23 | BN15 | CN23 |
| Co24 | BN15 | CN24 |
| Co25 | BN15 | CN25 |
| Co26 | BN15 | CN26 |
| Co27 | BN15 | CN27 |
| Co28 | BN15 | CN28 |
| Co29 | BN15 | CN29 |
| Co30 | BN15 | CN30 |
| Co31 | BN15 | CN31 |
| Co32 | BN15 | CN32 |
| Co33 | BN15 | CN33 |
| Co34 | BN15 | CN34 |
| Co35 | BN15 | CN35 |
| Co36 | BN15 | CN36 |
| Co37 | BN15 | CN37 |
| Co38 | BN15 | CN38 |
| Co39 | BN15 | CN39 |
| Co40 | BN15 | CN40 |
| Co41 | BN15 | CN41 |
| Co42 | BN15 | CN42 |
| Co43 | BN15 | CN43 |
| Co44 | BN15 | CN44 |
| Co45 | BN15 | CN45 |
| Co46 | BN15 | CN46 |
| Cp1 | BN16 | CN1 |
| Cp2 | BN16 | CN2 |
| Cp3 | BN16 | CN3 |
| Cp4 | BN16 | CN4 |
| Cp5 | BN16 | CN5 |
| Cp6 | BN16 | CN6 |
| Cp7 | BN16 | CN7 |
| Cp8 | BN16 | CN8 |
| Cp9 | BN16 | CN9 |
| Cp10 | BN16 | CN10 |
| Cp11 | BN16 | CN11 |
| Cp12 | BN16 | CN12 |
| Cp13 | BN16 | CN13 |
| Cp14 | BN16 | CN14 |
| Cp15 | BN16 | CN15 |
| Cp16 | BN16 | CN16 |
| Cp17 | BN16 | CN17 |
| Cp18 | BN16 | CN18 |
| Cp19 | BN16 | CN19 |
| Cp20 | BN16 | CN20 |
| Cp21 | BN16 | CN21 |
| Cp22 | BN16 | CN22 |
| Cp23 | BN16 | CN23 |
| Cp24 | BN16 | CN24 |
| Cp25 | BN16 | CN25 |
| Cp26 | BN16 | CN26 |
| Cp27 | BN16 | CN27 |
| Cp28 | BN16 | CN28 |
| Cp29 | BN16 | CN29 |
| Cp30 | BN16 | CN30 |
| Cp31 | BN16 | CN31 |
| Cp32 | BN16 | CN32 |
| Cp33 | BN16 | CN33 |
| Cp34 | BN16 | CN34 |
| Cp35 | BN16 | CN35 |
| Cp36 | BN16 | CN36 |
| Cp37 | BN16 | CN37 |
| Cp38 | BN16 | CN38 |
| Cp39 | BN16 | CN39 |
| Cp40 | BN16 | CN40 |
| Cp41 | BN16 | CN41 |
| Cp42 | BN16 | CN42 |
| Cp43 | BN16 | CN43 |
| Cp44 | BN16 | CN44 |
| Cp45 | BN16 | CN45 |
| Cp46 | BN16 | CN46 |
| Cq1 | BN17 | CN1 |
| Cq2 | BN17 | CN2 |
| Cq3 | BN17 | CN3 |
| Cq4 | BN17 | CN4 |
| Cq5 | BN17 | CN5 |
| Cq6 | BN17 | CN6 |
| Cq7 | BN17 | CN7 |
| Cq8 | BN17 | CN8 |
| Cq9 | BN17 | CN9 |
| Cq10 | BN17 | CN10 |
| Cq11 | BN17 | CN11 |
| Cq12 | BN17 | CN12 |
| Cq13 | BN17 | CN13 |
| Cq14 | BN17 | CN14 |
| Cq15 | BN17 | CN15 |
| Cq16 | BN17 | CN16 |
| Cq17 | BN17 | CN17 |
| Cq18 | BN17 | CN18 |
| Cq19 | BN17 | CN19 |
| Cq20 | BN17 | CN20 |
| Cq21 | BN17 | CN21 |
| Cq22 | BN17 | CN22 |
| Cq23 | BN17 | CN23 |
| Cq24 | BN17 | CN24 |
| Cq25 | BN17 | CN25 |
| Cq26 | BN17 | CN26 |
| Cq27 | BN17 | CN27 |
| Cq28 | BN17 | CN28 |
| Cq29 | BN17 | CN29 |
| Cq30 | BN17 | CN30 |
| Cq31 | BN17 | CN31 |
| Cq32 | BN17 | CN32 |
| Cq33 | BN17 | CN33 |
| Cq34 | BN17 | CN34 |
| Cq35 | BN17 | CN35 |
| Cq36 | BN17 | CN36 |
| Cq37 | BN17 | CN37 |
| Cq38 | BN17 | CN38 |
| Cq39 | BN17 | CN39 |
| Cq40 | BN17 | CN40 |
| Cq41 | BN17 | CN41 |
| Cq42 | BN17 | CN42 |
| Cq43 | BN17 | CN43 |
| Cq44 | BN17 | CN44 |
| Cq45 | BN17 | CN45 |
| Cq46 | BN17 | CN46 |
| Cs1 | BN18 | CN1 |
| Cs2 | BN18 | CN2 |
| Cs3 | BN18 | CN3 |
| Cs4 | BN18 | CN4 |
| Cs5 | BN18 | CN5 |
| Cs6 | BN18 | CN6 |
| Cs7 | BN18 | CN7 |
| Cs8 | BN18 | CN8 |
| Cs9 | BN18 | CN9 |
| Cs10 | BN18 | CN10 |
| Cs11 | BN18 | CN11 |
| Cs12 | BN18 | CN12 |
| Cs13 | BN18 | CN13 |
| Cs14 | BN18 | CN14 |
| Cs15 | BN18 | CN15 |
| Cs16 | BN18 | CN16 |
| Cs17 | BN18 | CN17 |
| Cs18 | BN18 | CN18 |
| Cs19 | BN18 | CN19 |
| Cs20 | BN18 | CN20 |
| Cs21 | BN18 | CN21 |
| Cs22 | BN18 | CN22 |
| Cs23 | BN18 | CN23 |
| Cs24 | BN18 | CN24 |
| Cs25 | BN18 | CN25 |
| Cs26 | BN18 | CN26 |
| Cs27 | BN18 | CN27 |
| Cs28 | BN18 | CN28 |
| Cs29 | BN18 | CN29 |
| Cs30 | BN18 | CN30 |
| Cs31 | BN18 | CN31 |
| Cs32 | BN18 | CN32 |
| Cs33 | BN18 | CN33 |
| Cs34 | BN18 | CN34 |
| Cs35 | BN18 | CN35 |
| Cs36 | BN18 | CN36 |
| Cs37 | BN18 | CN37 |
| Cs38 | BN18 | CN38 |
| Cs39 | BN18 | CN39 |
| Cs40 | BN18 | CN40 |
| Cs41 | BN18 | CN41 |
| Cs42 | BN18 | CN42 |
| Cs43 | BN18 | CN43 |
| Cs44 | BN18 | CN44 |
| Cs45 | BN18 | CN45 |
| Cs46 | BN18 | CN46 |
| Ct1 | BN19 | CN1 |
| Ct2 | BN19 | CN2 |
| Ct3 | BN19 | CN3 |
| Ct4 | BN19 | CN4 |
| Ct5 | BN19 | CN5 |
| Ct6 | BN19 | CN6 |
| Ct7 | BN19 | CN7 |
| Ct8 | BN19 | CN8 |
| Ct9 | BN19 | CN9 |
| Ct10 | BN19 | CN10 |
| Ct11 | BN19 | CN11 |
| Ct12 | BN19 | CN12 |
| Ct13 | BN19 | CN13 |
| Ct14 | BN19 | CN14 |
| Ct15 | BN19 | CN15 |
| Ct16 | BN19 | CN16 |
| Ct17 | BN19 | CN17 |
| Ct18 | BN19 | CN18 |
| Ct19 | BN19 | CN19 |
| Ct20 | BN19 | CN20 |
| Ct21 | BN19 | CN21 |
| Ct22 | BN19 | CN22 |
| Ct23 | BN19 | CN23 |
| Ct24 | BN19 | CN24 |
| Ct25 | BN19 | CN25 |
| Ct26 | BN19 | CN26 |
| Ct27 | BN19 | CN27 |
| Ct28 | BN19 | CN28 |
| Ct29 | BN19 | CN29 |
| Ct30 | BN19 | CN30 |
| Ct31 | BN19 | CN31 |
| Ct32 | BN19 | CN32 |
| Ct33 | BN19 | CN33 |
| Ct34 | BN19 | CN34 |
| Ct35 | BN19 | CN35 |
| Ct36 | BN19 | CN36 |
| Ct37 | BN19 | CN37 |
| Ct38 | BN19 | CN38 |
| Ct39 | BN19 | CN39 |
| Ct40 | BN19 | CN40 |
| Ct41 | BN19 | CN41 |
| Ct42 | BN19 | CN42 |
| Ct43 | BN19 | CN43 |
| Ct44 | BN19 | CN44 |
| Ct45 | BN19 | CN45 |
| Ct46 | BN19 | CN46 |
| Cu1 | BN20 | CN1 |
| Cu2 | BN20 | CN2 |
| Cu3 | BN20 | CN3 |
| Cu4 | BN20 | CN4 |
| Cu5 | BN20 | CN5 |
| Cu6 | BN20 | CN6 |
| Cu7 | BN20 | CN7 |
| Cu8 | BN20 | CN8 |
| Cu9 | BN20 | CN9 |
| Cu10 | BN20 | CN10 |
| Cu11 | BN20 | CN11 |
| Cu12 | BN20 | CN12 |
| Cu13 | BN20 | CN13 |
| Cu14 | BN20 | CN14 |
| Cu15 | BN20 | CN15 |
| Cu16 | BN20 | CN16 |
| Cu17 | BN20 | CN17 |
| Cu18 | BN20 | CN18 |
| Cu19 | BN20 | CN19 |
| Cu20 | BN20 | CN20 |
| Cu21 | BN20 | CN21 |
| Cu22 | BN20 | CN22 |
| Cu23 | BN20 | CN23 |
| Cu24 | BN20 | CN24 |
| Cu25 | BN20 | CN25 |
| Cu26 | BN20 | CN26 |
| Cu27 | BN20 | CN27 |
| Cu28 | BN20 | CN28 |
| Cu29 | BN20 | CN29 |
| Cu30 | BN20 | CN30 |
| Cu31 | BN20 | CN31 |
| Cu32 | BN20 | CN32 |
| Cu33 | BN20 | CN33 |
| Cu34 | BN20 | CN34 |
| Cu35 | BN20 | CN35 |
| Cu36 | BN20 | CN36 |
| Cu37 | BN20 | CN37 |
| Cu38 | BN20 | CN38 |
| Cu39 | BN20 | CN39 |
| Cu40 | BN20 | CN40 |
| Cu41 | BN20 | CN41 |
| Cu42 | BN20 | CN42 |
| Cu43 | BN20 | CN43 |
| Cu44 | BN20 | CN44 |
| Cu45 | BN20 | CN45 |
| Cu46 | BN20 | CN46 |
| Cv1 | BN21 | CN1 |
| Cv2 | BN21 | CN2 |
| Cv3 | BN21 | CN3 |
| Cv4 | BN21 | CN4 |
| Cv5 | BN21 | CN5 |
| Cv6 | BN21 | CN6 |
| Cv7 | BN21 | CN7 |
| Cv8 | BN21 | CN8 |
| Cv9 | BN21 | CN9 |
| Cv10 | BN21 | CN10 |
| Cv11 | BN21 | CN11 |
| Cv12 | BN21 | CN12 |
| Cv13 | BN21 | CN13 |
| Cv14 | BN21 | CN14 |
| Cv15 | BN21 | CN15 |
| Cv16 | BN21 | CN16 |
| Cv17 | BN21 | CN17 |
| Cv18 | BN21 | CN18 |
| Cv19 | BN21 | CN19 |
| Cv20 | BN21 | CN20 |
| Cv21 | BN21 | CN21 |
| Cv22 | BN21 | CN22 |
| Cv23 | BN21 | CN23 |
| Cv24 | BN21 | CN24 |
| Cv25 | BN21 | CN25 |
| Cv26 | BN21 | CN26 |
| Cv27 | BN21 | CN27 |
| Cv28 | BN21 | CN28 |
| Cv29 | BN21 | CN29 |
| Cv30 | BN21 | CN30 |
| Cv31 | BN21 | CN31 |
| Cv32 | BN21 | CN32 |
| Cv33 | BN21 | CN33 |
| Cv34 | BN21 | CN34 |
| Cv35 | BN21 | CN35 |
| Cv36 | BN21 | CN36 |
| Cv37 | BN21 | CN37 |
| Cv38 | BN21 | CN38 |
| Cv39 | BN21 | CN39 |
| Cv40 | BN21 | CN40 |
| Cv41 | BN21 | CN41 |
| Cv42 | BN21 | CN42 |
| Cv43 | BN21 | CN43 |
| Cv44 | BN21 | CN44 |
| Cv45 | BN21 | CN45 |
| Cv46 | BN21 | CN46 |

TABLE 4

| Name of compound | R_{D1} | R_{D2} |
|---|---|---|
| Da1 | BN1 | CN1 |
| Da2 | BN1 | CN2 |
| Da3 | BN1 | CN3 |
| Da4 | BN1 | CN4 |
| Da5 | BN1 | CN5 |
| Da6 | BN1 | CN6 |
| Da7 | BN1 | CN7 |
| Da8 | BN1 | CN8 |
| Da9 | BN1 | CN9 |
| Da10 | BN1 | CN10 |
| Da11 | BN1 | CN11 |
| Da12 | BN1 | CN12 |
| Da13 | BN1 | CN13 |
| Da14 | BN1 | CN14 |
| Da15 | BN1 | CN15 |
| Da16 | BN1 | CN16 |
| Da17 | BN1 | CN17 |
| Da18 | BN1 | CN18 |
| Da19 | BN1 | CN19 |
| Da20 | BN1 | CN20 |
| Da21 | BN1 | CN21 |
| Da22 | BN1 | CN22 |
| Da23 | BN1 | CN23 |
| Da24 | BN1 | CN24 |
| Da25 | BN1 | CN25 |
| Da26 | BN1 | CN26 |
| Da27 | BN1 | CN27 |
| Da28 | BN1 | CN28 |
| Da29 | BN1 | CN29 |
| Da30 | BN1 | CN30 |
| Da31 | BN1 | CN31 |
| Da32 | BN1 | CN32 |
| Da33 | BN1 | CN33 |
| Da34 | BN1 | CN34 |
| Da35 | BN1 | CN35 |
| Da36 | BN1 | CN36 |
| Da37 | BN1 | CN37 |
| Da38 | BN1 | CN38 |
| Da39 | BN1 | CN39 |
| Da40 | BN1 | CN40 |
| Da41 | BN1 | CN41 |
| Da42 | BN1 | CN42 |
| Da43 | BN1 | CN43 |
| Da44 | BN1 | CN44 |
| Da45 | BN1 | CN45 |
| Da46 | BN1 | CN46 |
| Db1 | BN2 | CN1 |
| Db2 | BN2 | CN2 |
| Db3 | BN2 | CN3 |
| Db4 | BN2 | CN4 |
| Db5 | BN2 | CN5 |
| Db6 | BN2 | CN6 |
| Db7 | BN2 | CN7 |
| Db8 | BN2 | CN8 |
| Db9 | BN2 | CN9 |
| Db10 | BN2 | CN10 |
| Db11 | BN2 | CN11 |
| Db12 | BN2 | CN12 |
| Db13 | BN2 | CN13 |
| Db14 | BN2 | CN14 |
| Db15 | BN2 | CN15 |
| Db16 | BN2 | CN16 |
| Db17 | BN2 | CN17 |
| Db18 | BN2 | CN18 |
| Db19 | BN2 | CN19 |
| Db20 | BN2 | CN20 |
| Db21 | BN2 | CN21 |
| Db22 | BN2 | CN22 |
| Db23 | BN2 | CN23 |
| Db24 | BN2 | CN24 |
| Db25 | BN2 | CN25 |
| Db26 | BN2 | CN26 |
| Db27 | BN2 | CN27 |
| Db28 | BN2 | CN28 |
| Db29 | BN2 | CN29 |
| Db30 | BN2 | CN30 |
| Db31 | BN2 | CN31 |
| Db32 | BN2 | CN32 |
| Db33 | BN2 | CN33 |
| Db34 | BN2 | CN34 |
| Db35 | BN2 | CN35 |
| Db36 | BN2 | CN36 |
| Db37 | BN2 | CN37 |
| Db38 | BN2 | CN38 |
| Db39 | BN2 | CN39 |
| Db40 | BN2 | CN40 |
| Db41 | BN2 | CN41 |
| Db42 | BN2 | CN42 |
| Db43 | BN2 | CN43 |
| Db44 | BN2 | CN44 |
| Db45 | BN2 | CN45 |
| Db46 | BN2 | CN46 |
| Dc1 | BN3 | CN1 |
| Dc2 | BN3 | CN2 |
| Dc3 | BN3 | CN3 |
| Dc4 | BN3 | CN4 |
| Dc5 | BN3 | CN5 |
| Dc6 | BN3 | CN6 |
| Dc7 | BN3 | CN7 |
| Dc8 | BN3 | CN8 |
| Dc9 | BN3 | CN9 |
| Dc10 | BN3 | CN10 |
| Dc11 | BN3 | CN11 |
| Dc12 | BN3 | CN12 |
| Dc13 | BN3 | CN13 |
| Dc14 | BN3 | CN14 |
| Dc15 | BN3 | CN15 |
| Dc16 | BN3 | CN16 |
| Dc17 | BN3 | CN17 |
| Dc18 | BN3 | CN18 |
| Dc19 | BN3 | CN19 |
| Dc20 | BN3 | CN20 |
| Dc21 | BN3 | CN21 |
| Dc22 | BN3 | CN22 |
| Dc23 | BN3 | CN23 |
| Dc24 | BN3 | CN24 |
| Dc25 | BN3 | CN25 |
| Dc26 | BN3 | CN26 |
| Dc27 | BN3 | CN27 |
| Dc28 | BN3 | CN28 |
| Dc29 | BN3 | CN29 |
| Dc30 | BN3 | CN30 |
| Dc31 | BN3 | CN31 |
| Dc32 | BN3 | CN32 |
| Dc33 | BN3 | CN33 |
| Dc34 | BN3 | CN34 |
| Dc35 | BN3 | CN35 |
| Dc36 | BN3 | CN36 |
| Dc37 | BN3 | CN37 |
| Dc38 | BN3 | CN38 |
| Dc39 | BN3 | CN39 |
| Dc40 | BN3 | CN40 |
| Dc41 | BN3 | CN41 |
| Dc42 | BN3 | CN42 |
| Dc43 | BN3 | CN43 |
| Dc44 | BN3 | CN44 |
| Dc45 | BN3 | CN45 |
| Dc46 | BN3 | CN46 |
| Dd1 | BN4 | CN1 |
| Dd2 | BN4 | CN2 |
| Dd3 | BN4 | CN3 |
| Dd4 | BN4 | CN4 |
| Dd5 | BN4 | CN5 |
| Dd6 | BN4 | CN6 |
| Dd7 | BN4 | CN7 |
| Dd8 | BN4 | CN8 |
| Dd9 | BN4 | CN9 |
| Dd10 | BN4 | CN10 |
| Dd11 | BN4 | CN11 |
| Dd12 | BN4 | CN12 |
| Dd13 | BN4 | CN13 |
| Dd14 | BN4 | CN14 |
| Dd15 | BN4 | CN15 |
| Dd16 | BN4 | CN16 |
| Dd17 | BN4 | CN17 |
| Dd18 | BN4 | CN18 |
| Dd19 | BN4 | CN19 |
| Dd20 | BN4 | CN20 |
| Dd21 | BN4 | CN21 |
| Dd22 | BN4 | CN22 |
| Dd23 | BN4 | CN23 |
| Dd24 | BN4 | CN24 |
| Dd25 | BN4 | CN25 |
| Dd26 | BN4 | CN26 |
| Dd27 | BN4 | CN27 |
| Dd28 | BN4 | CN28 |
| Dd29 | BN4 | CN29 |
| Dd30 | BN4 | CN30 |
| Dd31 | BN4 | CN31 |
| Dd32 | BN4 | CN32 |
| Dd33 | BN4 | CN33 |
| Dd34 | BN4 | CN34 |
| Dd35 | BN4 | CN35 |
| Dd36 | BN4 | CN36 |
| Dd37 | BN4 | CN37 |
| Dd38 | BN4 | CN38 |
| Dd39 | BN4 | CN39 |
| Dd40 | BN4 | CN40 |
| Dd41 | BN4 | CN41 |
| Dd42 | BN4 | CN42 |
| Dd43 | BN4 | CN43 |
| Dd44 | BN4 | CN44 |
| Dd45 | BN4 | CN45 |
| Dd46 | BN4 | CN46 |
| De1 | BN5 | CN1 |
| De2 | BN5 | CN2 |
| De3 | BN5 | CN3 |
| De4 | BN5 | CN4 |
| De5 | BN5 | CN5 |
| De6 | BN5 | CN6 |
| De7 | BN5 | CN7 |
| De8 | BN5 | CN8 |
| De9 | BN5 | CN9 |
| De10 | BN5 | CN10 |
| De11 | BN5 | CN11 |
| De12 | BN5 | CN12 |
| De13 | BN5 | CN13 |
| De14 | BN5 | CN14 |
| De15 | BN5 | CN15 |
| De16 | BN5 | CN16 |
| De17 | BN5 | CN17 |
| De18 | BN5 | CN18 |
| De19 | BN5 | CN19 |
| De20 | BN5 | CN20 |
| De21 | BN5 | CN21 |
| De22 | BN5 | CN22 |
| De23 | BN5 | CN23 |
| De24 | BN5 | CN24 |
| De25 | BN5 | CN25 |
| De26 | BN5 | CN26 |
| De27 | BN5 | CN27 |
| De28 | BN5 | CN28 |
| De29 | BN5 | CN29 |
| De30 | BN5 | CN30 |
| De31 | BN5 | CN31 |
| De32 | BN5 | CN32 |
| De33 | BN5 | CN33 |
| De34 | BN5 | CN34 |
| De35 | BN5 | CN35 |
| De36 | BN5 | CN36 |
| De37 | BN5 | CN37 |
| De38 | BN5 | CN38 |
| De39 | BN5 | CN39 |
| De40 | BN5 | CN40 |
| De41 | BN5 | CN41 |
| De42 | BN5 | CN42 |
| De43 | BN5 | CN43 |
| De44 | BN5 | CN44 |
| De45 | BN5 | CN45 |
| De46 | BN5 | CN46 |
| Df1 | BN6 | CN1 |
| Df2 | BN6 | CN2 |
| Df3 | BN6 | CN3 |
| Df4 | BN6 | CN4 |
| Df5 | BN6 | CN5 |
| Df6 | BN6 | CN6 |
| Df7 | BN6 | CN7 |
| Df8 | BN6 | CN8 |
| Df9 | BN6 | CN9 |
| Df10 | BN6 | CN10 |
| Df11 | BN6 | CN11 |
| Df12 | BN6 | CN12 |
| Df13 | BN6 | CN13 |
| Df14 | BN6 | CN14 |
| Df15 | BN6 | CN15 |
| Df16 | BN6 | CN16 |
| Df17 | BN6 | CN17 |
| Df18 | BN6 | CN18 |
| Df19 | BN6 | CN19 |
| Df20 | BN6 | CN20 |
| Df21 | BN6 | CN21 |
| Df22 | BN6 | CN22 |
| Df23 | BN6 | CN23 |
| Df24 | BN6 | CN24 |
| Df25 | BN6 | CN25 |
| Df26 | BN6 | CN26 |
| Df27 | BN6 | CN27 |
| Df28 | BN6 | CN28 |
| Df29 | BN6 | CN29 |
| Df30 | BN6 | CN30 |
| Df31 | BN6 | CN31 |
| Df32 | BN6 | CN32 |
| Df33 | BN6 | CN33 |
| Df34 | BN6 | CN34 |
| Df35 | BN6 | CN35 |
| Df36 | BN6 | CN36 |
| Df37 | BN6 | CN37 |
| Df38 | BN6 | CN38 |
| Df39 | BN6 | CN39 |
| Df40 | BN6 | CN40 |
| Df41 | BN6 | CN41 |
| Df42 | BN6 | CN42 |
| Df43 | BN6 | CN43 |
| Df44 | BN6 | CN44 |
| Df45 | BN6 | CN45 |
| Df46 | BN6 | CN46 |
| Dg1 | BN7 | CN1 |
| Dg2 | BN7 | CN2 |
| Dg3 | BN7 | CN3 |
| Dg4 | BN7 | CN4 |
| Dg5 | BN7 | CN5 |
| Dg6 | BN7 | CN6 |
| Dg7 | BN7 | CN7 |
| Dg8 | BN7 | CN8 |
| Dg9 | BN7 | CN9 |
| Dg10 | BN7 | CN10 |
| Dg11 | BN7 | CN11 |
| Dg12 | BN7 | CN12 |
| Dg13 | BN7 | CN13 |
| Dg14 | BN7 | CN14 |
| Dg15 | BN7 | CN15 |
| Dg16 | BN7 | CN16 |
| Dg17 | BN7 | CN17 |
| Dg18 | BN7 | CN18 |
| Dg19 | BN7 | CN19 |
| Dg20 | BN7 | CN20 |
| Dg21 | BN7 | CN21 |
| Dg22 | BN7 | CN22 |
| Dg23 | BN7 | CN23 |
| Dg24 | BN7 | CN24 |
| Dg25 | BN7 | CN25 |
| Dg26 | BN7 | CN26 |
| Dg27 | BN7 | CN27 |
| Dg28 | BN7 | CN28 |
| Dg29 | BN7 | CN29 |
| Dg30 | BN7 | CN30 |
| Dg31 | BN7 | CN31 |
| Dg32 | BN7 | CN32 |
| Dg33 | BN7 | CN33 |
| Dg34 | BN7 | CN34 |
| Dg35 | BN7 | CN35 |
| Dg36 | BN7 | CN36 |
| Dg37 | BN7 | CN37 |
| Dg38 | BN7 | CN38 |
| Dg39 | BN7 | CN39 |
| Dg40 | BN7 | CN40 |
| Dg41 | BN7 | CN41 |
| Dg42 | BN7 | CN42 |
| Dg43 | BN7 | CN43 |
| Dg44 | BN7 | CN44 |
| Dg45 | BN7 | CN45 |
| Dg46 | BN7 | CN46 |
| Dh1 | BN8 | CN1 |
| Dh2 | BN8 | CN2 |
| Dh3 | BN8 | CN3 |
| Dh4 | BN8 | CN4 |
| Dh5 | BN8 | CN5 |
| Dh6 | BN8 | CN6 |
| Dh7 | BN8 | CN7 |
| Dh8 | BN8 | CN8 |
| Dh9 | BN8 | CN9 |
| Dh10 | BN8 | CN10 |
| Dh11 | BN8 | CN11 |
| Dh12 | BN8 | CN12 |
| Dh13 | BN8 | CN13 |
| Dh14 | BN8 | CN14 |
| Dh15 | BN8 | CN15 |
| Dh16 | BN8 | CN16 |
| Dh17 | BN8 | CN17 |
| Dh18 | BN8 | CN18 |
| Dh19 | BN8 | CN19 |
| Dh20 | BN8 | CN20 |
| Dh21 | BN8 | CN21 |
| Dh22 | BN8 | CN22 |
| Dh23 | BN8 | CN23 |
| Dh24 | BN8 | CN24 |
| Dh25 | BN8 | CN25 |
| Dh26 | BN8 | CN26 |
| Dh27 | BN8 | CN27 |
| Dh28 | BN8 | CN28 |
| Dh29 | BN8 | CN29 |
| Dh30 | BN8 | CN30 |
| Dh31 | BN8 | CN31 |
| Dh32 | BN8 | CN32 |
| Dh33 | BN8 | CN33 |
| Dh34 | BN8 | CN34 |
| Dh35 | BN8 | CN35 |
| Dh36 | BN8 | CN36 |
| Dh37 | BN8 | CN37 |
| Dh38 | BN8 | CN38 |
| Dh39 | BN8 | CN39 |
| Dh40 | BN8 | CN40 |
| Dh41 | BN8 | CN41 |
| Dh42 | BN8 | CN42 |
| Dh43 | BN8 | CN43 |
| Dh44 | BN8 | CN44 |
| Dh45 | BN8 | CN45 |
| Dh46 | BN8 | CN46 |
| Di1 | BN9 | CN1 |
| Di2 | BN9 | CN2 |
| Di3 | BN9 | CN3 |
| Di4 | BN9 | CN4 |
| Di5 | BN9 | CN5 |
| Di6 | BN9 | CN6 |
| Di7 | BN9 | CN7 |
| Di8 | BN9 | CN8 |
| Di9 | BN9 | CN9 |
| Di10 | BN9 | CN10 |
| Di11 | BN9 | CN11 |
| Di12 | BN9 | CN12 |
| Di13 | BN9 | CN13 |
| Di14 | BN9 | CN14 |
| Di15 | BN9 | CN15 |
| Di16 | BN9 | CN16 |
| Di17 | BN9 | CN17 |
| Di18 | BN9 | CN18 |
| Di19 | BN9 | CN19 |
| Di20 | BN9 | CN20 |
| Di21 | BN9 | CN21 |
| Di22 | BN9 | CN22 |
| Di23 | BN9 | CN23 |
| Di24 | BN9 | CN24 |
| Di25 | BN9 | CN25 |
| Di26 | BN9 | CN26 |
| Di27 | BN9 | CN27 |
| Di28 | BN9 | CN28 |
| Di29 | BN9 | CN29 |
| Di30 | BN9 | CN30 |
| Di31 | BN9 | CN31 |
| Di32 | BN9 | CN32 |
| Di33 | BN9 | CN33 |
| Di34 | BN9 | CN34 |
| Di35 | BN9 | CN35 |
| Di36 | BN9 | CN36 |
| Di37 | BN9 | CN37 |
| Di38 | BN9 | CN38 |
| Di39 | BN9 | CN39 |
| Di40 | BN9 | CN40 |
| Di41 | BN9 | CN41 |
| Di42 | BN9 | CN42 |
| Di43 | BN9 | CN43 |
| Di44 | BN9 | CN44 |
| Di45 | BN9 | CN45 |
| Di46 | BN9 | CN46 |
| Dj1 | BN10 | CN1 |
| Dj2 | BN10 | CN2 |
| Dj3 | BN10 | CN3 |
| Dj4 | BN10 | CN4 |
| Dj5 | BN10 | CN5 |
| Dj6 | BN10 | CN6 |
| Dj7 | BN10 | CN7 |
| Dj8 | BN10 | CN8 |
| Dj9 | BN10 | CN9 |
| Dj10 | BN10 | CN10 |
| Dj11 | BN10 | CN11 |
| Dj12 | BN10 | CN12 |
| Dj13 | BN10 | CN13 |
| Dj14 | BN10 | CN14 |
| Dj15 | BN10 | CN15 |
| Dj16 | BN10 | CN16 |
| Dj17 | BN10 | CN17 |
| Dj18 | BN10 | CN18 |
| Dj19 | BN10 | CN19 |
| Dj20 | BN10 | CN20 |
| Dj21 | BN10 | CN21 |
| Dj22 | BN10 | CN22 |
| Dj23 | BN10 | CN23 |
| Dj24 | BN10 | CN24 |
| Dj25 | BN10 | CN25 |
| Dj26 | BN10 | CN26 |
| Dj27 | BN10 | CN27 |
| Dj28 | BN10 | CN28 |
| Dj29 | BN10 | CN29 |
| Dj30 | BN10 | CN30 |
| Dj31 | BN10 | CN31 |
| Dj32 | BN10 | CN32 |
| Dj33 | BN10 | CN33 |
| Dj34 | BN10 | CN34 |
| Dj35 | BN10 | CN35 |
| Dj36 | BN10 | CN36 |
| Dj37 | BN10 | CN37 |
| Dj38 | BN10 | CN38 |
| Dj39 | BN10 | CN39 |
| Dj40 | BN10 | CN40 |
| Dj41 | BN10 | CN41 |
| Dj42 | BN10 | CN42 |
| Dj43 | BN10 | CN43 |
| Dj44 | BN10 | CN44 |
| Dj45 | BN10 | CN45 |
| Dj46 | BN10 | CN46 |
| Dk1 | BN11 | CN1 |
| Dk2 | BN11 | CN2 |
| Dk3 | BN11 | CN3 |
| Dk4 | BN11 | CN4 |
| Dk5 | BN11 | CN5 |
| Dk6 | BN11 | CN6 |
| Dk7 | BN11 | CN7 |
| Dk8 | BN11 | CN8 |
| Dk9 | BN11 | CN9 |
| Dk10 | BN11 | CN10 |
| Dk11 | BN11 | CN11 |
| Dk12 | BN11 | CN12 |
| Dk13 | BN11 | CN13 |
| Dk14 | BN11 | CN14 |
| Dk15 | BN11 | CN15 |
| Dk16 | BN11 | CN16 |
| Dk17 | BN11 | CN17 |
| Dk18 | BN11 | CN18 |
| Dk19 | BN11 | CN19 |
| Dk20 | BN11 | CN20 |
| Dk21 | BN11 | CN21 |
| Dk22 | BN11 | CN22 |
| Dk23 | BN11 | CN23 |
| Dk24 | BN11 | CN24 |
| Dk25 | BN11 | CN25 |
| Dk26 | BN11 | CN26 |
| Dk27 | BN11 | CN27 |
| Dk28 | BN11 | CN28 |
| Dk29 | BN11 | CN29 |
| Dk30 | BN11 | CN30 |
| Dk31 | BN11 | CN31 |
| Dk32 | BN11 | CN32 |
| Dk33 | BN11 | CN33 |
| Dk34 | BN11 | CN34 |
| Dk35 | BN11 | CN35 |
| Dk36 | BN11 | CN36 |
| Dk37 | BN11 | CN37 |
| Dk38 | BN11 | CN38 |
| Dk39 | BN11 | CN39 |
| Dk40 | BN11 | CN40 |
| Dk41 | BN11 | CN41 |
| Dk42 | BN11 | CN42 |
| Dk43 | BN11 | CN43 |
| Dk44 | BN11 | CN44 |
| Dk45 | BN11 | CN45 |
| Dk46 | BN11 | CN46 |
| Dl1 | BN12 | CN1 |
| Dl2 | BN12 | CN2 |
| Dl3 | BN12 | CN3 |
| Dl4 | BN12 | CN4 |
| Dl5 | BN12 | CN5 |
| Dl6 | BN12 | CN6 |
| Dl7 | BN12 | CN7 |
| Dl8 | BN12 | CN8 |
| Dl9 | BN12 | CN9 |
| Dl10 | BN12 | CN10 |
| Dl11 | BN12 | CN11 |
| Dl12 | BN12 | CN12 |
| Dl13 | BN12 | CN13 |
| Dl14 | BN12 | CN14 |
| Dl15 | BN12 | CN15 |
| Dl16 | BN12 | CN16 |
| Dl17 | BN12 | CN17 |
| Dl18 | BN12 | CN18 |
| Dl19 | BN12 | CN19 |
| Dl20 | BN12 | CN20 |
| Dl21 | BN12 | CN21 |
| Dl22 | BN12 | CN22 |
| Dl23 | BN12 | CN23 |
| Dl24 | BN12 | CN24 |
| Dl25 | BN12 | CN25 |
| Dl26 | BN12 | CN26 |
| Dl27 | BN12 | CN27 |
| Dl28 | BN12 | CN28 |
| Dl29 | BN12 | CN29 |
| Dl30 | BN12 | CN30 |
| Dl31 | BN12 | CN31 |
| Dl32 | BN12 | CN32 |
| Dl33 | BN12 | CN33 |
| Dl34 | BN12 | CN34 |
| Dl35 | BN12 | CN35 |
| Dl36 | BN12 | CN36 |
| Dl37 | BN12 | CN37 |
| Dl38 | BN12 | CN38 |
| Dl39 | BN12 | CN39 |
| Dl40 | BN12 | CN40 |
| Dl41 | BN12 | CN41 |
| Dl42 | BN12 | CN42 |
| Dl43 | BN12 | CN43 |
| Dl44 | BN12 | CN44 |
| Dl45 | BN12 | CN45 |
| Dl46 | BN12 | CN46 |
| Dm1 | BN13 | CN1 |
| Dm2 | BN13 | CN2 |
| Dm3 | BN13 | CN3 |
| Dm4 | BN13 | CN4 |
| Dm5 | BN13 | CN5 |
| Dm6 | BN13 | CN6 |
| Dm7 | BN13 | CN7 |
| Dm8 | BN13 | CN8 |
| Dm9 | BN13 | CN9 |
| Dm10 | BN13 | CN10 |
| Dm11 | BN13 | CN11 |
| Dm12 | BN13 | CN12 |
| Dm13 | BN13 | CN13 |
| Dm14 | BN13 | CN14 |
| Dm15 | BN13 | CN15 |
| Dm16 | BN13 | CN16 |
| Dm17 | BN13 | CN17 |
| Dm18 | BN13 | CN18 |
| Dm19 | BN13 | CN19 |
| Dm20 | BN13 | CN20 |
| Dm21 | BN13 | CN21 |
| Dm22 | BN13 | CN22 |
| Dm23 | BN13 | CN23 |
| Dm24 | BN13 | CN24 |
| Dm25 | BN13 | CN25 |
| Dm26 | BN13 | CN26 |
| Dm27 | BN13 | CN27 |
| Dm28 | BN13 | CN28 |
| Dm29 | BN13 | CN29 |
| Dm30 | BN13 | CN30 |
| Dm31 | BN13 | CN31 |
| Dm32 | BN13 | CN32 |
| Dm33 | BN13 | CN33 |
| Dm34 | BN13 | CN34 |
| Dm35 | BN13 | CN35 |
| Dm36 | BN13 | CN36 |
| Dm37 | BN13 | CN37 |
| Dm38 | BN13 | CN38 |
| Dm39 | BN13 | CN39 |
| Dm40 | BN13 | CN40 |
| Dm41 | BN13 | CN41 |
| Dm42 | BN13 | CN42 |
| Dm43 | BN13 | CN43 |
| Dm44 | BN13 | CN44 |
| Dm45 | BN13 | CN45 |
| Dm46 | BN13 | CN46 |
| Dn1 | BN14 | CN1 |
| Dn2 | BN14 | CN2 |
| Dn3 | BN14 | CN3 |
| Dn4 | BN14 | CN4 |
| Dn5 | BN14 | CN5 |
| Dn6 | BN14 | CN6 |
| Dn7 | BN14 | CN7 |
| Dn8 | BN14 | CN8 |
| Dn9 | BN14 | CN9 |
| Dn10 | BN14 | CN10 |
| Dn11 | BN14 | CN11 |
| Dn12 | BN14 | CN12 |
| Dn13 | BN14 | CN13 |
| Dn14 | BN14 | CN14 |
| Dn15 | BN14 | CN15 |
| Dn16 | BN14 | CN16 |
| Dn17 | BN14 | CN17 |
| Dn18 | BN14 | CN18 |
| Dn19 | BN14 | CN19 |
| Dn20 | BN14 | CN20 |
| Dn21 | BN14 | CN21 |
| Dn22 | BN14 | CN22 |
| Dn23 | BN14 | CN23 |
| Dn24 | BN14 | CN24 |
| Dn25 | BN14 | CN25 |
| Dn26 | BN14 | CN26 |
| Dn27 | BN14 | CN27 |
| Dn28 | BN14 | CN28 |
| Dn29 | BN14 | CN29 |
| Dn30 | BN14 | CN30 |
| Dn31 | BN14 | CN31 |
| Dn32 | BN14 | CN32 |
| Dn33 | BN14 | CN33 |
| Dn34 | BN14 | CN34 |
| Dn35 | BN14 | CN35 |
| Dn36 | BN14 | CN36 |
| Dn37 | BN14 | CN37 |
| Dn38 | BN14 | CN38 |
| Dn39 | BN14 | CN39 |
| Dn40 | BN14 | CN40 |
| Dn41 | BN14 | CN41 |
| Dn42 | BN14 | CN42 |
| Dn43 | BN14 | CN43 |
| Dn44 | BN14 | CN44 |
| Dn45 | BN14 | CN45 |
| Dn46 | BN14 | CN46 |
| Do1 | BN15 | CN1 |
| Do2 | BN15 | CN2 |
| Do3 | BN15 | CN3 |
| Do4 | BN15 | CN4 |
| Do5 | BN15 | CN5 |
| Do6 | BN15 | CN6 |
| Do7 | BN15 | CN7 |
| Do8 | BN15 | CN8 |
| Do9 | BN15 | CN9 |
| Do10 | BN15 | CN10 |
| Do11 | BN15 | CN11 |
| Do12 | BN15 | CN12 |
| Do13 | BN15 | CN13 |
| Do14 | BN15 | CN14 |
| Do15 | BN15 | CN15 |
| Do16 | BN15 | CN16 |
| Do17 | BN15 | CN17 |
| Do18 | BN15 | CN18 |
| Do19 | BN15 | CN19 |
| Do20 | BN15 | CN20 |
| Do21 | BN15 | CN21 |
| Do22 | BN15 | CN22 |
| Do23 | BN15 | CN23 |
| Do24 | BN15 | CN24 |
| Do25 | BN15 | CN25 |
| Do26 | BN15 | CN26 |
| Do27 | BN15 | CN27 |
| Do28 | BN15 | CN28 |
| Do29 | BN15 | CN29 |
| Do30 | BN15 | CN30 |
| Do31 | BN15 | CN31 |
| Do32 | BN15 | CN32 |
| Do33 | BN15 | CN33 |
| Do34 | BN15 | CN34 |
| Do35 | BN15 | CN35 |
| Do36 | BN15 | CN36 |
| Do37 | BN15 | CN37 |
| Do38 | BN15 | CN38 |
| Do39 | BN15 | CN39 |
| Do40 | BN15 | CN40 |
| Do41 | BN15 | CN41 |
| Do42 | BN15 | CN42 |
| Do43 | BN15 | CN43 |
| Do44 | BN15 | CN44 |
| Do45 | BN15 | CN45 |
| Do46 | BN15 | CN46 |
| Dp1 | BN16 | CN1 |
| Dp2 | BN16 | CN2 |
| Dp3 | BN16 | CN3 |
| Dp4 | BN16 | CN4 |
| Dp5 | BN16 | CN5 |
| Dp6 | BN16 | CN6 |
| Dp7 | BN16 | CN7 |
| Dp8 | BN16 | CN8 |
| Dp9 | BN16 | CN9 |
| Dp10 | BN16 | CN10 |
| Dp11 | BN16 | CN11 |
| Dp12 | BN16 | CN12 |
| Dp13 | BN16 | CN13 |
| Dp14 | BN16 | CN14 |
| Dp15 | BN16 | CN15 |
| Dp16 | BN16 | CN16 |
| Dp17 | BN16 | CN17 |
| Dp18 | BN16 | CN18 |
| Dp19 | BN16 | CN19 |
| Dp20 | BN16 | CN20 |
| Dp21 | BN16 | CN21 |
| Dp22 | BN16 | CN22 |
| Dp23 | BN16 | CN23 |
| Dp24 | BN16 | CN24 |
| Dp25 | BN16 | CN25 |
| Dp26 | BN16 | CN26 |
| Dp27 | BN16 | CN27 |
| Dp28 | BN16 | CN28 |
| Dp29 | BN16 | CN29 |
| Dp30 | BN16 | CN30 |
| Dp31 | BN16 | CN31 |
| Dp32 | BN16 | CN32 |
| Dp33 | BN16 | CN33 |
| Dp34 | BN16 | CN34 |
| Dp35 | BN16 | CN35 |
| Dp36 | BN16 | CN36 |
| Dp37 | BN16 | CN37 |
| Dp38 | BN16 | CN38 |
| Dp39 | BN16 | CN39 |
| Dp40 | BN16 | CN40 |
| Dp41 | BN16 | CN41 |
| Dp42 | BN16 | CN42 |
| Dp43 | BN16 | CN43 |
| Dp44 | BN16 | CN44 |
| Dp45 | BN16 | CN45 |
| Dp46 | BN16 | CN46 |
| Dq1 | BN17 | CN1 |
| Dq2 | BN17 | CN2 |
| Dq3 | BN17 | CN3 |
| Dq4 | BN17 | CN4 |
| Dq5 | BN17 | CN5 |
| Dq6 | BN17 | CN6 |
| Dq7 | BN17 | CN7 |
| Dq8 | BN17 | CN8 |
| Dq9 | BN17 | CN9 |
| Dq10 | BN17 | CN10 |
| Dq11 | BN17 | CN11 |
| Dq12 | BN17 | CN12 |
| Dq13 | BN17 | CN13 |
| Dq14 | BN17 | CN14 |
| Dq15 | BN17 | CN15 |
| Dq16 | BN17 | CN16 |
| Dq17 | BN17 | CN17 |
| Dq18 | BN17 | CN18 |
| Dq19 | BN17 | CN19 |
| Dq20 | BN17 | CN20 |
| Dq21 | BN17 | CN21 |
| Dq22 | BN17 | CN22 |
| Dq23 | BN17 | CN23 |
| Dq24 | BN17 | CN24 |
| Dq25 | BN17 | CN25 |
| Dq26 | BN17 | CN26 |
| Dq27 | BN17 | CN27 |
| Dq28 | BN17 | CN28 |
| Dq29 | BN17 | CN29 |
| Dq30 | BN17 | CN30 |
| Dq31 | BN17 | CN31 |
| Dq32 | BN17 | CN32 |
| Dq33 | BN17 | CN33 |
| Dq34 | BN17 | CN34 |
| Dq35 | BN17 | CN35 |
| Dq36 | BN17 | CN36 |
| Dq37 | BN17 | CN37 |
| Dq38 | BN17 | CN38 |
| Dq39 | BN17 | CN39 |
| Dq40 | BN17 | CN40 |
| Dq41 | BN17 | CN41 |
| Dq42 | BN17 | CN42 |
| Dq43 | BN17 | CN43 |
| Dq44 | BN17 | CN44 |
| Dq45 | BN17 | CN45 |
| Dq46 | BN17 | CN46 |
| Ds1 | BN18 | CN1 |
| Ds2 | BN18 | CN2 |
| Ds3 | BN18 | CN3 |
| Ds4 | BN18 | CN4 |
| Ds5 | BN18 | CN5 |
| Ds6 | BN18 | CN6 |
| Ds7 | BN18 | CN7 |
| Ds8 | BN18 | CN8 |
| Ds9 | BN18 | CN9 |
| Ds10 | BN18 | CN10 |
| Ds11 | BN18 | CN11 |
| Ds12 | BN18 | CN12 |
| Ds13 | BN18 | CN13 |
| Ds14 | BN18 | CN14 |
| Ds15 | BN18 | CN15 |
| Ds16 | BN18 | CN16 |
| Ds17 | BN18 | CN17 |
| Ds18 | BN18 | CN18 |
| Ds19 | BN18 | CN19 |
| Ds20 | BN18 | CN20 |
| Ds21 | BN18 | CN21 |
| Ds22 | BN18 | CN22 |
| Ds23 | BN18 | CN23 |
| Ds24 | BN18 | CN24 |
| Ds25 | BN18 | CN25 |
| Ds26 | BN18 | CN26 |
| Ds27 | BN18 | CN27 |
| Ds28 | BN18 | CN28 |
| Ds29 | BN18 | CN29 |
| Ds30 | BN18 | CN30 |
| Ds31 | BN18 | CN31 |
| Ds32 | BN18 | CN32 |
| Ds33 | BN18 | CN33 |
| Ds34 | BN18 | CN34 |
| Ds35 | BN18 | CN35 |
| Ds36 | BN18 | CN36 |
| Ds37 | BN18 | CN37 |
| Ds38 | BN18 | CN38 |
| Ds39 | BN18 | CN39 |
| Ds40 | BN18 | CN40 |
| Ds41 | BN18 | CN41 |
| Ds42 | BN18 | CN42 |
| Ds43 | BN18 | CN43 |
| Ds44 | BN18 | CN44 |
| Ds45 | BN18 | CN45 |
| Ds46 | BN18 | CN46 |
| Dt1 | BN19 | CN1 |
| Dt2 | BN19 | CN2 |
| Dt3 | BN19 | CN3 |
| Dt4 | BN19 | CN4 |
| Dt5 | BN19 | CN5 |
| Dt6 | BN19 | CN6 |
| Dt7 | BN19 | CN7 |
| Dt8 | BN19 | CN8 |
| Dt9 | BN19 | CN9 |
| Dt10 | BN19 | CN10 |
| Dt11 | BN19 | CN11 |
| Dt12 | BN19 | CN12 |
| Dt13 | BN19 | CN13 |
| Dt14 | BN19 | CN14 |
| Dt15 | BN19 | CN15 |
| Dt16 | BN19 | CN16 |
| Dt17 | BN19 | CN17 |
| Dt18 | BN19 | CN18 |
| Dt19 | BN19 | CN19 |
| Dt20 | BN19 | CN20 |
| Dt21 | BN19 | CN21 |
| Dt22 | BN19 | CN22 |
| Dt23 | BN19 | CN23 |
| Dt24 | BN19 | CN24 |
| Dt25 | BN19 | CN25 |
| Dt26 | BN19 | CN26 |
| Dt27 | BN19 | CN27 |
| Dt28 | BN19 | CN28 |
| Dt29 | BN19 | CN29 |
| Dt30 | BN19 | CN30 |
| Dt31 | BN19 | CN31 |
| Dt32 | BN19 | CN32 |
| Dt33 | BN19 | CN33 |
| Dt34 | BN19 | CN34 |
| Dt35 | BN19 | CN35 |
| Dt36 | BN19 | CN36 |
| Dt37 | BN19 | CN37 |
| Dt38 | BN19 | CN38 |
| Dt39 | BN19 | CN39 |
| Dt40 | BN19 | CN40 |
| Dt41 | BN19 | CN41 |
| Dt42 | BN19 | CN42 |
| Dt43 | BN19 | CN43 |
| Dt44 | BN19 | CN44 |
| Dt45 | BN19 | CN45 |
| Dt46 | BN19 | CN46 |
| Du1 | BN20 | CN1 |
| Du2 | BN20 | CN2 |
| Du3 | BN20 | CN3 |
| Du4 | BN20 | CN4 |
| Du5 | BN20 | CN5 |
| Du6 | BN20 | CN6 |
| Du7 | BN20 | CN7 |
| Du8 | BN20 | CN8 |
| Du9 | BN20 | CN9 |
| Du10 | BN20 | CN10 |
| Du11 | BN20 | CN11 |
| Du12 | BN20 | CN12 |
| Du13 | BN20 | CN13 |
| Du14 | BN20 | CN14 |
| Du15 | BN20 | CN15 |
| Du16 | BN20 | CN16 |
| Du17 | BN20 | CN17 |
| Du18 | BN20 | CN18 |
| Du19 | BN20 | CN19 |
| Du20 | BN20 | CN20 |
| Du21 | BN20 | CN21 |
| Du22 | BN20 | CN22 |
| Du23 | BN20 | CN23 |
| Du24 | BN20 | CN24 |
| Du25 | BN20 | CN25 |
| Du26 | BN20 | CN26 |
| Du27 | BN20 | CN27 |
| Du28 | BN20 | CN28 |
| Du29 | BN20 | CN29 |
| Du30 | BN20 | CN30 |
| Du31 | BN20 | CN31 |
| Du32 | BN20 | CN32 |
| Du33 | BN20 | CN33 |
| Du34 | BN20 | CN34 |
| Du35 | BN20 | CN35 |
| Du36 | BN20 | CN36 |
| Du37 | BN20 | CN37 |
| Du38 | BN20 | CN38 |
| Du39 | BN20 | CN39 |
| Du40 | BN20 | CN40 |
| Du41 | BN20 | CN41 |
| Du42 | BN20 | CN42 |
| Du43 | BN20 | CN43 |
| Du44 | BN20 | CN44 |
| Du45 | BN20 | CN45 |
| Du46 | BN20 | CN46 |
| Dv1 | BN21 | CN1 |
| Dv2 | BN21 | CN2 |
| Dv3 | BN21 | CN3 |
| Dv4 | BN21 | CN4 |
| Dv5 | BN21 | CN5 |
| Dv6 | BN21 | CN6 |
| Dv7 | BN21 | CN7 |
| Dv8 | BN21 | CN8 |
| Dv9 | BN21 | CN9 |
| Dv10 | BN21 | CN10 |
| Dv11 | BN21 | CN11 |
| Dv12 | BN21 | CN12 |
| Dv13 | BN21 | CN13 |
| Dv14 | BN21 | CN14 |
| Dv15 | BN21 | CN15 |
| Dv16 | BN21 | CN16 |
| Dv17 | BN21 | CN17 |
| Dv18 | BN21 | CN18 |
| Dv19 | BN21 | CN19 |
| Dv20 | BN21 | CN20 |
| Dv21 | BN21 | CN21 |
| Dv22 | BN21 | CN22 |
| Dv23 | BN21 | CN23 |
| Dv24 | BN21 | CN24 |
| Dv25 | BN21 | CN25 |
| Dv26 | BN21 | CN26 |
| Dv27 | BN21 | CN27 |
| Dv28 | BN21 | CN28 |
| Dv29 | BN21 | CN29 |
| Dv30 | BN21 | CN30 |
| Dv31 | BN21 | CN31 |
| Dv32 | BN21 | CN32 |
| Dv33 | BN21 | CN33 |
| Dv34 | BN21 | CN34 |
| Dv35 | BN21 | CN35 |
| Dv36 | BN21 | CN36 |
| Dv37 | BN21 | CN37 |
| Dv38 | BN21 | CN38 |
| Dv39 | BN21 | CN39 |
| Dv40 | BN21 | CN40 |
| Dv41 | BN21 | CN41 |
| Dv42 | BN21 | CN42 |
| Dv43 | BN21 | CN43 |
| Dv44 | BN21 | CN44 |
| Dv45 | BN21 | CN45 |
| Dv46 | BN21 | CN46 |

As used herein, Compounds Aa1 to Av46 shown in Table 1 may be referred to as Compound Group A, Compounds Ba1 to Bv46 shown in Table 2 may be referred to as Compound Group B, Compounds Cal to Cv46 shown in Table 3 may be referred to as Compound Group C, and Compounds Dal to Dv46 shown in Table 4 may be referred to as Compound Group D. The amine compound of one or more embodiments may be represented by one of compounds of Compound Group A, Compound Group B, Compound Group C, and Compound Group D. The hole transport region HTR of the light emitting element ED of one or more embodiments may include at least one of the amine compounds disclosed in Compound Group A, Compound Group B, Compound Group C, and Compound Group D. The hole transport layer HTL of the light emitting element ED may include at least one of the amine compounds disclosed in Compound Group A, Compound Group B, Compound Group C, and Compound Group D. For example, the amine compound of one or more embodiments may include at least one of the amine compounds disclosed in Compound Group 1. Compounds 1 to 12 disclosed in Compound Group 1 indicate Compound Aa16, Compound Ab16, Compound Ac16, Compound Ad16, Compound Ae4, Compound Ae16, Compound Ae24, Compound Ae29, Compound Ae34, Compound Ae43, Compound A₁ 6, and Compound As16 of Compound Group A described herein, respectively, and Compounds 13 to 15 disclosed in Compound Group 1 indicate Be34 of Compound Group B described herein, Ce34 of Compound Group C described herein, and De34 of Compound Group D described herein, respectively.

Accordingly, Compound Group 1 serves as a representative subset of amine compounds selected from the broader structural families defined by Compound Groups A through D. These representative compounds illustrate specific combinations of substituents that have been identified as particularly suitable for use in the hole transport region of light emitting elements. By referencing Compound Group 1, the disclosure highlights practical examples of how the general structural frameworks and substituent options described above may be applied to yield compounds with desirable performance characteristics for optoelectronic applications.

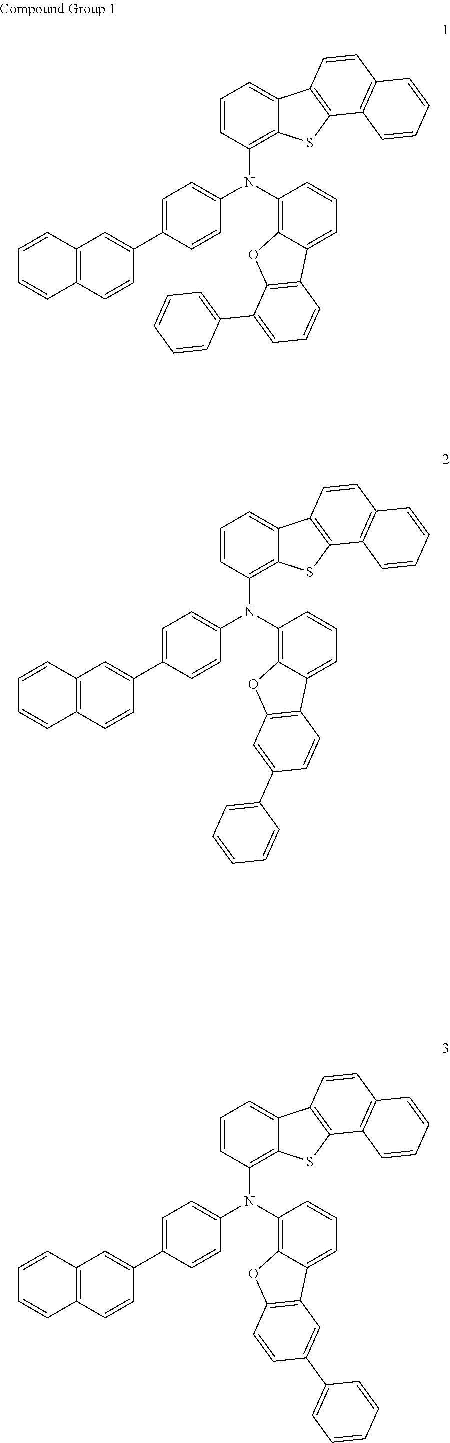 [SMILES: c1ccc(-c2cccc3c2oc2c(N(c4ccc(-c5ccc6ccccc6c5)cc4)c4cccc5c4sc4c6ccccc6ccc54)cccc23)cc1 ; c1ccc(-c2ccc3c(c2)oc2c(N(c4ccc(-c5ccc6ccccc6c5)cc4)c4cccc5c4sc4c6ccccc6ccc54)cccc23)cc1 ; c1ccc(-c2ccc3oc4c(N(c5ccc(-c6ccc7ccccc7c6)cc5)c5cccc6c5sc5c7ccccc7ccc65)cccc4c3c2)cc1]

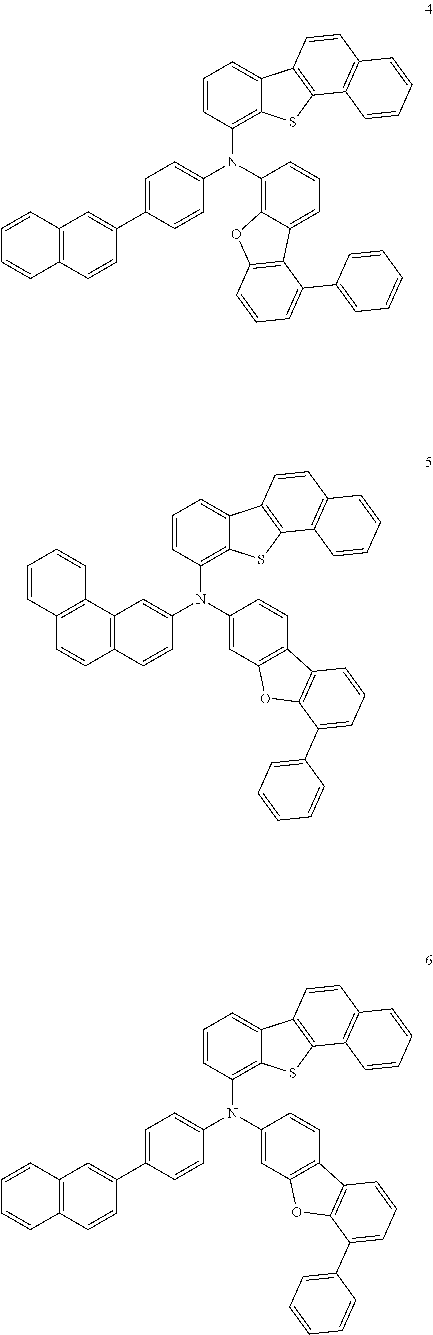 [SMILES: c1ccc(-c2cccc3oc4c(N(c5ccc(-c6ccc7ccccc7c6)cc5)c5cccc6c5sc5c7ccccc7ccc65)cccc4c23)cc1 ; c1ccc(-c2cccc3c2oc2cc(N(c4ccc5ccc6ccccc6c5c4)c4cccc5c4sc4c6ccccc6ccc54)ccc23)cc1 ; c1ccc(-c2cccc3c2oc2cc(N(c4ccc(-c5ccc6ccccc6c5)cc4)c4cccc5c4sc4c6ccccc6ccc54)ccc23)cc1]

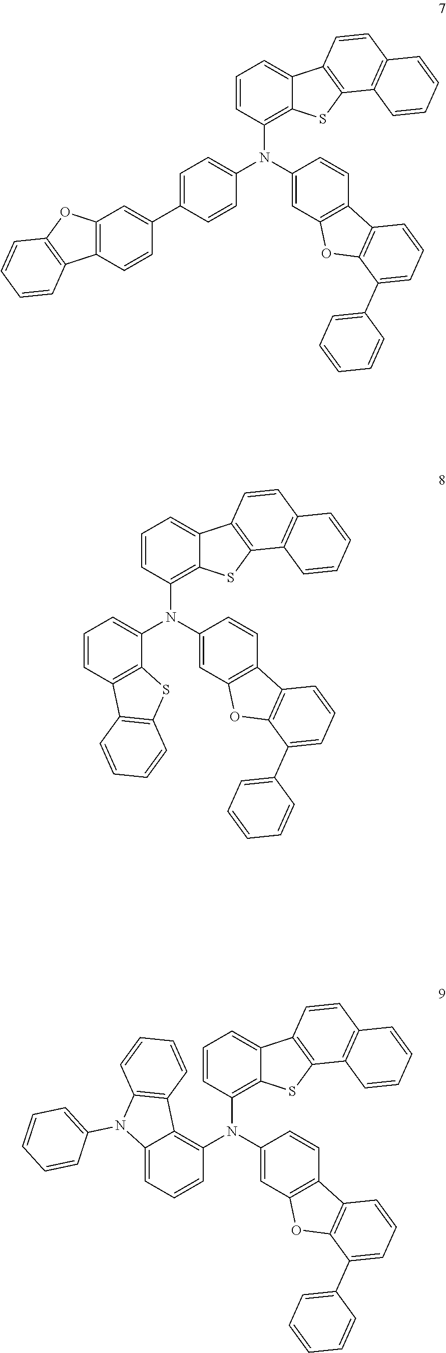 [SMILES: c1ccc(-c2cccc3c2oc2cc(N(c4ccc(-c5ccc6c(c5)oc5ccccc56)cc4)c4cccc5c4sc4c6ccccc6ccc54)ccc23)cc1 ; c1ccc(-c2cccc3c2oc2cc(N(c4cccc5c4sc4ccccc45)c4cccc5c4sc4c6ccccc6ccc54)ccc23)cc1 ; c1ccc(-c2cccc3c2oc2cc(N(c4cccc5c4sc4c6ccccc6ccc54)c4cccc5c4c4ccccc4n5-c4ccccc4)ccc23)cc1]

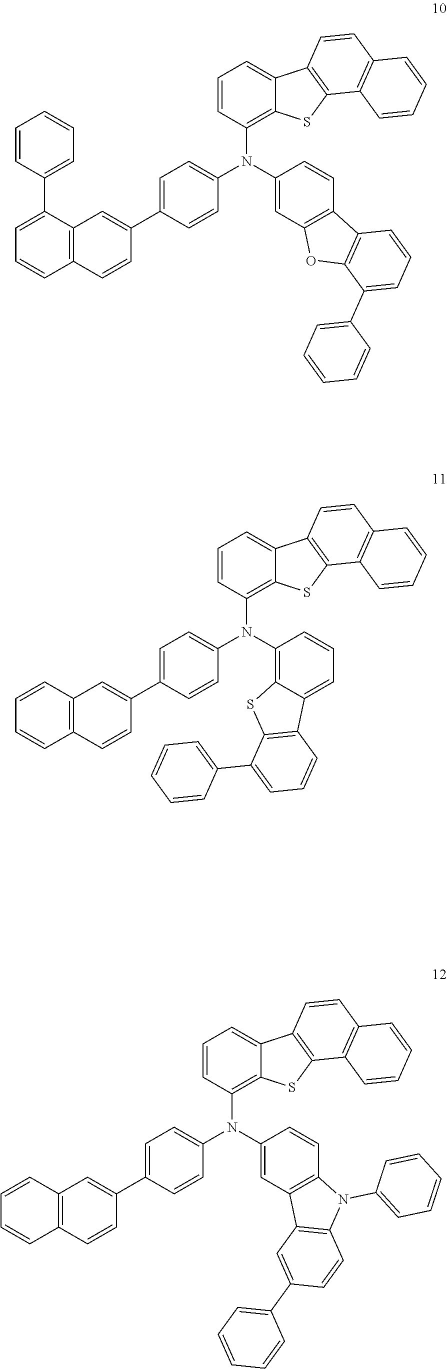 [SMILES: c1ccc(-c2cccc3ccc(-c4ccc(N(c5ccc6c(c5)oc5c(-c7ccccc7)cccc56)c5cccc6c5sc5c7ccccc7ccc65)cc4)cc23)cc1 ; c1ccc(-c2cccc3c2sc2c(N(c4ccc(-c5ccc6ccccc6c5)cc4)c4cccc5c4sc4c6ccccc6ccc54)cccc23)cc1 ; c1ccc(-c2ccc3c(c2)c2cc(N(c4ccc(-c5ccc6ccccc6c5)cc4)c4cccc5c4sc4c6ccccc6ccc54)ccc2n3-c2ccccc2)cc1]

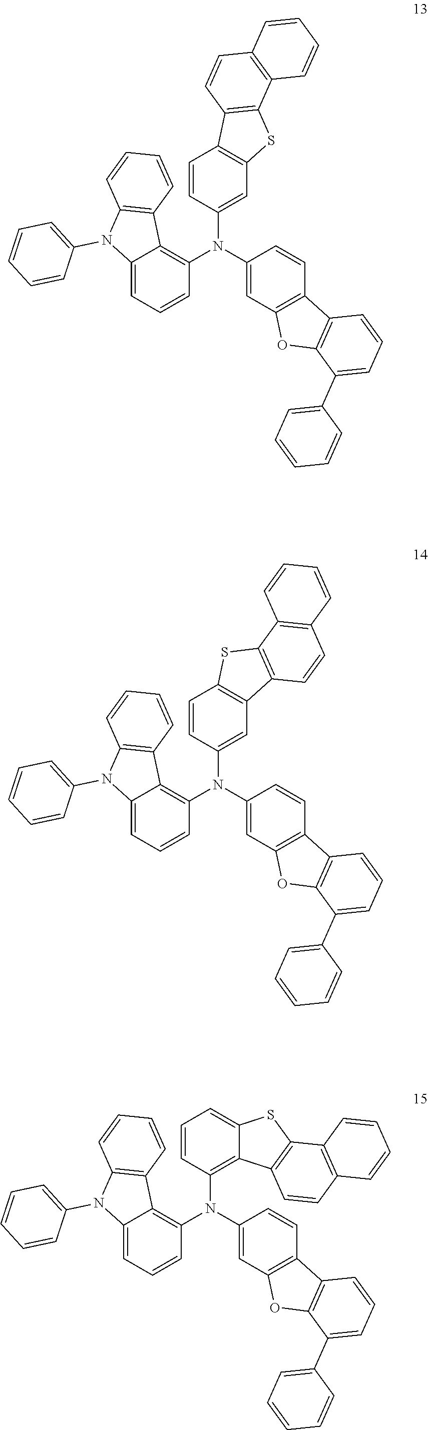 [SMILES: c1ccc(-c2cccc3c2oc2cc(N(c4ccc5c(c4)sc4c6ccccc6ccc54)c4cccc5c4c4ccccc4n5-c4ccccc4)ccc23)cc1 ; c1ccc(-c2cccc3c2oc2cc(N(c4ccc5sc6c7ccccc7ccc6c5c4)c4cccc5c4c4ccccc4n5-c4ccccc4)ccc23)cc1 ; c1ccc(-c2cccc3c2oc2cc(N(c4cccc5sc6c7ccccc7ccc6c45)c4cccc5c4c4ccccc4n5-c4ccccc4)ccc23)cc1]

The amine compound according to one or more embodiments includes the first substituent, the second substituent, and the third substituent, and may thus allow a light emitting element to obtain long lifespan.

The amine compound of one or more embodiments includes an amine group, and the first to third substituents have a structure that bonds with the amine group of the amine compound of one or more embodiments. In this case, the first substituent includes a benzonaphthothiophene moiety. The second substituent includes any one of (e.g., selected from among) a dibenzofuran moiety, a dibenzothiophene moiety, and a carbazole moiety. The second substituent includes a first benzene moiety and a second benzene moiety that are linked to each other via one first heteroatom, the first benzene moiety of the second substituent is bonded to an amine group, and the second substituent may further include a phenyl group linked to the second benzene moiety. The third substituent may be selected from among a substituted or unsubstituted aryl group having 6 to 30 ring-forming carbon atoms, and/or a substituted or unsubstituted heteroaryl group having 2 to 30 ring-forming carbon atoms. However, a case where the third substituent is a substituted or unsubstituted benzofuran group and a substituted or unsubstituted benzothiophene group is excluded.

The amine compound of one or more embodiments may exhibit excellent or suitable stability through the introduction of such a substituent. For example, the amine compound of one or more embodiments including the first substituent may exhibit improved lifespan due to stacking via intermolecular interaction related to the first substituent in the amine compound of one or more embodiments, and intermolecular orientation. In some embodiments, the amine compound of one or more embodiments including the second substituent may exhibit improved lifespan due to stacking via intermolecular interaction related to the second substituent in the amine compound of one or more embodiments, and intermolecular orientation. When the amine compound according to one or more embodiments of the disclosure is applied to the hole transport region HTR of a light emitting element ED, the light emitting element exhibiting long lifespan may be achieved.

In the light emitting element ED of one or more embodiments, the hole transport region HTR may further include a compound represented by Formula H-1.

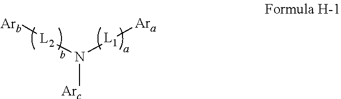

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. For example, a and b may each independently be an integer of 0 to 10. When a or b is an integer of 2 or greater, a plurality of L₁'s and L₂'s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Arₐ and Ar_{b} may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar_{c} may be a substituted or unsubstituted aryl group having 6 to 30 ring-forming carbon atoms.

A compound represented by Formula H-1 may be a monoamine compound. In one or more embodiments, the compound represented by Formula H-1 may be a diamine compound in which at least one of Arₐ to Ar_{c} includes an amine group as a substituent. In some embodiments, the compound represented by Formula H-1 may be a carbazole-based compound including a substituted or unsubstituted carbazole group in at least one of Arₐ or Ar_{b} or a substituted or unsubstituted fluorene-based group in at least one of Arₐ or Ar_{b}.

The compound represented by Formula H-1 may be represented by any one (selected from) among compounds from (in) Compound Group H. However, the compounds listed in Compound Group H are presented as an example, and the compound represented by Formula H-1 is not limited to the those listed in Compound Group H.

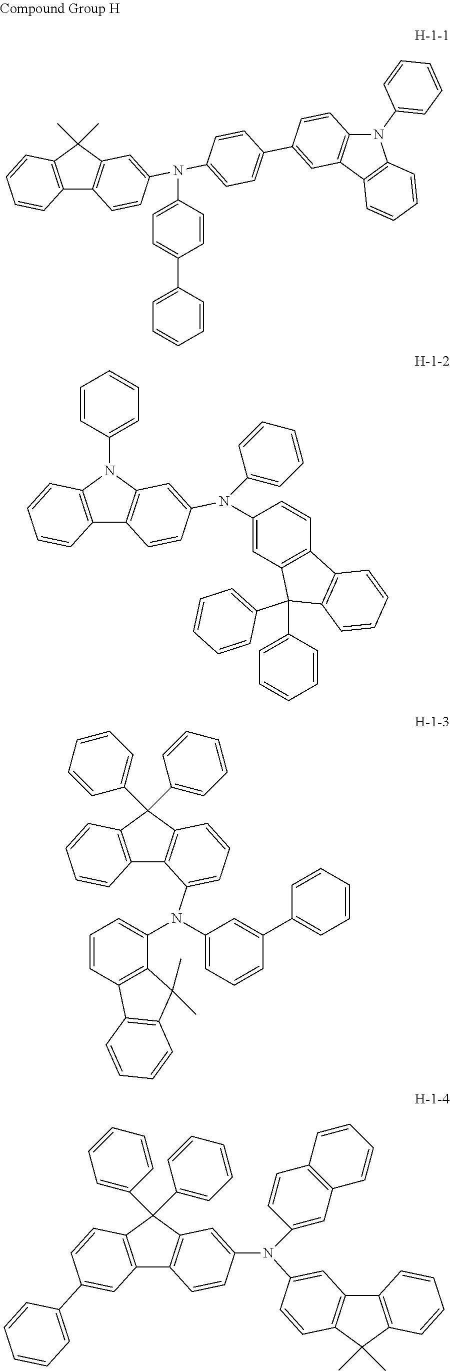 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

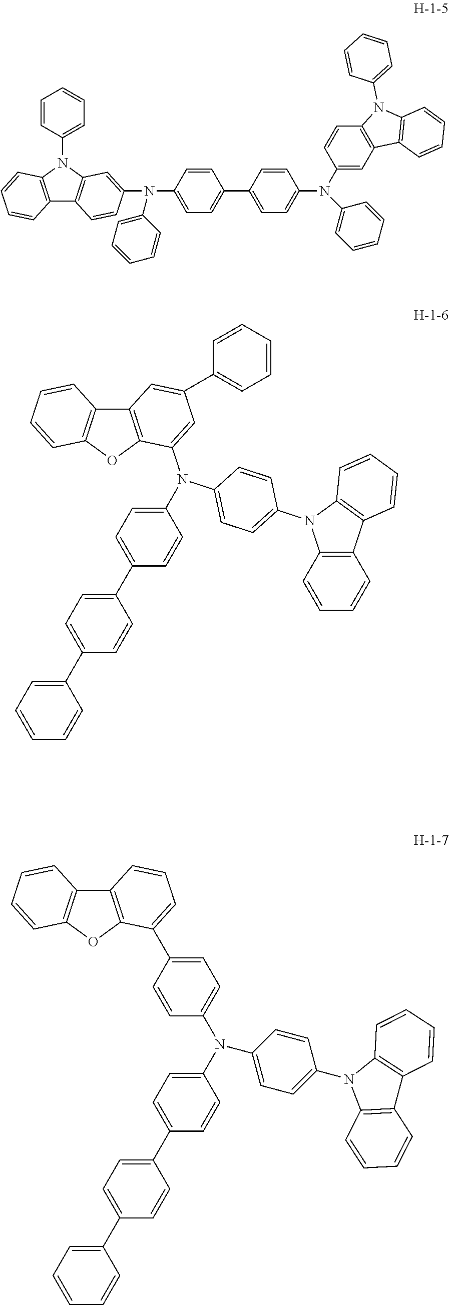 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

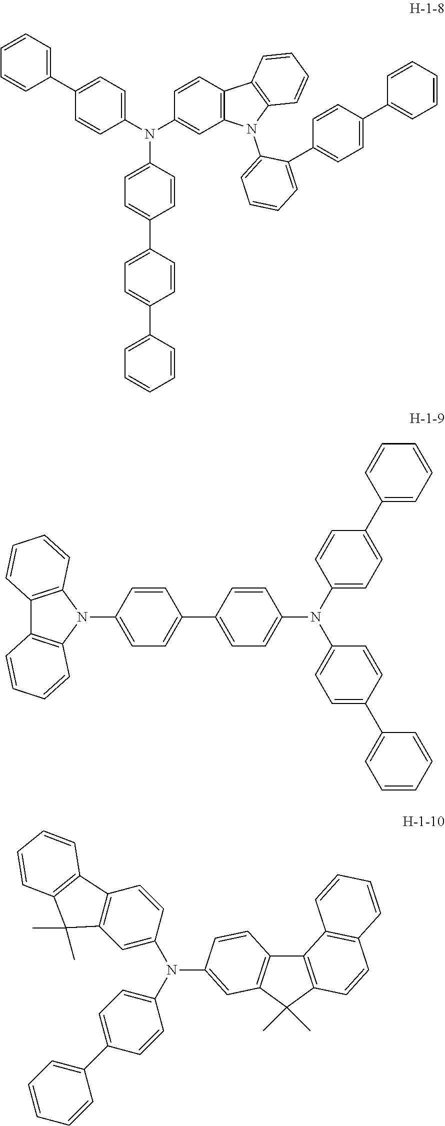 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

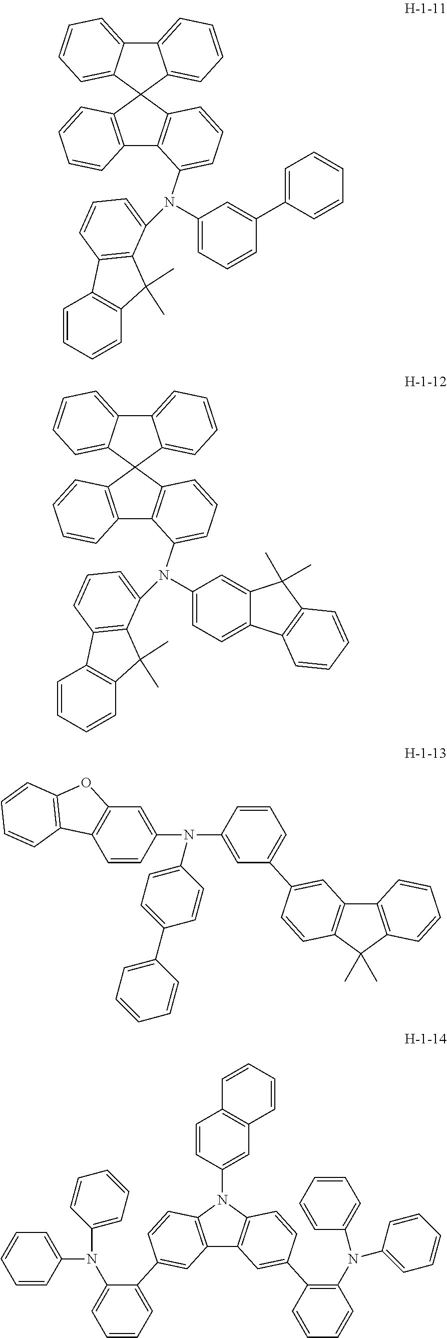

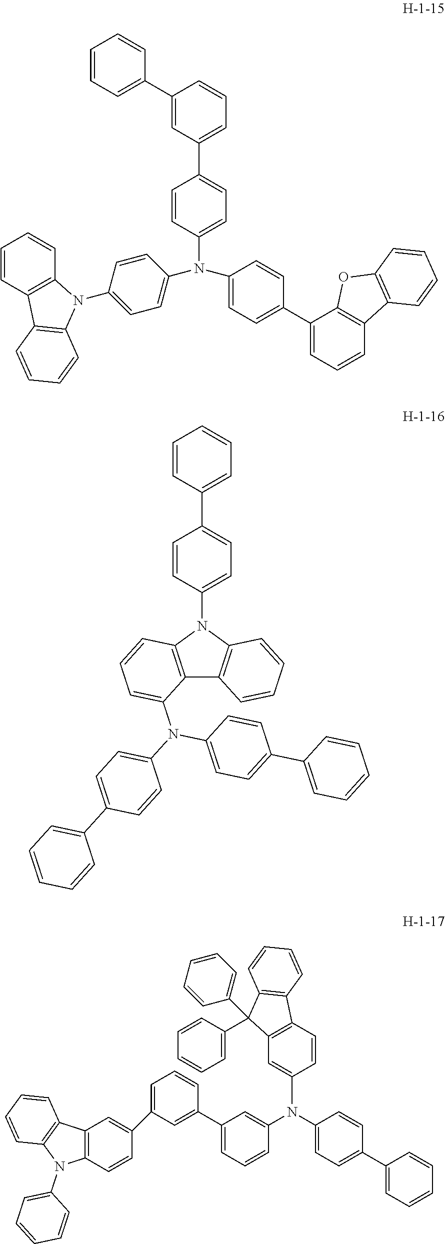 [SMILES: c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1]

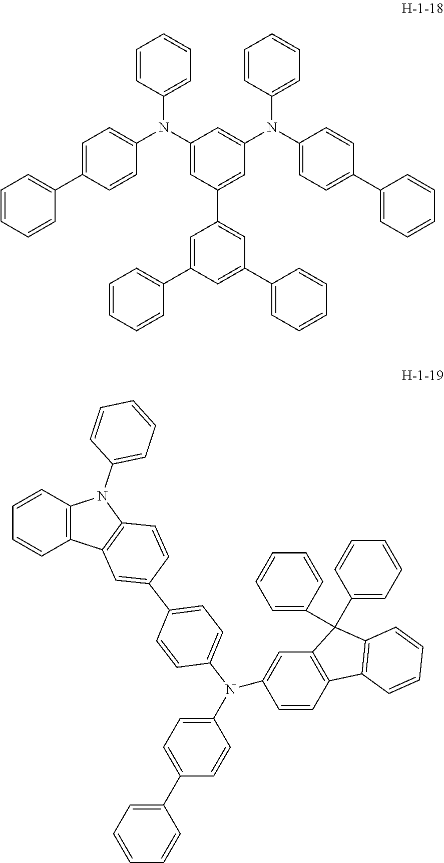 [SMILES: c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

In some embodiments, the hole transport region HTR may further include a suitable hole transport material.

For example, the hole transport region HTR may include a phthalocyanine compound such as copper phthalocyanine, N¹,N^{1′}—([1,1′-biphenyl]-4,4′-diyl)bis(N¹-phenyl-N4,N4-di-m-tolylbenzene-1,4-diamine) (DNTPD), 4,4′,4″-[tris(3-methylphenyl)phenylamino]triphenylamine (m-MTDATA), 4,4′4″-tris(N,N-diphenylamino)triphenylamine (TDATA), 4,4′,4″-tris[N-(1-naphthyl)-N-phenylamino]-triphenylamine (1-TNATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonicacid (PANI/CSA), polyaniline/poly(4-styrenesulfonate) (PANI/PSS), N,N′-di(naphthalene-I-yl)-N,N′-diphenyl-benzidine (NPB or NPD), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), and/or the like.

The hole transport region HTR may include carbazole-based derivatives such as N-phenyl carbazole and polyvinyl carbazole, fluorene-based derivatives, triphenylamine-based derivatives such as 4,4′,4″-tris(N-carbazolyl)triphenylamine (TCTA), N,N′-bis(3-methylphenyl)-N,N′-diphenyl-[1,1′-biphenyl]-4,4′-diamine (TPD), N,N′-di(naphthalene-1-yl)-N,N′-diphenyl-benzidine (NPB), 4,4′-cyclohexylidene bis[N,N-bis(4-methylphenyl)benzenamine](TAPC), 4,4′-bis[N,N′-(3-tolyl)amino]-3,3′-dimethylbiphenyl (HMTPD), 1,3-bis(N-carbazolyl)benzene (mCP), and/or the like.

In some embodiments, the hole transport region HTR may further include 9-(4-tert-Butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include the compounds of the hole transport region described herein in at least one among the hole injection layer HIL, the hole transport layer HTL, and the electron blocking layer EBL.

The hole transport region HTR may have a thickness of about 100 Å to about 10000 Å, for example, about 100 Å to about 5000 Å. When the hole transport region HTR includes the hole injection layer HIL, the hole injection layer HIL may have a thickness of, for example, about 30 Å to about 1000 Å. When the hole transport region HTR includes the hole transport layer HTL, the hole transport layer HTL may have a thickness of about 30 Å to about 1000 Å. When the hole transport region HTR includes the electron blocking layer EBL, the electron blocking layer EBL may have a thickness of, for example, about 10 Å to about 1000 Å. When the thicknesses of the hole transport region HTR, the hole injection layer HIL, the hole transport layer HTL, and the electron blocking layer EBL satisfy the herein-described ranges, satisfactory hole transport properties may be obtained without a substantial increase in driving voltage.

The hole transport region HTR may further include, in addition to the herein-described materials, a charge generation material to increase conductivity. The charge generation material may be uniformly (e.g., substantially uniformly) or non-uniformly (e.g., substantially uniformly) dispersed in the hole transport region HTR. The charge generation material may be, for example, a p-dopant. The p-dopant may include at least one of halogenated metal compounds, quinone derivatives, metal oxides, or cyano group-containing compounds, but the present disclosure is not limited thereto. For example, the p-dopant may include halogenated metal compounds such as CuI and RbI, quinone derivatives such as tetracyanoquinodimethane (TCNQ) and 2,3,5,6-tetrafluoro-7,7,8,8-tetracyanoquinodimethane (F4-TCNQ), metal oxides such as tungsten oxides and molybdenum oxides, cyano group-containing compounds such as dipyrazino[2,3-f: 2′,3′-h]quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like, but the present disclosure is not limited thereto.

As described herein, the hole transport region HTR may further include a buffer layer in addition to the hole injection layer HIL, the hole transport layer HTL, and the electron blocking layer EBL. The buffer layer may compensate a resonance distance according to wavelengths of light emitted from an emission layer EML, and may thus increase light emitting efficiency. Materials which may be included in the hole transport region HTR may be used as materials included in the buffer layer.

The emission layer EML is provided on the hole transport region HTR. The emission layer EML may have, for example, a thickness of about 100 Å to about 1000 A or about 100 Å to about 300 Å. The emission layer EML may have a single layer formed of a single material, a single layer formed of a plurality of different materials, or a multilayer structure having a plurality of layers formed of a plurality of different materials.

In the light emitting element ED of one or more embodiments, the emission layer EML may be to emit blue light. The light emitting element ED of one or more embodiments includes the amine compound of one or more embodiments described herein in the hole transport region HTR, and may thus exhibit long lifespan in a blue light emitting region. However, the embodiment is not limited thereto.

In the light emitting device ED of one or more embodiments, the emission layer EML may include an anthracene derivative, a pyrene derivative, a fluoranthene derivative, a chrysene derivative, a dihydrobenzanthracene derivative, or a triphenylene derivative. For example, the emission layer EML may include the anthracene derivative or the pyrene derivative.

In each light emitting device ED of embodiments illustrated in FIGS. 3 to 6, the emission layer EML may further include a suitable host and dopant besides the herein-described host and dopant, and for example the emission layer EML may include a compound represented by Formula E-1. The compound represented by Formula E-1 may be used as a fluorescent host material.

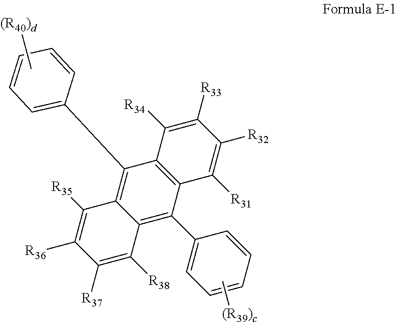

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. R₃₁ to R₄₀ may be bonded to an adjacent group to form a saturated hydrocarbon ring or an unsaturated hydrocarbon ring, a saturated heterocycle, or an unsaturated heterocycle.

In Formula E-1, c and d may each independently be an integer of 0 to 5.

Formula E-1 may be represented by any one among (e.g., selected from among) Compound E1 to Compound E19:

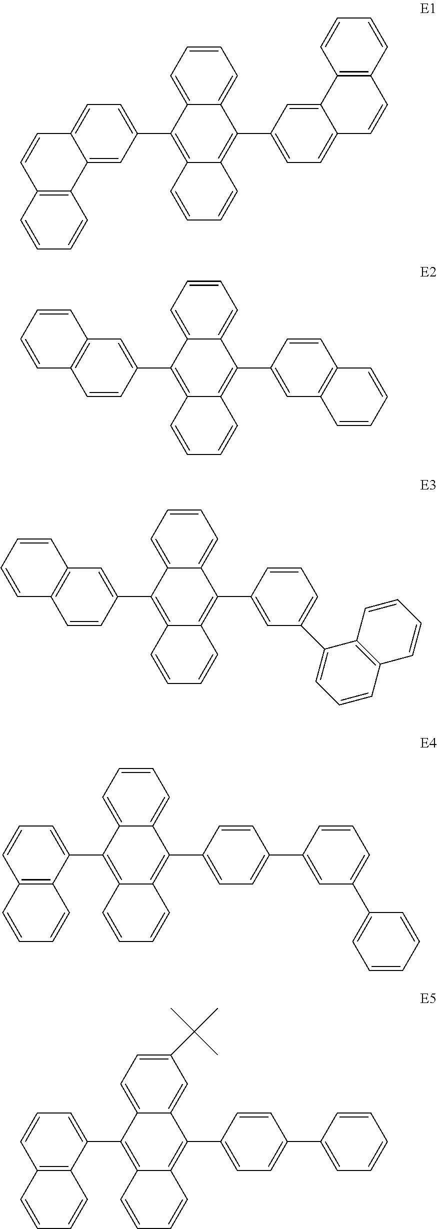

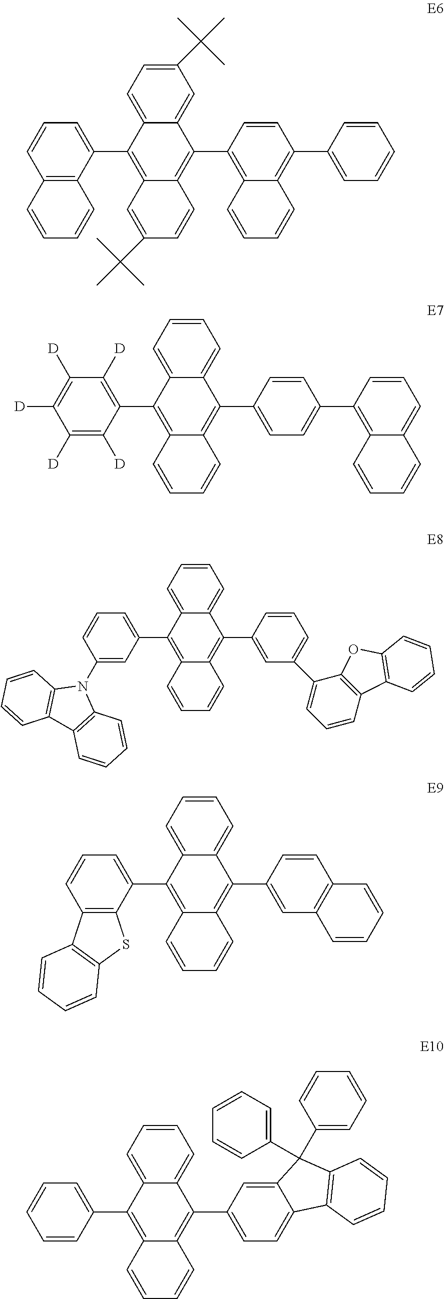

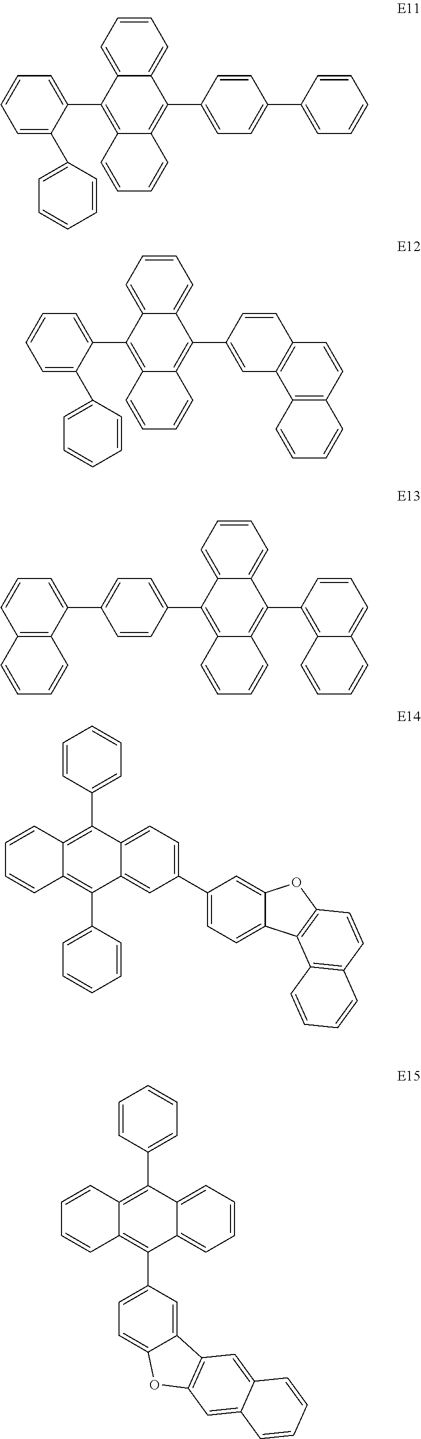

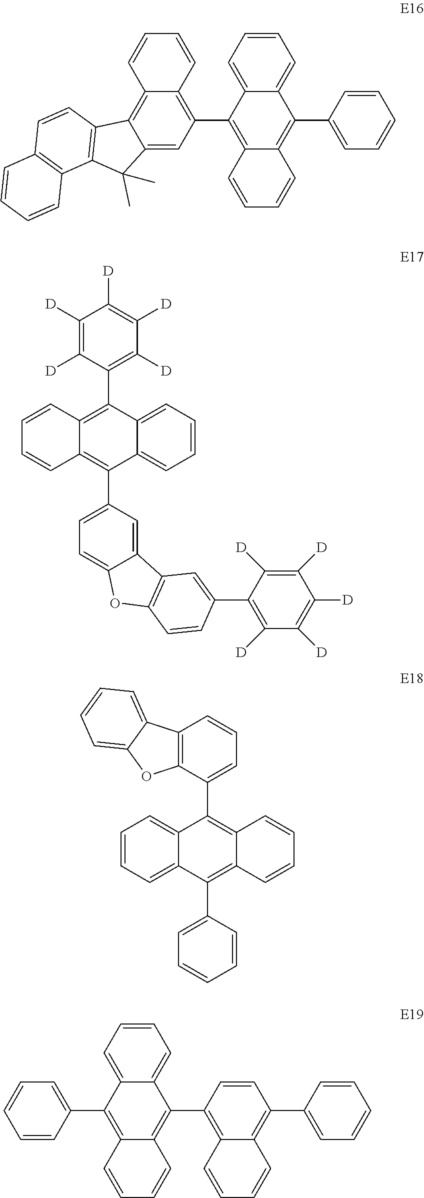

In one or more embodiments, the emission layer EML may include a compound represented by Formula E-2a or Formula E-2b. The compound represented by Formula E-2a or Formula E-2b may be used as a phosphorescent host material.

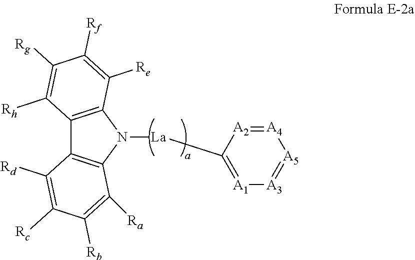

In Formula E-2a, a may be an integer of 0 to 10, and La may be a direct linkage, a substituted or unsubstituted arylene group having 6 to 30 ring-forming carbon atoms, or a substituted or unsubstituted heteroarylene group having 2 to 30 ring-forming carbon atoms. When a is an integer of 2 or greater, a plurality of Lₐ's may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may each independently be N or CRᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Rₐ to Rᵢ may be bonded to an adjacent group to form a hydrocarbon ring or a heterocycle containing N, O, S, and/or the like, as a ring-forming atom.

In Formula E-2a, two or three selected from among A₁ to A₅ may be N, and the rest may be CRᵢ.

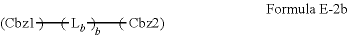

In Formula E-2b, Cbz1 and Cbz2 may each independently be an unsubstituted carbazole group, or a carbazole group substituted with an aryl group having 6 to 30 ring-forming carbon atoms. L_{b} is a direct linkage, a substituted or unsubstituted arylene group having 6 to 30 ring-forming carbon atoms, or a substituted or unsubstituted heteroarylene group having 2 to 30 ring-forming carbon atoms. b is an integer of 0 to 10, and if (e.g., when) b is an integer of 2 or more, a plurality of L_{b}'s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one among (e.g., selected from among) the compounds of Compound Group E-2. However, the compounds listed in Compound Group E-2 are merely examples, and the compound represented by Formula E-2a or Formula E-2b is not limited to those represented in Compound Group E-2.

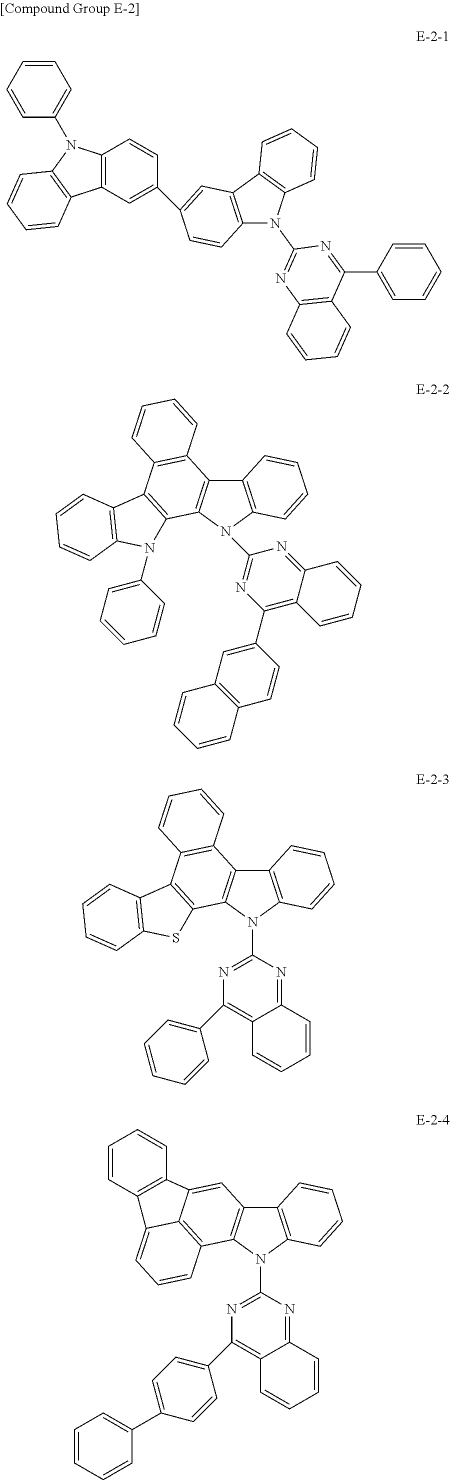

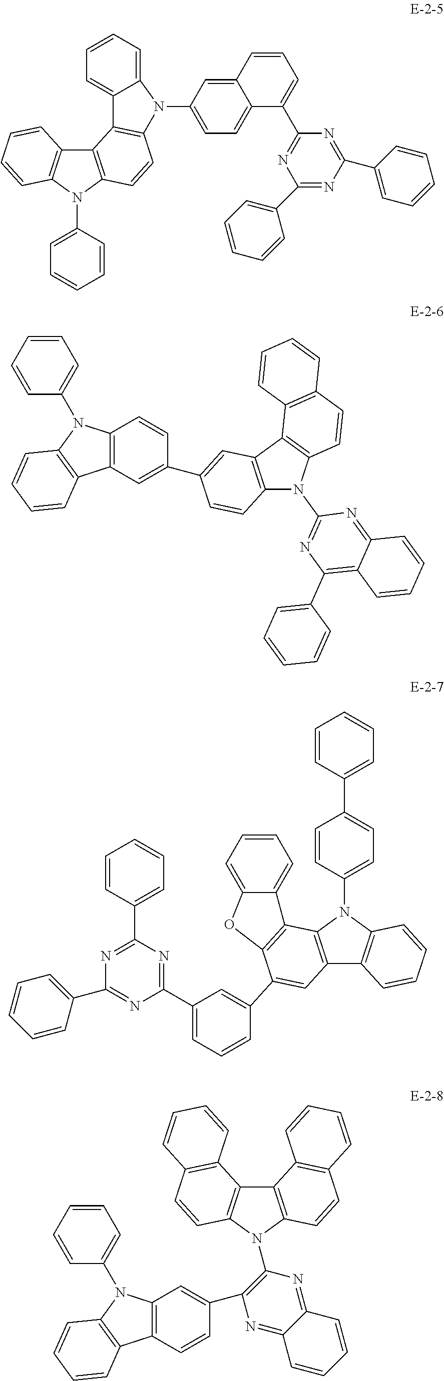 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

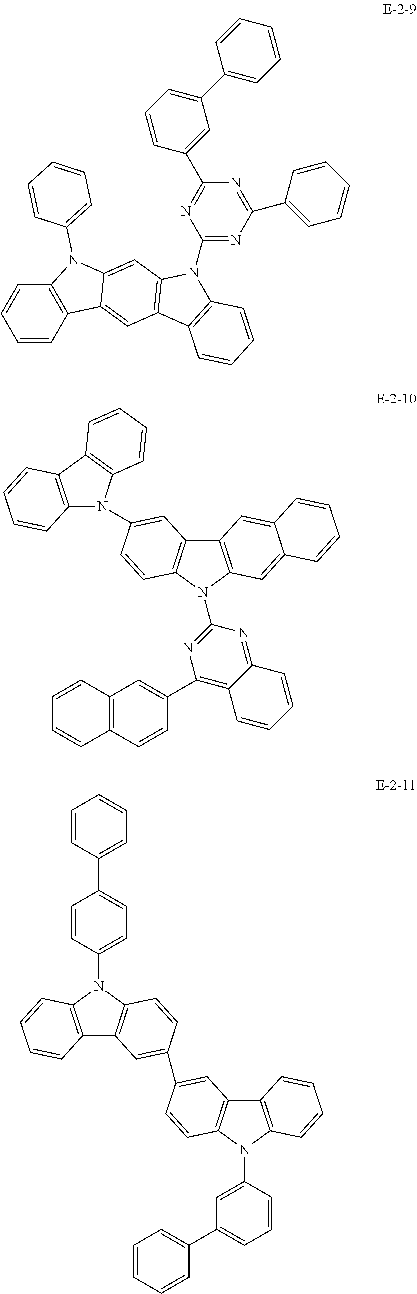 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

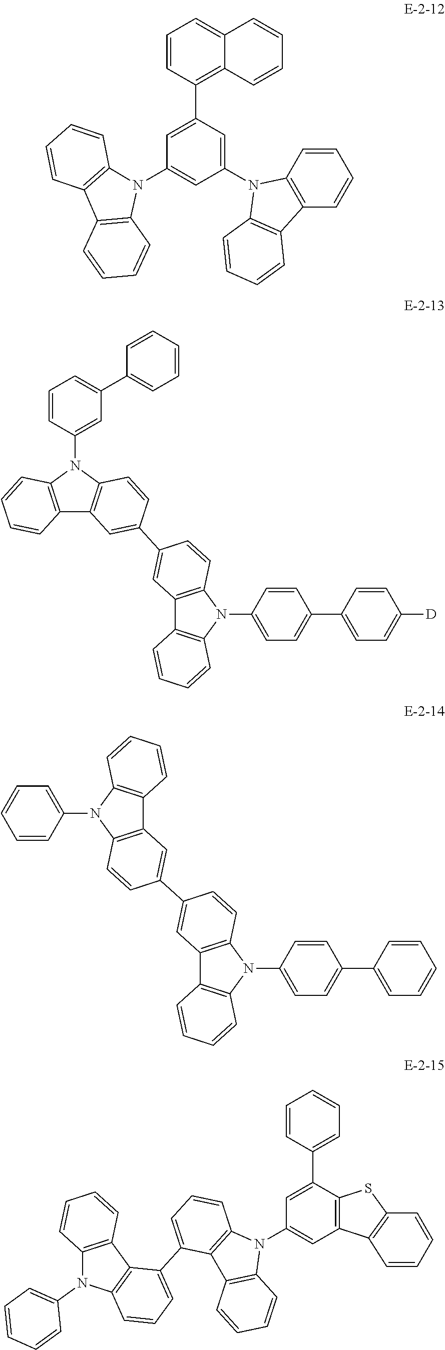 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

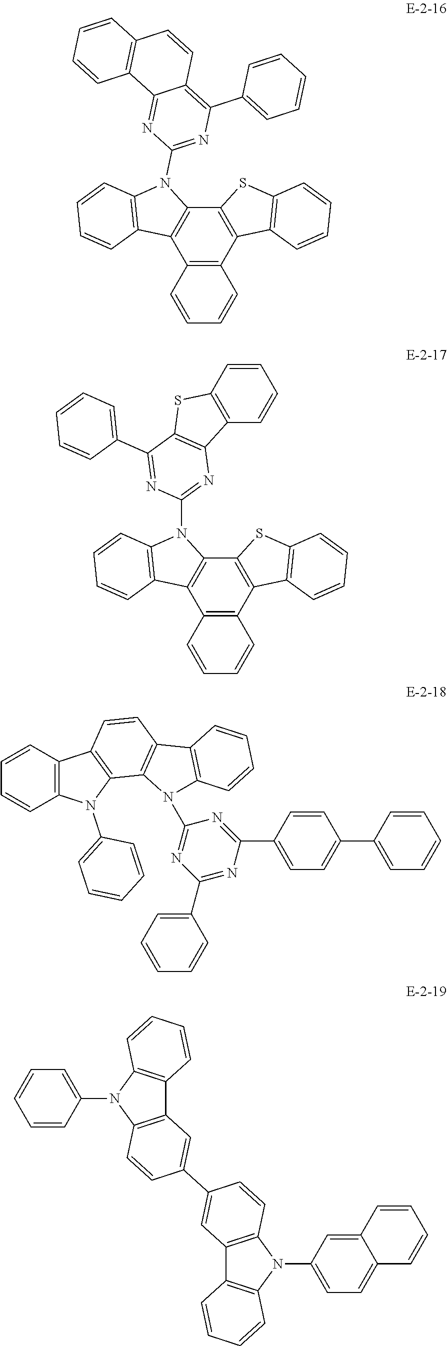

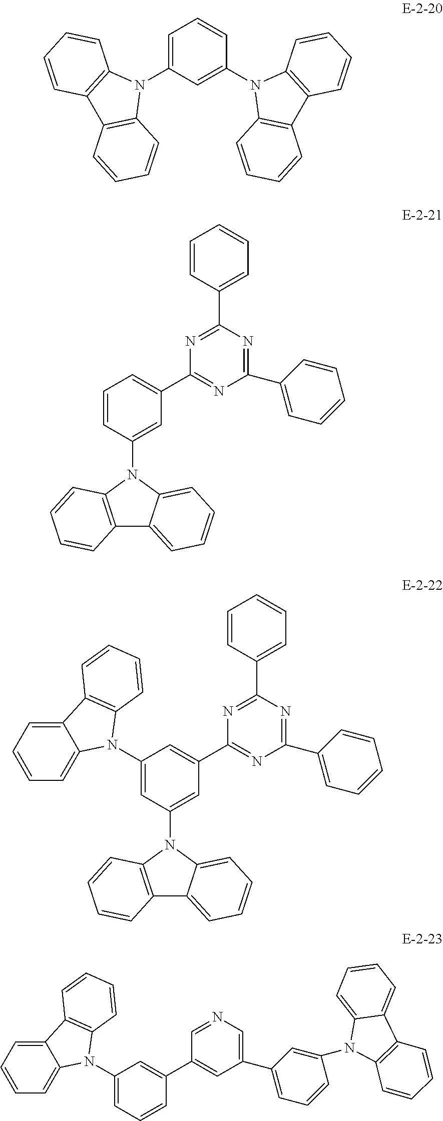 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

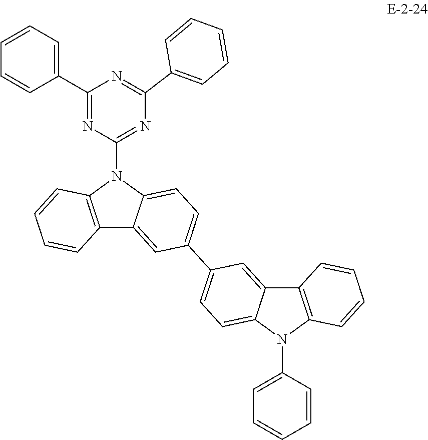 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a general material suitable in the art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, the embodiment of the disclosure is not limited thereto, for example, tris(8-hydroxyquinolino)aluminum (Alq3), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), and/or the like may be used as a host material.

The emission layer EML may include the compound represented by Formula M-a. The compound represented by Formula M-a may be used as a phosphorescent dopant material.

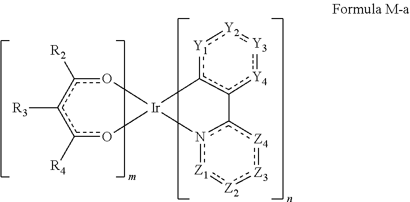

In Formula M-a, Y₁ to Y₄ and Z₁ to Z₄ may each independently be CR₁ or N, R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula M-a, m is 0 or 1, and n is 2 or 3. In Formula M-a, if (e.g., when) m is 0, n is 3, and if (e.g., when) m is 1, n is 2.

The compound represented by Formula M-a may be used as a phosphorescent dopant.

The compound represented by Formula M-a may be represented by any one among Compound M-a1 to Compound M-a25. However, Compounds M-a1 to M-a25 are examples, and the compound represented by Formula M-a is not limited to those represented by Compounds M-a1 to M-a25.

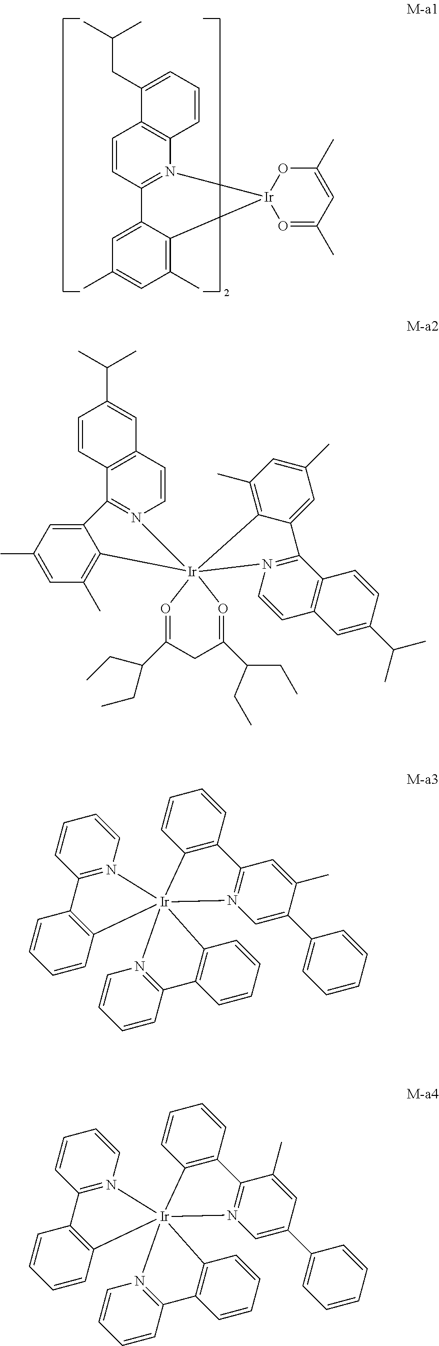

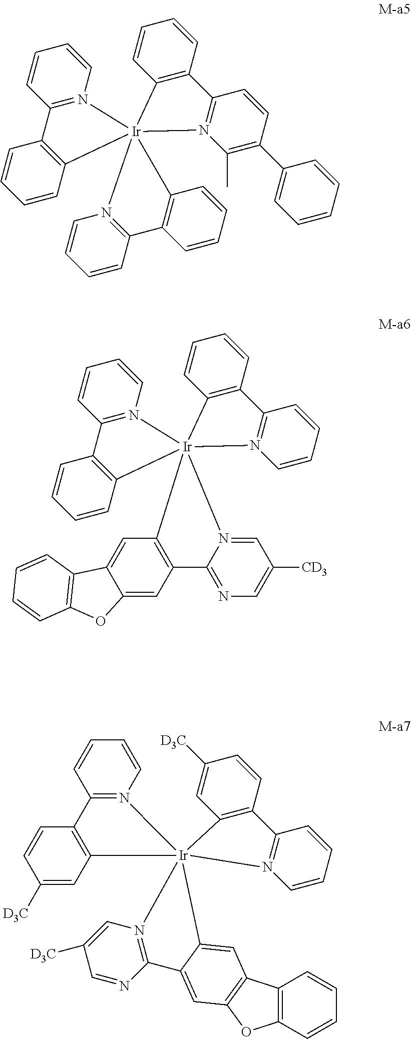

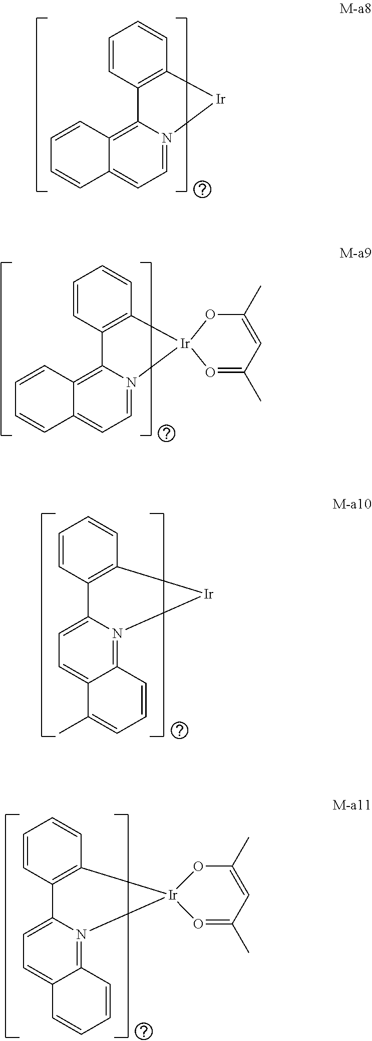

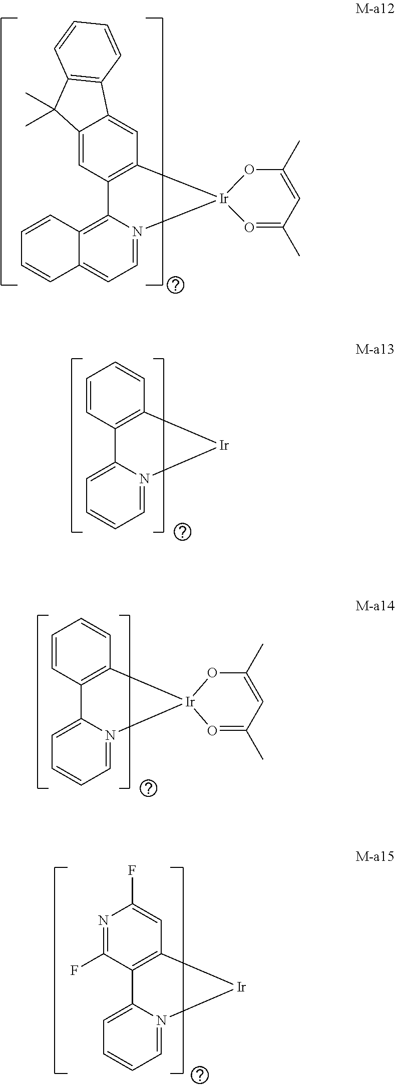

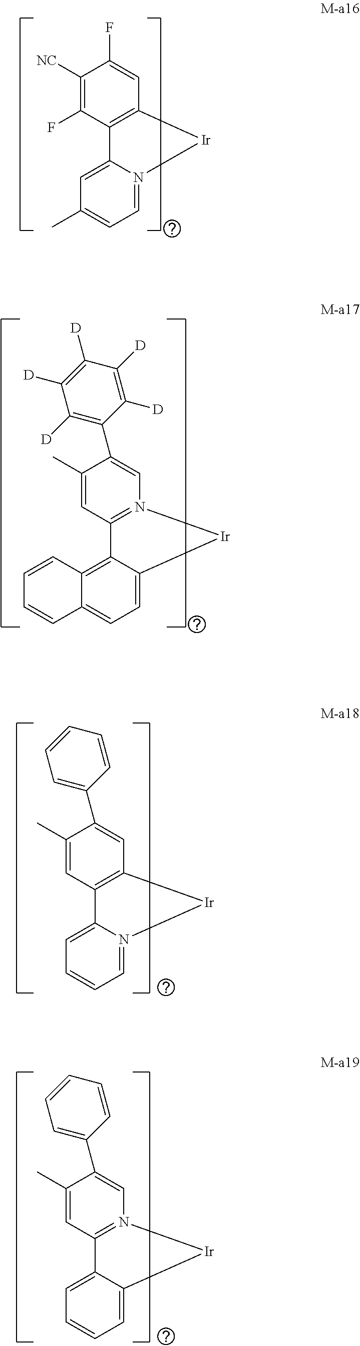

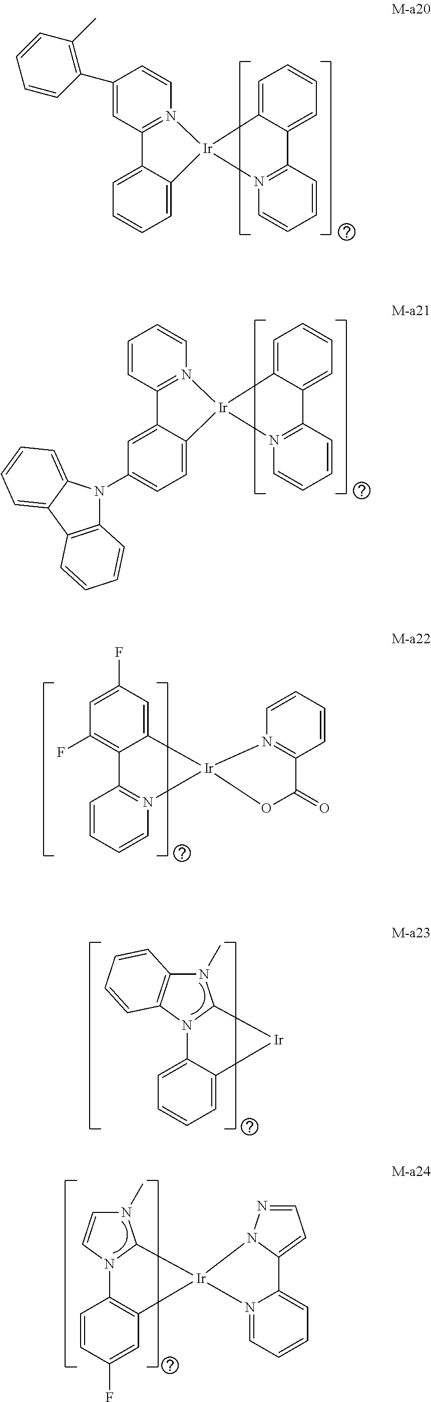

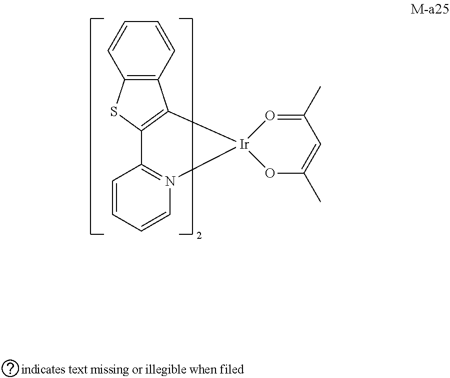

The compound M-a1 and the compound M-a2 may be used as a red dopant material, and the compound M-a3 to the compound M-a7 may be used as a green dopant material.

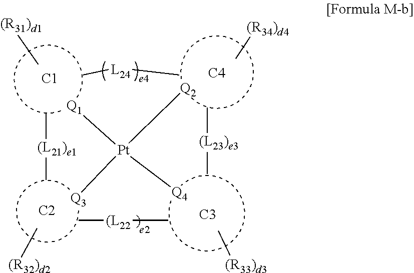

In Formula M-b, Q₁ to Q₄ may each independently be C or N, and C1 to C4 may each independently be a substituted or unsubstituted hydrocarbon ring having 5 to 30 ring-forming carbon atoms, or a substituted or unsubstituted hetero ring having 2 to 30 ring-forming carbon atoms. L₂₁ to L₂₄ may each independently be a direct linkage,

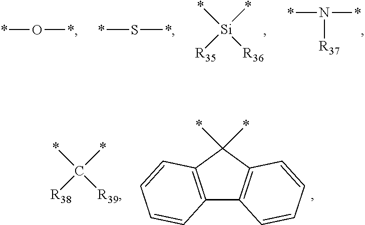

a substituted or unsubstituted alkyl group having 1 to 20 carbons, a substituted or unsubstituted arylene group having 6 to 30 ring-forming carbon atoms, or a substituted or unsubstituted heteroarylene group having 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms, or forms a ring by being coupled to an adjacent group, and d1 to d4 may each independently be an integer of 0 to 4.

The compound represented by Formula M-b may be used as a blue phosphorescent dopant or a green phosphorescent dopant. In some embodiments, the compound represented by Formula M-b may further be included in the light emitting layer EML as an auxiliary dopant in one or more embodiments.

The compound represented by Formula M-b may be represented by any one of (e.g., selected from among) compound M-b-1 to compound M-b-11. However, the compounds are only examples, and the compound represented by Formula M-b is not limited to the compound M-b-1 to the compound M-b-11.

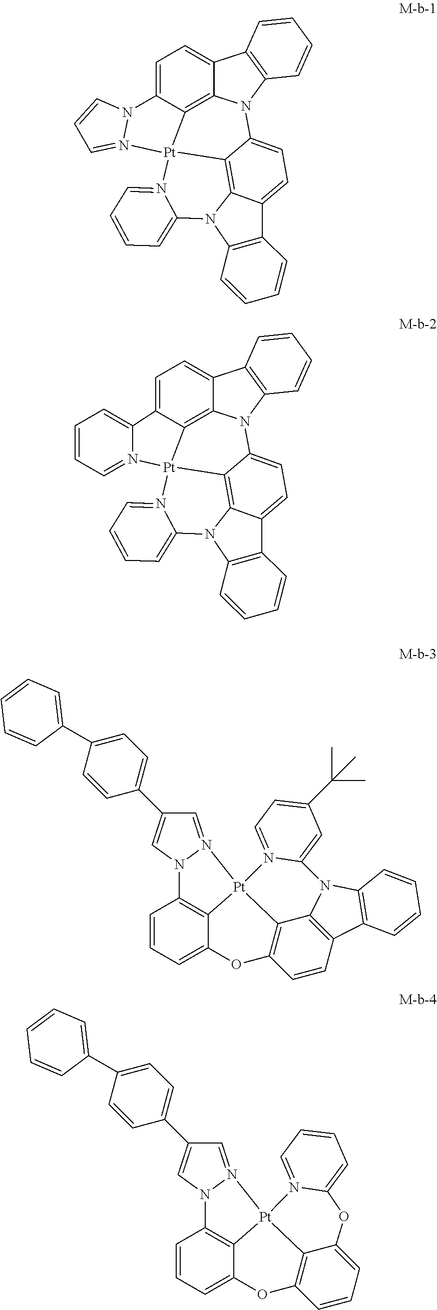

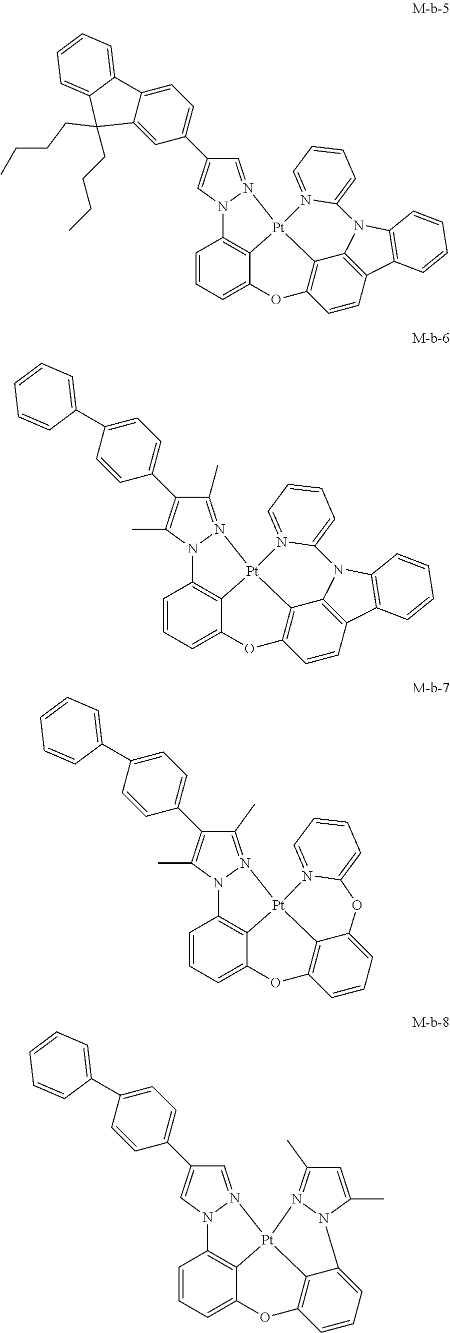

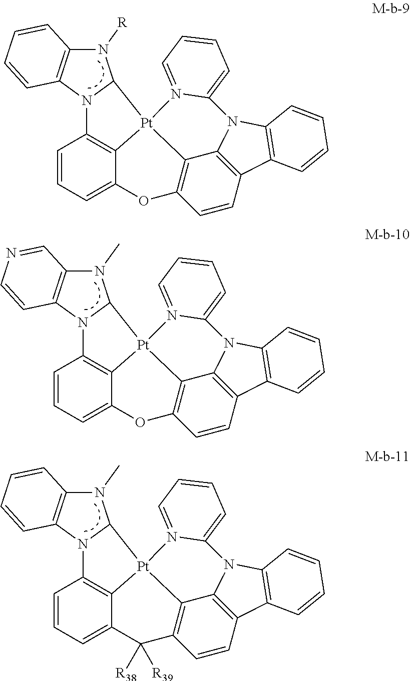

In the compounds herein, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbons, a substituted or unsubstituted aryl group having 6 to 30 ring-forming carbon atoms, or a substituted or unsubstituted heteroaryl group having 2 to 30 ring-forming carbon atoms.

The emission layer EML may include a compound represented by any one among Formula F-a to Formula F-c. The compound represented by Formula F-a to Formula F-c may be used as a fluorescence dopant material.

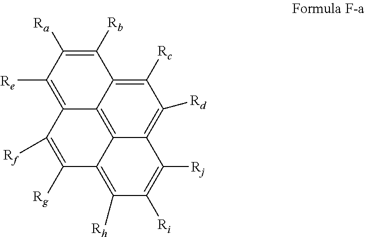

In Formula F-a, two selected from among Rₐ to Rⱼ may each independently be substituted with *—NAr₁Ar₂. The others, which are not substituted with *—NAr₁Ar₂, among Rₐ to Rⱼ may be may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In *—NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

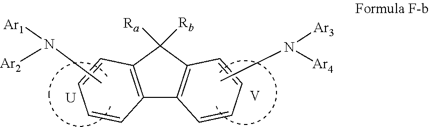

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms. At least one among Ar₁ to Ar₄ may be a heteroaryl group containing O or S as a ring-forming atom.

In Formula F-b, the number of rings represented by U and V may each independently be 0 or 1. For example, in Formula F-b, it refers to that if (e.g., when) the number of U or V is 1, one ring constitutes a fused ring at a portion indicated by U or V, and if (e.g., when) the number of U or V is 0, a ring indicated by U or V does not exist. For example, if (e.g., when) the number of U is 0 and the number of V is 1, or if (e.g., when) the number of U is 1 and the number of V is 0, the fused ring having a fluorene core in Formula F-b may be a cyclic compound having four rings. In some embodiments, if (e.g., when) each number of U and V is 0, the fused ring in Formula F-b may be a cyclic compound having three rings. In some embodiments, if (e.g., when) each number of U and V is 1, the fused ring having a fluorene core in Formula F-b may be a cyclic compound having five rings.

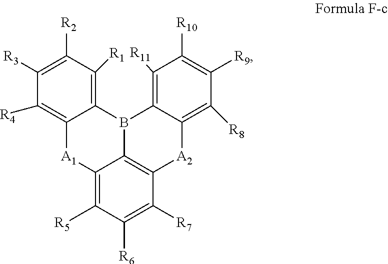

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring.

In Formula F-c, A₁ and A₂ may each independently be bonded to substituents of an adjacent ring to form a fused ring. For example, if (e.g., when) A₁ and A₂ may each independently be NRₘ, A₁ may be bonded to R₄ or R₅ to form a ring. In some embodiments, A₂ may be bonded to R₇ or R₈ to form a ring.

In one or more embodiments, the emission layer EML may further include, as a suitable dopant material, a styryl derivative (e.g., 1,4-bis[2-(3-N-ethylcarbazolyl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenamine (N-BDAVBi), 4,4′-bis[2-(4-(N,N-diphenylamino)phenyl)vinyl]biphenyl (DPAVBi), perylene and a derivative thereof (e.g., 2,5,8,11-tetra-t-butylperylene (TBP)), pyrene and a derivative thereof (e.g., 1,1-dipyrene, 1,4-dipyrenylbenzene, 1,4-bis(N,N-diphenylamino)pyrene), and/or the like.

The emission layer EML may further include a suitable phosphorescence dopant material. For example, a metal complex containing iridium (Ir), platinum (Pt), osmium (Os), aurum (Au), titanium (Ti), zirconium (Zr), hafnium (Hf), europium (Eu), terbium (Tb), or thulium (Tm) may be used as a phosphorescent dopant. For example, iridium(III) bis(4,6-difluorophenylpyridinato-N,C2) (Flrpic), bis(2,4-difluorophenylpyridinato)-tetrakis(1-pyrazolyl)borate iridium(III) (Fir6), or platinum octaethyl porphyrin (PtOEP) may be used as a phosphorescent dopant. However, the embodiment of the disclosure is not limited thereto.

In one or more embodiments, the emission layer EML may include a hole transporting host and an electron transporting host. In some embodiments, the emission layer EML may include an auxiliary dopant and a light emitting dopant. The auxiliary dopant may include a phosphorescent dopant material or a thermally activated delayed fluorescent dopant material. For example, in one or more embodiments, the emission layer EML may include a hole transporting host, an electron transporting host, an auxiliary dopant, and a light emitting dopant.

In some embodiments, in the emission layer EML, the hole transporting host and the electron transporting host may form an exciplex. In this case, the triplet energy of the exciplex formed by the hole transporting host and the electron transporting host may correspond to T1, which is a gap between lowest unoccupied molecular orbital (LUMO) energy level of the electron transporting host and highest occupied molecular orbital (HOMO) energy level of the hole transporting host.

In one or more embodiments, the triplet energy level T1 of the exciplex formed by the hole transporting host and the electron transporting host may be about 2.4 electron volt (eV) to about 3.0 eV. In some embodiments, the triplet energy of the exciplex may have a value smaller than the energy gap of each host material. Accordingly, the exciplex may have a triplet energy of 3.0 eV or less, which is an energy gap between the hole transporting host and the electron transporting host.

The emission layer EML may include a quantum dot material. The core of the quantum dot may be selected from among a Group II-VI compound, a Group III-VI compound, a Group I-III-IV compound, a Group III-V compound, a Group III-II-V compound, a Group IV-VI compound, a Group IV element, a Group IV compound, or a combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and a mixture thereof.

The Group III-VI compound may include a binary compound such as In₂S₃ or In₂Se₃, a ternary compound such as InGaS₃ or InGaSe₃, or any combination thereof.

The Group I-III-VI compound may be selected from among a ternary compound selected from the group consisting of AgInS, AgInS₂, CuInS, CuInS₂, AgGaS₂, CuGaS₂ CuGaO₂, AgGaO₂, AgAlO₂, and a mixture thereof, or a quaternary compound such as AgInGaS₂ or CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and a mixture thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a mixture thereof. The Group III-V compound may further include a Group II metal. For example, InZnP, and/or the like, may be selected as a Group III-II-V compound.

The Group IV-VI compound may be selected from the group consisting of a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The Group IV element may be selected from the group consisting of Si, Ge, and a mixture thereof. The Group IV compound may be a binary compound selected from the group consisting of SiC, SiGe, and a mixture thereof.

Each element included in a polynary compound such as the binary compound, the ternary compound, or the quaternary compound may be present in a particle with a substantially uniform or non-substantially uniform concentration distribution. For example, the formulae refer to the types (kinds) of elements included in the compounds, and the elemental ratio in the compound may be different. For example, AgInGaS₂ may refer to AgInₓGa₁₋ₓS₂ (where x is a real number of 0 to 1).

The quantum dot may have a single structure or a double structure of core-shell in which the concentration of each element included in the quantum dot is substantially uniform. For example, the material included in the core may be different from the material included in the shell.

The shell of the quantum dot may serve as a protection layer to prevent or reduce the chemical deformation of the core to maintain semiconductor properties, and/or a charging layer to impart electrophoresis properties to the quantum dot. The shell may be a single layer or multiple layers. An interface between the core and the shell may have a concentration gradient in which the concentration of an element present in the shell becomes lower towards the center.

In some embodiments, the quantum dot may have the herein-described core/shell structure including a core containing nanocrystals and a shell around (e.g., surrounding) the core. The shell of the quantum dot may serve as a protection layer to prevent or reduce the chemical deformation of the core to maintain semiconductor properties, and/or a charging layer to impart electrophoresis properties to the quantum dot. The shell may be a single layer or multiple layers. An example of the shell of the quantum dots may include a metal or non-metal oxide, a semiconductor compound, or a combination thereof.

For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO, or a ternary compound such as MgAl₂O₄, CoFe₂O₄, NiFe₂O₄, or CoMn₂O₄, but the embodiment of the disclosure is not limited thereto.

Also, examples of the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and/or the like, but the embodiment of the disclosure is not limited thereto.

Each element included in a polynary compound such as the binary compound, or the ternary compound may be present in a particle with a substantially uniform or non-substantially uniform concentration distribution. For example, the formulae refer to the types (kinds) of elements included in the compounds, and the elemental ratio in the compound may be different.

The quantum dot may have a full width of half maximum (FWHM) of a light emitting wavelength spectrum of about 45 nanometer (nm) or less, about 40 nm or less, and more about 30 nm or less, and color purity or color reproducibility may be improved in the preceding range. In some embodiments, light emitted through such quantum dot is emitted in all directions so that a wide viewing angle may be improved.

In some embodiments, although the form of the quantum dot is not particularly limited as long as it is a form commonly used in the art, more specifically, the quantum dot in the form of spherical, pyramidal, multi-arm, or cubic nanoparticles, nanotubes, nanowires, nanofibers, nanoplate particles, and/or the like may be used.

As the size of the quantum dot is adjusted or the elemental ratio in the quantum dot compound is adjusted, it is possible to control the energy band gap, and thus light in one or more suitable wavelength ranges may be obtained in the quantum dot emission layer. Therefore, the quantum dot as described (using different sizes of quantum dots or different elemental ratios in the quantum dot compound) is used, and thus the light emitting device, which emits light in one or more suitable wavelengths, may be implemented. For example, the adjustment of the size of the quantum dot or the elemental ratio in the quantum dot compound may be selected to emit red, green, and/or blue light. In some embodiments, the quantum dots may be configured to emit white light by combining one or more suitable colors of light.

In each of the light emitting devices ED of embodiments illustrated in FIGS. 3 to 6, the electron transport region ETR is provided on the emission layer EML. The electron transport region ETR may include at least one of the hole blocking layer HBL, the electron transport layer ETL, or the electron injection layer EIL, but the embodiment of the disclosure is not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In some embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL are stacked in order from the emission layer EML, but the embodiment of the disclosure is not limited thereto. The electron transport region ETR may have a thickness, for example, from about 1,000 Å to about 1,500 Å.

The electron transport region ETR may be formed using one or more suitable methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-1:

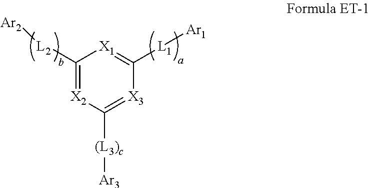

In Formula ET-1, at least one among X₁ to X₃ is N, and the rest are CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may each independently be an integer of 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. When a to c may each independently be an integer of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, the embodiment of the disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,08)-(1,1′-biphenyl-4-olato)aluminum (BAIq), beryllium bis(benzoquinolin-10-olate) (Bebq₂), 9,10-di(naphthalen-2-yl)anthracene (ADN), 1,3-bis[3,5-di(pyridin-3-yl)phenyl]benzene (BmPyPhB), or a mixture thereof.

The electron transport region ETR may include at least one among (e.g., selected from among) Compound ET1 to Compound ET36:

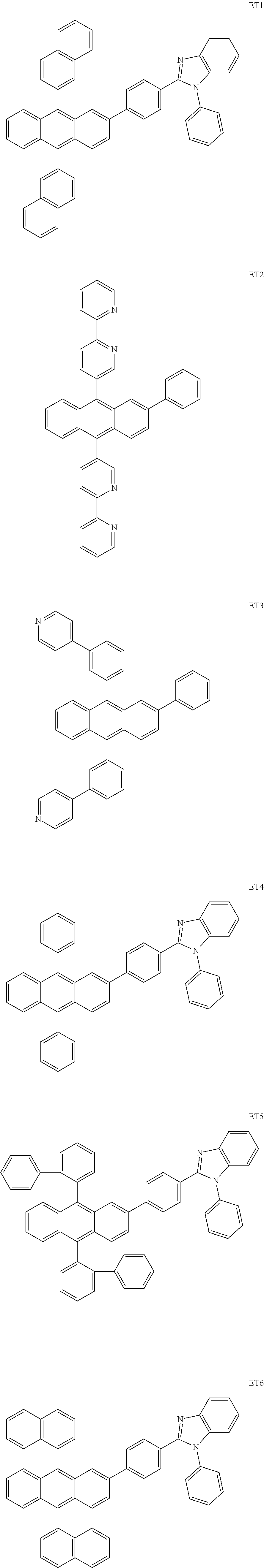

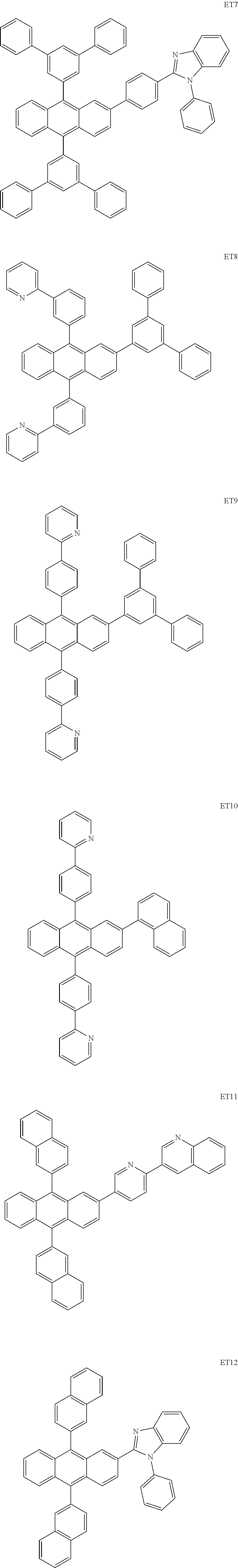

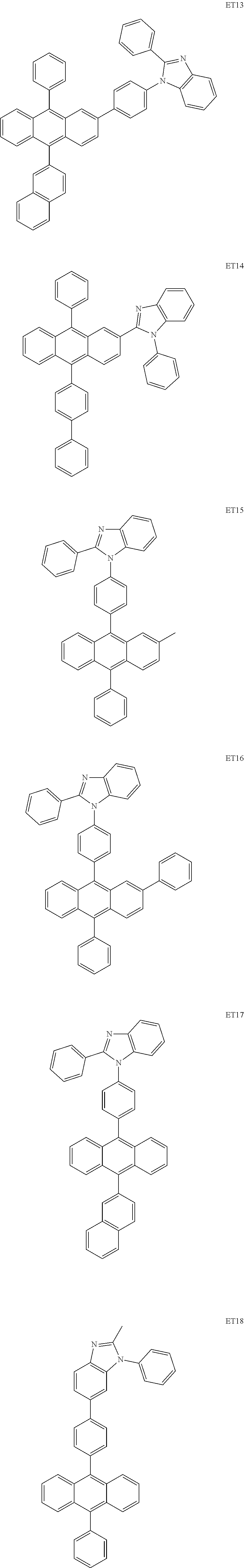 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

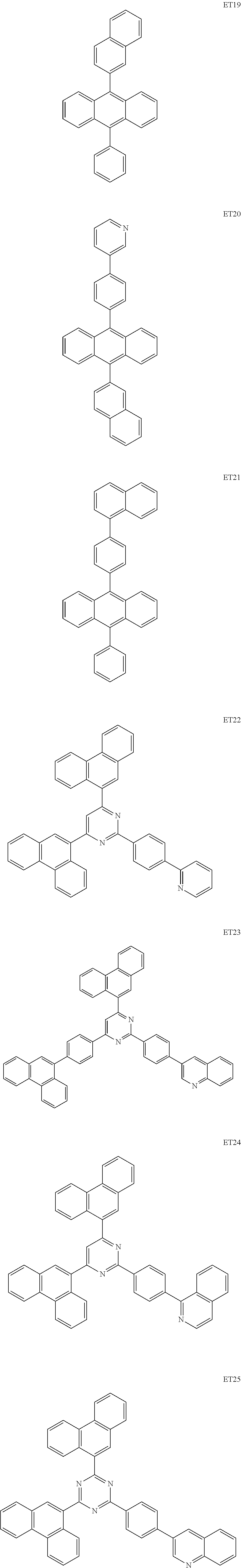

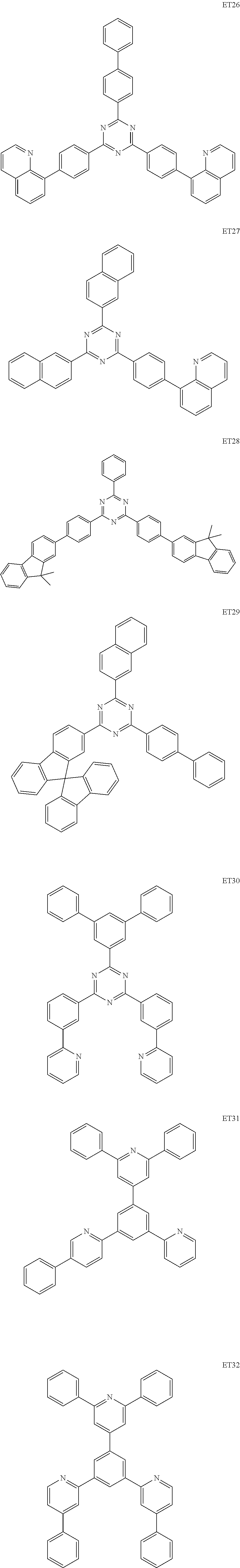

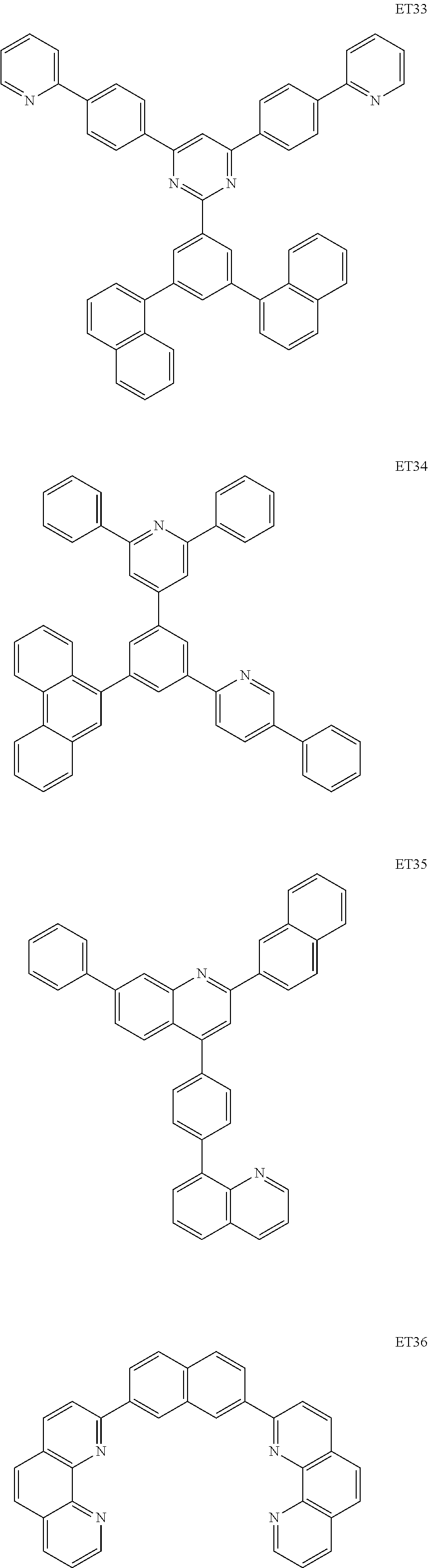 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI, and KI, a lanthanide metal such as Yb, and a co-deposited material of the metal halide and the lanthanide metal. For example, the electron transport region ETR may include KI:Yb, RbI:Yb, LiF:Yb, and/or the like, as a co-deposited material. The electron transport region ETR may be formed using a metal oxide such as Li₂O or BaO, or 8-hydroxyl-lithium quinolate (Liq), and/or the like, but the embodiment of the disclosure is not limited thereto. The electron transport region ETR may also be formed of a mixture material of an electron transport material and an insulating organometallic salt. The organometallic salt may be a material having an energy band gap of about 4 eV or more. For example, the organometallic salt may include, for example, a metal acetate, a metal benzoate, a metal acetoacetate, a metal acetylacetonate, or a metal stearate.

The electron transport region ETR may further include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to the herein-described materials, but the embodiment of the disclosure is not limited thereto.

The electron transport region ETR may include the herein-described compounds of the hole transport region in at least one of the electron injection layer EIL, the electron transport layer ETL, or the hole blocking layer HBL.

When the electron transport region ETR includes the electron transport layer ETL, the electron transport layer ETL may have a thickness of about 100 Å to about 1,000 Å, for example, about 150 Å to about 500 Å. If the thickness of the electron transport layer ETL satisfies the aforementioned range, satisfactory electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. If the thickness of the electron injection layer EIL satisfies the herein-described range, satisfactory electron injection characteristics may be obtained without a substantial increase in driving voltage.

The second electrode EL2 is provided on the electron transport region ETR. The second electrode EL2 may be a common electrode. The second electrode EL2 may be a cathode or an anode, but the embodiment of the disclosure is not limited thereto. For example, if (e.g., when) the first electrode EL1 is an anode, the second electrode EL2 may be a cathode, and if (e.g., when) the first electrode EL1 is a cathode, the second electrode EL2 may be an anode.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), and/or the like.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, or a compound or mixture thereof (e.g., AgMg, AgYb, or MgAg). In one or more embodiments, the second electrode EL2 may have a multilayer structure including a reflective film or a transflective film formed of the herein-described materials, and a transparent conductive film formed of ITO, IZO, ZnO, ITZO, and/or the like. For example, the second electrode EL2 may include the herein-described metal materials, combinations of at least two metal materials of the herein-described metal materials, oxides of the herein-described metal materials, and/or the like.

The second electrode EL2 may be connected with an auxiliary electrode. If the second electrode EL2 is connected with the auxiliary electrode, the resistance of the second electrode EL2 may be decreased.

A capping layer CPL may further be arranged on the second electrode EL2 of the light emitting device ED of one or more embodiments. The capping layer CPL may include a multilayer or a single layer.

In one or more embodiments, the capping layer CPL may be an organic layer or an inorganic layer. For example, if (e.g., when) the capping layer CPL contains an inorganic material, the inorganic material may include an alkaline metal compound (e.g., LiF), an alkaline earth metal compound (e.g., MgF₂), SiON, SiNx, SiOy, and/or the like.

For example, if (e.g., when) the capping layer CPL includes an organic material, the organic material may include α-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol-9-yl)triphenylamine (TCTA), and/or the like, or an epoxy resin, or acrylate such as methacrylate. However, the embodiment of the disclosure is not limited thereto, and the capping layer CPL may include at least one among (e.g., selected from among) Compounds P1 to P5:

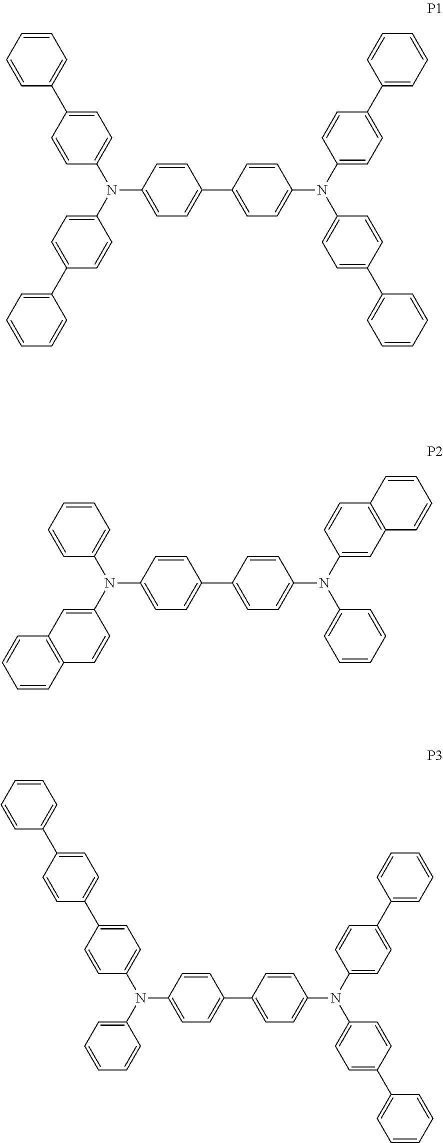 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

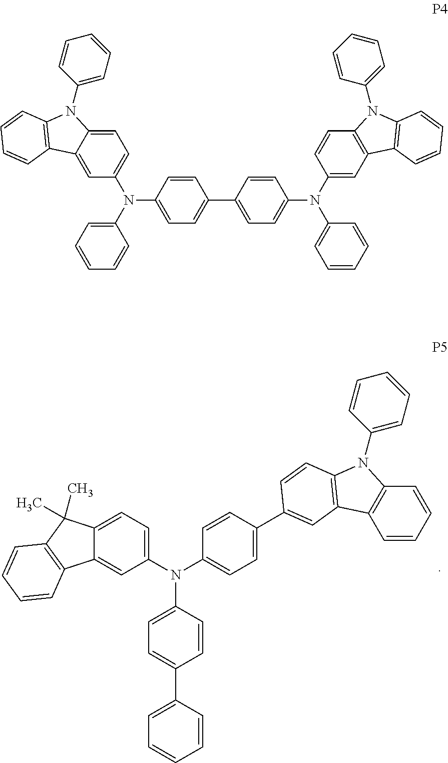 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

The refractive index of the capping layer CPL may be about 1.6 or more. For example, the refractive index of the capping layer CPL may be about 1.6 or more with respect to light in a wavelength range of about 550 nm to about 660 nm.

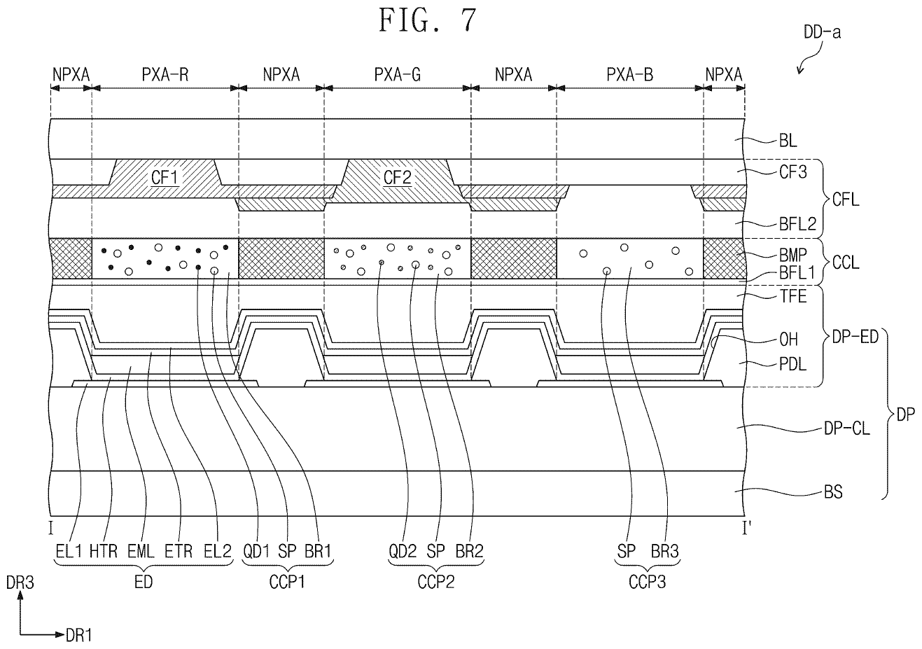

Each of FIGS. 7 to 10 is a cross-sectional view of a display apparatus according to one or more embodiments of the disclosure. Hereinafter, in describing the display apparatuses of embodiments with reference to FIGS. 7 to 10, the duplicated features which have been described in FIGS. 1 to 6 are not described again, but their differences will be mainly described.

Referring to FIG. 7, the display apparatus DD-a according to one or more embodiments may include a display panel DP including a display device layer DP-ED, a light control layer CCL arranged on the display panel DP, and a color filter layer CFL. In one or more embodiments illustrated in FIG. 7, the display panel DP may include a base layer BS, a circuit layer DP-CL provided on the base layer BS, and the display device layer DP-ED, and the display device layer DP-ED may include a light emitting device ED.

The light emitting device ED may include a first electrode EL1, a hole transport region HTR arranged on the first electrode EL1, an emission layer EML arranged on the hole transport region HTR, an electron transport region ETR arranged on the emission layer EML, and a second electrode EL2 arranged on the electron transport region ETR. The structures of the light emitting devices of FIGS. 3 to 6 as described herein may be equally applied to the structure of the light emitting device ED illustrated in FIG. 7.

The hole transport region HTR of the light emitting element ED included in a display device DD-a according to one or more embodiments may include the amine compound of one or more embodiments, which is described herein.

Referring to FIG. 7, the emission layer EML may be arranged in an opening OH defined in a pixel defining film PDL. For example, the emission layer EML which is divided (e.g., defined) by the pixel defining film PDL and provided corresponding to each light emitting regions PXA-R, PXA-G, and PXA-B may be to emit light in substantially the same wavelength range. In the display apparatus DD-a of one or more embodiments, the emission layer EML may be to emit blue light. Unlike the configuration illustrated, in one or more embodiments, the emission layer EML may be provided as a common layer in the entire light emitting regions PXA-R, PXA-G, and PXA-B.

The light control layer CCL may be arranged on the display panel DP. The light control layer CCL may include a light conversion body. The light conversion body may be a quantum dot, a phosphor, and/or the like. The light conversion body may be to emit provided light by converting the wavelength thereof. For example, the light control layer CCL may a layer containing the quantum dot or a layer containing the phosphor.

The light control layer CCL may include a plurality of light control parts CCP1, CCP2 and CCP3. The light control parts CCP1, CCP2, and CCP3 may be spaced and/or apart from each other.

Referring to FIG. 7, divided patterns BMP may be arranged between the light control parts CCP1, CCP2 and CCP3 which are spaced and/or apart from each other, but the embodiment of the disclosure is not limited thereto. FIG. 7 illustrates that the divided patterns BMP do not overlap the light control parts CCP1, CCP2 and CCP3, but at least a portion of the edges of the light control parts CCP1, CCP2 and CCP3 may overlap the divided patterns BMP.

The light control layer CCL may include a first light control part CCP1 containing a first quantum dot QD1 which converts first color light provided from the light emitting device ED into second color light, a second light control part CCP2 containing a second quantum dot QD2 which converts the first color light into third color light, and a third light control part CCP3 which transmits the first color light.

In one or more embodiments, the first light control part CCP1 may provide red light that is the second color light, and the second light control part CCP2 may provide green light that is the third color light. The third light control part CCP3 may provide blue light by transmitting the blue light that is the first color light provided from the light emitting device ED. For example, the first quantum dot QD1 may be a red quantum dot, and the second quantum dot QD2 may be a green quantum dot. The same as described herein may be applied with respect to the quantum dots QD1 and QD2.

In some embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but include the scatterer SP.

The scatterer SP may be inorganic particles. For example, the scatterer SP may include at least one of TiO₂, ZnO, Al₂O₃, SiO₂, or hollow sphere silica. The scatterer SP may include any one among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 each may include base resins BR1, BR2, and BR3 in which the quantum dots QD1 and QD2 and the scatterer SP are dispersed. In one or more embodiments, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may be acrylic-based resins, urethane-based resins, silicone-based resins, epoxy-based resins, and/or the like. The base resins BR1, BR2, and BR3 may be transparent resins. In one or more embodiments, the first base resin BR1, the second base resin BR2, and the third base resin BR3 may be the same as or different from each other.

The light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may block the light control parts CCP1, CCP2 and CCP3 from being exposed to moisture/oxygen. The barrier layer BFL1 may cover the light control parts CCP1, CCP2, and CCP3. In some embodiments, the barrier layer BFL2 may be provided between the light control parts CCP1, CCP2, and CCP3 and the color filter layer CFL.

The barrier layers BFL1 and BFL2 may include at least one inorganic layer. For example, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, and/or the like. The barrier layers BFL1 and BFL2 may further include an organic film. The barrier layers BFL1 and BFL2 may be formed of a single layer or a plurality of layers.

In the display apparatus DD-a of one or more embodiments, the color filter layer CFL may be arranged on the light control layer CCL. For example, the color filter layer CFL may be directly arranged on the light control layer CCL. In this case, the barrier layer BFL2 may not be provided.

The color filter layer CFL may include color filters CF1, CF2, and CF3. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 each may include a polymeric photosensitive resin and a pigment or dye. The first filter CF1 may include a red pigment or dye, the second filter CF2 may include a green pigment or dye, and the third filter CF3 may include a blue pigment or dye.

The embodiment of the disclosure is not limited thereto, and the third filter CF3 may not include (e.g., may exclude) a pigment or dye. The third filter CF3 may include a polymeric photosensitive resin and may not include (e.g., may exclude) a pigment or dye. The third filter CF3 may be transparent. The third filter CF3 may be formed of a transparent photosensitive resin.

Furthermore, in one or more embodiments, the first filter CF1 and the second filter CF2 may be a yellow filter. The first filter CF1 and the second filter CF2 may not be separated but be provided as one filter.

In one or more embodiments, the color filter layer CFL may further include a light shielding part. The light shielding part may be a black matrix. The light shielding part may include an organic light shielding material or an inorganic light shielding material containing a black pigment or dye. The light shielding part may prevent or reduce light leakage, and may separate boundaries between the adjacent filters CF1, CF2, and CF3.

The first to third filters CF1, CF2, and CF3 may be arranged corresponding to the red light emitting region PXA-R, the green light emitting region PXA-G, and the blue light emitting region PXA-B, respectively.

A base substrate BL may be arranged on the color filter layer CFL. The base substrate BL may be a member which provides a base surface in which the color filter layer CFL, the light control layer CCL, and/or the like are arranged. The base substrate BL may be a glass substrate, a metal substrate, a plastic substrate, and/or the like. However, the embodiment of the disclosure is not limited thereto, and the base substrate BL may be an inorganic layer, an organic layer, or a composite material layer. In some embodiments, unlike the configuration illustrated, in one or more embodiments, the base substrate BL may not be provided.

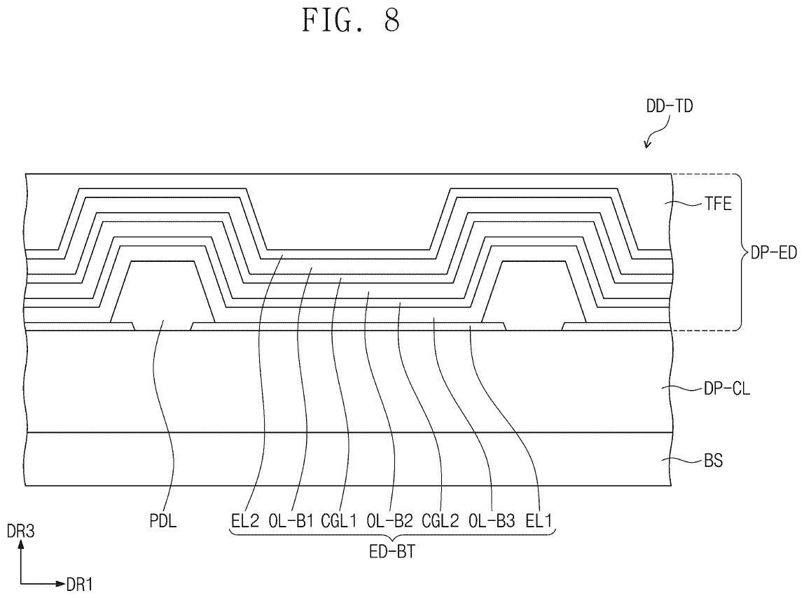

FIG. 8 is a cross-sectional view illustrating a portion of a display apparatus according to one or more embodiments. In the display apparatus DD-TD of one or more embodiments, the light emitting device ED-BT may include a plurality of light emitting structures OL-B1, OL-B2, and OL-B3. The light emitting device ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2. The light emitting structures OL-B1, OL-B2, and OL-B3 each may include an emission layer EML (FIG. 7) and a hole transport region HTR and an electron transport region ETR arranged with the emission layer EML (FIG. 7) located therebetween.

For example, the light emitting device ED-BT included in the display apparatus DD-TD of one or more embodiments may be a light emitting device having a tandem structure and including a plurality of emission layers.

In one or more embodiments illustrated in FIG. 8, all light beams respectively emitted from the light emitting structures OL-B1, OL-B2, and OL-B3 may be blue light. However, the embodiment of the disclosure is not limited thereto, and the light beams respectively emitted from the light emitting structures OL-B1, OL-B2, and OL-B3 may have wavelength ranges different from each other. For example, the light emitting device ED-BT including the plurality of light emitting structures OL-B1, OL-B2, and OL-B3 which emit light beams having wavelength ranges different from each other may be to emit white light.

Charge generation layers CGL1 and CGL2 may be respectively arranged between two of the neighboring light emitting structures OL-B1, OL-B2, and OL-B3. The charge generation layers CGL1 and CGL2 may include a p-type (kind) charge generation layer and/or an n-type (kind) charge generation layer.

At least one of the light emitting structures OL-B1, OL-B2, and OL-B3 included in the display device DD-TD of one or more embodiments may include the amine compound of one or more embodiments described herein.

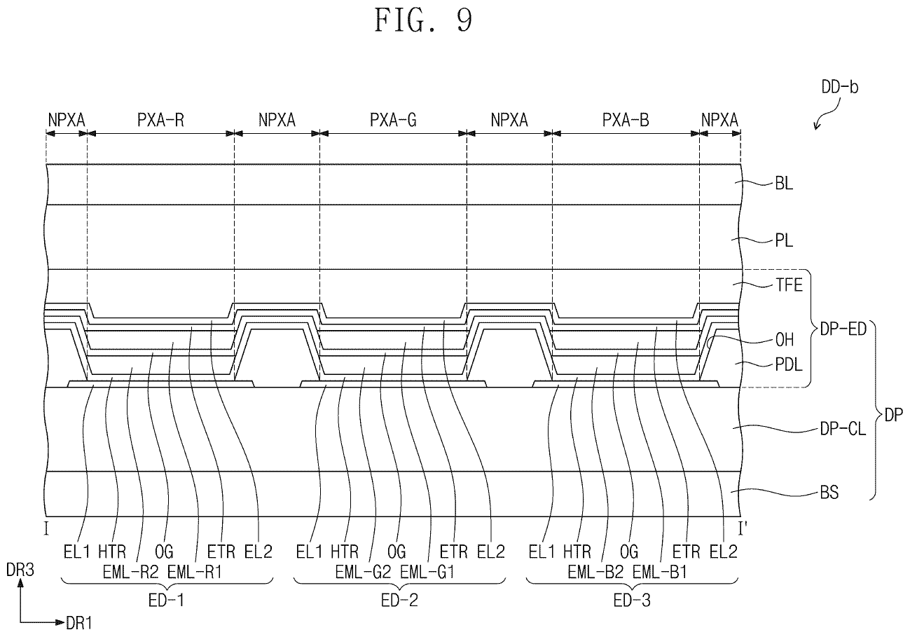

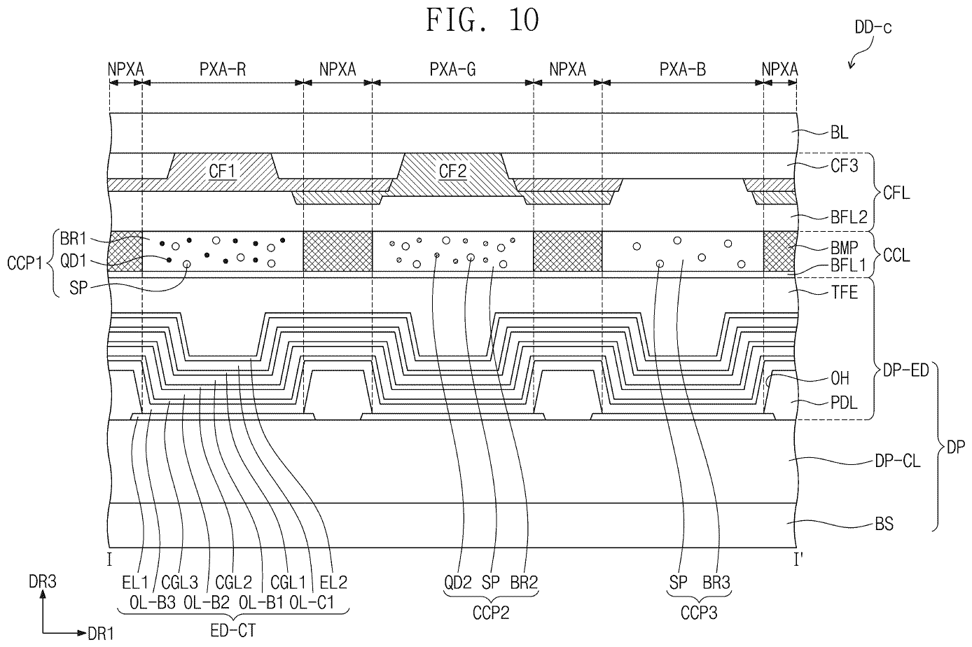

Referring to FIG. 9, the display apparatus DD-b according to one or more embodiments may include light emitting devices ED-1, ED-2, and ED-3 in which two emission layers are stacked. Compared with the display apparatus DD of one or more embodiments illustrated in FIG. 2, one or more embodiments illustrated in FIG. 9 has a difference in that the first to third light emitting devices ED-1, ED-2, and ED-3 each include two emission layers stacked in the thickness direction. In each of the first to third light emitting devices ED-1, ED-2, and ED-3, the two emission layers may be to emit light in substantially the same wavelength region.

The first light emitting device ED-1 may include a first red emission layer EML-R1 and a second red emission layer EML-R2. The second light emitting device ED-2 may include a first green emission layer EML-G1 and a second green emission layer EML-G2. In some embodiments, the third light emitting device ED-3 may include a first blue emission layer EML-B1 and a second blue emission layer EML-B2. An emission auxiliary part OG may be arranged between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. More specifically, the emission auxiliary part OG may include an electron transport region, a charge generation layer, and a hole transport region that are sequentially stacked. The emission auxiliary part OG may be provided as a common layer in the whole of the first to third light emitting devices ED-1, ED-2, and ED-3. However, the embodiment of the disclosure is not limited thereto, and the emission auxiliary part OG may be provided by being patterned within the openings OH defined in the pixel defining film PDL.

The first red emission layer EML-R1, the first green emission layer EML-G1, and the first blue emission layer EML-B1 may be arranged between the hole transport region HTR and the emission auxiliary part OG. The second red emission layer EML-R2, the second green emission layer EML-G2, and the second blue emission layer EML-B2 may be arranged between the emission auxiliary part OG and the electron transport region ETR.

For example, the first light emitting device ED-1 may include the first electrode EL1, the hole transport region HTR, the second red emission layer EML-R2, the emission auxiliary part OG, the first red emission layer EML-R1, the electron transport region ETR, and the second electrode EL2 that are sequentially stacked. The second light emitting device ED-2 may include the first electrode EL1, the hole transport region HTR, the second green emission layer EML-G2, the emission auxiliary part OG, the first green emission layer EML-G1, the electron transport region ETR, and the second electrode EL2 that are sequentially stacked. The third light emitting device ED-3 may include the first electrode EL1, the hole transport region HTR, the second blue emission layer EML-B2, the emission auxiliary part OG, the first blue emission layer EML-B1, the electron transport region ETR, and the second electrode EL2 that are sequentially stacked.

An optical auxiliary layer PL may be arranged on the display device layer DP-ED. The optical auxiliary layer PL may include a polarizing layer. The optical auxiliary layer PL may be arranged on the display panel DP and control reflected light in the display panel DP due to external light. Unlike the configuration illustrated, the optical auxiliary layer PL in the display apparatus according to one or more embodiments may not be provided.

Unlike FIGS. 8 and 9, FIG. 10 illustrates that a display apparatus DD-c includes four light emitting structures OL-B1, OL-B2, OL-B3, and OL-C1. A light emitting device ED-CT may include a first electrode EL1 and a second electrode EL2 which face each other, and first to fourth light emitting structures OL-B1, OL-B2, OL-B3, and OL-C1 that are sequentially stacked in the thickness direction between the first electrode EL1 and the second electrode EL2. Charge generation layers CGL1, CGL2, and CGL3 may be arranged between the first to fourth light emitting structures OL-B1, OL-B2, OL-B3, and OL-C1. Among the four light emitting structures, the first to third light emitting structures OL-B1, OL-B2, and OL-B3 may be to emit blue light, and the fourth light emitting structure OL-C1 may be to emit green light. However, the embodiment of the disclosure is not limited thereto, and the first to fourth light emitting structures OL-B1, OL-B2, OL-B3, and OL-C1 may be to emit light beams in different wavelength regions.

The charge generation layers CGL1, CGL2, and CGL3 arranged between adjacent light emitting structures OL-B1, OL-B2, OL-B3, and OL-C1 may include a p-type (kind) charge generation layer and/or an n-type (kind) charge generation layer.

At least one of (e.g., selected from among) the light emitting structures OL-B1, OL-B2, OL-B3, and OL-C1 included in the display device DD-c of one or more embodiments may include the amine compound of one or more embodiments described herein.

The light emitting diode ED according to one or more embodiments of the disclosure includes the amine compound of one or more embodiments described herein in at least one functional layer arranged between the first electrode EL1 and the second electrode EL2, and may thus exhibit improved lifespan characteristics. The light emitting element ED according to one or more embodiments may include the amine compound of one or more embodiments described herein in at least one of the hole transport region HTR, the emission layer EML, or the electron transport region ETR arranged between the first electrode EL1 and the second electrode EL2, or in a capping layer CPL. For example, the amine compound according to one or more embodiments may be included in the hole transport region HTR of the light emitting element ED of one or more embodiments, and the light emitting element of one or more embodiments may exhibit long lifespan characteristics.

The amine compound of one or more embodiments described herein includes a first core, and second and third substituents, and may thus increase stability of materials and improve hole transport properties. Accordingly, the light emitting element including the amine compound of one or more embodiments may have increased lifespan. In some embodiments, the light emitting element of one or more embodiments includes the amine compound according to one or more embodiments in a hole transport layer and may thus exhibit lifespan characteristics.

In one or more embodiments, an electronic apparatus (e.g., an electronic device) may include a display device (e.g., a display apparatus) including a plurality of light emitting devices, and a control part which controls the display apparatus. The electronic apparatus of one or more embodiments may be a device that is activated according to an electrical signal. The electronic apparatus may include display apparatuses of one or more suitable embodiments. For example, the electronic apparatus may include not only large-sized electronic apparatuses such as a television set, a monitor, or an outdoor billboard but also include small- and medium-sized electronic apparatuses such as a personal computer, a laptop computer, a personal digital terminal, a display apparatus for a vehicle, a game console, a portable electronic device, or a camera.

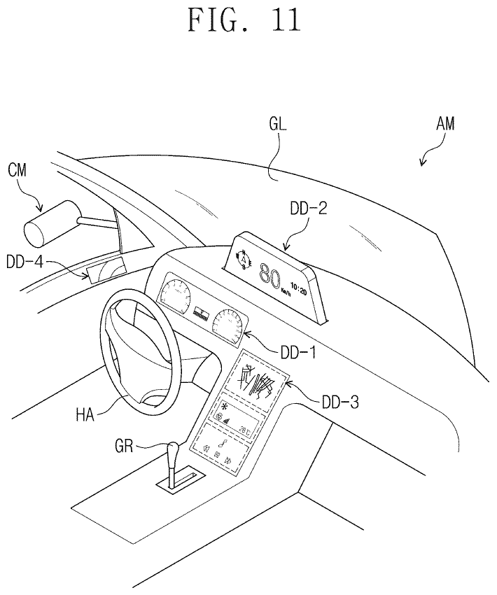

FIG. 11 is a view illustrating a vehicle AM in which first to fourth display apparatuses DD-1, DD-2, DD-3, and DD-4 are arranged. At least one among the first to fourth display apparatuses DD-1, DD-2, DD-3, and DD-4 may include the same configuration as the display apparatuses DD, DD-TD, DD-a, DD-b, and DD-c as described with reference to FIGS. 1, and 2, and 7 to 10.

FIG. 11 illustrates a vehicle AM, but this is an example, and the first to fourth display apparatuses DD-1, DD-2, DD-3, and DD-4 may be arranged in another transportation refers to such as bicycles, motorcycles, trains, ships, and airplanes. In some embodiments, at least one among the first to fourth display apparatuses DD-1, DD-2, DD-3, and DD-4 including the same configuration as the display apparatuses DD, DD-TD, DD-a, DD-b, and DD-c of one or more embodiments may be employed in a personal computer, a laptop computer, a personal digital terminal, a game console, a portable electronic device, a television, a monitor, an outdoor billboard, and/or the like. In some embodiments, these are merely provided as embodiments, and thus may be employed in other electronic apparatuses unless departing from the disclosure.

At least one among the first to fourth display apparatuses DD-1, DD-2, DD-3, and DD-4 may include the light emitting device ED of one or more embodiments as described with reference to FIGS. 3 to 6.

Referring to FIG. 11, the vehicle AM may include a steering wheel HA and a gear GR for driving the vehicle AM. In some embodiments, the vehicle AM may include a front window GL arranged so as to face the driver.

The first display apparatus DD-1 may be arranged in a first region overlapping the steering wheel HA. For example, the first display apparatus DD-1 may be a digital cluster which displays first information of the vehicle AM. The first information may include a first scale which indicates a driving speed of the vehicle AM, a second scale which indicates an engine speed (that is, revolutions per minute (RPM)), an image which indicates a fuel state, and/or the like. A first scale and a second scale may be indicated as a digital image.

The second display apparatus DD-2 may be arranged in a second region opposite to (e.g., facing) the driver's seat and overlapping the front window GL. The driver's seat may be a seat in which the steering wheel HA is arranged. For example, the second display apparatus DD-2 may be a head up display (HUD) which displays second information of the vehicle AM. The second display apparatus DD-2 may be optically transparent. The second information may include digital numbers which indicate a driving speed, and may further include information such as the current time. Unlike the configuration illustrated, the second information of the second display apparatus DD-2 may be projected to the front window GL to be displayed.

The third display apparatus DD-3 may be arranged in a third region adjacent to the gear GR. For example, the third display apparatus DD-3 may be arranged between the driver's seat and the passenger seat and may be a center information display (CID) for a vehicle for displaying third information. The passenger seat may be a seat spaced and/or apart from the driver's seat with the gear GR arranged therebetween. The third information may include information about traffic (e.g., navigation information), playing music or radio or a video (or an image), temperatures inside the vehicle AM, and/or the like.

The fourth display apparatus DD-4 may be spaced and/or apart from the steering wheel HA and the gear GR, and may be arranged in a fourth region adjacent to the side of the vehicle AM. For example, the fourth display apparatus DD-4 may be a digital side-view mirror which displays fourth information. The fourth display apparatus DD-4 may display an image outside the vehicle AM taken by a camera module CM arranged outside the vehicle AM. The fourth information may include an image outside the vehicle AM.

The herein-described first to fourth information may be examples, and the first to fourth display apparatuses DD-1, DD-2, DD-3, and DD-4 may further display information about the inside and outside of the vehicle AM. The first to fourth information may include different information. However, the embodiment of the disclosure is not limited thereto, and a part of the first to fourth information may include the same information as one another.

Terms such as “substantially,” “about,” and “approximately” are used as relative terms and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or ±30%, 20%, 10%, 5% of the stated value.

Also, it should be understood that, even if the terms “about,” “approximately,” or “substantially” are not expressly recited in a given element (e.g., a claim element), the scope of such element is intended to include variations that are insubstantial or within the understanding of one of ordinary skill in the art. For example, numerical values and ranges provided herein are intended to include tolerances and measurement uncertainties that would be recognized by those skilled in the art, and the elements (e.g., claim elements) should be construed accordingly to encompass such equivalents.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6. Applicant therefore reserves the right to amend this specification, including the claims, to expressly recite any sub-range subsumed within the ranges expressly recited herein.

The electronic device, the electronic apparatus, the display device, the display apparatus, a device of manufacturing thereof, and/or any other relevant devices or components according to embodiments of the present disclosure described herein may be implemented utilizing any suitable hardware, firmware (e.g., an application-specific integrated circuit), software, or a combination of software, firmware, and hardware. For example, the one or more suitable components of the display device and/or apparatus may be formed on one integrated circuit (IC) chip or on separate IC chips. Further, the one or more suitable components of the display device and/or apparatus may be implemented on a flexible printed circuit film, a tape carrier package (TCP), a printed circuit board (PCB), or formed on one substrate. Further, the one or more suitable components of the display device and/or apparatus may be a process or thread, running on one or more processors, in one or more computing devices, executing computer program instructions and interacting with other system components for performing the one or more suitable functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of one or more suitable computing devices may be combined or integrated into a single computing device, or the functionality of a particular computing device may be distributed across one or more other computing devices without departing from the scope of the embodiments of the present disclosure.

Hereinafter, with reference to Examples and Comparative Examples, a fused polycyclic compound and a light emitting element of one or more embodiments of the disclosure will be specifically described. In some embodiments, Examples are shown only for the understanding of the disclosure, and the scope of the disclosure is not limited thereto.

### EXAMPLES

### 1. Synthesis of Amine Compounds

First, a method of synthesizing an amine compound according to one or more embodiments will be described in detail by providing a method of synthesizing Compounds 1 to 12 disclosed in Table 5 as an example. In addition, a process of synthesizing an amine compound, which will be described hereinafter, is provided as an example, and thus the process of synthesizing an amine compound according to one or more embodiments of the disclosure is not limited to Examples.

### Methods of Synthesizing Intermediates Used in the Synthesis of Compounds Synthesis of Intermediate A3

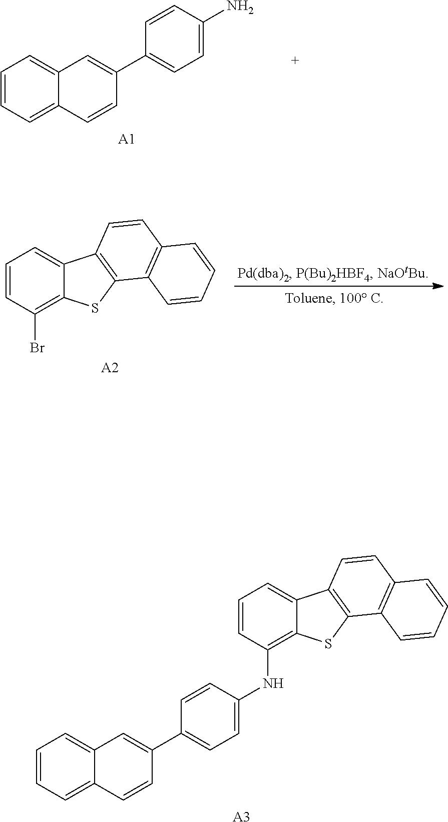

Toluene (200 mL) was added to Intermediate A1 (10 mmol), Intermediate A2 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Intermediate A3 (9.1 mmol, 91%, MS: 451.1).

### Synthesis of Intermediate A5

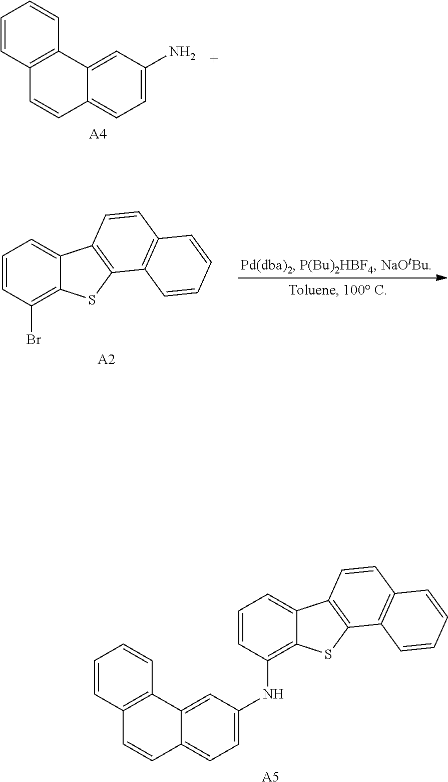

Toluene (200 mL) was added to Intermediate A4 (10 mmol), Intermediate A2 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Intermediate A5 (8.8 mmol, 88%, MS: 451.1).

### Synthesis of Intermediate A7

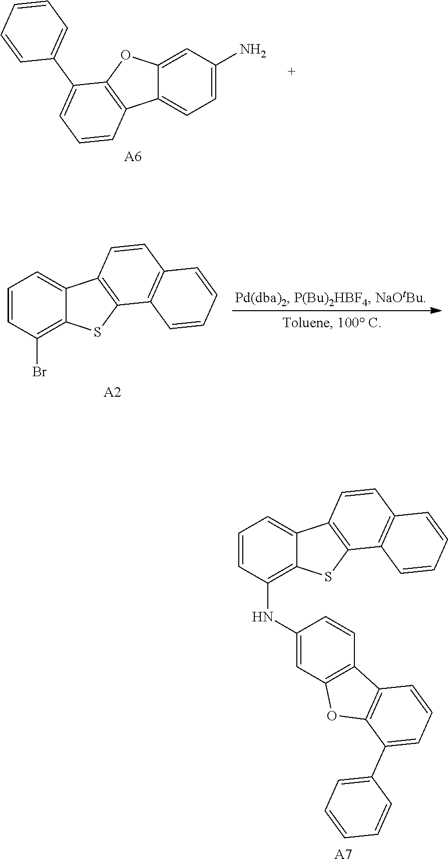

Toluene (200 mL) was added to Intermediate A6 (10 mmol), Intermediate A2 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Intermediate A7 (8.6 mmol, 86%, MS: 491.1).

### Synthesis of Intermediate A10

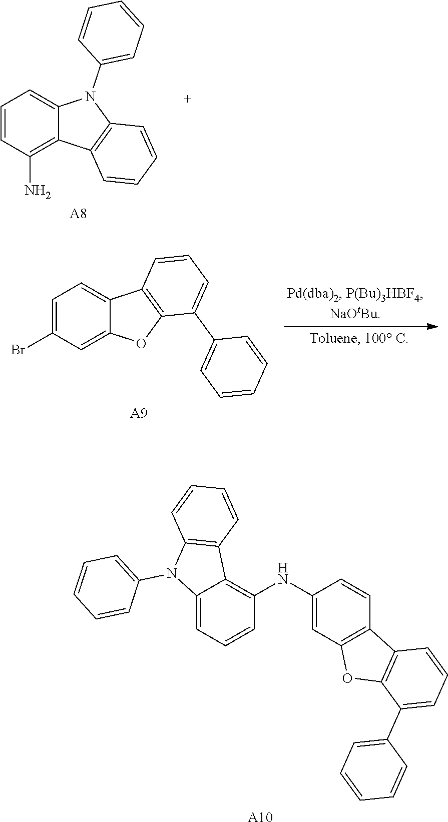

Toluene (200 mL) was added to Intermediate A8 (10 mmol), Intermediate A9 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Intermediate A10 (8.7 mmol, 87%, MS: 500.2).

### Methods of Synthesizing Compounds

### 1) Synthesis of Compound 1

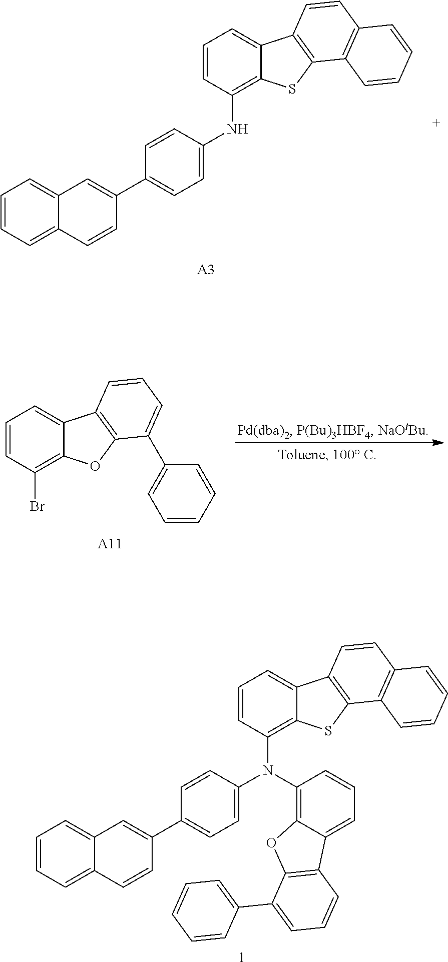

Toluene (200 mL) was added to Intermediate A3 (10 mmol), Intermediate A11 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Compound 1 (8.1 mmol, 81%, MS: 693.2). 2) Synthesis of Compound 2

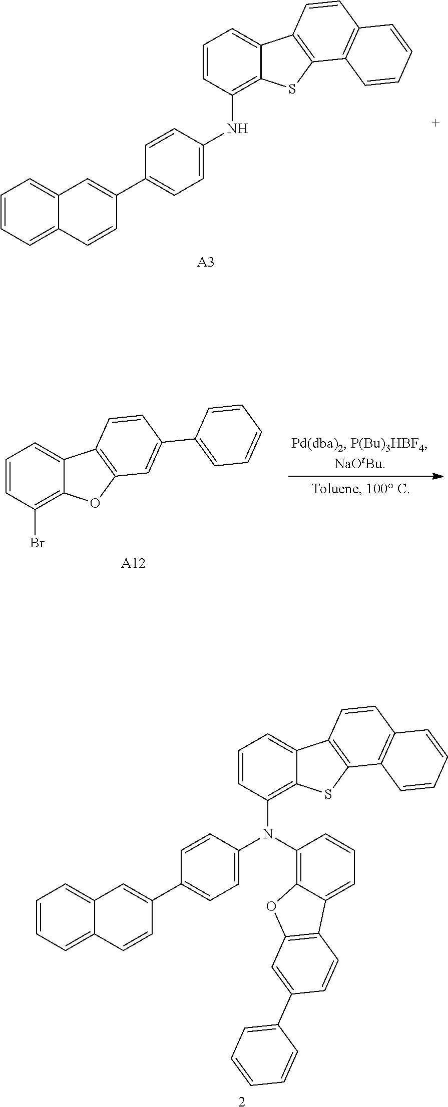

Toluene (200 mL) was added to Intermediate A3 (10 mmol), Intermediate A12 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Compound 2 (78 mmol, 78%, MS: 693.2). 3) Synthesis of Compound 3

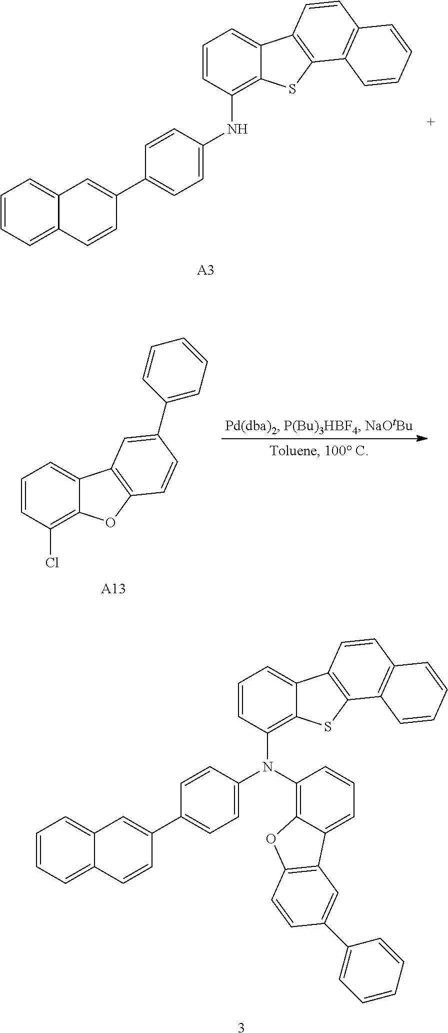 [SMILES: c1ccc2cc(-c3ccc(Nc4cccc5c4sc4c6ccccc6ccc54)cc3)ccc2c1 ; "A3" ; Clc1cccc2c1oc1ccc(-c3ccccc3)cc12 ; "A13" ; c1ccc(-c2ccc3oc4c(N(c5ccc(-c6ccc7ccccc7c6)cc5)c5cccc6c5sc5c7ccccc7ccc65)cccc4c3c2)cc1 ; "3"]

Toluene (200 mL) was added to Intermediate A3 (10 mmol), Intermediate A13 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Compound 3 (6.5 mmol, 65%, MS: 693.2).

### 4) Synthesis of Compound 4

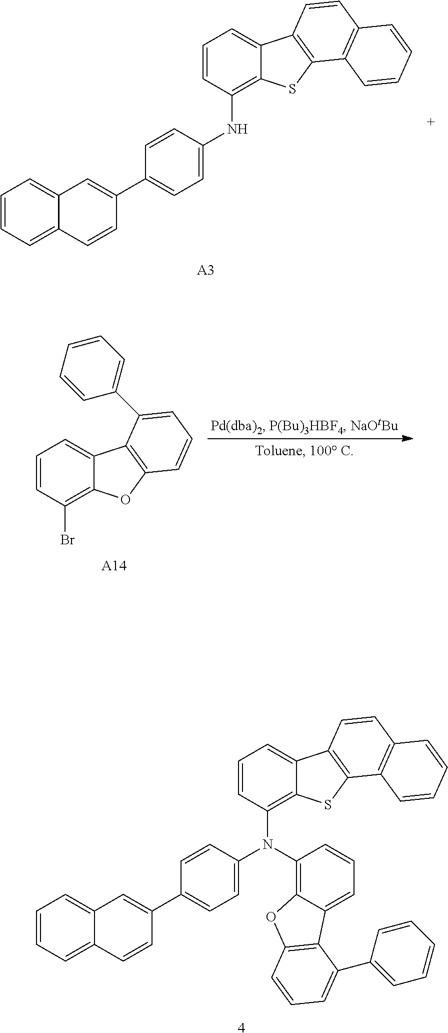 [SMILES: c1ccc2cc(-c3ccc(Nc4cccc5c4sc4c6ccccc6ccc54)cc3)ccc2c1 ; "A3" ; Brc1cccc2c1oc1cccc(-c3ccccc3)c12 ; "A14" ; c1ccc(-c2cccc3oc4c(N(c5ccc(-c6ccc7ccccc7c6)cc5)c5cccc6c5sc5c7ccccc7ccc65)cccc4c23)cc1 ; "4"]

Toluene (200 mL) was added to Intermediate A3 (10 mmol), Intermediate A14 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Compound 4 (6.1 mmol, 61%, MS: 693.2).

### 5) Synthesis of Compound 5

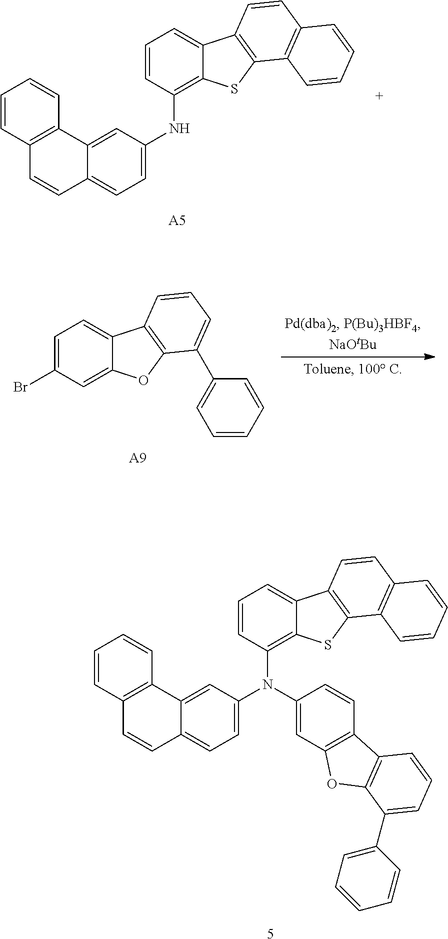 [SMILES: c1ccc2c(c1)ccc1ccc(Nc3cccc4c3sc3c5ccccc5ccc43)cc12 ; "A5" ; Brc1ccc2c(c1)oc1c(-c3ccccc3)cccc12 ; "A9" ; c1ccc(-c2cccc3c2oc2cc(N(c4ccc5ccc6ccccc6c5c4)c4cccc5c4sc4c6ccccc6ccc54)ccc23)cc1 ; "5"]

Toluene (200 mL) was added to Intermediate A5 (10 mmol), Intermediate A9 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Compound 5 (8.3 mmol, 83%, MS: 667.2).

### 6) Synthesis of Compound 6

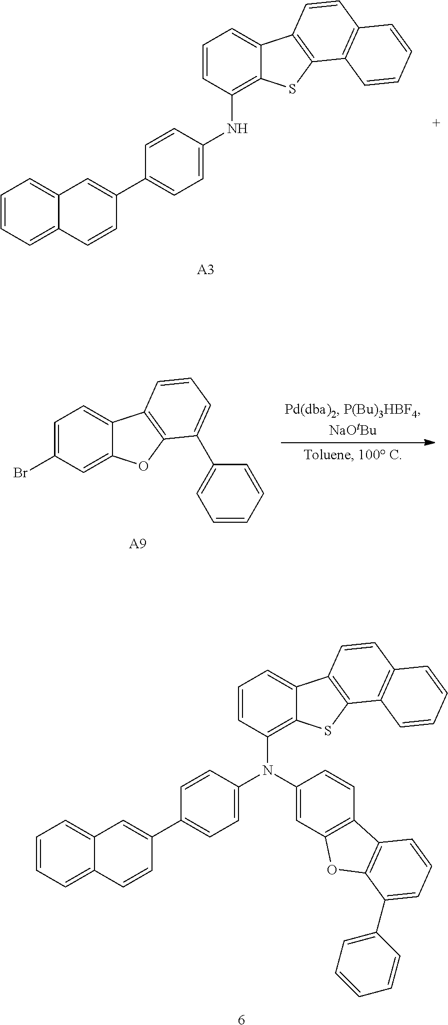 [SMILES: c1ccc2cc(-c3ccc(Nc4cccc5c4sc4c6ccccc6ccc54)cc3)ccc2c1 ; "A3" ; Brc1ccc2c(c1)oc1c(-c3ccccc3)cccc12 ; "A9" ; c1ccc(-c2cccc3c2oc2cc(N(c4ccc(-c5ccc6ccccc6c5)cc4)c4cccc5c4sc4c6ccccc6ccc54)ccc23)cc1 ; "6"]

Toluene (200 mL) was added to Intermediate A3 (10 mmol), Intermediate A9 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Compound 6 (7.8 mmol, 78%, MS: 693.2).

### 7) Synthesis of Compound 7

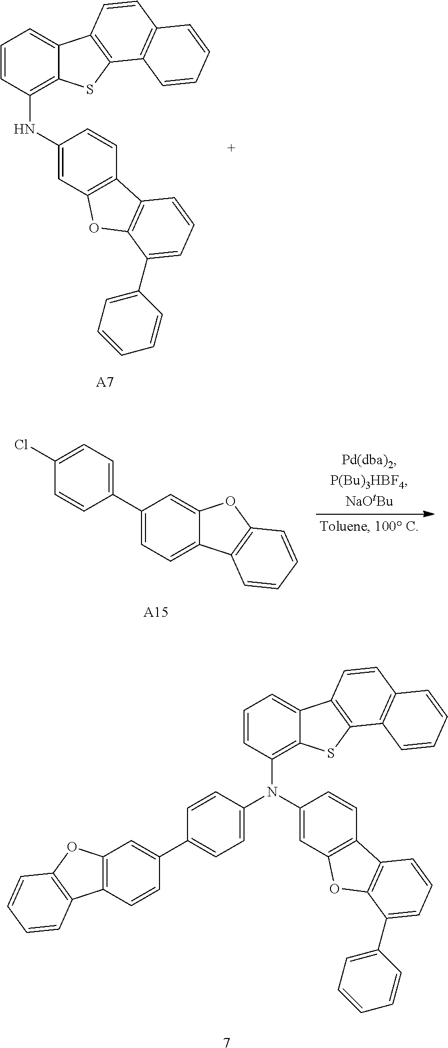

Toluene (200 mL) was added to Intermediate A7 (10 mmol), Intermediate A15 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Compound 7 (6.4 mmol, 64%, MS: 733.2).

### 8) Synthesis of Compound 8

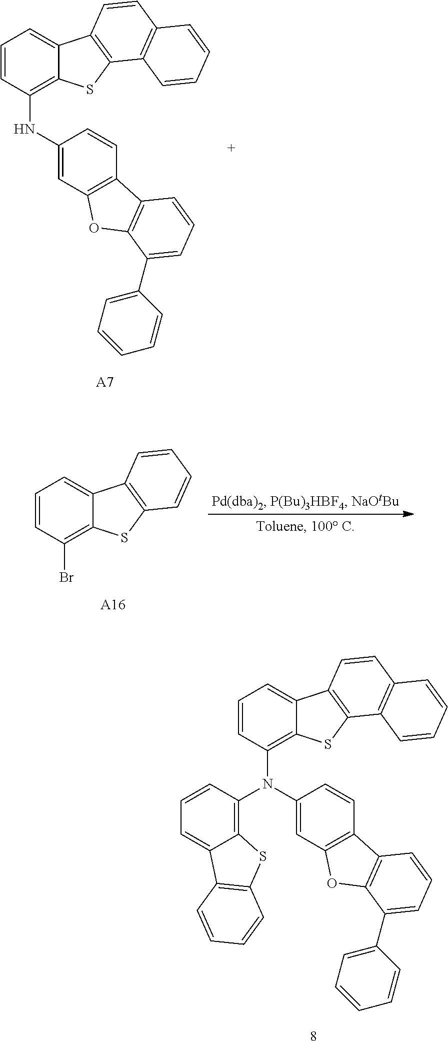

Toluene (200 mL) was added to Intermediate A7 (10 mmol), Intermediate A16 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Compound 8 (7.7 mmol, 77%, MS: 673.2).

### 9) Synthesis of Compound 9

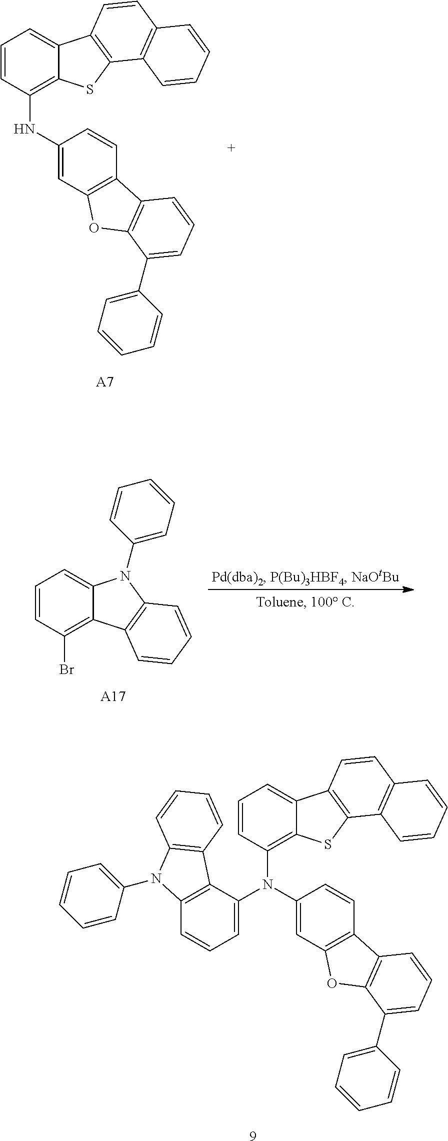

Toluene (200 mL) was added to Intermediate A7 (10 mmol), Intermediate A17 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Compound 9 (8.3 mmol, 83%, MS: 732.9).

### 10) Synthesis of Compound 10

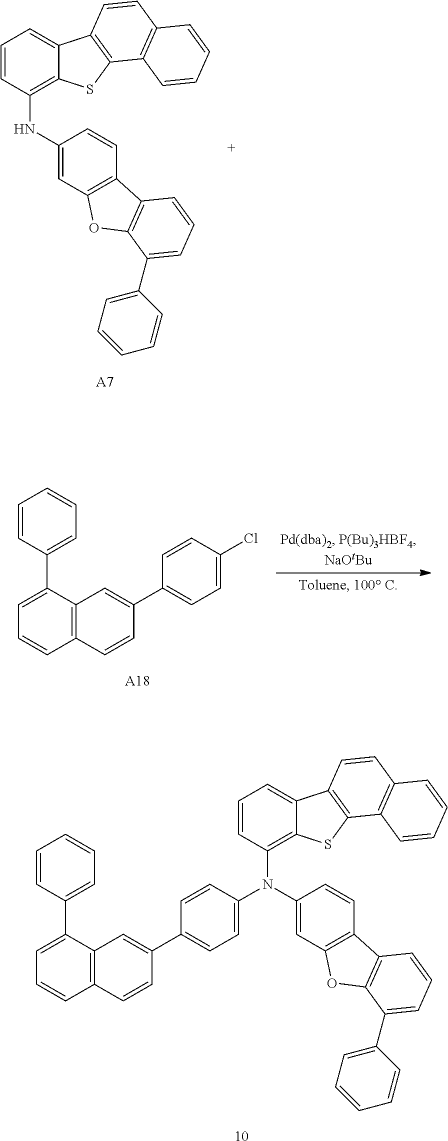

Toluene (200 mL) was added to Intermediate A7 (10 mmol), Intermediate A18 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Compound 10 (8.2 mmol, 82%, MS: 769.2).

### 11) Synthesis of Compound 11

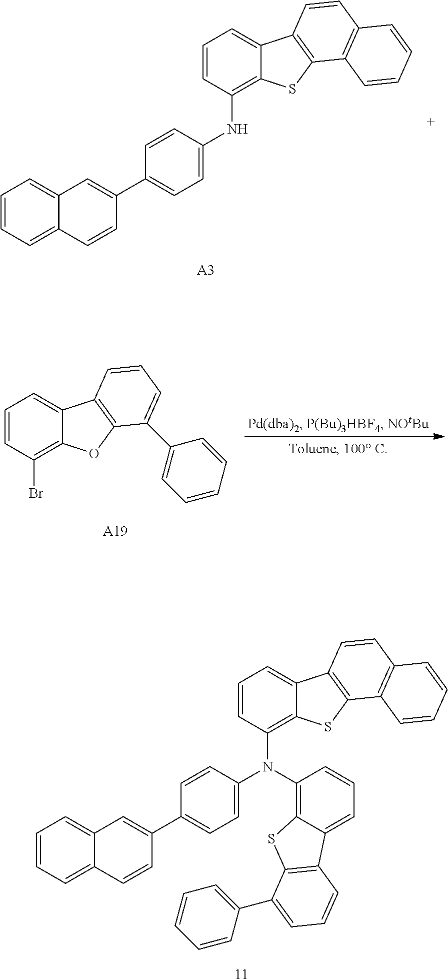

Toluene (200 mL) was added to Intermediate A3 (10 mmol), Intermediate A19 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Compound 11 (8.1 mmol, 81%, MS: 709.2).

### 12) Synthesis of Compound 12

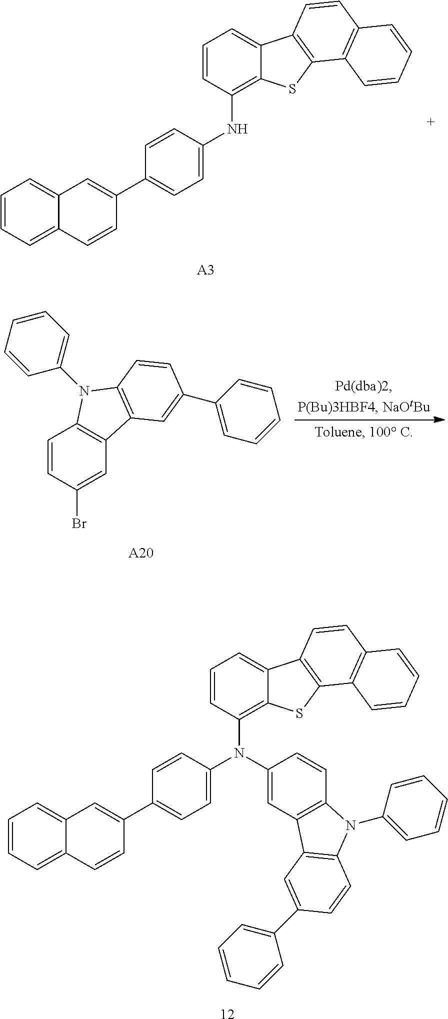

Toluene (200 mL) was added to Intermediate A3 (10 mmol), Intermediate A20 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Compound 12 (7.9 mmol, 79%, MS: 679.0). 13) Synthesis of Compound 13

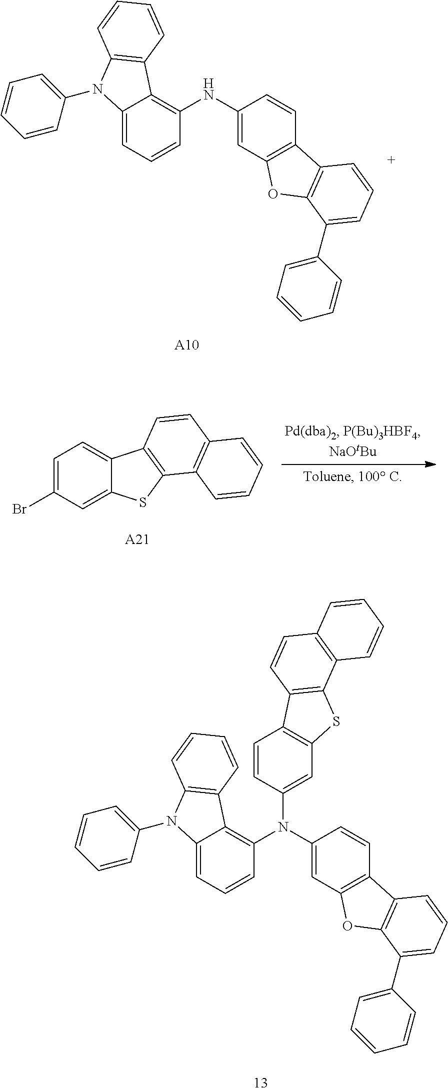

Toluene (200 mL) was added to Intermediate A10 (10 mmol), Intermediate A21 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Compound 13 (7.6 mmol, 76%, MS: 732.2).

### 14) Synthesis of Compound 14

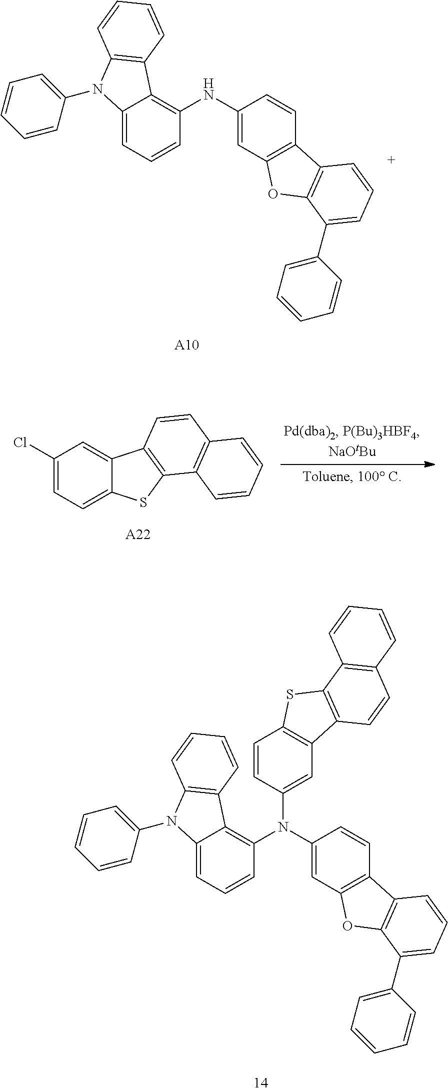

Toluene (200 mL) was added to Intermediate A10 (10 mmol), Intermediate A22 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Compound 14 (6.7 mmol, 67%, MS: 732.2).

### 15) Synthesis of Compound 15

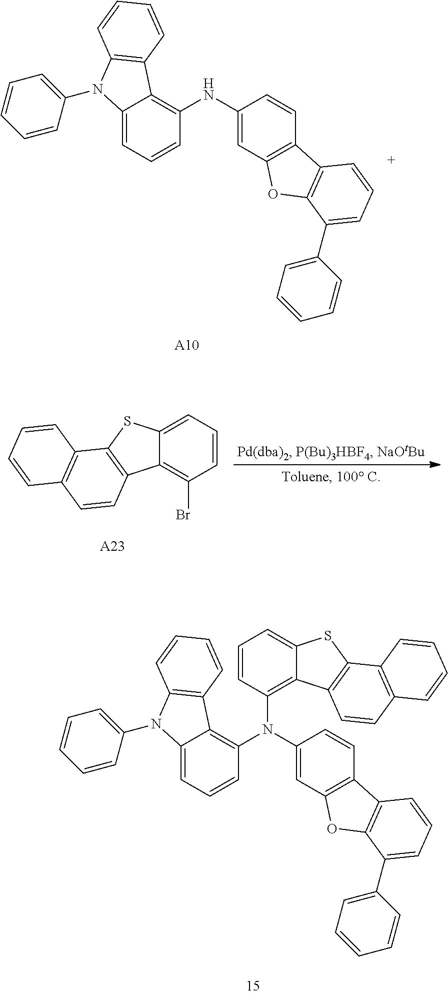

Toluene (200 mL) was added to Intermediate A10 (10 mmol), Intermediate A23 (10 mmol), NaOtBu (10 mmol), and P(Bu)₃HBF₄ (1 mmol), and the mixture was degassed. Bis(dibenzylideneacetone)palladium (0.5 mmol) was added in an argon atmosphere, and heated and stirred at 100° C. for 6 hours. The reaction solution was cooled to room temperature, extracted with toluene, washed with H₂O and brine, and dried over Na₂SO₄. The obtained solution was concentrated and purified through column chromatography to obtain Compound 15 (6.9 mmol, 69%, MS: 732.2).

### 2. Preparation and Evaluation of Light Emitting Elements

Light emitting elements of one or more embodiments including an amine compound of one or more embodiments in a hole transport layer were prepared using the following method. Light emitting elements of Examples 1 to 15 were prepared using amine compounds of Compounds 1 to 15, which are Example Compounds described herein, as hole transport layer materials. Comparative Examples 1 to 12 correspond to light emitting elements prepared using Comparative Example Compounds R1 to R12 as hole transport layer materials.

### Example Compounds

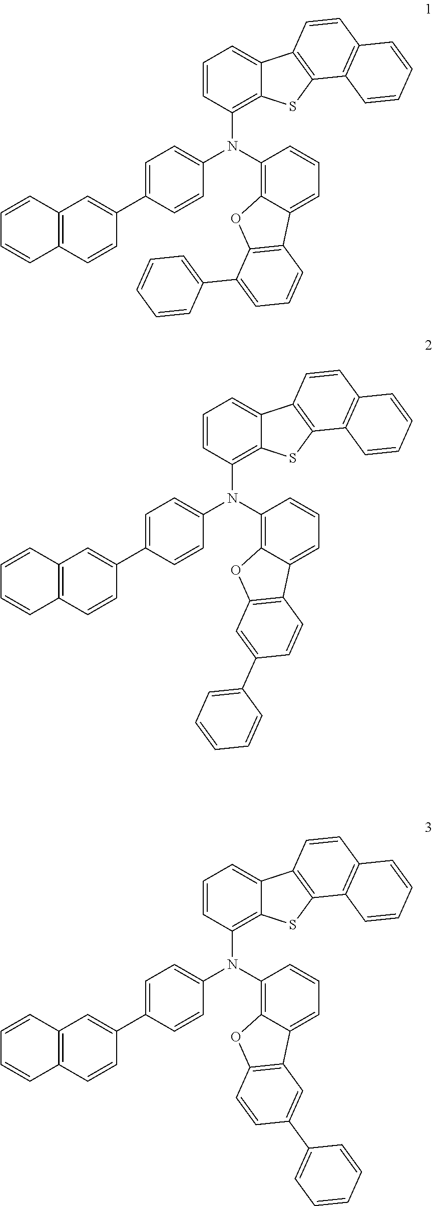 [SMILES: c1ccc(-c2cccc3c2oc2c(N(c4ccc(-c5ccc6ccccc6c5)cc4)c4cccc5c4sc4c6ccccc6ccc54)cccc23)cc1 ; c1ccc(-c2ccc3c(c2)oc2c(N(c4ccc(-c5ccc6ccccc6c5)cc4)c4cccc5c4sc4c6ccccc6ccc54)cccc23)cc1 ; c1ccc(-c2ccc3oc4c(N(c5ccc(-c6ccc7ccccc7c6)cc5)c5cccc6c5sc5c7ccccc7ccc65)cccc4c3c2)cc1]

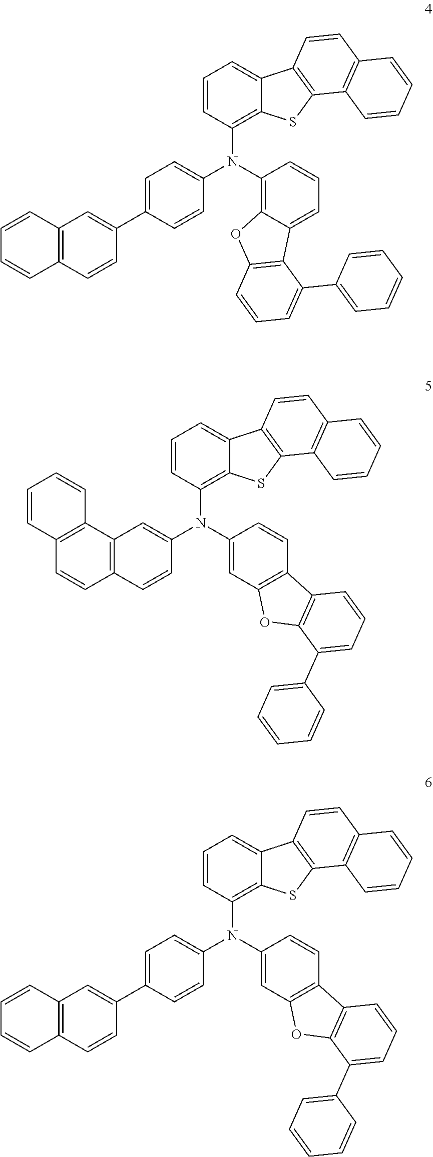 [SMILES: c1ccc(-c2cccc3oc4c(N(c5ccc(-c6ccc7ccccc7c6)cc5)c5cccc6c5sc5c7ccccc7ccc65)cccc4c23)cc1 ; c1ccc(-c2cccc3c2oc2cc(N(c4ccc5ccc6ccccc6c5c4)c4cccc5c4sc4c6ccccc6ccc54)ccc23)cc1 ; c1ccc(-c2cccc3c2oc2cc(N(c4ccc(-c5ccc6ccccc6c5)cc4)c4cccc5c4sc4c6ccccc6ccc54)ccc23)cc1]

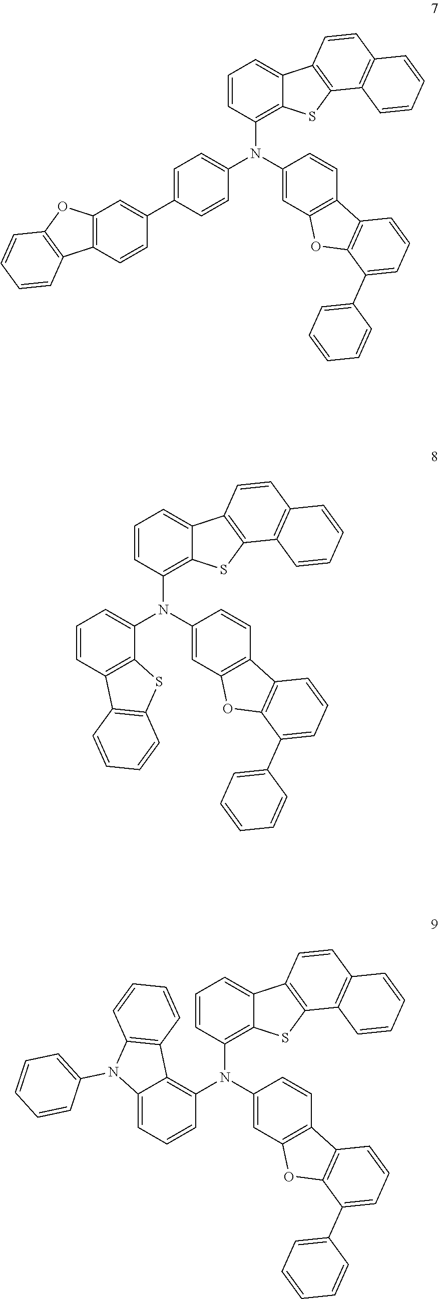 [SMILES: c1ccc(-c2cccc3c2oc2cc(N(c4ccc(-c5ccc6c(c5)oc5ccccc56)cc4)c4cccc5c4sc4c6ccccc6ccc54)ccc23)cc1 ; c1ccc(-c2cccc3c2oc2cc(N(c4cccc5c4sc4ccccc45)c4cccc5c4sc4c6ccccc6ccc54)ccc23)cc1 ; c1ccc(-c2cccc3c2oc2cc(N(c4cccc5c4sc4c6ccccc6ccc54)c4cccc5c4c4ccccc4n5-c4ccccc4)ccc23)cc1]

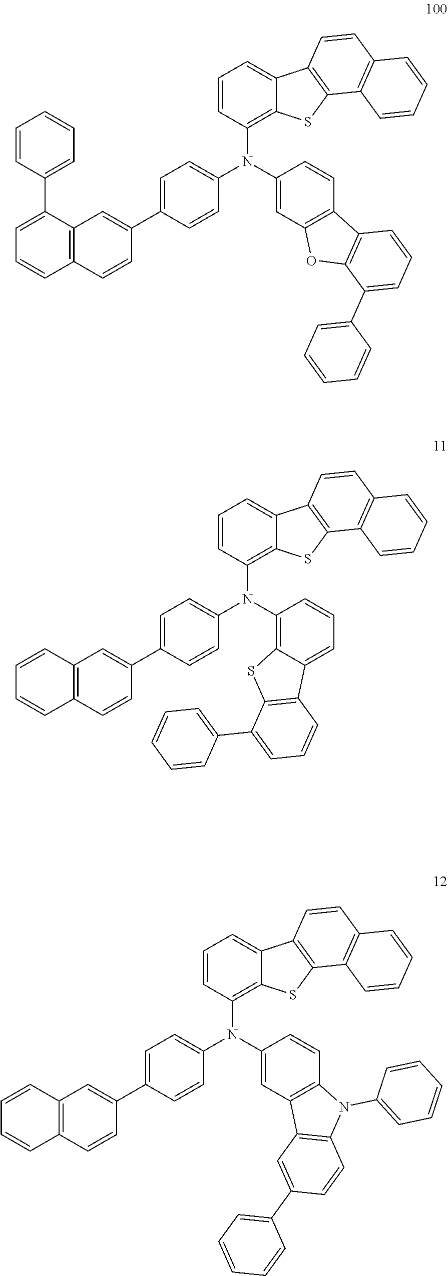 [SMILES: c1ccc(-c2cccc3ccc(-c4ccc(N(c5ccc6c(c5)oc5c(-c7ccccc7)cccc56)c5cccc6c5sc5c7ccccc7ccc65)cc4)cc23)cc1 ; c1ccc(-c2cccc3c2sc2c(N(c4ccc(-c5ccc6ccccc6c5)cc4)c4cccc5c4sc4c6ccccc6ccc54)cccc23)cc1 ; c1ccc(-c2ccc3c(c2)c2cc(N(c4ccc(-c5ccc6ccccc6c5)cc4)c4cccc5c4sc4c6ccccc6ccc54)ccc2n3-c2ccccc2)cc1]

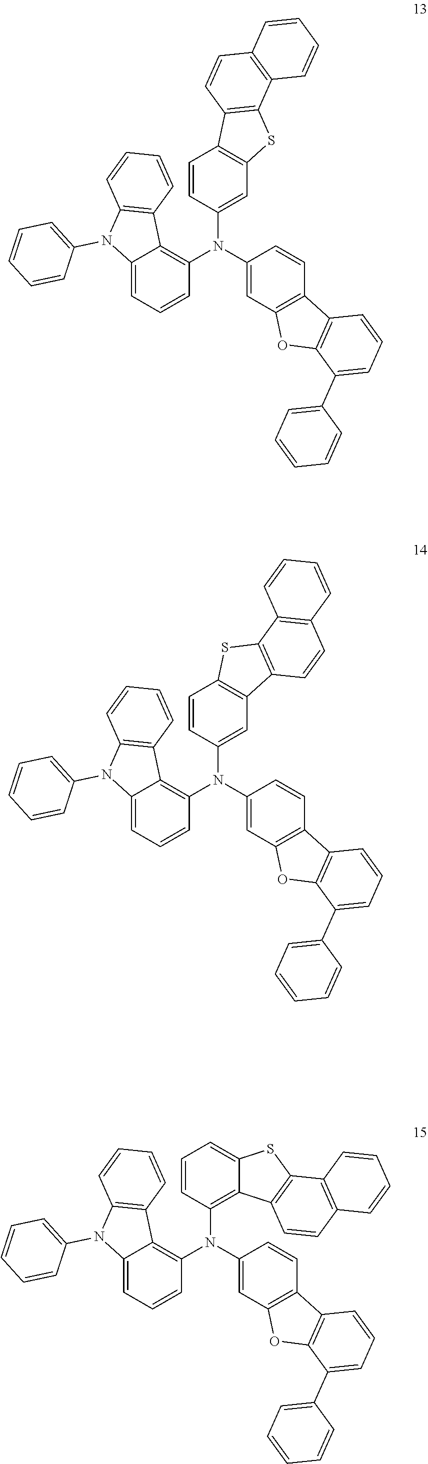 [SMILES: c1ccc(-c2cccc3c2oc2cc(N(c4ccc5c(c4)sc4c6ccccc6ccc54)c4cccc5c4c4ccccc4n5-c4ccccc4)ccc23)cc1 ; c1ccc(-c2cccc3c2oc2cc(N(c4ccc5sc6c7ccccc7ccc6c5c4)c4cccc5c4c4ccccc4n5-c4ccccc4)ccc23)cc1 ; c1ccc(-c2cccc3c2oc2cc(N(c4cccc5sc6c7ccccc7ccc6c45)c4cccc5c4c4ccccc4n5-c4ccccc4)ccc23)cc1]

### Comparative Example Compounds

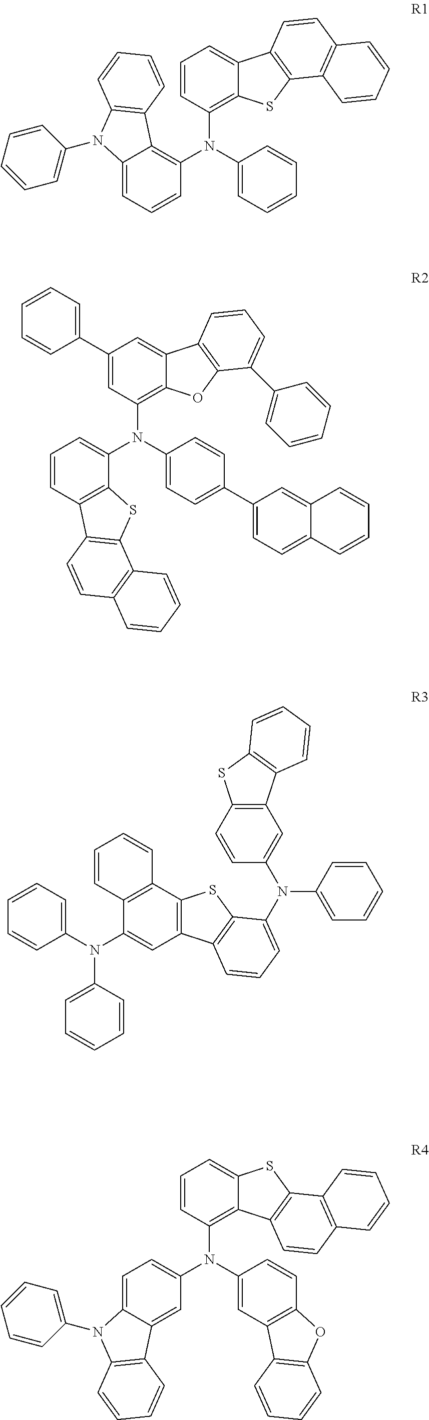 [SMILES: c1ccc(N(c2cccc3c2sc2c4ccccc4ccc32)c2cccc3c2c2ccccc2n3-c2ccccc2)cc1 ; c1ccc(-c2cc(N(c3ccc(-c4ccc5ccccc5c4)cc3)c3cccc4c3sc3c5ccccc5ccc43)c3oc4c(-c5ccccc5)cccc4c3c2)cc1 ; c1ccc(N(c2ccccc2)c2cc3c4cccc(N(c5ccccc5)c5ccc6sc7ccccc7c6c5)c4sc3c3ccccc23)cc1 ; c1ccc(-n2c3ccccc3c3cc(N(c4ccc5oc6ccccc6c5c4)c4cccc5sc6c7ccccc7ccc6c45)ccc32)cc1]

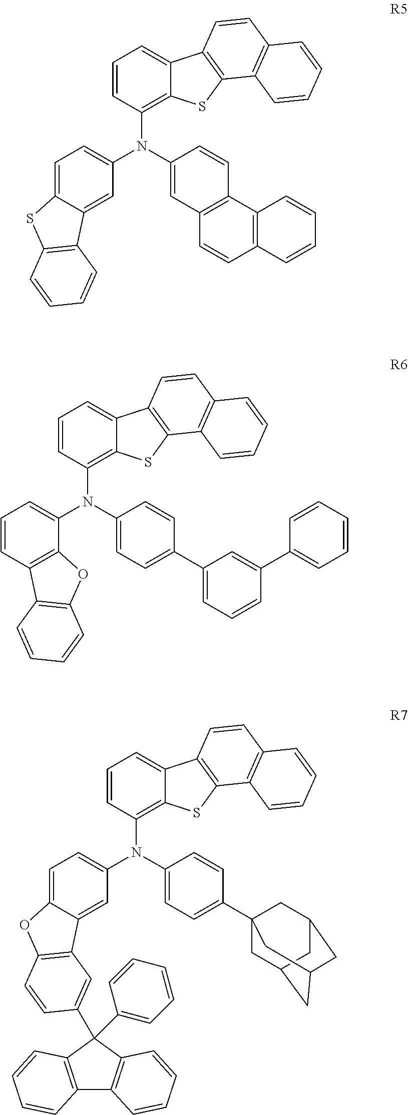 [SMILES: c1ccc2c(c1)ccc1cc(N(c3ccc4sc5ccccc5c4c3)c3cccc4c3sc3c5ccccc5ccc43)ccc12 ; c1ccc(-c2cccc(-c3ccc(N(c4cccc5c4oc4ccccc45)c4cccc5c4sc4c6ccccc6ccc54)cc3)c2)cc1 ; c1ccc(C2(c3ccc4oc5ccc(N(c6ccc(C78CC9CC(CC(C9)C7)C8)cc6)c6cccc7c6sc6c8ccccc8ccc76)cc5c4c3)c3ccccc3-c3ccccc32)cc1]

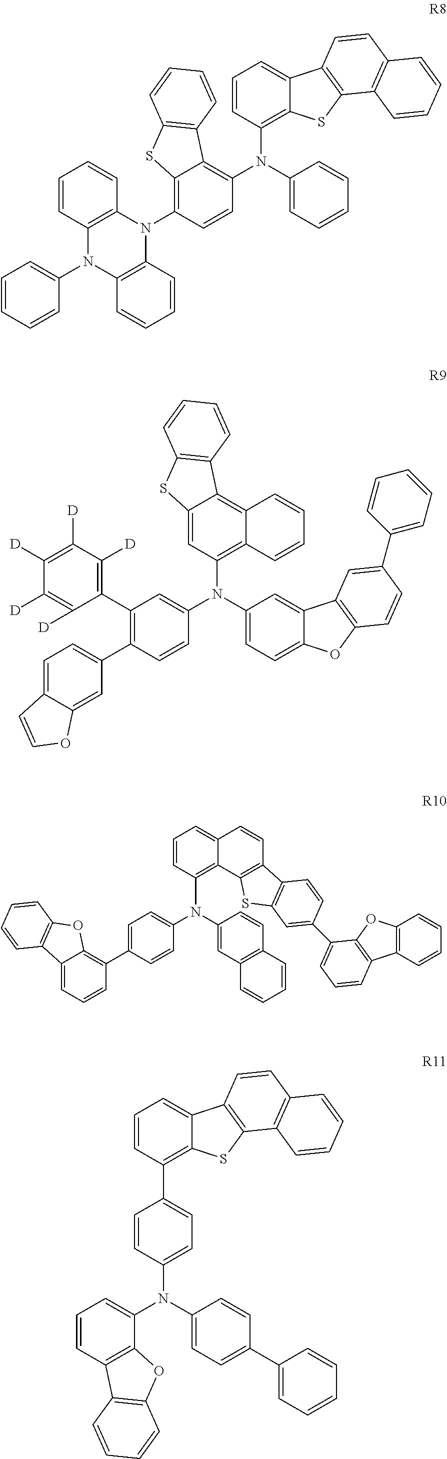 [SMILES: c1ccc(N2c3ccccc3N(c3ccc(N(c4ccccc4)c4cccc5c4sc4c6ccccc6ccc54)c4c3sc3ccccc34)c3ccccc32)cc1 ; [2H]c1c([2H])c([2H])c(-c2cc(N(c3ccc4oc5ccc(-c6ccccc6)cc5c4c3)c3cc4sc5ccccc5c4c4ccccc34)ccc2-c2ccc3ccoc3c2)c([2H])c1[2H] ; c1ccc2cc(N(c3ccc(-c4cccc5c4oc4ccccc45)cc3)c3cccc4ccc5c6ccc(-c7cccc8c7oc7ccccc78)cc6sc5c34)ccc2c1 ; c1ccc(-c2ccc(N(c3ccc(-c4cccc5c4sc4c6ccccc6ccc54)cc3)c3cccc4c3oc3ccccc34)cc2)cc1]

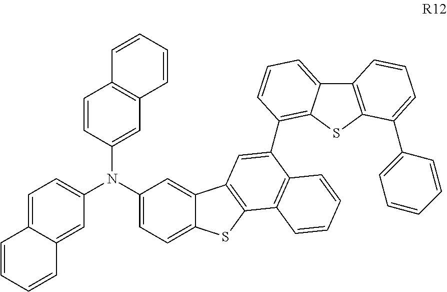 [SMILES: c1ccc(-c2cccc3c2sc2c(-c4cc5c6cc(N(c7ccc8ccccc8c7)c7ccc8ccccc8c7)ccc6sc5c5ccccc45)cccc23)cc1]

### Preparation of Light Emitting Elements

A glass substrate on which an ITO having a thickness of 150 nanometer (nm) was patterned as a first electrode was subjected to ultrasonic cleaning using isopropyl alcohol and pure water each for 5 minutes. The glass substrate was irradiated with UV for 30 minutes, and ozone-treated. Thereafter, a hole injection layer was formed through the deposition of 2-TNATA with a thickness of 60 nm. On the hole injection layer, a hole transport layer was formed through the deposition of Example compounds or Comparative Example compounds with a thickness of 30 nm.

On the hole transport layer, an emission layer was formed through the co-deposition of TBP and ADN with a thickness of 25 nm. TBP and ADN were subjected to the co-deposition at a weight ratio of 3:97. Thereafter, an electron transport region was formed through the sequential deposition of Alq₃ with a thickness of 25 nm and LiF with a thickness of 1 nm.

Then, a second electrode was formed through the deposition of Al with a thickness of 100 nm.

In one or more embodiments, the hole transport region, the emission layer, the electron transport region, and the second electrode were formed using a vacuum deposition apparatus.

The compounds used to manufacture the light emitting elements are as follows.

### Materials Used in Preparation of Light Emitting Elements

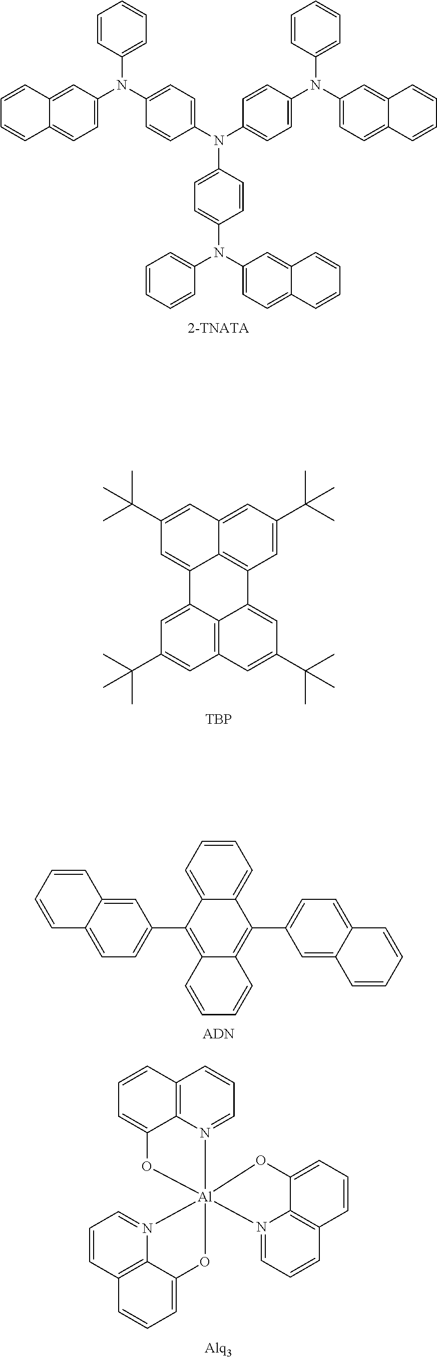

### (2) Evaluation of Light Emitting Element

Table 5 shows evaluation results of the light emitting elements of Examples and Comparative Examples. Table 5 shows the element lifespan for the light emitting elements of Examples and Comparative Examples. The element lifespan was evaluated using a C9920-11 luminance orientation characteristic measuring device from Hamamatsu Photonics, and the time taken for luminance to degrade to 50% from an initial luminance value during substantially continuous operation was shown relatively, with respect to a value of Comparative Example 6 set as 100%.

TABLE 5

| Preparation example | Hole transport layer | Element lifespan |
|---|---|---|
| of element | material | (LT50) |
| Example 1 | Compound 1 | 115% |
| Example 2 | Compound 2 | 113% |
| Example 3 | Compound 3 | 112% |
| Example 4 | Compound 4 | 109% |
| Example 5 | Compound 5 | 129% |
| Example 6 | Compound 6 | 131% |
| Example 7 | Compound 7 | 111% |
| Example 8 | Compound 8 | 116% |
| Example 9 | Compound 9 | 109% |
| Example 10 | Compound 10 | 121% |
| Example 11 | Compound 11 | 119% |
| Example 12 | Compound 12 | 110% |
| Example 13 | Compound 13 | 106% |
| Example 14 | Compound 14 | 107% |
| Example 15 | Compound 15 | 110% |
| Comparative Example 1 | Comparative Example | 79% |
| | Compound R1 | |
| Comparative Example 2 | Comparative Example | 95% |
| | Compound R2 | |
| Comparative Example 3 | Comparative Example | 68% |
| | Compound R3 | |
| Comparative Example 4 | Comparative Example | 73% |
| | Compound R4 | |
| Comparative Example 5 | Comparative Example | 97% |
| | Compound R5 | |
| Comparative Example 6 | Comparative Example | 100% |
| | Compound R6 | |
| Comparative Example 7 | Comparative Example | 83% |
| | Compound R7 | |
| Comparative Example 8 | Comparative Example | 69% |
| | Compound R8 | |
| Comparative Example 9 | Comparative Example | 39% |
| | Compound R9 | |
| Comparative Example 10 | Comparative Example | 41% |
| | Compound R10 | |
| Comparative Example 11 | Comparative Example | 81% |
| | Compound R11 | |
| Comparative Example 12 | Comparative Example | 49% |
| | Compound R12 | |

Referring to the results in Table 5, Examples 1 to 13 exhibited long lifespan element characteristics compared to Comparative Examples 1 to 12. Example compounds include a first substituent of a benzonaphthothiophene moiety bonded to an amine group, a second substituent of a moiety such as phenyldibenzofuran, and a third substituent selected from among an aryl group and/or a heteroaryl group. Depending on these specific substituent groups and substitution positions, the Example compounds may exhibit excellent or suitable material stability compared to other Comparative Example compounds. For example, it is determined that Example compounds have excellent or suitable charge balance due to the molecular structural characteristics of the Example compounds that are distinguished from Comparative Example compounds. Accordingly, the light emitting elements of Examples including the amine compound of Examples in the hole transport layer exhibit excellent or suitable long lifespan characteristics.

Comparative Example Compounds R1, Comparative Example Compound R4, Comparative Example Compounds R5, and Comparative Example Compounds R6 include the first substituent and the third substituent that are bonded to an amine group, but do not include the second substituent proposed herein, indicating that the effects related to stacking via intermolecular interaction and intermolecular orientation are reduced. Accordingly, it is determined that Comparative Example 1, Comparative Example 4, Comparative Example 5, and Comparative Example 6, including Comparative Example Compound R1, Comparative Example Compound R4, Comparative Example Compound R5, and Comparative Example Compound R6, respectively, have relatively reduced element lifespan compared to Example compounds. In the case of Comparative Example Compound R1 and Comparative Example Compound R4, the phenyl group other than the second substituent includes an unsubstituted 9-phenylcarbazole group, in the case of Comparative Example Compound R5, the phenyl group other than the second substituent includes an unsubstituted dibenzothiophene group, and in the case of Comparative Example Compound R6, the phenyl group other than the second substituent includes an unsubstituted dibenzofuran group, and thus it is considered that the light emitting elements of Comparative Examples 1, 4, 5, and 6, which include Comparative Example Compound R1, Comparative Example Compound R4, Comparative Example Compound R5, and Comparative Example Compound R6, have reduced element lifespan.

Comparative Example R2, Comparative Example Compound R7, and Comparative Example Compounds R8 include the first substituent and the third substituent that are bonded to an amine group, but do not include the second substituent proposed herein, and thus it is considered that the effects related to stacking via intermolecular interaction and intermolecular orientation are reduced. Accordingly, it is determined that Comparative Example 2, Comparative Example 7, and Comparative Example 8, including Comparative Example Compound R2, Comparative Example Compound R7, and Comparative Example Compound R8, respectively, have relatively reduced element lifespan compared to Example compounds. Comparative Example Compound R2 includes a diphenyldibenzofuran group instead of the second substituent, and thus it is considered that the light emitting element of Comparative Example 2, which includes Comparative Example Compound R2, has reduced element lifespan. Comparative Example Compound R7 does not include the second substituent and includes a dibenzofuran group substituted with a fluorene group having a large steric volume, resulting in suppressed or reduced intermolecular interaction, and thus it is considered that the light emitting element of Comparative Example 7, which includes Comparative Example Compound R7, has reduced element lifespan. Comparative Example Compound R8 does not include the second substituent and includes a dibenzofuran group substituted with a phenazine group having a high electron donating property, resulting in strengthened intermolecular interaction, and thus it is considered that the light emitting element of Comparative Example 8, which includes Comparative Example Compound R8, has reduced element lifespan.

Comparative Example Compound R3, Comparative Example Compound R11, and Comparative Example Compound R12 include the third substituent, but do not include the first substituent and the second substituent proposed herein, and thus it is considered that the effects related to stacking via intermolecular interaction and intermolecular orientation are reduced. Accordingly, it is determined that Comparative Example 3, Comparative Example 11, and Comparative Example 12, including Comparative Example Compound R3, Comparative Example Compound R11, and Comparative Example Compound R12, respectively, have relatively reduced element lifespan compared to Example compounds. Comparative Example Compound R3 does not include the first substituent and includes a benzonaphthothiophene group substituted with an arylamine group, and accordingly, electronic effects and steric effects around two amine groups in a diamine compound are different from those of Example compounds, and thus it is considered that the light emitting element of Comparative Example 3 including Comparative Example Compound R3 has reduced element lifespan. In the case of Comparative Example Compound R11, the first substituent is not included and a benzonaphthothiophene group is linked to an amine group via a phenylene linker, and thus it is considered that the light emitting element of Comparative Example 11 including Comparative Example Compound R11 has reduced element lifespan. Comparative Example Compound R12 does not include the first substituent and includes a benzonaphthothiophene group substituted with a dibenzofuran group having a large steric volume, resulting in suppressed or reduced intermolecular interaction, and thus it is considered that the light emitting element of Comparative Example 12, which includes Comparative Example Compound R12, has reduced element lifespan.

Comparative Example Compound R9 includes the second substituent, but does not include the first substituent and the third substituent proposed herein, and thus it is considered that the effects related to stacking via intermolecular interaction and intermolecular orientation are reduced. Accordingly, it is determined that Comparative Example 9 including Comparative Example Compound C9 has relatively reduced element lifespan compared to Example compounds. Comparative Example Compound R9 has a structure in which a naphthalene moiety of a benzonaphthothiophene moiety, unlike the first substituent, is linked to a core nitrogen atom, and includes a benzofuran group instead of the third substituent, and accordingly, electronic effects and steric effects are different from those of Example compounds, and thus it is considered that the light emitting element of Comparative Example 9 including Comparative Example Compound R9 has reduced element lifespan.

Comparative Example Compound R10 includes the third substituent, but does not include the first substituent and the second substituent proposed herein, and thus it is considered that the effects related to stacking via intermolecular interaction and intermolecular orientation are reduced. Accordingly, it is determined that Comparative Example 9 including Comparative Example Compound R10 has relatively reduced element lifespan compared to Example compounds. Comparative Example Compound R10 has a structure in which a naphthalene moiety of a benzonaphthothiophene moiety, unlike the first substituent, is linked to a core nitrogen atom, and includes a dibenzofuran group in which a phenyl group is not substituted instead of the second substituent, and thus it is considered that the light emitting element of Comparative Example 10 including Comparative Example Compound R10 has reduced element lifespan.

In summary, the structural analysis and comparative performance data indicate that the presence and specific arrangement of the first, second, and third substituents in the amine compounds play a critical role in determining the operational lifespan of the light emitting elements. The absence or modification of any one of these substituents, as seen in the Comparative Example compounds, leads to diminished intermolecular interactions and suboptimal charge transport properties. In contrast, the amine compounds of the Example embodiments, when incorporated into the hole transport layer of a light emitting element, contribute to enhanced stability and extended device lifespan.

A light emitting element according to one or more embodiments may include an amine compound as described throughout this disclosure, particularly within a functional layer such as the hole transport layer. These amine compounds, characterized by specific combinations of first, second, and third substituents, are designed to enhance charge transport, molecular stability, and intermolecular interactions. When incorporated into a light emitting element, these compounds contribute to improved charge balance and enhanced structural integrity under operational conditions. As a result, display devices and electronic apparatuses that include such light emitting elements may exhibit significantly extended operational lifespans, making them well-suited for high-performance and long-duration applications such as OLED displays, lighting panels, and/or wearable electronics.

The amine compounds disclosed in one or more embodiments may be engineered with molecular architectures that promote favorable electronic properties, such as high hole mobility and/or appropriate energy level alignment with adjacent layers. These compounds also exhibit steric and electronic features that enhance thermal and morphological stability, reducing degradation over time. By carefully tuning the substituent groups and their positions, the compounds may achieve a balance between rigidity and flexibility, which supports efficient charge transport while minimizing crystallization or phase separation. When applied to light emitting elements, these structural advantages translate into devices with longer lifespans, higher reliability, and/or improved overall performance.

In the context of the present application and unless otherwise defined, the terms “use,” “using,” and “used” may be considered synonymous with the terms “utilize,” “utilizing,” and “utilized,” respectively.

A person of ordinary skill in the art, in view of the present disclosure in its entirety, would appreciate that each suitable feature of the one or more suitable embodiments of the present disclosure may be combined or combined with each other, partially or entirely, and may be technically interlocked and operated in one or more suitable ways, and each embodiment may be implemented independently of each other or in conjunction with each other in any suitable manner unless otherwise stated or implied.

In the preceding, description has been made with reference to one or more embodiments of the disclosure, but those skilled or of ordinary skill in the art may understand that one or more suitable modifications and changes may be made to the disclosure insofar as such modifications and changes do not depart from the spirit and technical scope of the disclosure set forth in the claims to be described later. Therefore, the technical scope of the disclosure is not to be limited to the contents stated in the detailed description of the specification, but should be determined by the claims and equivalents thereof.

What is claimed is:

1. A light emitting element comprising:
a first electrode;
a second electrode on the first electrode; and
at least one functional layer between the first electrode and the second electrode and comprising an amine compound, the amine compound being represented by Formula 1: 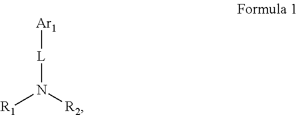
wherein in Formula 1,
L is a direct linkage, a substituted or unsubstituted arylene group having 6 to 30 ring-forming carbon atoms, or a substituted or unsubstituted heteroarylene group having 2 to 30 ring-forming carbon atoms,
Ar₁ is a substituted or unsubstituted aryl group having 6 to 30 ring-forming carbon atoms, or a substituted or unsubstituted heteroaryl group having 2 to 30 ring-forming carbon atoms, where Ar₁ does not comprise a substituted or unsubstituted benzofuran group and a substituted or unsubstituted benzothiophene group,
R₁ is represented by Formula 2, and
R₂ is represented by Formula 3: 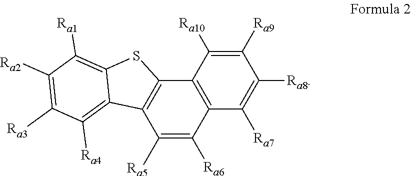
wherein in Formula 2,
any one selected from among Rₐ₁ to Rₐ₄ is connected to Formula 1, and any remaining Rₐ₁ to Rₐ₄ is each independently a hydrogen atom, a deuterium atom, a halogen atom, a cyano group, or a substituted or unsubstituted alkyl group having 1 to 10 carbon atoms, and
Rₐ₅ to Rₐ₁₀ are each independently a hydrogen atom, a deuterium atom, a halogen atom, a cyano group, or a substituted or unsubstituted alkyl group having 1 to 10 carbon atoms, 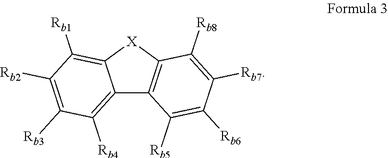
and
wherein in Formula 3,
X is O, S, or NAr₂,
Ar₂ is a substituted or unsubstituted aryl group having 6 to 30 ring-forming carbon atoms,
any one selected from among R_{b1} to R_{b4} is connected to Formula 1, and any remaining R_{b1} to R_{b4} is each independently a hydrogen atom, a deuterium atom, a halogen atom, or a substituted or unsubstituted alkyl group having 1 to 10 carbon atoms,
any one selected from among R_{b5} to R_{b8} is a substituted or unsubstituted aryl group having 6 to 30 ring-forming carbon atoms, wherein R_{b5} to R_{b8} do not comprise a substituted or unsubstituted fluorene group, and
any remaining R_{b5} to R_{b8} is each independently a hydrogen atom, a deuterium atom, a halogen atom, or a substituted or unsubstituted alkyl group having 1 to 10 carbon atoms.

2. The light emitting element of claim 1, wherein the at least one functional layer comprises an emission layer, a hole transport region between the first electrode and the emission layer, and an electron transport region between the emission layer and the second electrode,
the hole transport region comprising the amine compound.

5. The light emitting element of claim 1, wherein in Formula 1, L is a direct linkage, or a substituted or unsubstituted phenylene group.

6. The light emitting element of claim 1, wherein in Formula 1, Ar₁ is a substituted or unsubstituted naphthyl group, a substituted or unsubstituted phenanthrene group, a substituted or unsubstituted dibenzofuran group, a substituted or unsubstituted dibenzothiophene group, or a substituted or unsubstituted carbazole group.

7. The light emitting element of claim 1, wherein the amine compound is represented by Formula 1-1: 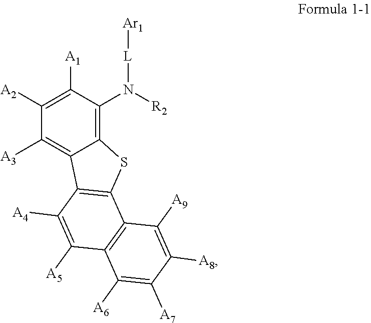
and
wherein in Formula 1-1,
A₁ to A₉ are each independently a hydrogen atom, a deuterium atom, a halogen atom, or a substituted or unsubstituted alkyl group having 1 to 10 carbon atoms, and
L, Ar₁, and R₂ are as defined in Formula 1.

8. The light emitting element of claim 1, wherein the amine compound is represented by any one selected from among Formulas 1-2 to 1-5: 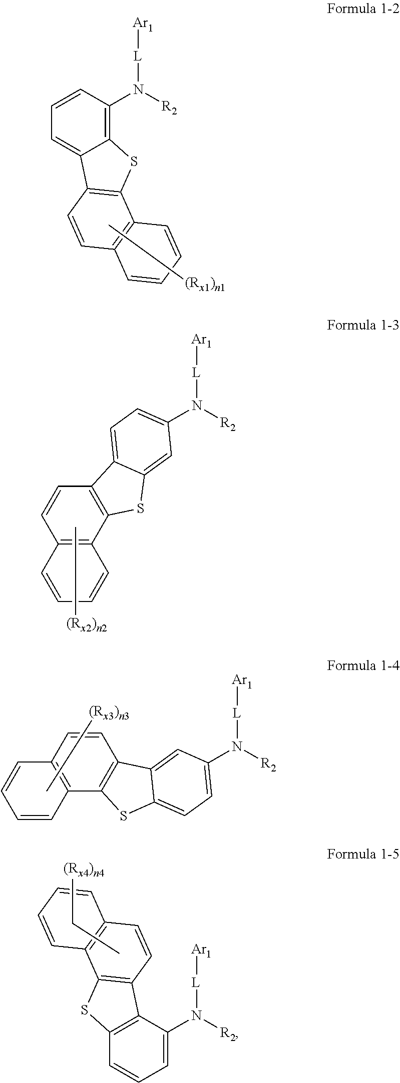
and
wherein in Formulas 1-2 to 1-5,
Rₓ₁ to Rₓ₄ are each independently a hydrogen atom, a deuterium atom, or a substituted or unsubstituted alkyl group having 1 to 5 carbon atoms,
n1 to n4 are each independently an integer of 0 to 6, and
L, Ar₁, and R₂ are as defined in Formula 1.

9. The light emitting element of claim 1, wherein in Formula 3, any one selected from among R_{b1} to R_{b4} is connected to Formula 1, and any remaining R_{b1} to R_{b4} is each independently a hydrogen atom or a deuterium atom.

10. The light emitting element of claim 1, wherein the amine compound is represented by any one selected from among Formulas 1-6 to 1-9: 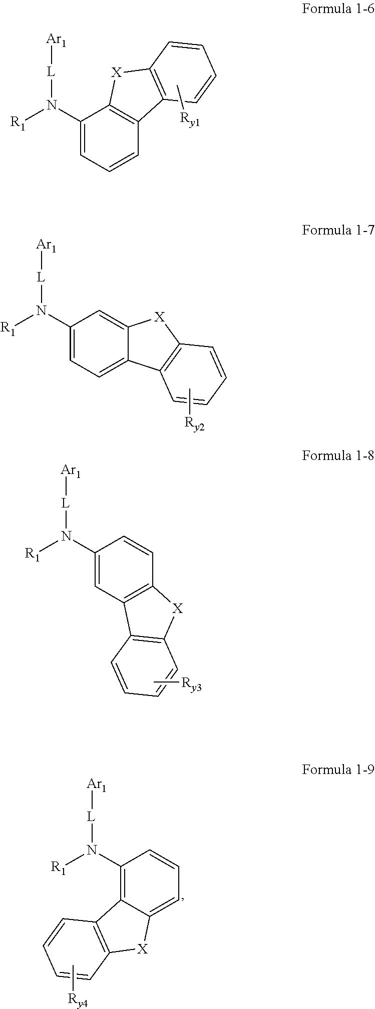
and
wherein in Formulas 1-6 to 1-9,
R_{y1} to R_{y4} are each independently a substituted or unsubstituted aryl group having 6 to 12 ring-forming carbon atoms, and
L, Ar₁, and R₁ are as defined in Formula 1.

11. The light emitting element of claim 1, wherein a substituent represented by Formula 3 is represented by any one selected from among Formulas 3-1 to 3-3: 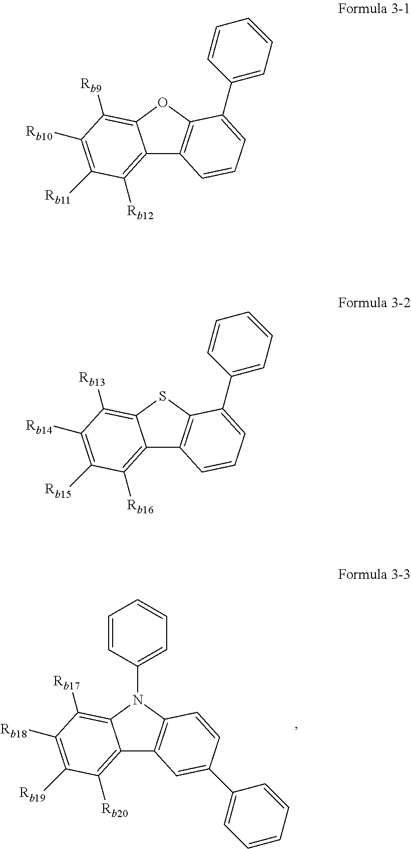
wherein in Formula 3-1, any one selected from among R_{b9} to R_{b12} is connected to Formula 1, and any remaining R_{b9} to R_{b12} is each independently a hydrogen atom or a deuterium atom,
wherein in Formula 3-2, any one selected from among R_{b13} to R_{b16} is connected to Formula 1, and any remaining R_{b13} to R_{b16} is each independently a hydrogen atom or a deuterium atom, and
wherein in Formula 3-3, any one selected from among R_{b17} to R_{b20} is connected to Formula 1, and any remaining R_{b17} to R_{b20} is each independently a hydrogen atom or a deuterium atom.

12. The light emitting element of claim 1, wherein the amine compound is represented by any one selected from among compounds from Compound Group 1:

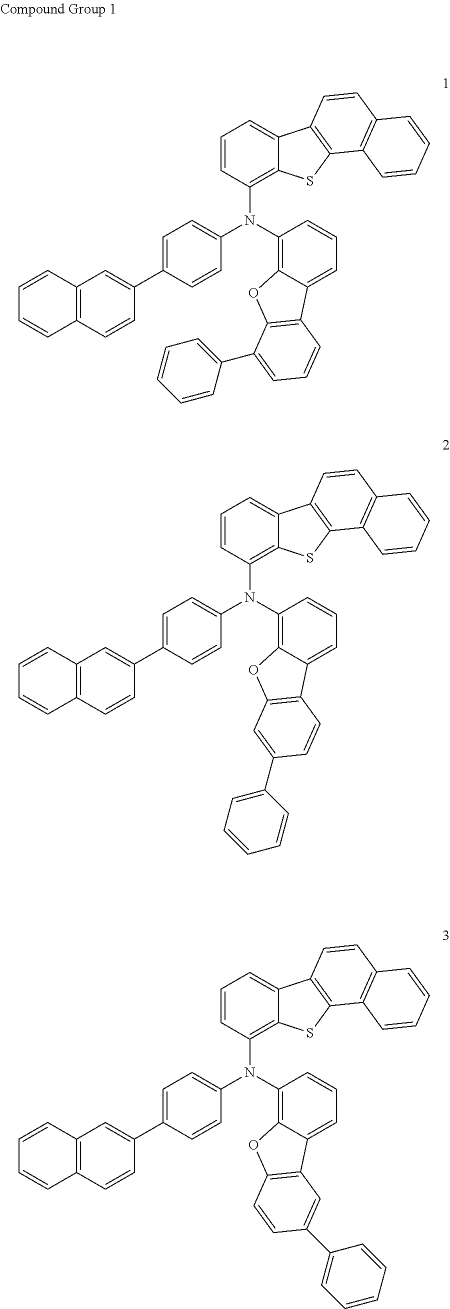 [SMILES: c1ccc(-c2cccc3c2oc2c(N(c4ccc(-c5ccc6ccccc6c5)cc4)c4cccc5c4sc4c6ccccc6ccc54)cccc23)cc1 ; c1ccc(-c2ccc3c(c2)oc2c(N(c4ccc(-c5ccc6ccccc6c5)cc4)c4cccc5c4sc4c6ccccc6ccc54)cccc23)cc1 ; c1ccc(-c2ccc3oc4c(N(c5ccc(-c6ccc7ccccc7c6)cc5)c5cccc6c5sc5c7ccccc7ccc65)cccc4c3c2)cc1]

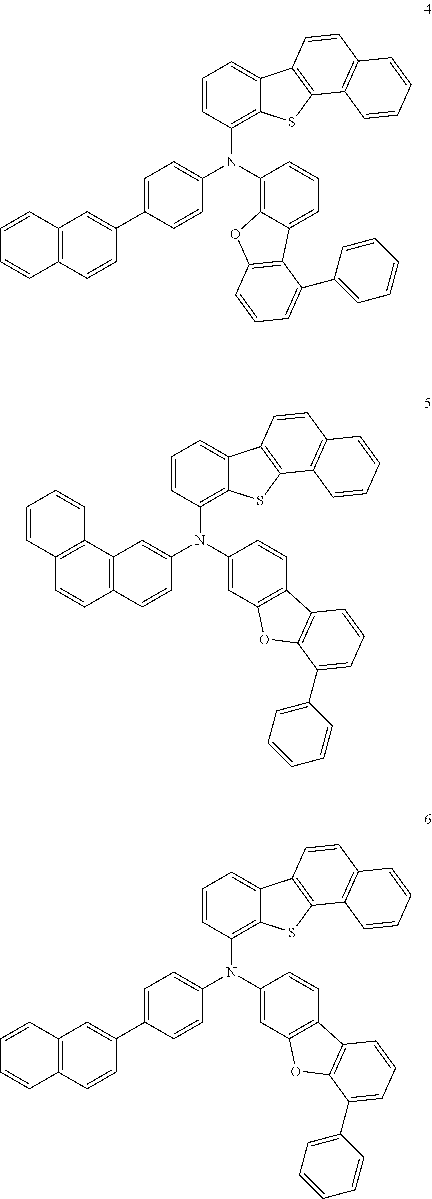 [SMILES: c1ccc(-c2cccc3oc4c(N(c5ccc(-c6ccc7ccccc7c6)cc5)c5cccc6c5sc5c7ccccc7ccc65)cccc4c23)cc1 ; c1ccc(-c2cccc3c2oc2cc(N(c4ccc5ccc6ccccc6c5c4)c4cccc5c4sc4c6ccccc6ccc54)ccc23)cc1 ; c1ccc(-c2cccc3c2oc2cc(N(c4ccc(-c5ccc6ccccc6c5)cc4)c4cccc5c4sc4c6ccccc6ccc54)ccc23)cc1]

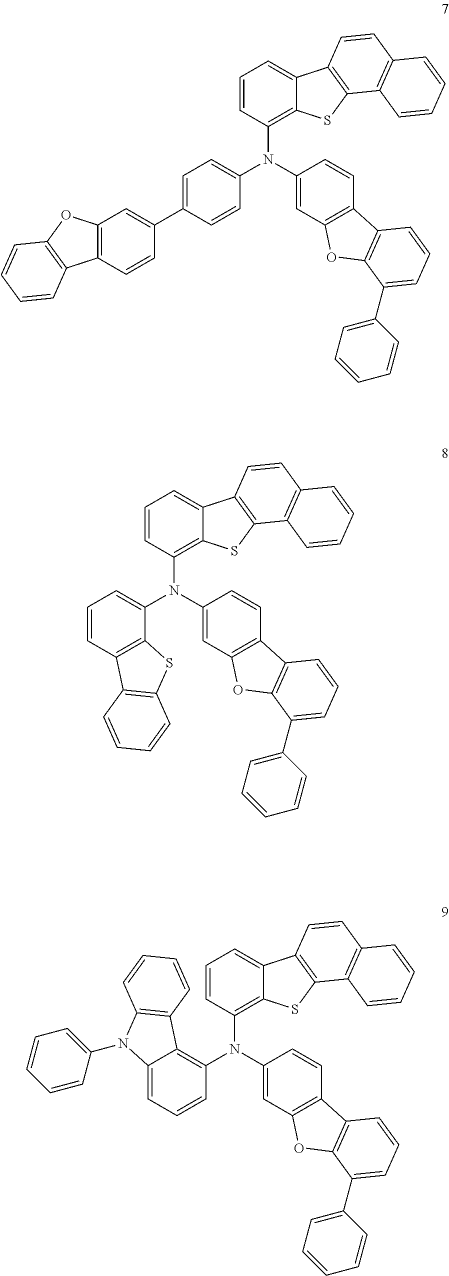 [SMILES: c1ccc(-c2cccc3c2oc2cc(N(c4ccc(-c5ccc6c(c5)oc5ccccc56)cc4)c4cccc5c4sc4c6ccccc6ccc54)ccc23)cc1 ; c1ccc(-c2cccc3c2oc2cc(N(c4cccc5c4sc4ccccc45)c4cccc5c4sc4c6ccccc6ccc54)ccc23)cc1 ; c1ccc(-c2cccc3c2oc2cc(N(c4cccc5c4sc4c6ccccc6ccc54)c4cccc5c4c4ccccc4n5-c4ccccc4)ccc23)cc1]

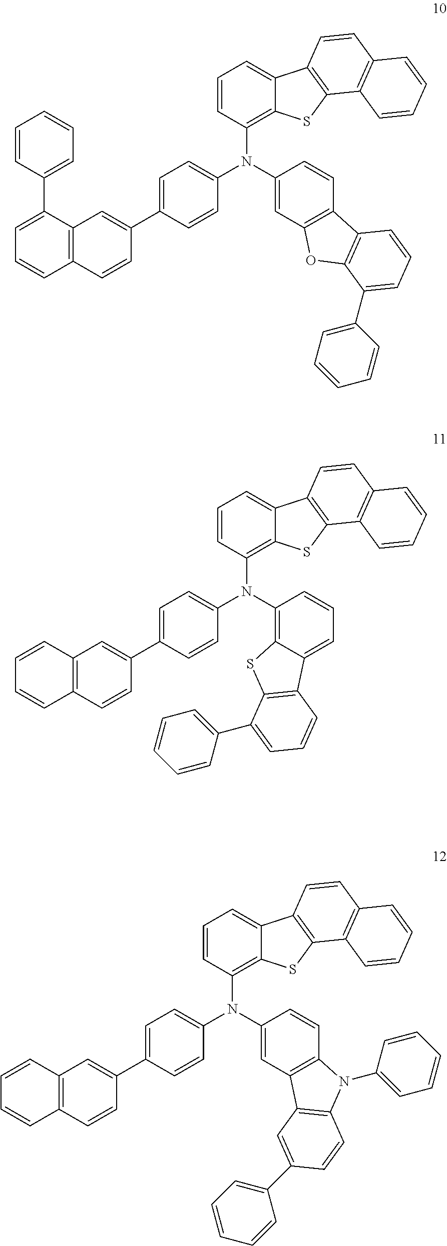 [SMILES: c1ccc(-c2cccc3ccc(-c4ccc(N(c5ccc6c(c5)oc5c(-c7ccccc7)cccc56)c5cccc6c5sc5c7ccccc7ccc65)cc4)cc23)cc1 ; c1ccc(-c2cccc3c2sc2c(N(c4ccc(-c5ccc6ccccc6c5)cc4)c4cccc5c4sc4c6ccccc6ccc54)cccc23)cc1 ; c1ccc(-c2ccc3c(c2)c2cc(N(c4ccc(-c5ccc6ccccc6c5)cc4)c4cccc5c4sc4c6ccccc6ccc54)ccc2n3-c2ccccc2)cc1]

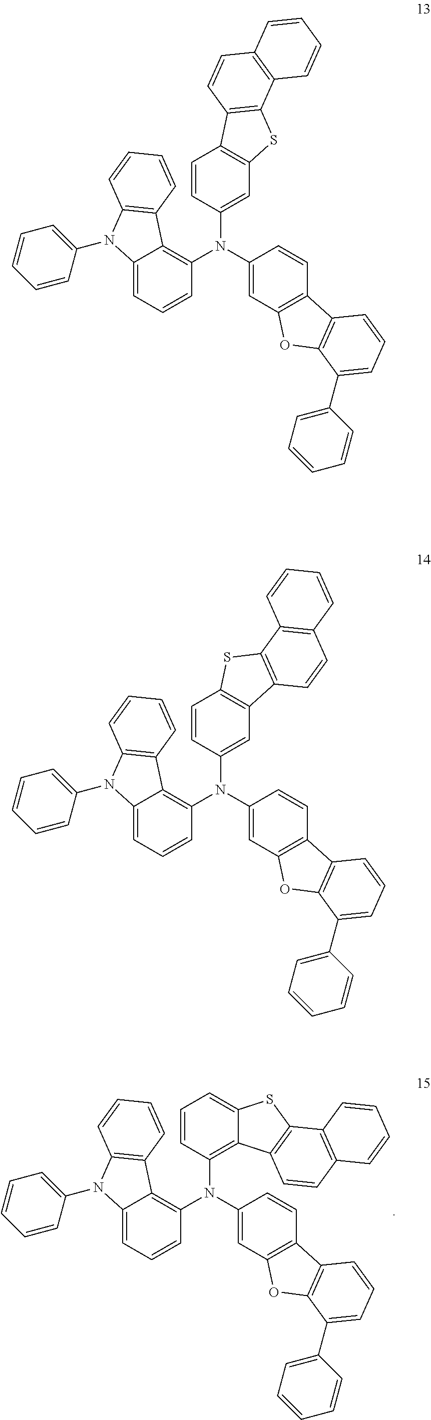 [SMILES: c1ccc(-c2cccc3c2oc2cc(N(c4ccc5c(c4)sc4c6ccccc6ccc54)c4cccc5c4c4ccccc4n5-c4ccccc4)ccc23)cc1 ; c1ccc(-c2cccc3c2oc2cc(N(c4ccc5sc6c7ccccc7ccc6c5c4)c4cccc5c4c4ccccc4n5-c4ccccc4)ccc23)cc1 ; c1ccc(-c2cccc3c2oc2cc(N(c4cccc5sc6c7ccccc7ccc6c45)c4cccc5c4c4ccccc4n5-c4ccccc4)ccc23)cc1]

13. An amine compound, the amine compound being represented by Formula 1: 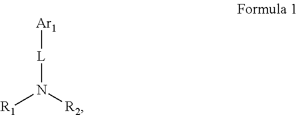
wherein in Formula 1,
L is a direct linkage, a substituted or unsubstituted arylene group having 6 to 30 ring-forming carbon atoms, or a substituted or unsubstituted heteroarylene group having 2 to 30 ring-forming carbon atoms,
Ar₁ is a substituted or unsubstituted aryl group having 6 to 30 ring-forming carbon atoms, or a substituted or unsubstituted heteroaryl group having 2 to 30 ring-forming carbon atoms, where Ar₁ does not comprise a substituted or unsubstituted benzofuran group and a substituted or unsubstituted benzothiophene group,
R₁ is represented by Formula 2, and
R₂ is represented by Formula 3: 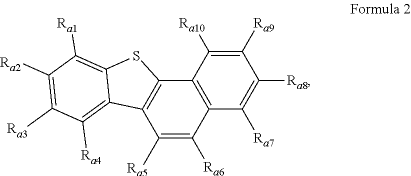
wherein in Formula 2,
any one selected from among Rₐ₁ to Rₐ₄ is connected to Formula 1, and any remaining Rₐ₁ to Rₐ₄ is each independently a hydrogen atom, a deuterium atom, a halogen atom, a cyano group, or a substituted or unsubstituted alkyl group having 1 to 10 carbon atoms, and
Rₐ₅ to Rₐ₁₀ are each independently a hydrogen atom, a deuterium atom, a halogen atom, a cyano group, or a substituted or unsubstituted alkyl group having 1 to 10 carbon atoms, 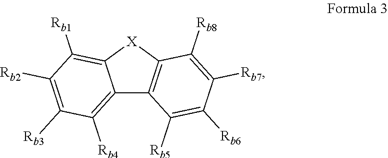
and
wherein in Formula 3,
X is O, S, or NAr₂,
Ar₂ is a substituted or unsubstituted aryl group having 6 to 30 ring-forming carbon atoms,
any one selected from among R_{b1} to R_{b4} is connected to Formula 1, and any remaining R_{b1} to R_{b4} is each independently a hydrogen atom, a deuterium atom, a halogen atom, or a substituted or unsubstituted alkyl group having 1 to 10 carbon atoms,
any one selected from among R_{b5} to R_{b8} is a substituted or unsubstituted aryl group having 6 to 30 ring-forming carbon atoms, where R_{b5} to R_{b8} do not comprise a substituted or unsubstituted fluorene group, and
any remaining R_{b5} to R_{b8} is each independently a hydrogen atom, a deuterium atom, a halogen atom, or a substituted or unsubstituted alkyl group having 1 to 10 carbon atoms.

14. The amine compound of claim 13, wherein the amine compound is represented by Formula 1-1: 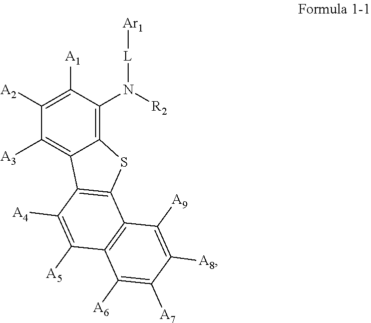
and
wherein in Formula 1-1,
A₁ to A₉ are each independently a hydrogen atom, a deuterium atom, a halogen atom, or a substituted or unsubstituted alkyl group having 1 to 10 carbon atoms, and
L, Ar₁, and R₂ are as defined in Formula 1.

15. The amine compound of claim 13, wherein the amine compound is represented by any one selected from among Formulas 1-2 to 1-5: 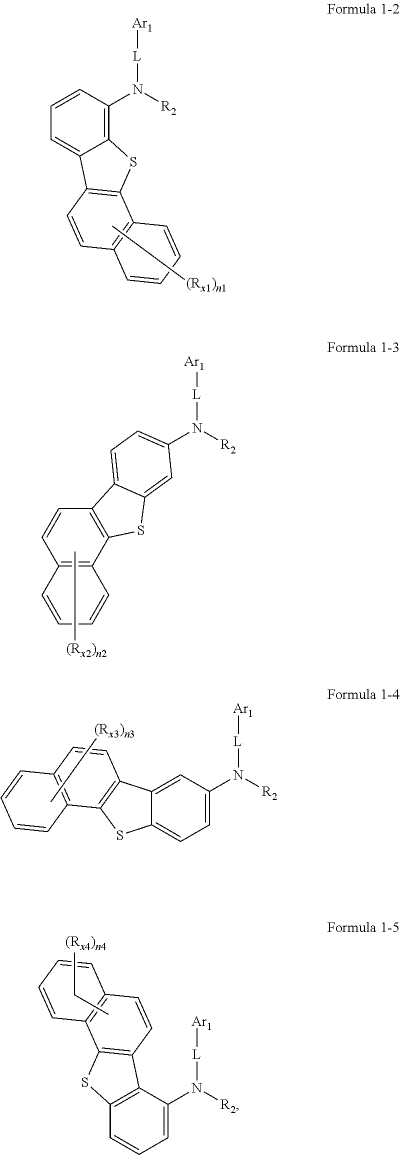
and
wherein in Formulas 1-2 to 1-5,
Rₓ₁ to Rₓ₄ are each independently a hydrogen atom, a deuterium atom, or a substituted or unsubstituted alkyl group having 1 to 5 carbon atoms,
n1 to n4 are each independently an integer of 0 to 5, and
L, Ar₁, and R₂ are as defined in Formula 1.

16. The amine compound of claim 13, wherein in Formula 3, any one selected from among R_{b1} to R_{b4} is a position connected to Formula 1, and any remaining R_{b1} to R_{b4} is each independently a hydrogen atom or a deuterium atom.

17. The amine compound of claim 13, wherein the amine compound is represented by any one selected from among Formulas 1-6 to 1-9: 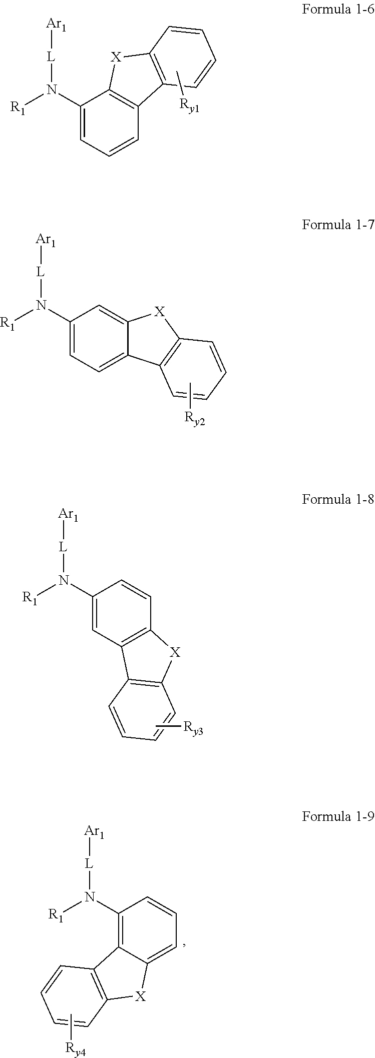
and
wherein in Formulas 1-6 to 1-9,
R_{y1} to R_{y4} are each independently a substituted or unsubstituted aryl group having 6 to 12 ring-forming carbon atoms, and
L, Ar₁, and R₁ are as defined in Formula 1.

18. The amine compound of claim 13, wherein a substituent represented by Formula 3 is represented by any one selected from among Formulas 3-1 to 3-3: 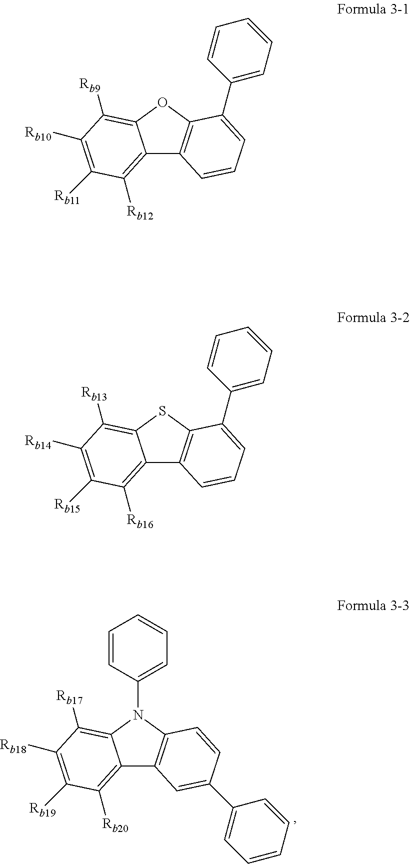
wherein in Formula 3-1, any one selected from among R_{b9} to R_{b12} is a position connected to Formula 1, and any remaining R_{b9} to R_{b12} is each independently a hydrogen atom or a deuterium atom,
wherein in Formula 3-2, any one selected from among R_{b13} to R_{b16} is a position connected to Formula 1, and any remaining R_{b13} to R_{b16} is each independently a hydrogen atom or a deuterium atom, and
wherein in Formula 3-3, any one selected from among R_{b17} to R_{b20} is a position connected to Formula 1, and any remaining R_{b17} to R_{b20} is each independently a hydrogen atom or a deuterium atom.

19. The amine compound of claim 13, wherein the amine compound is represented by any one selected from among compounds from Compound Group 1:

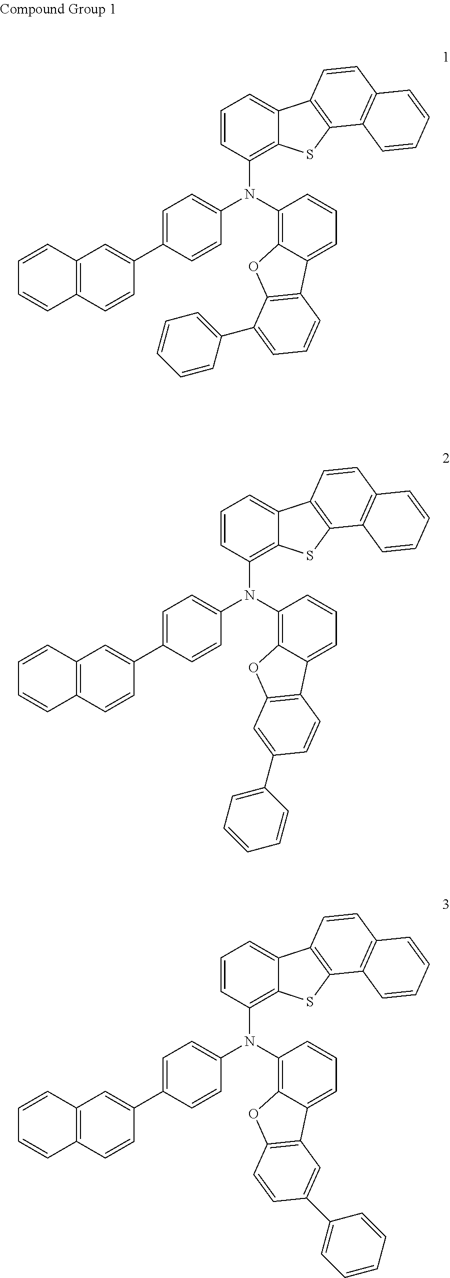 [SMILES: c1ccc(-c2cccc3c2oc2c(N(c4ccc(-c5ccc6ccccc6c5)cc4)c4cccc5c4sc4c6ccccc6ccc54)cccc23)cc1 ; c1ccc(-c2ccc3c(c2)oc2c(N(c4ccc(-c5ccc6ccccc6c5)cc4)c4cccc5c4sc4c6ccccc6ccc54)cccc23)cc1 ; c1ccc(-c2ccc3oc4c(N(c5ccc(-c6ccc7ccccc7c6)cc5)c5cccc6c5sc5c7ccccc7ccc65)cccc4c3c2)cc1]

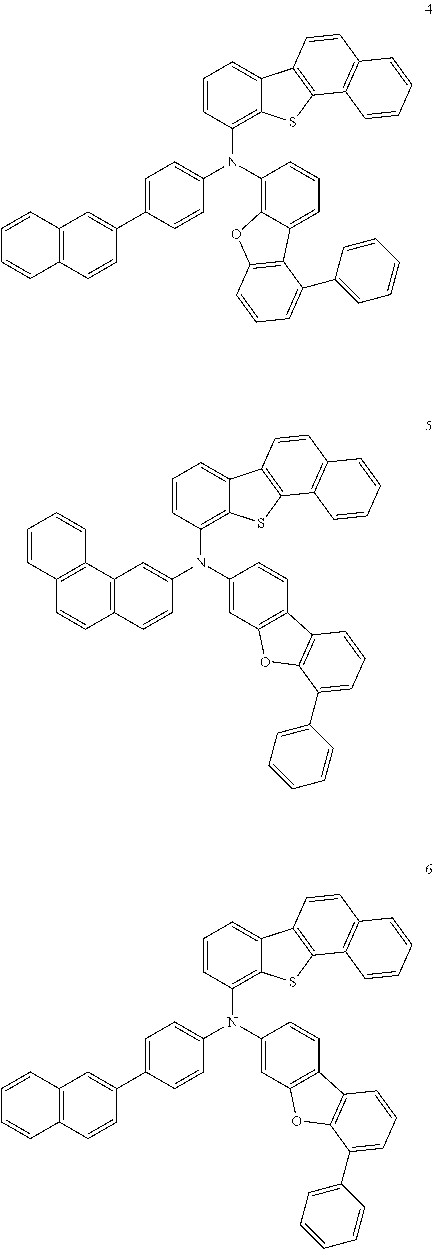 [SMILES: c1ccc(-c2cccc3oc4c(N(c5ccc(-c6ccc7ccccc7c6)cc5)c5cccc6c5sc5c7ccccc7ccc65)cccc4c23)cc1 ; c1ccc(-c2cccc3c2oc2cc(N(c4ccc5ccc6ccccc6c5c4)c4cccc5c4sc4c6ccccc6ccc54)ccc23)cc1 ; c1ccc(-c2cccc3c2oc2cc(N(c4ccc(-c5ccc6ccccc6c5)cc4)c4cccc5c4sc4c6ccccc6ccc54)ccc23)cc1]

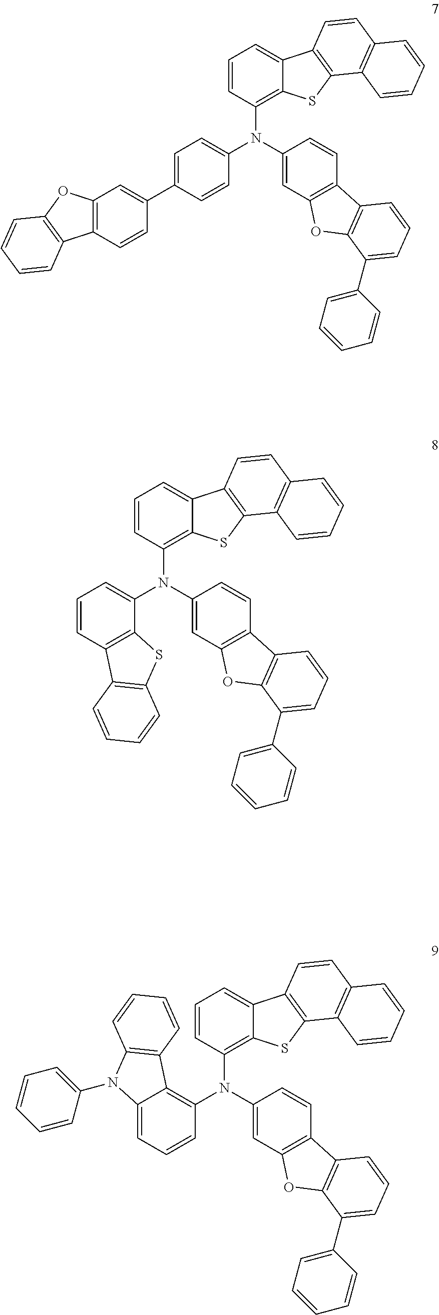 [SMILES: c1ccc(-c2cccc3c2oc2cc(N(c4ccc(-c5ccc6c(c5)oc5ccccc56)cc4)c4cccc5c4sc4c6ccccc6ccc54)ccc23)cc1 ; c1ccc(-c2cccc3c2oc2cc(N(c4cccc5c4sc4ccccc45)c4cccc5c4sc4c6ccccc6ccc54)ccc23)cc1 ; c1ccc(-c2cccc3c2oc2cc(N(c4cccc5c4sc4c6ccccc6ccc54)c4cccc5c4c4ccccc4n5-c4ccccc4)ccc23)cc1]

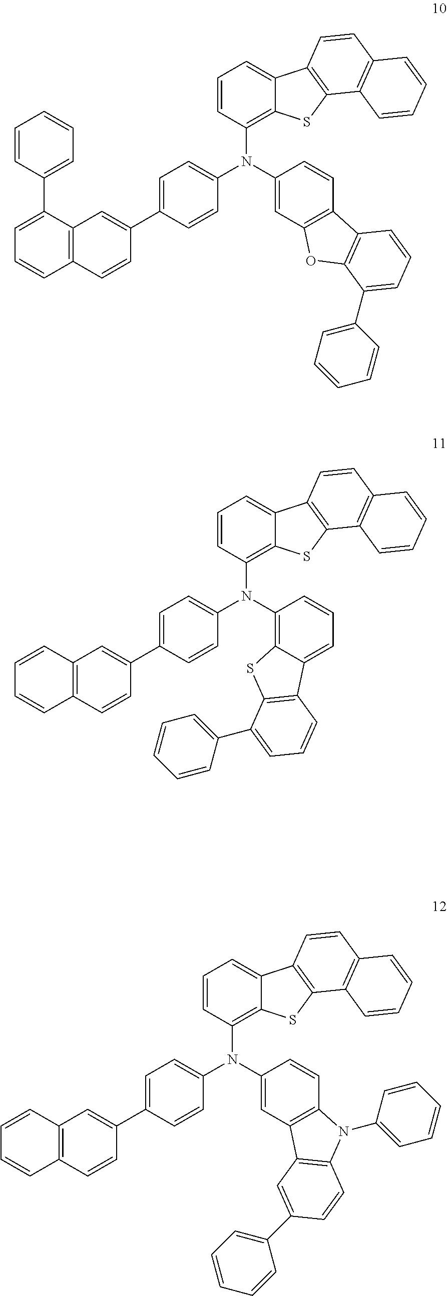 [SMILES: c1ccc(-c2cccc3ccc(-c4ccc(N(c5ccc6c(c5)oc5c(-c7ccccc7)cccc56)c5cccc6c5sc5c7ccccc7ccc65)cc4)cc23)cc1 ; c1ccc(-c2cccc3c2sc2c(N(c4ccc(-c5ccc6ccccc6c5)cc4)c4cccc5c4sc4c6ccccc6ccc54)cccc23)cc1 ; c1ccc(-c2ccc3c(c2)c2cc(N(c4ccc(-c5ccc6ccccc6c5)cc4)c4cccc5c4sc4c6ccccc6ccc54)ccc2n3-c2ccccc2)cc1]

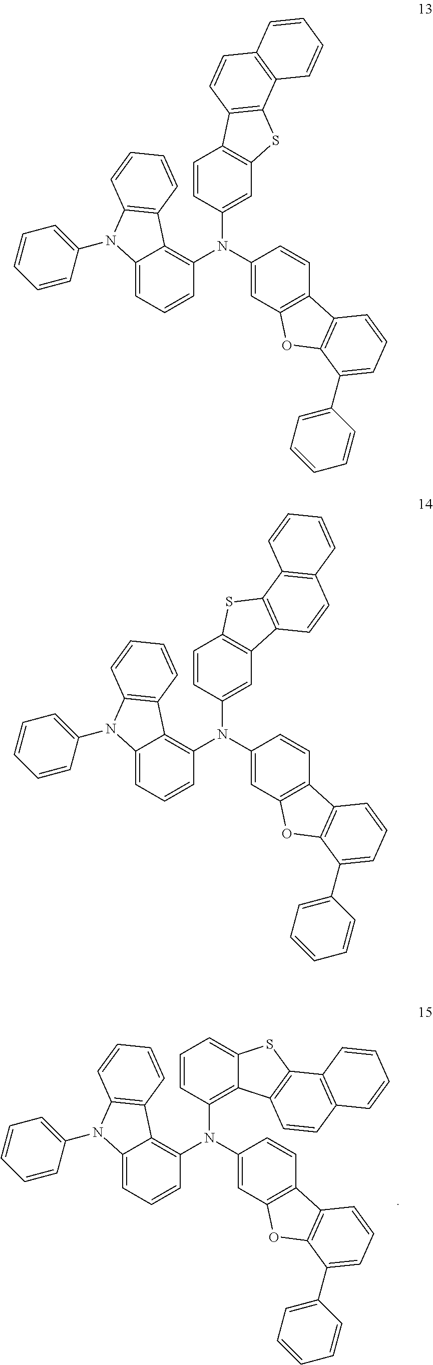

20. A display device comprising the amine compound according to claim 13.

21. An electronic apparatus comprising a display device, the display device comprising:
a base layer;
a circuit layer on the base layer; and
a display element layer on the circuit layer and comprising a light emitting element,
wherein the light emitting element comprises a first electrode, a second electrode on the first electrode, and a hole transport region between the first electrode and the second electrode and comprising an amine compound, the amine compound being represented by Formula 1: 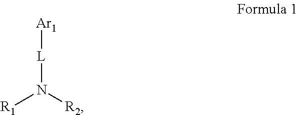
wherein in Formula 1,
L is a direct linkage, a substituted or unsubstituted arylene group having 6 to 30 ring-forming carbon atoms, or a substituted or unsubstituted heteroarylene group having 2 to 30 ring-forming carbon atoms,
Ar₁ is a substituted or unsubstituted aryl group having 6 to 30 ring-forming carbon atoms, or a substituted or unsubstituted heteroaryl group having 2 to 30 ring-forming carbon atoms, where Ar₁ does not comprise a substituted or unsubstituted benzofuran group and a substituted or unsubstituted benzothiophene group,
R₁ is represented by Formula 2, and
R₂ is represented by Formula 3: 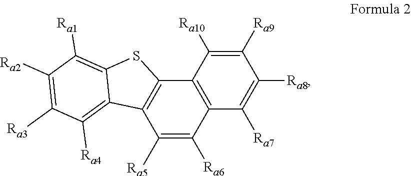
wherein in Formula 2,
any one selected from among Rₐ₁ to Rₐ₄ is connected to Formula 1, and any remaining Rₐ₁ to Rₐ₄ is each independently a hydrogen atom, a deuterium atom, a halogen atom, a cyano group, or a substituted or unsubstituted alkyl group having 1 to 10 carbon atoms, and
Rₐ₅ to Rₐ₁₀ are each independently a hydrogen atom, a deuterium atom, a halogen atom, a cyano group, or a substituted or unsubstituted alkyl group having 1 to 10 carbon atoms, 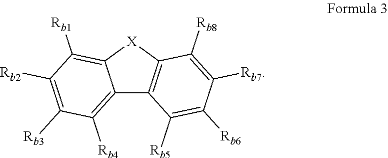
and
wherein in Formula 3,
X is O, S, or NAr₂,
Ar₂ is a substituted or unsubstituted aryl group having 6 to 30 ring-forming carbon atoms,
any one selected from among R_{b1} to R_{b4} is a position connected to Formula 1, and any remaining R_{b1} to R_{b4} is each independently a hydrogen atom, a deuterium atom, a halogen atom, or a substituted or unsubstituted alkyl group having 1 to 10 carbon atoms,
any one selected from among R_{b5} to R_{b8} is a substituted or unsubstituted aryl group having 6 to 30 ring-forming carbon atoms, where R_{b5} to R_{b8} do not comprise a substituted or unsubstituted fluorene group, and
any remaining R_{b5} to R_{b8} is each independently a hydrogen atom, a deuterium atom, a halogen atom, or a substituted or unsubstituted alkyl group having 1 to 10 carbon atoms.

22. An electronic apparatus comprising the light emitting element according to claim 1.
